(19) United States
(12) Patent Application Publication
(10) Pub. No.: US 2003/0232363 A1
(43) Pub. Date: Dec. 18, 2003

(54) Fatty acid transport proteins

(76) Inventors: Andreas Stahl, Allston, MA (US); David J. Hirsch, Jamaica Plain, MA (US); Harvey F. Lodish, Brookline, MA (US); Ruth E. Gimeno, Wellesley, MA (US); Louis A. Tartaglia, Newton, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/405,877

(22) Filed: Apr. 1, 2003

Related U.S. Application Data
(62) Division of application No. 09/611,197, filed on Jul. 6, 2000, now abandoned.
Application No. 09/611,197 is a continuation-in-part of application No. 09/506,252, filed on Feb. 17, 2000, now abandoned.
Application No. 09/506,252 is a continuation-in-part of application No. 09/465,280, filed on Dec. 16, 1999, now abandoned.
Application No. 09/506,252 is a continuation-in-part of application No. 09/405,504, filed on Sep. 23, 1999.
Application No. 09/506,252 is a continuation-in-part of application No. 09/405,505, filed on Sep. 23, 1999, now abandoned.
Application No. 09/506,252 is a continuation-in-part of application No. 09/232,197, filed on Jan. 14, 1999, now Pat. No. 6,300,096.
Application No. 09/506,252 is a continuation-in-part of application No. 09/232,200, filed on Jan. 14, 1999, now Pat. No. 6,288,213.
Application No. 09/506,252 is a continuation-in-part of application No. 09/232,201, filed on Jan. 14, 1999, now Pat. No. 6,348,321.
Application No. 09/506,252 is a continuation-in-part of application No. 09/232,195, filed on Jan. 14, 1999.
Application No. 09/506,252 is a continuation-in-part of application No. 09/232,191, filed on Jan. 14, 1999, now Pat. No. 6,284,487.
(60) Provisional application No. 60/110,941, filed on Dec. 4, 1998.
(60) Provisional application No. 60/093,491, filed on Jul. 20, 1998.
(60) Provisional application No. 60/071,374, filed on Jan. 15, 1998.

(51) Int. Cl. C12Q 1/68; C07H 21/04; C12P 21/02; C12N 5/06; C07K 14/47

(52) U.S. Cl. 435/6; 435/69.1; 435/320.1; 435/325; 530/350; 536/23.5

(57) ABSTRACT

A family of fatty acid transport proteins (FATPs) mediate transport of long chain fatty acids (LCFAs) across cell membranes into cells. These proteins exhibit different expression patterns among the organs of mammals. Nucleic acids encoding FATPs of this family, vectors comprising these nucleic acids, as well as the production of FATP proteins in host cells are described. Also described are methods to test FATPs for fatty acid transport function, and methods to identify inhibitors or enhancers of transport function. The altering of LCFA uptake by administering to the mammal an inhibitor or enhancer of FATP transport function of a FATP in the small intestine can decrease or increase calories available as fats, and can decrease or increase circulating fatty acids. The organ specificity of FATP distribution can be exploited in methods to direct drugs, diagnostic indicators and so forth to an organ such as the heart.

### RELATED APPLICATIONS

This application is a divisional of U.S. application Ser. No. 09/611,197 filed Jul. 6, 2000 which is a continuation-in-part of U.S. patent application Ser. No. 09/506,252 filed Feb. 17, 2000 which is a continuation-in-part of U.S. patent application Ser. No. 09/465,280 filed Dec. 16, 1999, a continuation-in-part of U.S. patent application Ser. No. 09/405,504 filed Sep. 23, 1999, a continuation-in-part of U.S. patent application Ser. No. 09/405,505 filed Sep. 23, 1999, a continuation-in-part of U.S. patent application Ser. No. 09/232,197 filed Jan. 14, 1999, now U.S. Pat. No. 6,300,096, a continuation-in-part of U.S. patent application Ser. No. 09/232,200 filed Jan. 14, 1999, now U.S. Pat. No. 6,288,213, a continuation-in-part of U.S. patent application Ser. No. 09/232,201 filed Jan. 14, 1999, now U.S. Pat. No. 6,348,321, a continuation-in-part of U.S. patent application Ser. No. 09/232,195 filed Jan. 14, 1999, a continuation-in-part of U.S. patent application Ser. No. 09/232,191 filed Jan. 14, 1999, now U.S. Pat. No. 6,284,487, each of which claims the benefit of U.S. Provisional Application No. 60/110,941 filed Dec. 4, 1998; U.S. Provisional Application No. 60/093,491 filed Jul. 20, 1998; and U.S. Provisional Application No. 60/071,374 filed Jan. 15, 1998. The teachings of each of these referenced applications are incorporated herein by reference in their entirety.

### GOVERNMENT SUPPORT

The invention was supported, in whole or in part, by a grant from the National Heart, Lung, and Blood Institute (HL41484), by National Institutes of Health Grant DK 47618 and National Institutes of Health Grant 5 T32 CA 09541. The United States Government has certain rights in the invention.

### BACKGROUND OF THE INVENTION

Long chain fatty acids (LCFAs) are an important source of energy for most organisms. They also function as blood hormones, regulating key metabolic functions such as hepatic glucose production. Although LCFAs can diffuse through the hydrophobic core of the plasma membrane into cells, this nonspecific transport cannot account for the high affinity and specific transport of LCFAs exhibited by cells such as cardiac muscle, hepatocytes, enterocytes, and adipocytes. The molecular mechanisms of LCFA transport remains largely unknown. Identifying these mechanisms can lead to pharmaceuticals that modulate fatty acid uptake by the intestine and by other organs, thereby alleviating certain medical conditions (e.g. obesity).

### SUMMARY OF THE INVENTION

Described herein is a diverse family of fatty acid transport proteins (FATPs) which are evolutionarily conserved; these FATPs are plasma membrane proteins which mediate transport of LCFAs across the membranes and into cells. Members of the FATP family described herein are present in a wide variety of organisms, from mycobacteria to humans, and exhibit very different expression patterns in tissues among the organisms. FATP family members are expressed in prokaryotic and eukaryotic organisms and comprise characteristic amino acid domains or sequences which are highly conserved across family members. In addition, the function of the FATP gene family is conserved throughout evolution, as shown by the fact that the Caenorhabditis (C). elegans and mycobacterial FATPs described herein facilitate LCFA uptake when they are overexpressed in COS cells or Escherichia(E.) coli, respectively. FATPs are expressed in a wide variety of tissues, including all tissues which are important to fatty acid metabolism (uptake and processing).

In specific embodiments, FATPs of the present invention are from such diverse organisms as humans (Homo (H.) sapiens), mice, (Mus (M.) musculus), F. rubripes, C. elegans, Drosophila (D.) melanogaster, Saccharomyces (S.) cerevisiae, Aspergillus nidulans, Cochliobolu heterostrophus, Magnaporthe grisea and Mycobacterium (M.), such as M. tuberculosis. As described herein, four novel mouse FATPs, referred to as mmFATP2, mmFATP3, mmFATP4 and mmFATP5, and six human FATPs, referred to as hsFATP1, hsFATP2, hsFATP3, hsFATP4, hsFATP5 and hsFATP6, have been identified. All four novel murine FATPs (mmFATP2-5) and a previously identified murine FATP (renamed herein FATP1) have orthologs in humans (hsFATP1-5); the sixth human FATP (hsFATP6) does not as yet have a mouse ortholog. The expression patterns of these FATPs vary, as described in detail below.

The present invention relates to FATP family members from prokaryotes and eukaryotes, nucleic acids (DNA, RNA) encoding FATPs, and nucleic acids which are useful as probes or primers (e.g., for use in hybridization methods, amplification methods) for example, in methods of detecting FATP-encoding genes, producing FATPs, and purifying or isolating FATP-encoding DNA or RNA. Also the subject of this invention are antibodies (polyclonal or monoclonal) which bind an FATP or FATPs; methods of identifying additional FATP family members (for example, orthologs of those FATPs described herein by amino acid sequence) and variant alleles of known FATP genes; methods of identifying compounds which bind to an FATP, or modulate or alter (enhance or inhibit) FATP function; compounds which modulate or alter FATP function; methods of modulating or altering (enhancing or inhibiting) FATP function and, thus, LCFA uptake into tissues of a mammal (e.g. human) by administering a compound or molecule (a drug or agent) which increases or reduces FATP activity; and methods of targeting compounds to tissues by administering a complex of the compound to be targeted to tissues and a component which is bound by an FATP present on cells of the tissues to which the compound is to be targeted. For example, a complex of a drug to be delivered to the liver and a component which is bound by an FATP present on liver cells (e.g., FATP5) can be administered.

In one embodiment, the present invention relates to modulating or altering (enhancing or inhibiting/reducing) LCFA uptake in the small intestine and, thus, increasing or reducing the number of calories in the form of fats available to an individual. In another embodiment, the present invention relates to inhibiting or reducing LCFA uptake in the small intestine in order to reduce circulating fatty acid levels; that is, LCFA uptake in the small intestine is reduced and, therefore, circulating (blood) levels are not as high as they otherwise would be. FATP4 has been shown to be expressed in epithelial cells of the small intestine and particularly in the brush border layer of the small intestine. FATP2 has also been shown to be expressed at low levels in epithelial cells of the small intestine, particularly in the duodenum. In contrast, FATP1, FATP3, FATP5 and FATP6 were not detected in any of the intestinal tissues. Thus, also described herein are FATPs which are present in the epithelial cell layer of the small intestine where they mediate LCFA uptake. These FATPs, particularly FATP4 and also FATP2, are targets for methods and drugs which block their function or activity and are useful in treating obesity, diabetes and heart disease. The ability of these FATPs to mediate fat uptake can be modulated or altered (enhanced or inhibited), thus modulating fat uptake in the small intestine. This can be done, for example, by administering to an individual, such as a human or other animal, a drug which blocks interaction of LCFAs with FATP4 and/or FATP2 in the small intestine, thus inhibiting LCFA passage into the cells of the small intestine. As a result, fat absorption is reduced and, although the individual has consumed a certain quantity of fat, the LCFAs are not absorbed to the same extent they would have been in the absence of the compound administered.

Thus, one embodiment of this invention is a method of reducing LCFA uptake (absorption) in the small intestine and, as a result, reducing caloric uptake in the form of fat. A further embodiment is a compound (drug) useful in inhibiting or reducing fat absorption in the small intestine. In another embodiment, the invention is a method of reducing circulating fatty acid levels by administering to an individual a compound which blocks interactions of LCFAs with FATP4 and/or FATP2 in the small intestine, thus inhibiting LCFA passage into cells of the small intestine. As a result, fatty acids pass into the circulatory system at a diminished level and/or rate, and circulating fatty acid levels are lower than they would be in the absence of the compound administered. This method is particularly useful for therapy in individuals who are at risk for or have hyperlipidemia. That is, it can be used to prevent the occurrence of elevated levels of lipids in the blood or to treat an individual in whom blood lipid levels are elevated. Also the subject of this invention is a method of identifying compounds which alter FATP function (and thus, in the case of FATP2 and/or FATP4, alter LCFA uptake in the small intestine).

In another embodiment, the present invention relates to a method of modulating or altering (enhancing or inhibiting) the function of FATP6, which is expressed at high levels in the heart. A method of inhibiting FATP6 function is useful, for example, in individuals with heart disease, such as ischemia, since reducing LCFA uptake into heart muscle in an individual who has ischemic heart disease, which may be manifested by, for example, angina or heart attack, can reduce symptoms or reduce the extent of damage caused by the ischemia. In this embodiment, a drug which inhibits FATP6 function is administered to an individual who has had or is having a heart attack, to reduce LCFA uptake by the individual's heart and, as a result, reduce the damage caused by ischemia. In a further embodiment, this invention is a method of targeting a compound, such as a therapeutic drug or an imaging reagent, to heart tissue by administering to an individual (e.g., a human) a complex of the compound and a component (e.g., a LCFA or LCFA-like compound) which is bound by an FATP (e.g., FATP6) present in cells of heart tissue.

In a further embodiment, LCFA uptake by the liver is modulated or altered (enhanced or reduced), in an individual. For example, a drug which inhibits the function of an FATP present in liver (e.g., FATP5) is administered to an individual who is diabetic, in order to reduce LCFA uptake by liver cells and, thus reduce insulin resistance.

The present invention, thus, provides methods which are useful to alter, particularly reduce, LCFA uptake in individuals and, as a result, to alter (particularly reduce), availability of the LCFAs for further metabolism. In a specific embodiment, the present invention provides methods useful to reduce LCFA uptake and, thus, fatty acid metabolism in individuals, with the result that caloric availability from fats is reduced, and circulating fatty acid levels are lower than they otherwise would be. These methods are useful, for example, as a means of weight control in individuals, (e.g., humans) and as a means of preventing elevated serum lipid levels or reducing serum lipid levels in humans. FATPs expressed in the small intestine, such as FATP4, are useful targets to be blocked in treating obesity (e.g., chronic obesity) or to be enhanced in treating conditions in which enhanced LCFA uptake is desired (e.g., malabsorption syndrome or other wasting conditions).

The identification of this evolutionarily conserved fatty acid transporter family will allow a better understanding of the mechanisms whereby LCFAs traverse the lipid bilayer as well as yield insight into the control of energy homeostasis and its dysregulation in diseases such as diabetes and obesity.

### BRIEF DESCRIPTION OF THE DRAWINGS

The file of this patent contains at least one color photograph. Copies of this patent with color photographs will be provided by the Patent and Trademark Office upon request and payment of necessary fee.

FIG. 1 shows the amino acid sequence alignment of FATPs: mmFATP1 (SEQ ID NO:92), mmFATP2 (SEQ ID NO:93), mmFATP3 (SEQ ID NO:94), mmFATP4 (SEQ ID NO:95), mmFATP5 (SEQ ID NO:96), ceFATPa (SEQ ID NO:97), scFATP (SEQ ID NO:98) and mtFATP (SEQ ID NO:99). The underlining (amino acid residues 204-212 of mtFATP) indicates an AMP binding motif which is found in many classes of proteins; the underlining at amino acid residues 204-507 of the mtFATP sequence indicates the FATP 360 amino acid signature sequence.

FIGS. 2A-2D show results of LCFA uptake assays. FIGS. 2A-2D: COS cells were cotransfected using the DEAE-dextran method with the mammalian expression vectors pCDNA-CD2 either alone (control; FIG. 2A) or in combination with one of the FATP-containing expression vectors (pCDNA-mmFATP1, FIG. 2B; pCDNA-mmFATP2, FIG. 2C; or pCMV-SPORT2-mmFATP5, FIG. 2D) as described in Materials and Methods for Example 2. COS cells were gated on forward scatter (FSC) and side scatter (SS), and the results shown represent >10,000 cells. Cells exhibiting >300 CD2 fluorescence units (vertical line) representing 15% of all cells were deemed CD2 positive.

FIG. 3 is a graph of fluorescence of cells expressing a FATP gene. As in FIGS. 2A-2D, COS cells were cotransfected with pCDNA-CD2 either alone (control) or in combination with one of the FATP-containing expression vectors (pCDNA-mmFATP1, pCDNA-mmFATP2, pCMV-SPORT2-mmFATP5, or pCDNA-ceFATPb). The mean BODIPY-FA fluorescence of the CD2-positive cells is plotted; results shown represent the average of three experiments, each consisting of greater than 50,000 COS cells. Note that a logarithmic scale is used on the ordinate.

FIG. 4 is a graph of the uptake of palmitate with time. The full-length coding region of mtFATP (squares) or a control protein (TFE3; circles) was subcloned into the inducible, prokaryotic expression vector pET (Novagen, Madison, Wis.). Expression from the resulting plasmid was induced (solid symbols) in transformed E. coli cells with 1 mM isopropyl-β-D-thiogalactoside (IPTG) for 1 hour, or cells were left uninduced (open symbols). Data points were done in triplicate and counts were normalized to the number of bacteria as determined by OD₆₀₀.

FIG. 5 is a phylogenetic tree produced by aligning complete and partial sequences for FATP genes from human, rat, mouse, puffer fish, D. melanogaster, C. elegans, S. cerevisiae, and M. tuberculosis using ClustalX and using these data to produce a phylogenetic tree using TreeViewPPC. The bar indicates the number of substitutions per residue, i.e., 0.1 corresponds to a distance of 10 substitutions per 100 residues.

FIG. 6 shows a comparison of the FATP signature sequences of mmFATP1 (SEQ ID NO:1), mmFATP5, (SEQ ID NO:2), ceFATPa (SEQ ID NO:3), scFATP (SEQ ID NO:4) and mtFATP (SEQ ID NO:5).

FIG. 7 shows the sequence identity among the FATP family members and VLACs, based on the 360 amino acid signature sequence of FATP from FIG. 1.

FIGS. 8A and 8B are the mmFATP3 DNA sequence (SEQ ID NO:6).

FIG. 9 is the mmFATP3 protein sequence (SEQ ID NO:7).

FIGS. 10A and 10B are the mmFATP4 DNA sequence (SEQ ID NO:8).

FIG. 11 is the mmFATP4 protein sequence (SEQ ID NO:9).

FIGS. 12A and 12B are the mmFATP5 DNA sequence (SEQ ID NO:10).

FIG. 13 is the mmFATP5 protein sequence (SEQ ID NO:11).

FIGS. 14A and 14B are the hsFATP2 DNA sequence (SEQ ID NO:12).

FIG. 15 is the hsFATP2 protein sequence (SEQ ID NO:13).

FIGS. 16A and 16B are the hsFATP3 DNA sequence (SEQ ID NO:14).

FIG. 17 is the hsFATP3 protein sequence (SEQ ID NO:15).

FIGS. 18A and 18B are the hsFATP4 DNA sequence (SEQ ID NO:16).

FIG. 19 is the hsFATP4 protein sequence (SEQ ID NO:17).

FIGS. 20A and 20B are the hsFATP5 DNA sequence (SEQ ID NO:18).

FIG. 21 is the hsFATP5 protein sequence (SEQ ID NO:19).

FIGS. 22A and 22B are the hsFATP6 DNA sequence (SEQ ID NO:20).

FIG. 23 is the hsFATP6 protein sequence (SEQ ID NO:21).

FIGS. 24A and 24B are the mtFATP DNA sequence (SEQ ID NO:22).

FIG. 25 is the mtFATP protein sequence (SEQ ID NO:23).

FIG. 26 shows the DNA sequence (SEQ ID NO:24) and predicted amino acid sequence (SEQ ID NO:25) of human FATP1.

FIG. 27 shows the DNA sequence (SEQ ID NO:26) and predicted amino acid sequence (SEQ ID NO:27) of human FATP4.

FIG. 28A is a hydrophobicity plot for hsFATP1, showing that it has multiple membrane-spanning domains.

FIG. 28B is the amino acid composition of hsFATP1.

FIG. 28C is a hydrophilicity plot for hsFATP 1, made using the Kyte-Doolittle method, averaging hydrophilicity values for 18 amino acid residues at a time.

FIG. 29A is a hydrophobicity plot for hsFATP4, showing that it has multiple membrane-spanning domains.

FIG. 29B is a listing of the amino acid composition of hsFATP4.

FIG. 29C is a hydrophilicity plot for hsFATP4, made using the Kyte-Doolittle method, averaging hydrophilicity values for 18 amino acid residues at a time.

FIGS. 30A and 30B show a comparison of the nucleotide sequence of human FATP1 (SEQ ID NO:28) and the nucleotide sequence of mouse FATP1 (SEQ ID NO:29).

FIGS. 31A and 31B show a comparison of the nucleotide sequence of human FATP4 (SEQ ID NO:30) and the nucleotide sequence of mouse FATP4 (SEQ ID NO:31).

FIG. 32 shows a comparison of the amino acid sequence of human FATP1 (SEQ ID NO:32) and the amino acid sequence of mouse FATP1 (SEQ ID NO:33). Shaded amino acid residues match the consensus sequence exactly.

FIG. 33 shows a comparison at the amino acid level of human FATP4 (SEQ ID NO:34) and mouse FATP4 (SEQ ID NO:35). Shaded amino acid residues match the consensus sequence exactly.

FIG. 34 shows the nucleotide sequence (SEQ ID NO:36) and predicted amino acid sequence (SEQ ID NO:37) of hsFATP6.

FIG. 35A is a hydrophobicity plot for hsFATP6, showing that it has multiple membrane-spanning domains.

FIG. 35B is a listing of the amino acid composition of hsFATP6.

FIG. 35C is a hydrophilicity plot for hsFATP6, made using the Kyte-Doolittle method, averaging hydrophilicity values for 18 amino acid residues at a time.

FIG. 36 shows an alignment of the amino acid sequences of hsFATP1 (SEQ ID NO:38), hsFATP4 (SEQ ID NO:39) and hsFATP6 (SEQ ID NO:40). Shaded amino acid residues match the consensus sequence exactly.

FIG. 37 shows results of assessment of fatty acid uptake by human FATP1 and human FATP4. The percent of CD2-positive cells exhibiting a BODIPY-fluorescence of more than 300 arbitrary units is plotted for the three different conditions tested.

FIG. 38 is a graph showing uptake of tritiated oleate, with time, by 293 cells transfected with either (diamonds) a plasmid for expression of human FATP4 or (squares) a control plasmid.

FIG. 39 is an illustration of the amino acid sequences of human FATP4 (SEQ ID NO:41) and mouse FATP4 (SEQ ID NO:42) compared to human FATP1 (SEQ ID NO:43). Shown by underlining are the FATP consensus sequence (236-556 of hsFATP1) and the AMP-binding motif (246-254 of hsFATP1). The human FATPs were cloned by screening libraries with sequences from ESTs (expressed sequence tags). Mouse FATP4 was cloned by PCR using degenerate primers.

FIG. 40 is a graph showing the uptake, with time, of tritiated oleate by mouse enterocytes in the presence of no oligonucleotide (squares), sense oligonucleotide (circles) or antisense oligonucleotide (diamonds).

FIG. 41 is a bar graph showing uptake of tritiated oleate, by mouse enterocytes in the presence of various concentrations of antisense (solid bars), mismatch (stippled bars) or sense (lined bars) oligonucleotides.

FIG. 42 is a bar graph showing uptake of tritiated oleate and uptake of ³⁵S-labeled methionine by mouse enterocytes to which were added no oligonucleotide, the antisense oligonucleotide, or the mismatch oligonucleotide.

FIG. 43A is the nucleotide sequence of the gene encoding mouse FATP4 (SEQ ID NO:44).

FIG. 43B is the amino acid sequence of mouse FATP4 protein (SEQ ID NO:45).

FIGS. 44A, 44B, and 44C are the hsFATP1 DNA sequence (SEQ ID NO:46). Coding region: 175-2115 (1941 nt).

FIG. 45 is the hsFATP1 protein sequence (SEQ ID NO:47).

FIGS. 46A and 46B are the hsFATP2 DNA sequence (SEQ ID NO:48). Coding region: 223-2085 (1863 nt).

FIG. 47 is the hsFATP2 protein sequence (SEQ ID NO:49).

FIG. 48 is the partial DNA sequence of hsFATP3 (SEQ ID NO:50). Coding region: 1-993.

FIG. 49 is the partial protein sequence of hsFATP3 (SEQ ID NO:51).

FIGS. 50A, 50B, and 50C are the hsFATP4 DNA sequence (SEQ ID NO:52). Coding region: 208-2139 (1932 nt).

FIG. 51 is the hsFATP4 protein sequence (SEQ ID NO:53).

FIG. 52 is the hsFATP5 partial DNA sequence (SEQ ID NO:54). Coding region: 1-1062.

FIG. 53 is the hsFATP5 partial protein sequence (SEQ ID NO:55).

FIGS. 54A, 54B, and 54C are the hsFATP6 DNA sequence (SEQ ID NO:56). Coding region: 643-2502 (1860 nt).

FIG. 55 is the hsFATP6 protein sequence (SEQ ID NO:57).

FIGS. 56A, 56B, and 56C are the rnFATP1 DNA sequence (rn=Rattus norvegicus; (SEQ ID NO:58). Coding region: 75-2015 (1941 nt).

FIG. 57 is the rnFATP1 protein sequence (SEQ ID NO:59).

FIGS. 58A, 58B, and 58C are the rnFATP2 DNA sequence (SEQ ID NO:60). Coding region: 795-2657 (1863 nt).

FIG. 59 is the rnFATP2 protein sequence (SEQ ID NO:61).

FIGS. 60A and 60B are the mFATP4 partial DNA sequence (SEQ ID NO:62). Coding region: 1-1218.

FIG. 61 is the rnFATP4 partial DNA sequence (SEQ ID NO:63).

FIGS. 62A, 62B, and 62C are the mmFATP1 DNA sequence (SEQ ID NO:64). Coding region: 1-1944.

FIG. 63 is the mmFATP1 protein sequence (SEQ ID NO:65).

FIGS. 64A and 64B are the mmFATP2 DNA sequence (SEQ ID NO:66). Coding region: 121-1992 (1872 nt).

FIG. 65 is the mmFATP2 protein sequence (SEQ ID NO:67).

FIGS. 66A and 66B are the mmFATP3 partial DNA sequence (SEQ ID NO:68). Coding region: 1-1830.

FIG. 67 is the mmFATP3 partial protein sequence (SEQ ID NO:69).

FIGS. 68A, 68B, and 68C are the mmFATP4 DNA sequence (SEQ ID NO:70). Coding region: 1-1932.

FIG. 69 is the mmFATP4 protein sequence (SEQ ID NO:71).

FIGS. 70A and 70B are the mmFATP5 DNA sequence (SEQ ID NO:72). Coding region: 60-2129.

FIG. 71 is the mmFATP5 protein sequence (SEQ ID NO:73).

FIGS. 72A and 72B are the dmFATP partial DNA sequence (dm=Drosophila melanogaster; SEQ ID NO:74). Coding region: 1-1773.

FIG. 73 is the dmFATP partial protein sequence (SEQ ID NO:75).

FIG. 74 is the drFATP partial DNA sequence (dr=Danio rerio, zebrafish; SEQ ID NO:76) Coding region: 1-173.

FIG. 75 is the drFATP partial protein sequence (SEQ ID NO:77).

FIGS. 76A and 76B are the ceFATPa DNA sequence (SEQ ID NO:78). Coding region: 1-1953.

FIG. 77 is the ceFATPa protein sequence (SEQ ID NO:79).

FIGS. 78A and 78B are the ceFATPb DNA sequence (SEQ ID NO:80). Coding region: 1-1968.

FIG. 79 is the ceFATPb protein sequence (SEQ ID NO:81).

FIGS. 80A and 80B are the chFATP DNA sequence (SEQ ID NO:82; ch=Cochliobolu heterostrophus). Coding region: 1-1932.

FIG. 81 is the chFATP protein sequence (SEQ ID NO:83).

FIG. 82 is the anFATP partial protein sequence (an=Aspergillus nidulans; SEQ ID NO:84). Coding region: 1-597.

FIG. 83 is the anFATP partial protein sequence (SEQ ID NO:85).

FIG. 84 is the mgFATP partial DNA sequence (mg=Magnaporthe grisea, rice blast; SEQ ID NO:86). Coding region: 1-522.

FIG. 85 is the mgFATP partial protein sequence (SEQ ID NO:87).

FIGS. 86A and 86B are the scFATP DNA sequence (SEQ ID NO:88). Coding region: 1-1872.

FIG. 87 is the scFATP protein sequence (SEQ ID NO:89).

FIGS. 88A and 88B are the mtFATP DNA sequence (SEQ ID NO:90).

FIG. 89 is the mtFATP protein sequence (SEQ ID NO:91). Coding region: 1-1794.

FIG. 90 is a consensus sequence of the FATP signature sequence (SEQ ID NO:100), based on 23 independent sequences aligned in ClustalX. The height of the bar at each amino acid residue position indicates the degree of conservation at that position. Gaps have been inserted to maintain the strength of the alignment.

FIG. 91 is a hydrophilicity plot for hsFATP2, made using the Kyte-Doolittle method, averaging hydrophilicity values for 18 amino acid residues at a time.

FIG. 92 is a hydrophilicity plot for the hsFATP3 partial protein, made using the Kyte-Doolittle method, averaging hydrophilicity values for 18 amino acid residues at a time.

FIG. 93 is a hydrophilicity plot for the hsFATP5 partial protein, made using the Kyte-Doolittle method, averaging hydrophilicity values for 18 amino acid residues at a time.

FIGS. 94A and 94B are a representation of the DNA sequence (SEQ ID NO:101) of the hsFATP3 gene, and the amino acid sequence (SEQ ID NO:102) of the hsFATP3 protein.

FIG. 95 shows that mammalian expression constructs containing either hsFATP4 (squares and triangles) or empty control vector (circles) were stably transfected into 293 cells. Short-term uptake of Bodipy-FA in the presence of BSA was determined by FACS. The mean fluorescence of the viable cell population is expressed in arbitrary fluorescence units. FATP4 protein expression was determined by densitometry of anti-FATP4 Western blots, and is expressed in arbitrary units.

FIG. 96 is a bar graph illustrating short-term uptake of Bodipy-palmitate (1 μM), either by control cells (black bars) or FATP4-expressing cells (hatched bars), was measured in the presence of 0, 10, 100 μM unlabeled palmitate. FA uptake was quantified by FACS and expressed in arbitrary fluorescence units.

FIG. 97 shows the rate of [²H]palmitate uptake by 293 cells, which were stably transfected with a construct for either human FATP4 (diamonds) or an empty vector (circles), compared to that of isolated enterocytes (squares).

FIG. 98 is a bar graph illustrating the results when isolated enterocytes were incubated for 48 h with increasing concentrations of the FATP4 antisense oligonucleotide or with 100 μM of a randomized control oligonucleotide with identical nucleotide composition to the FATP4 antisense oligonucleotide. The uptake of oleate by the enterocytes was then measured over a 5 min time interval (solid bars). In parallel, the levels of FATP4 protein and, as a loading control, β-catenin, were determined by Western blotting and quantitated using densitometry (hatched bars). FA uptake and FATP4 protein levels were normalized to that of untreated cells. The averages and standard deviations of 4 independent experiments are shown.

FIG. 99 is a bar graph illustrating the uptake rates of [³H]oleate, [³H]palmitate and [³⁵S]methionine by primary enterocytes were measured after 48 h incubation with either 100 μM FATP4 antisense (solid bars) or 100 μM randomized control oligonucleotide (hatched bars) and expressed as % of untreated cells.

FIG. 100 is a bar graph illustrating that 8 kb of FATP5 genomic sequence SEQ ID NO.: 106 is sufficient for liver specific transcription in vitro. A luciferase reporter construct containing 8 kb upstream of the FATP5 initiator methionine was transfected into various cell lines using calcium phosphate as described in Example 17. Forty-eight hours after transfection, luciferase activity was measured and normalized to β-galactosidase activity. For each cell line, fold induction was determined by dividing the relative luciferase activity of the 8 kb construct by that of the promoter-less luciferase reporter vector. The data shown represent the mean of three experiments done in triplicate. Error bars indicate the SEM.

FIG. 101 is a bar graph illustrating deletion analysis of the FATP5 promoter. Constructs containing deletions of the FATP5 promoter were transfected into HepG2 cells, assayed for luciferase activity, and normalized to β-galactosidase (RLU). The labels on the vertical axis correspond to the length of the promoter segment as measured from the initiator methionine. The data shown represents the mean of three experiments done in triplicate. Error bars indicate the SEM.

FIG. 102 is a bar graph illustrating that 271 base pairs upstream of the FATP5 initiator methionine are sufficient for liver specific luciferase activity. A luciferase reporter construct containing 271 base pairs upstream of the FATP5 initiator methionine was transfected into various cell lines using calcium phosphate as described in Methods Example 17. Forty eight hours after transfection, luciferase activity was measured and normalized to β-galactosidase activity. For each cell line, fold induction was determined by dividing the relative luciferase activity of the −271 base pair construct by that of the promoter-less luciferase reporter vector. The data shown represent the mean of three experiments done in triplicate. Error bars indicate the SEM.

FIGS. 103A and 103B illustrate mutations of the GC box which abolish transcriptional activity. A: Schematic of mutations in the GC box aligned with the normal sequence (SEQ ID NO.: 106, SEQ ID NO.: 107, SEQ ID NO.: 108). The GC box consensus sequence is underlined. B: Constructs containing 271 base pairs upstream of the FATP5 initiator methionine with the mutations in the GC box depicted in part A were transfected into HepG2 cells, assayed for luciferase activity, and normalized to -galactosidase (RLU). The data shown represent the mean of three experiments done in triplicate. Error bars indicate the SEM.

FIG. 104 shows a gel shift analysis of the GC box with HepG2 nuclear extracts. Schematic showing the sequence of the oligonucleotides used in gel shift studies. The numbering reflects the distance from the initiator methionine. The two pairs of oligonucleotides are indicated by the lines and labeled AF-1 (SEQ ID NO.: 111, SEQ ID NO.: 112) and AF-2 (SEQ ID NO.: 109, SEQ ID NO.: 110).

FIG. 105 is a bar graph illustrating that 30 bp internal deletions of the FATP5 promoter identify another region required for luciferase activity in HepG2 cells. Reporter constructs were transfected into HepG2 cells. Luciferase activity was measured and normalized to β-galactosidase activity (RLU). The labels on the horizontal axis correspond to the nucleotides that were deleted and the numbering on the vertical axis represents the distance from the initiator methionine. The data shown represent the mean of three experiments done in triplicate. Error bars indicate the SEM. Note that the five fold higher RLU activity in this figure relative to FIGS. 101 and 103 is the result of a manufacturer change in the β-galactosidase reagent.

FIG. 106 is a bar graph illustrating that a linker scan of the FATP5 promoter identifies two additional elements required for transcription in HepG2 cells. Reporter constructs were transfected into HepG2 cells. Luciferase activity was measured and normalized to β-galactosidase activity (RLU). The labels on the horizontal axis correspond to the constructs in part A. The data shown represent the mean of three experiments done in triplicate. Error bars indicate the SEM. Please note that the lower RLU activity in this figure relative to FIGS. 101 and 103 is also the result of a manufacturer change in the β-galactosidase reagent.

FIG. 107 is a schematic of the FATP5 promoter (SEQ ID NO.: 113). The GC box and two motifs identified in the linker scan are boxed and labeled. An arrow indicates the translational initiator of the FATP5 protein. The two halves of the palindrome contained in the novel motifs and referred to in the discussion are underlined.

FIG. 108 is a photograph showing FATP2 expression in the mouse gall bladder epithelium.

FIG. 109 is a photograph showing FATP2 expression in chimpanzee liver.

FIG. 110 is a photograph showing FATP5 expression in chimpanzee liver.

FIGS. 111A and 111B represent the DNA sequence (SEQ ID NO:116) of human FATP3.

FIG. 112 represents the amino acid sequence (SEQ ID NO:1 17) of human FATP3.

FIG. 113 is a bar graph showing the results of an experiment comparing fatty acid transport between cells transfected with SEQ ID NO:116 and untransfected cells.

FIGS. 114A, 114B, 114C and 114D represent portions of the amino acid sequence of mmFATP4 which were produced as fusion polypeptides in E. coli cells.

FIG. 115 is a schematic illustrating certain components of the fusion polypeptides depicted in FIGS. 114A-D. The schematic shows the lipocalin domain as well as other identified motifs and notes the relative location of each in the mmFATP4 fusion polypeptide.

FIG. 116 is a bar graph illustrating the results of an experiment comparing the binding capabilities of the fusion polypeptides shown in FIGS. 114A-D for an oleate fatty acid.

FIG. 117 is a bar graph showing the results of an experiment comparing binding of various fatty acids between two of the fusion polypeptides depicted in FIGS. 114A-D.

FIGS. 118A-G illustrates the consensus sequence of hsFATP1, hsFATP2, hsFATP3, hsFATP4, hsFATP5 and hsFATP6 with the lipocalin domain and AMP-binding domain of each noted.

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

### DETAILED DESCRIPTION OF THE INVENTION

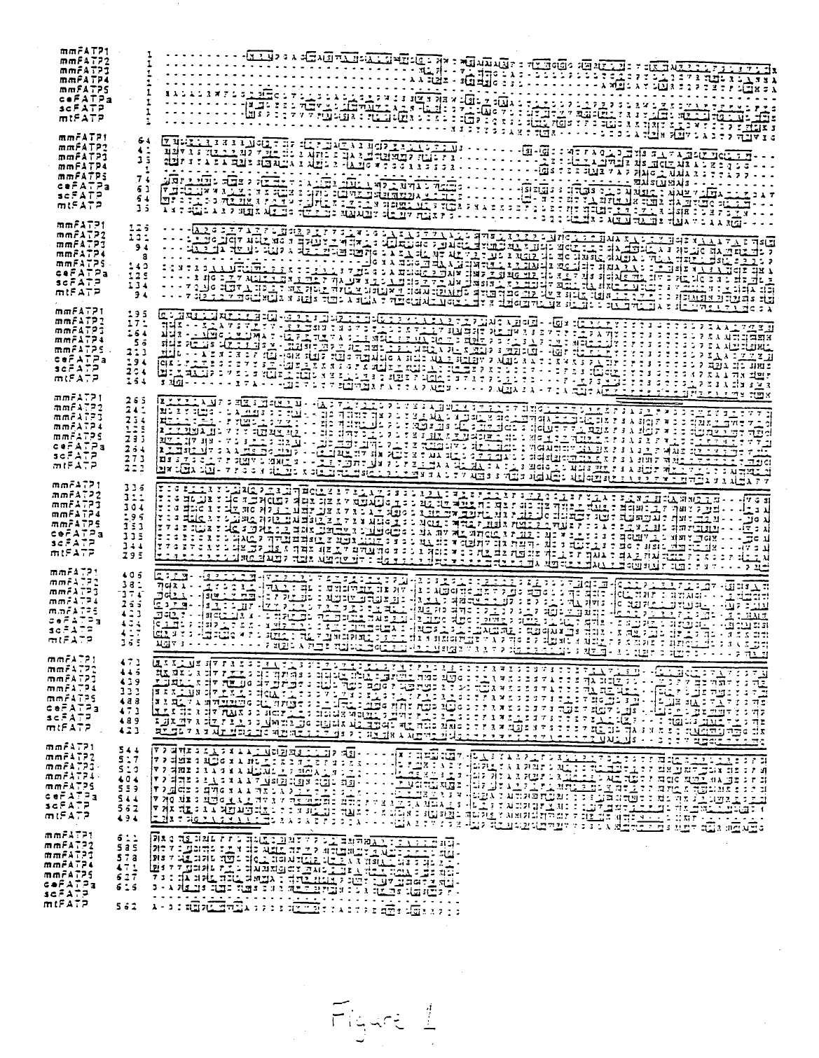

As described herein, FATPs are a large evolutionarily conserved family of proteins that mediate the transport of LCFAs into cells. The family includes proteins which are conserved from mycobacteria to humans and exhibit very different expression patterns in tissues. Specific embodiments described include FATPs from mice, humans, nematodes, fungi and mycobacteria which have been shown to be functional LCFA transporters. The term “fatty acid transport proteins” (“FATPs”) as used herein, refers to the proteins described herein as FATP1, FATP2, FATP3, FATP4, FATP5 and FATP6, which have been described in one or more species of mammals, as well as mtFATP, ceFATP, scFATP, anFATP, mgFATP, and chFATP, and other proteins sharing at least about 50% amino acid sequence similarity, preferably at least about 60% sequence similarity, more preferably at least about 70% sequence similarity, and still more preferably, at least about 80% sequence similarity, and most preferably, at least about 90% sequence similarity in the approximately 360 amino acid signature sequence. The approximately 360 amino acid FATP signature sequence is shown in FIG. 1. The consensus sequence of the signature sequence is shown in FIG. 90. The nomenclature used herein to refer to FATPs includes a species-specific prefix (e.g., mm, Mus musculus; hs or h, Homo sapiens or human; mt M. tuberculosis; dm, D. melanogaster; ce, C. elegans; sc, Saccharomyces cerevisiae) and a number such that mammalian homologues in different species share the same number. For example, six human and five mouse FATP genes which are expressed in a variety of tissues are described herein and are referred to, respectively, as hsFATP1-hsFATP6 and mmFATP1-mmFATP5; for example, hsFATP4 and mmFATP4 are the human and mouse orthologs.

Expression patterns of human and mouse FATPs have been assessed and are described below. Briefly, results of these assessments show that FATP5 is a liver-specific gene. FATP2 is highly expressed in liver, kidney and gall bladder epithelium. Both of these proteins, as well as FATP4 and FATPs from nematodes and mycobacteria, have been shown to be functional LCFA transporters. Results have also shown that FATP4 mRNA is present at high levels in epithelial cells of two regions of the small intestine (the jejunum and ileum) and at lower, but significant, levels in a third region (the duodenum). They further showed that FATP2 mRNA is present in epithelial cells of the duodenum at a level similar to that of FATP4 mRNA levels, but is present at lower levels in the jejunum and ileum. FATP4 mRNA was absent from other cell types of the small intestine and no FATP4 mRNA could be detected in any cells of the colon. No signals above background could be detected for FATP 1, FATP3 and FATP5 in any of the intestinal tissues. Thus, FATP4 is the major FATP in the mouse small intestine, which supports a major role for FATP4 (along with FATP2 to a lesser extent) in absorption of free fatty acids. hsFATP4 was clearly expressed in the jejunum and ileum; expression was absent in the stomach. This, too, is consistent with a major role for FATP4 in absorption of fatty acids in the human gut. Analysis of FATP expression in human tissues, also described in detail below, showed that hsFATP6, which has no mouse ortholog as yet, is expressed at high levels in the heart and at low levels in the placenta, but is undetectable in the other tissues assessed (Example 9). This is consistent with a major role for FATP6 in absorption of fatty acids in the heart.

Analysis of FATP3 expression in murine tissues, also described in detail below, showed that expression occurs at detectable levels in liver, spleen, heart, kidney, testis, white adipose tissue, exocrine and endocrine pancreatic cells, and also in lung tissues. FATP3 is expressed at high levels in type-II pneumocytes, a cell type noted for secretion a surfactant, a phospholipid-rich film critical for lung function (Example 19).

Long chain fatty acids (LCFAs) are an important energy source for pro- and eukaryotes and are involved in diverse cellular processes, such as membrane synthesis, intracellular signaling, protein modification, and transcriptional regulation. In developed Western countries, human dietary lipids are mainly di- and triglycerides and account for approximately 40% of caloric intake (Weisburger, J. H. (1997) J. Am. Diet. Assoc. 97:S16-S23). These lipids are broken down into fatty acids and glycerol by pancreatic lipases in the small intestine (Chapus, C., Rovery, M., Sarda, L & Verger, R. (1988) Biochimie 70:1223-34); LCFAs are then transported into brush border cells, where the majority is re-esterified and secreted into the lymphatic system as chylomicrons (Green, P. H. & Riley, J. W. (1981) Aust. N.Z.J. Med. 11:84-90). Fatty acids are liberated from lipoproteins by the enzyme lipoprotein lipase, which is bound to the luminal side of endothelial cells (Scow, R. O. & Blachette-Mackie, E. J. (1992) Mol. Cell. Biochem 116:181-191). “Free” fatty acids in the circulation are bound to serum albumin (Spector, A. A. (1984) Clin. Physiol. Biochem 2:123-134) and are rapidly incorporated by adipocytes, hepatocytes, and cardiac muscle cells. The latter derive 60-90% of their energy through the oxidation of LCFAs (Neely, J. F. Rovetto, M. J. & Oram, J. F. (1972) Prog. Cardiovasc. Dis: 15:289-329). Although saturable and specific uptake of LCFAs has been demonstrated for intestinal cells, hepatocytes, cardiac myocytes, and adipocytes, the molecular mechanisms of LCFA transport across the plasma membrane have remained controversial (Hui, T. Y. & Bernlohr, D. A. (1997) Front. Biosci. 15:d222-3 1-d231; Schaffer, J. E. & Lodish, H. F, (1995) Trends Cardiovasc. Med. 5:218-224). Described herein is a large family of highly homologous mammalian LCFA transporters which show wide expression, including in all tissues relevant to fatty acid metabolism. Further described are novel members of this family in other species, including mycobacterial and nematode FATPs which, like their mammalian counterparts, are functional fatty acid transporters.

The discovery of a diverse but highly homologous family of FATPs is reminiscent of the glucose transporter family. In a manner similar to the FATPs, the glucose transporters have very divergent patterns of tissue expression (McGowan, K. M., Long, S. D. & Pekala, P. H. (1995) Pharmacol. Ther. 66:465-505). The FATPs, like glucose transporters, may also differ in their substrate specificities, uptake kinetics, and hormonal regulation (Thorens, B. (1996) Am. J. Physiol. 270:G541-G553). Indeed, the levels of fatty acids in the blood, like those of glucose, can be regulated by insulin and are dysregulated in diseases such as noninsulin-dependent diabetes and obesity (Boden, G. (1997) Diabetes 46:3-10). The underlying mechanisms for the regulation of free fatty acid concentrations in the blood are not understood, but could be explained by hormonal modulation of FATPs.

Insulin-resistance is thought to be the major defect in non insulin-dependent diabetes mellitus (NIDDM) and is one of the earliest manifestations of NIDDM (McGarry (1992) Science 258:766-770). Free fatty acids (FFAs) may provide an explanation for why obesity is a risk factor for NIDDM. Plasma levels of FFAs are elevated in diabetic patients (Reaven et al. (1988) Diabetes 37:1020). Elevated plasma free fatty acids (FFAs) have been demonstrated to induce insulin-resistance in whole animals and humans (Boden (1998) Front. Biosci. 3:D169-D175). This insulin-resistance is likely mediated by effects of FFAs on a variety of issues. FFAs added to adipocytes in vitro induce insulin resistance in this cell type as evidenced by inhibition of insulin-induced glucose transport (Van Epps-Fung et al. (1997) Endocrinology 138:4338-4345). Rats fed a high fat diet developed skeletal muscle insulin resistance as evidenced by a decrease in insulin-induced glucose uptake by skeletal muscle (Han et al., (1997) Diabetes 46:1761-1767). In addition, elevated plasma FFAs increase insulin-suppressed endogenous glucose production in the liver (Boden (1998) Front. Biosci. 3:D169-D175), thus increasing hepatic glucose output. It has been postulated that the adverse effects of plasma free fatty acids are due to the FFAs being taken up into the cell, leading to an increase in intracellular long chain fatty acyl .CoA; intracellular long chain acyl CoAs are thought to mediate the effects of FFAs inside the cell. Thus, fatty acid induced insulin-resistance may be prevented by blocking uptake of FFAs into select tissues, in particular liver (by blocking FATP2 and/or FATP5), adipocyte (by blocking FATP1), and skeletal muscle (by blocking FATP1). Blocking intestinal fat absorption (by blocking FATP4) is also expected to reduce plasma FFA levels and thus improve insulin resistance.

During the pathogenesis of NIDDM insulin-resistance can initially be counteracted by increasing insulin output by the pancreatic beta cell. Ultimately, this compensation fails, beta cell function decreases and overt diabetes results (McGarry (1992) Science 258: 766-770). Manipulating beta cell function is a second point where fatty acid transporter blockers may be beneficial for diabetes. While no FATP homolog has been identified so far that is expressed in the beta cell of the pancreas, the data described below suggest the existence of such a transporter and the sequence information included herein provides the means to identify such a transporter by degenerate PCR, using primers to regions conserved in all FATP family members or by low stringency hybridization. It has been demonstrated that exposure of pancreatic beta-cells to FFAs increases the basal rate of insulin secretion; this in turn leads to a decrease in the intracellular stores of insulin, resulting in decreased capacity for insulin secretion after chronic exposure (Bollheimer et al., (1998) J. Clin. Invest. 10 1:1094-1101). The effects of FFAs are again likely to be mediated by intracellular long chain fatty acyl CoA molecules (Liu et al., (1998) J. Clin. Invest. 101:1870-1875). FFAs have also been demonstrated to increase beta cell apoptosis (Shimabukuro et al., (1998) Proc. Nat. Acad. Sci. USA 95:2498-2502), possibly contributing to the decrease in beta cell numbers in late stage NIDDM.

Another finding with potentially broad implications is the identification of a FATP homologue in M. tuberculosis. Tuberculosis causes more deaths. worldwide than any other infectious agent and drug-resistant tuberculosis is re-emerging as a problem in industrialized nations (Bloom, B. R. & Small, P. M. (1998) N. Engl. J. Med. 338:677-678). Mycobacterium tuberculosis has about 250 enzymes involved in fatty acid metabolism, compared with only about 50 in E. coli. It has been suggested that, living as a pathogen, the mycobacteria are largely lipolytic, rather than lipogenic, relying on the lipids within mammalian cells and the tubercle (Cole, S. T. et al., Nature 393:537-544 (1998)). The de novo synthesis of fatty acids in Mycobacterium leprae is insufficient to maintain growth (Wheeler, P. R., Bulmer, K & Ratledge, C. (1990) J. Gene. Microbiol. 136:211-217). Thus, it is reasonable to expect that inhibitors of mtFATP will serve as therapeutics for tuberculosis. FATPs expressed in mycobacteria can be targeted to reduce or prevent replication of mycobacteria (e.g., to reduce or prevent replication of M. tuberculosis) and, thus, reduce or prevent their adverse effects. For example, a FATP or FATPs expressed by M. tuberculosis can be targeted and inhibited, thus reducing or preventing growth of this pathogen (and tuberculosis in humans and other mammals). An inhibitor of an M. tuberculosis FATP can be identified, using methods described herein (e.g., expressing the FATP in an appropriate host cell, such as E. coli or COS cells; contacting the cells with an agent or drug to be assessed for its ability to inhibit the FATP and, as a result, mycobacterial growth, and assessing its effects on growth). A drug or agent identified in this manner can be further tested for its ability to inhibit a M. tuberculosis FATP and M. tuberculosis infection in an appropriate animal model or in humans. A method of inhibiting mycobacterial growth, particularly growth of M. tuberculosis, and compounds useful as drugs for doing so are also the subject of this invention.

An isolated polynucleotide encoding mtFATP, like other polynucleotides encoding FATPs of the FATP family, can be incorporated into vectors, nucleic acids of viruses, and other nucleic acid constructs that can be used in various types of host cells to produce mtFATP. This mtFATP can be used, as it appears on the surface of cells, or in various artificial membrane systems, to assess fatty acid transport function, to identify ligands and molecules that are modulators of fatty acid transport activity. Molecules found to be inhibitors of mtFATP function can be incorporated into pharmaceutical compositions to administer to a human for the treatment of tuberculosis.

Particular embodiments of the invention are polynucleotides encoding a FATP of Cochliobolus (Helminthosporium) heterostrophus or portions or variants thereof, the isolated or recombinantly produced FATP, methods for assessing whether an agent binds to the chFATP, and further methods for assessing the effect of an agent being tested for its ability to modulate fatty acid transport activity. Cochliobolus heterostrophus is an ascomycete that is the cause of southern corn leaf blight, an economically important threat to the corn crop in the United States. The related species C. sativus causes crown rot and common root rot in wheat and barley. One or more FATPs of C. heterostrophus can be targeted for the identification of an inhibitor of chFATP function, which can be then be used as an agent effective against infection of plants by C. heterostrophus and related organisms. Methods described herein that were applied in studying the expression of a FATP gene and the function of the FATP in its natural site of expression or in a host cell, can be used in the study of the chFATP gene and protein.

Magnaporthe grisea (rice blast) is an economically important fungal pathogen of rice. Further embodiments of the invention are nucleic acid molecules encoding a FATP of Magnaporthe grisea, portions thereof, or variants thereof, isolated mgFATP, nucleic acid constructs, and engineered cells expressing mgFATP. Other aspects of the invention are assays to identify an agent which binds to mgFATP and assays to identify an agent which modulates the function of mgFATP in cells in which mgFATP is expressed or in artificial membrane systems. Agents identified as inhibiting mgFATP activity can be developed into anti-fungal agents to be used to treat rice infected with rice blast.

Caenorhabditis elegans is a nematode related to plant pathogens and human parasites. An isolated polynucleotide which encodes ceFATP, like other polynucleotides encoding FATPs of the FATP family described herein, can be incorporated into nucleic acid vectors and other constructs that can be used in various types of cells to produce ceFATP. ceFATP as it occurs in cells or as it can be isolated or incorporated into various artificial or reconstructed membrane systems, can be used to assess fatty acid transport, and to identify ligands and agents that modulate fatty acid transport activity. Agents found by such assays to be inhibitors of ceFATP activity can be incorporated into compositions for the treatment of diseases caused by genetically related organisms with a FATP of similar sensitivity to the agents.

Aspergillus nidulans is one of a family of fungal species that can infect humans. Further embodiments of the invention of the family of polynucleotides encoding FATPs are polynucleotides encoding a FATP of Aspergillus nidulans, and vectors and host cells that can be constructed to comprise such polynucleotides. Further embodiments are a polypeptide encoded by such polynucleotides, portions thereof having one or more functions characteristic of a FATP, and various methods. The methods include those for identifying agents that bind to a FATP and those for assessing the effect of an agent being tested for its ability to modulate fatty acid transport activity. Those agents found to inhibit fatty acid transport function can be used in compositions as anti-fungal pharmaceuticals, or can be modified for greater effectiveness as a pharmaceutical.

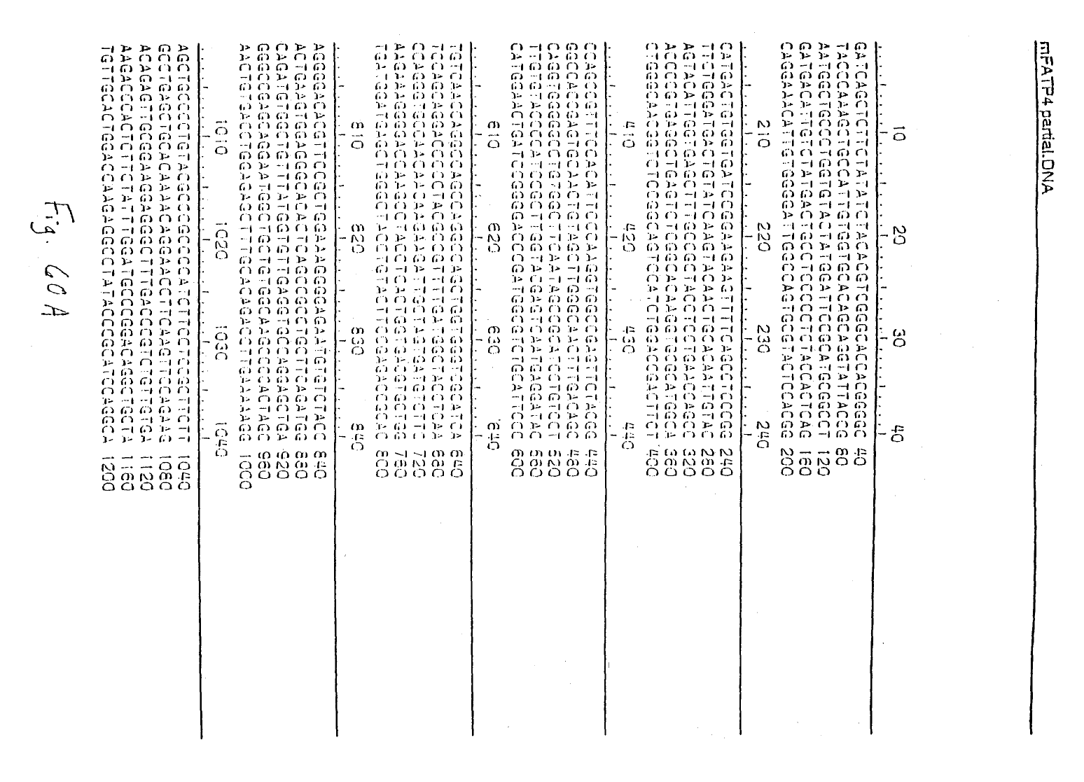

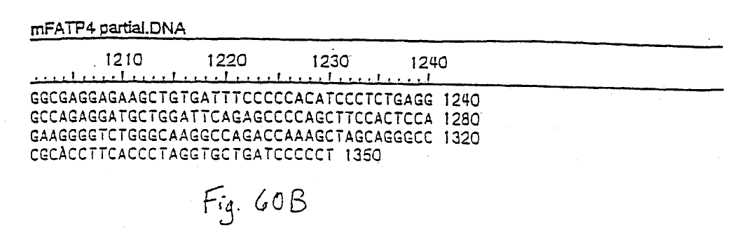

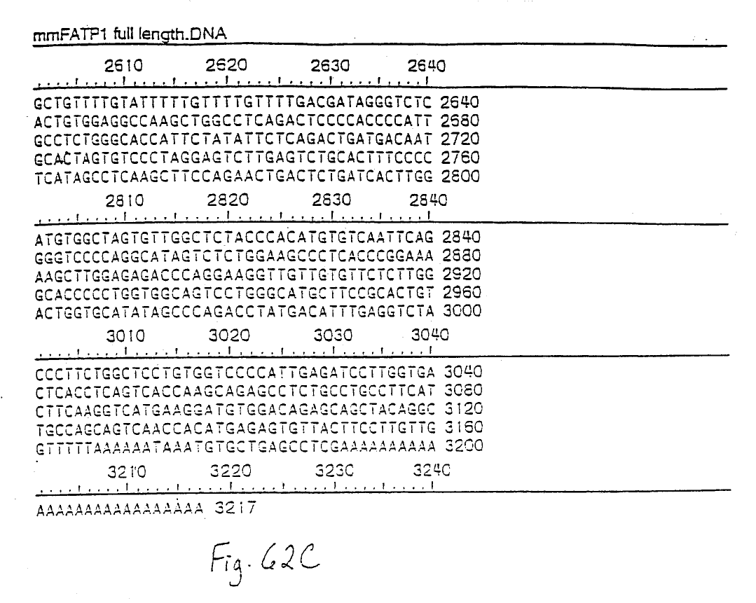

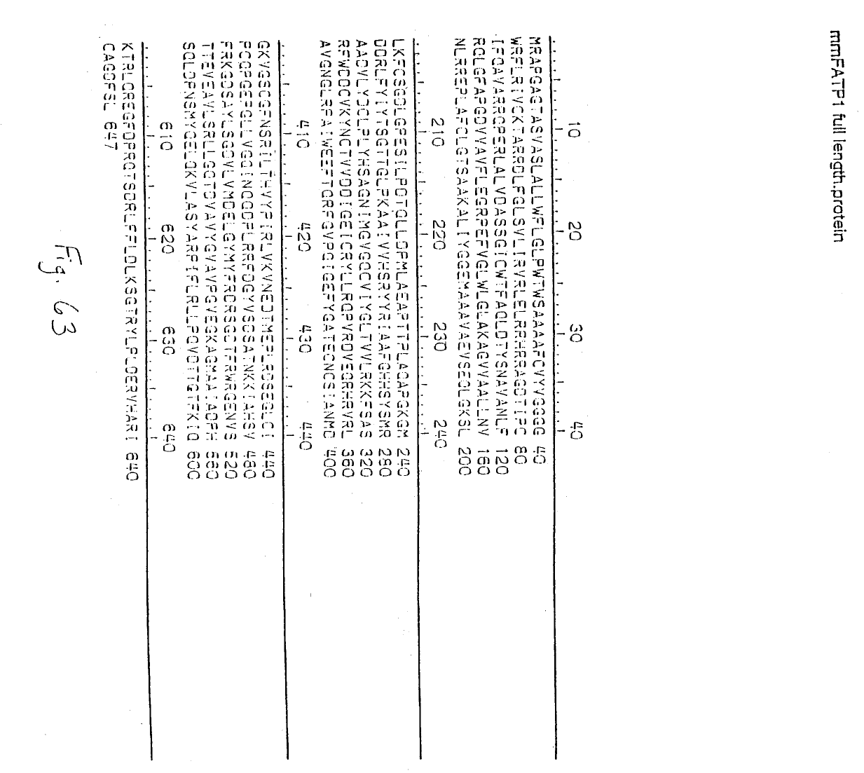

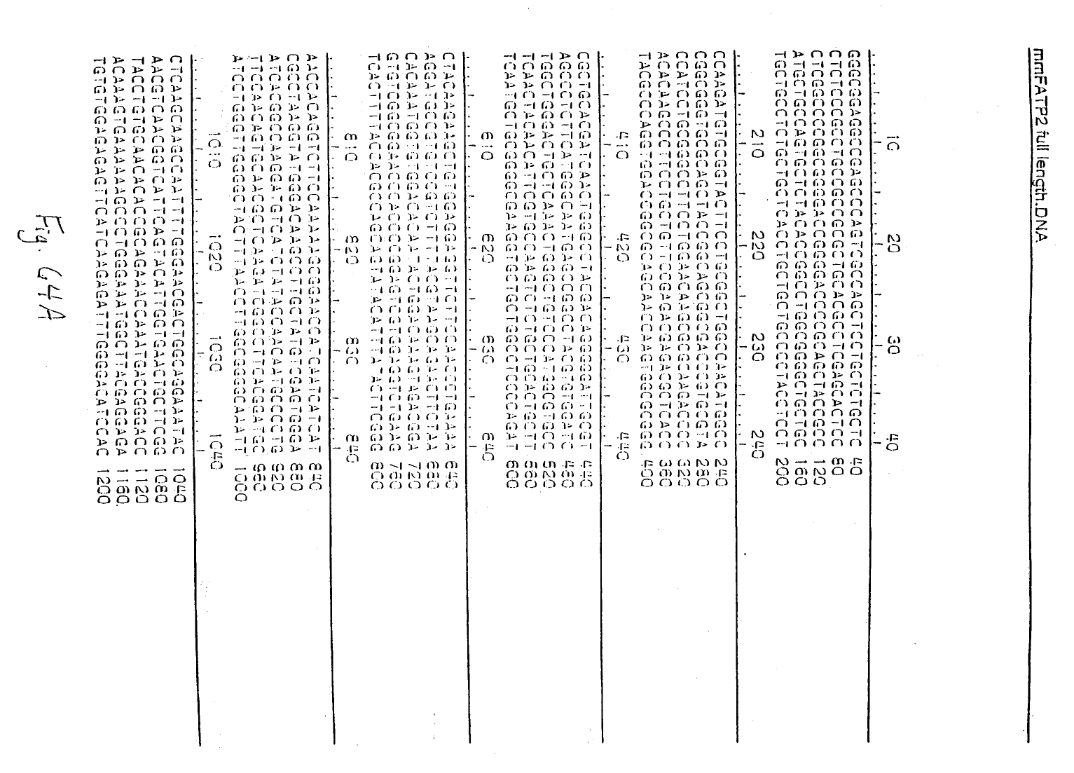

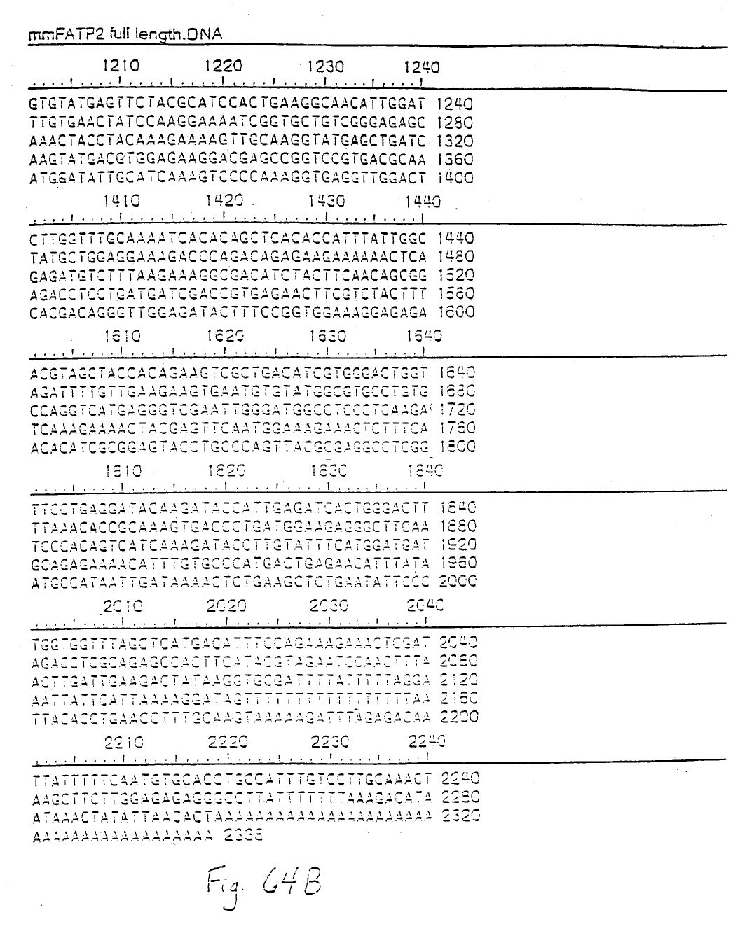

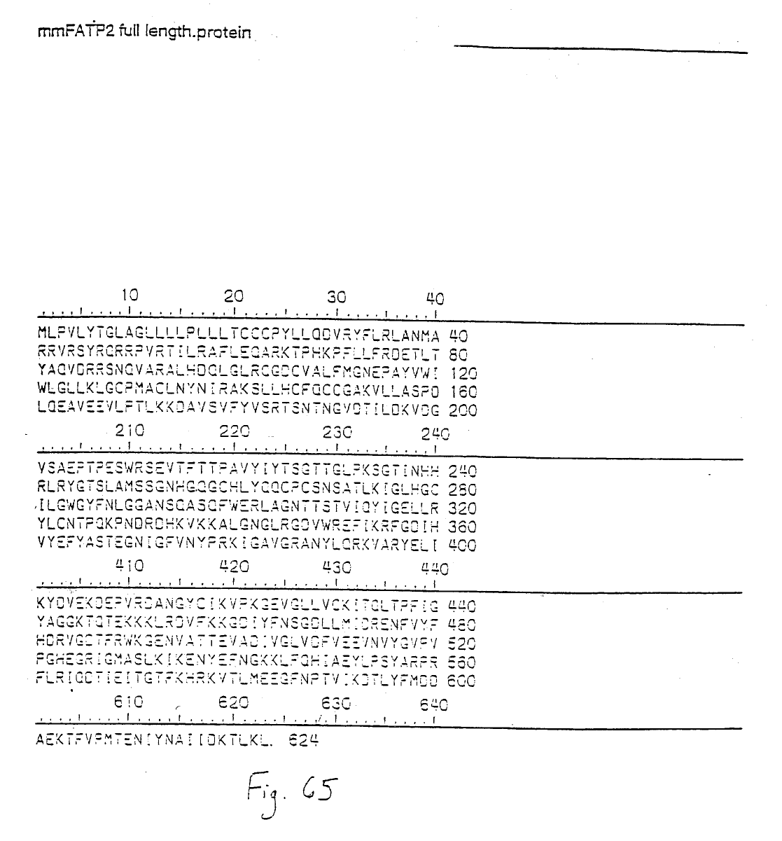

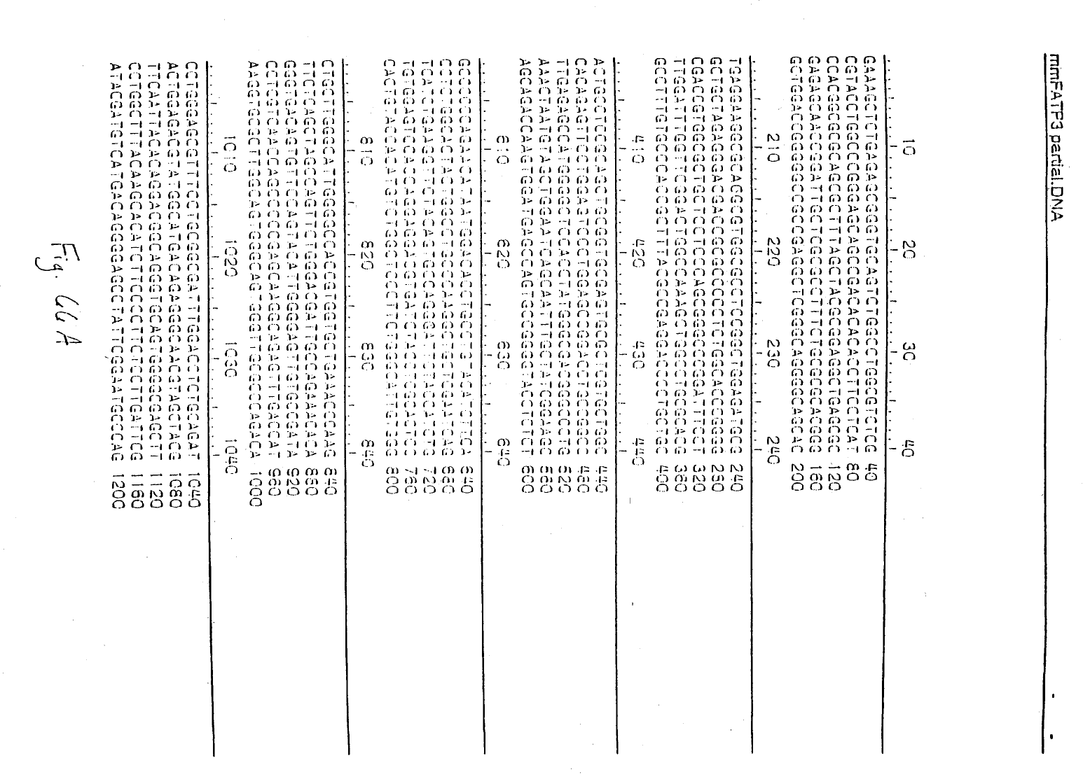

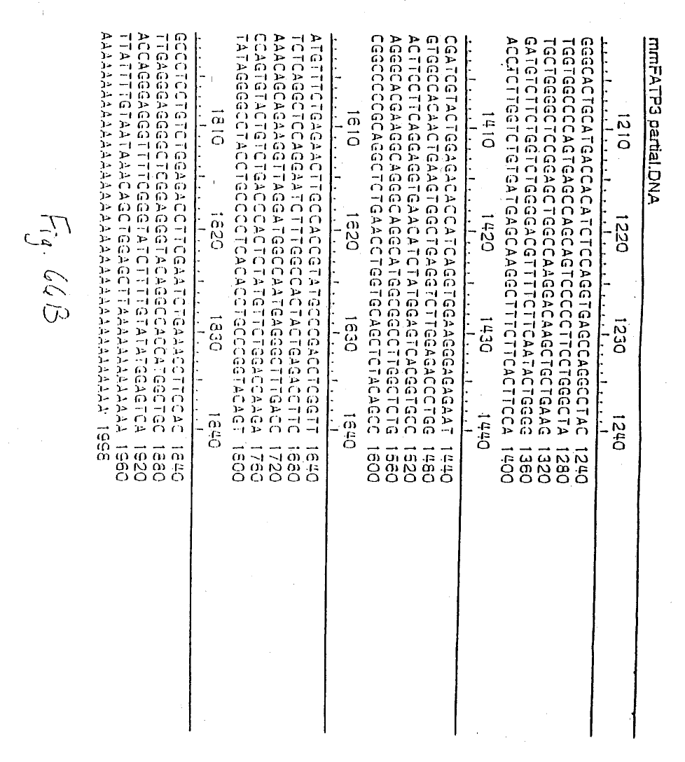

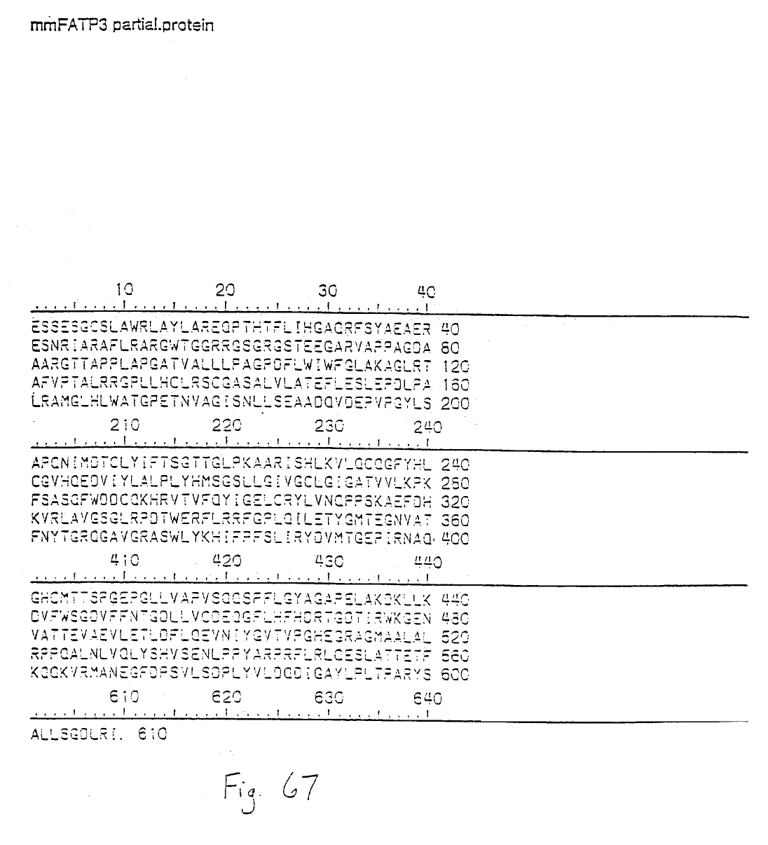

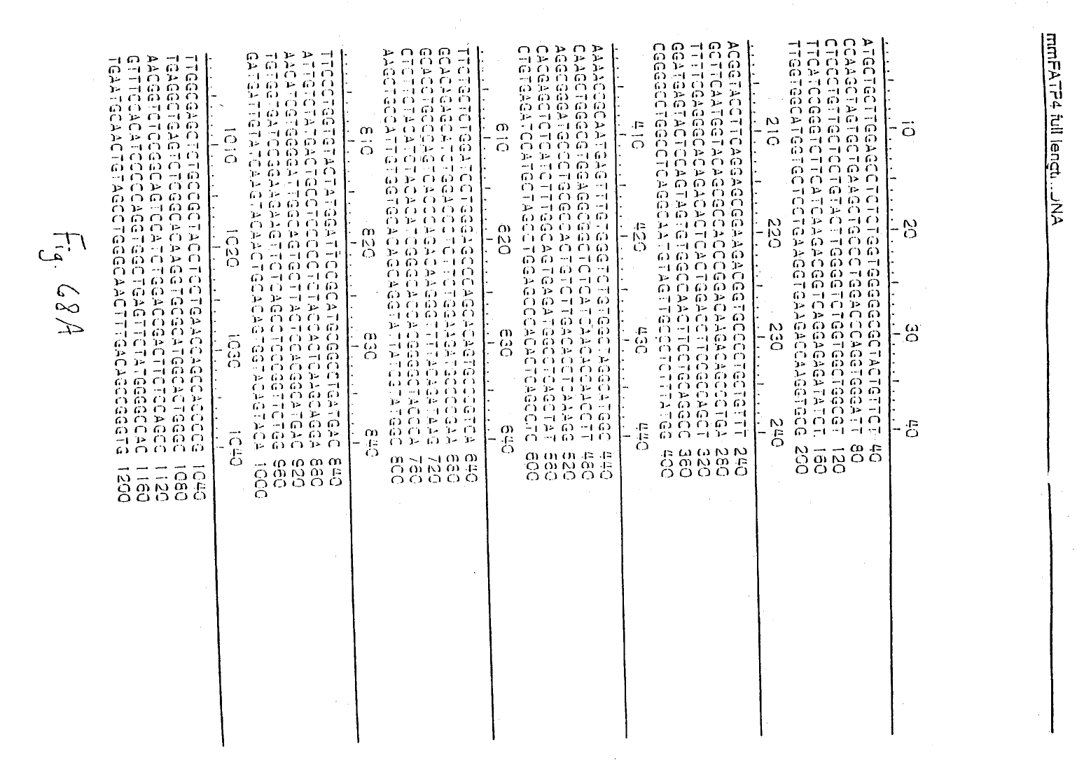

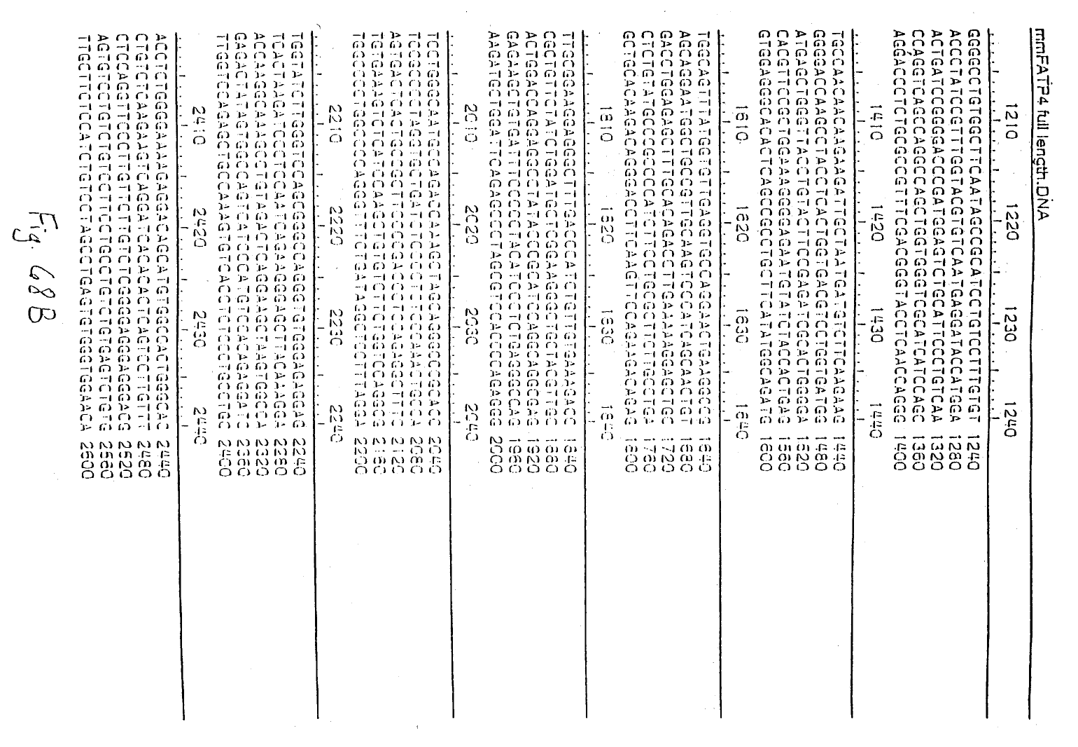

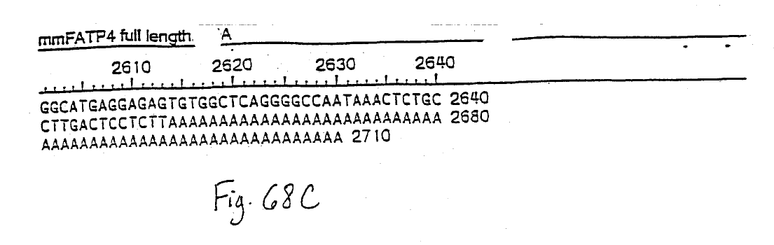

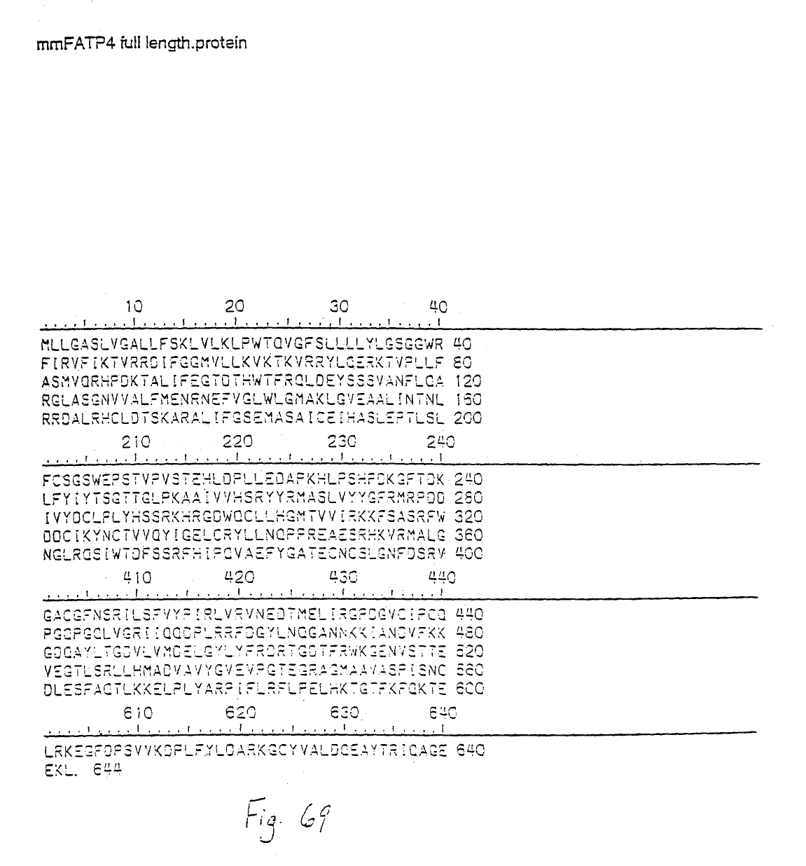

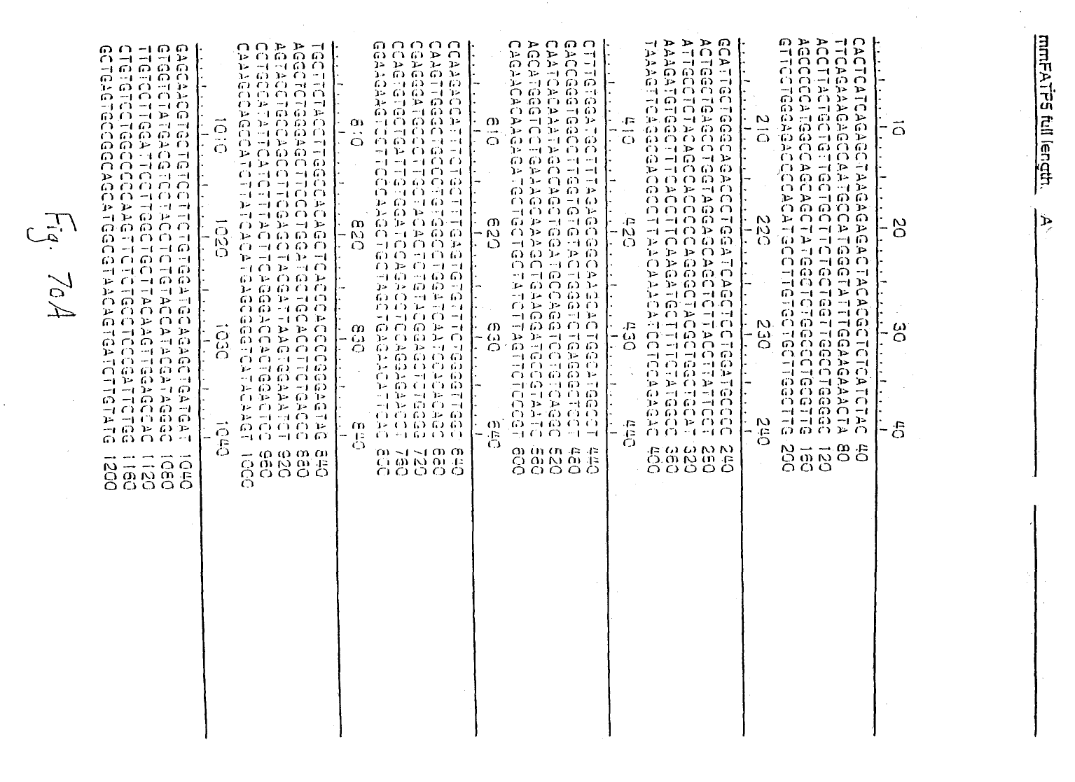

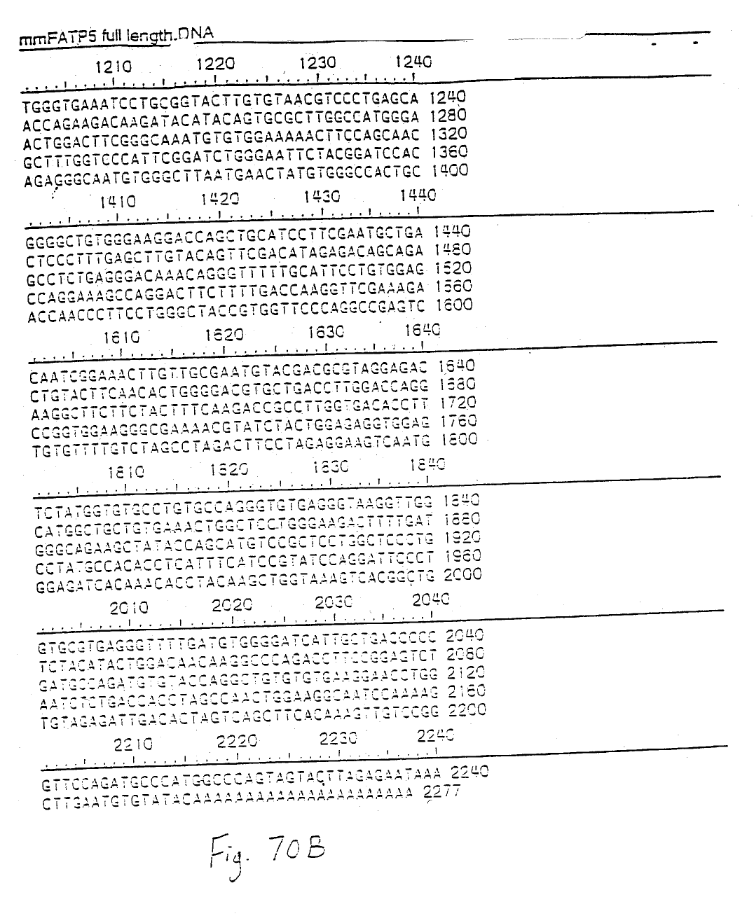

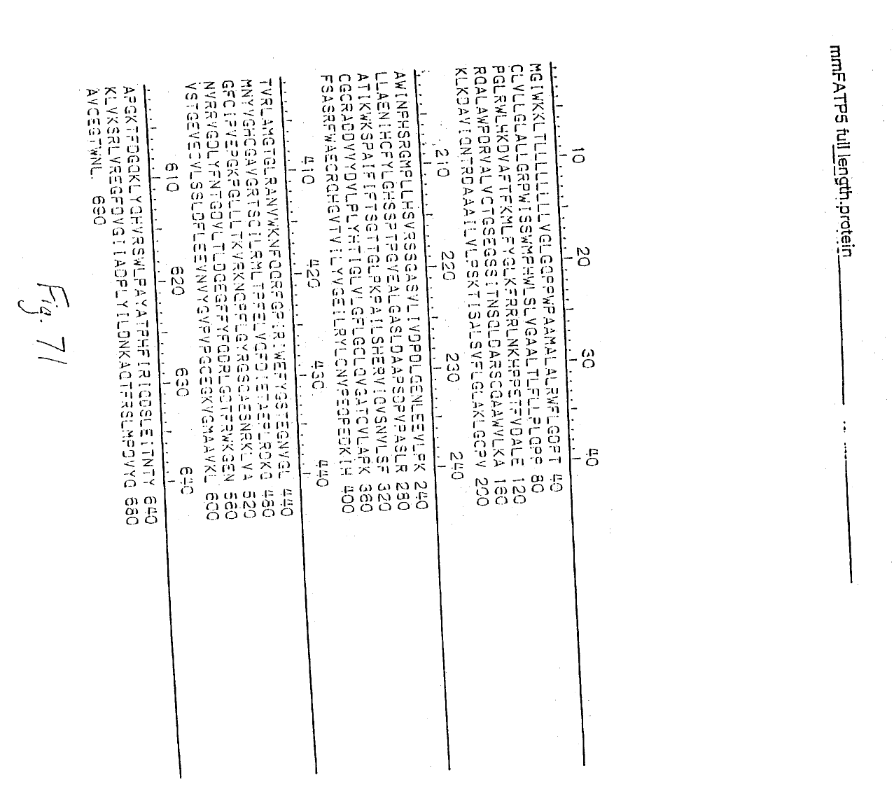

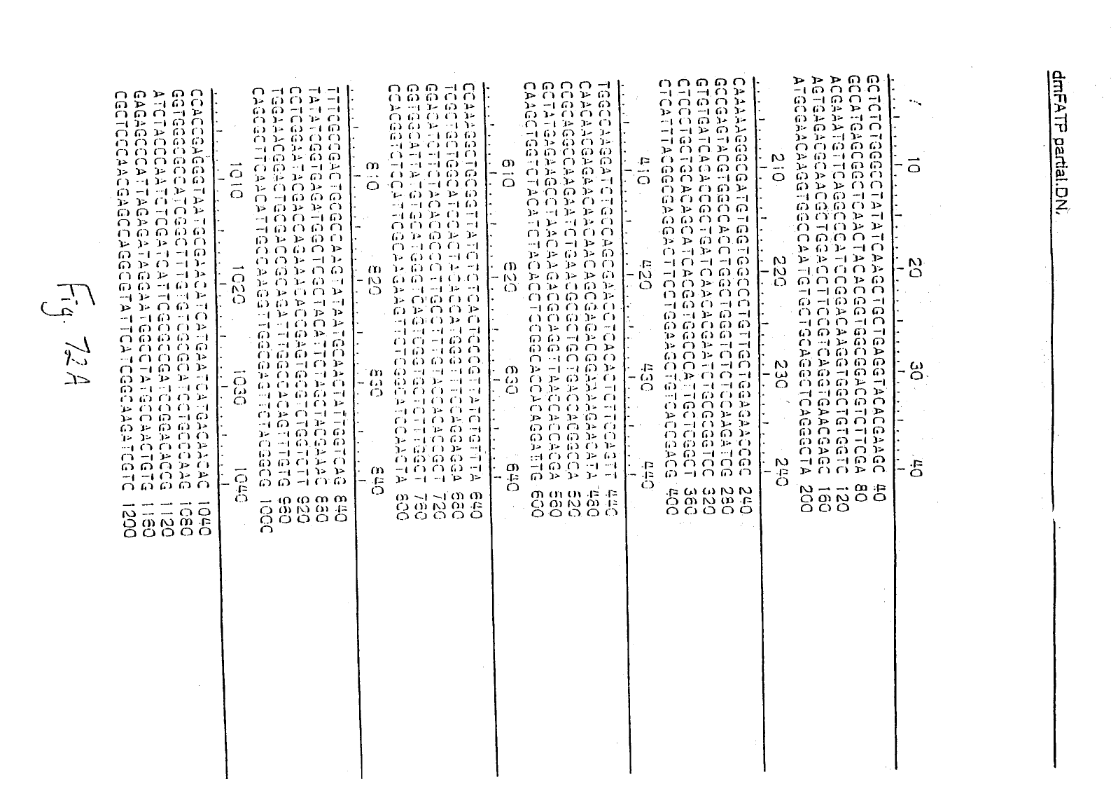

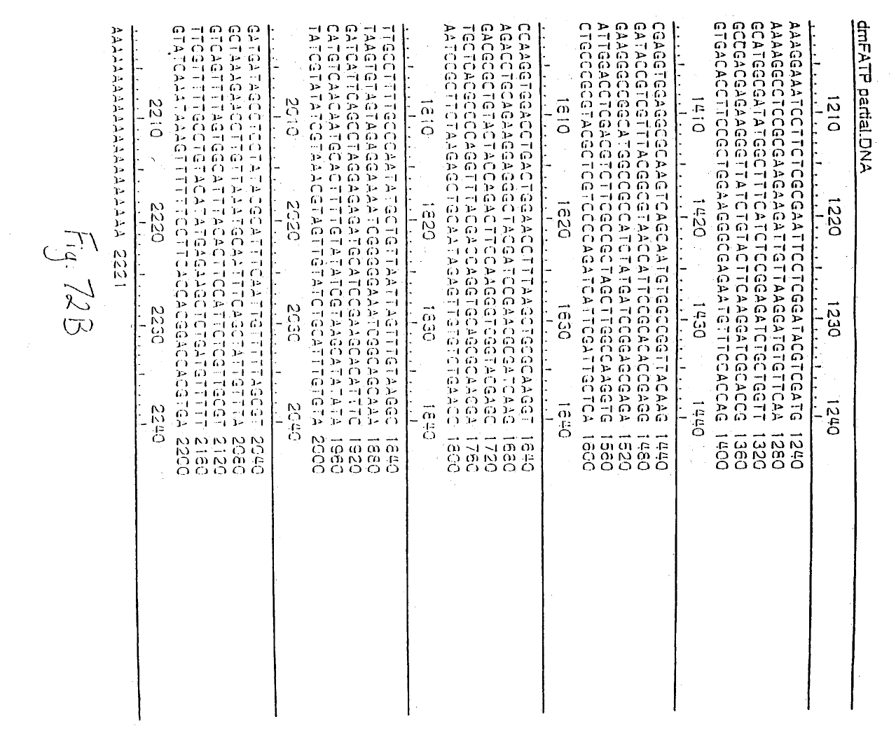

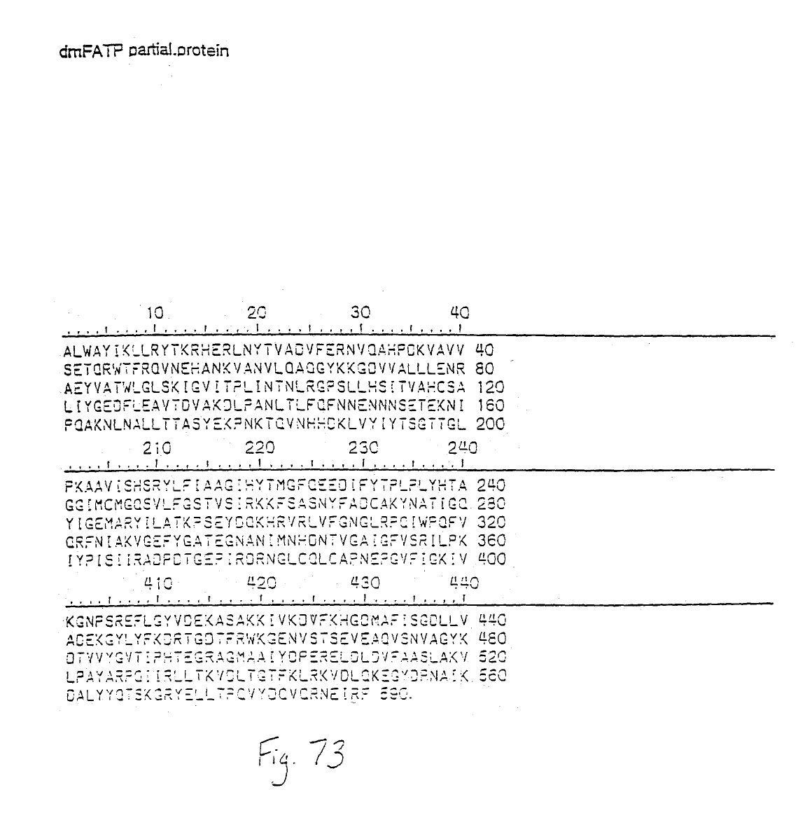

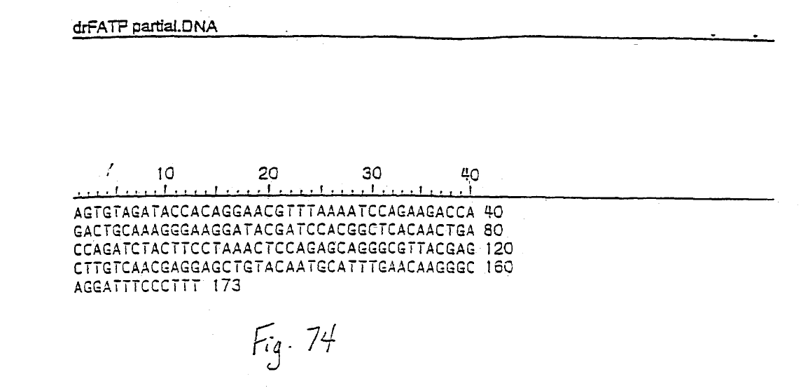

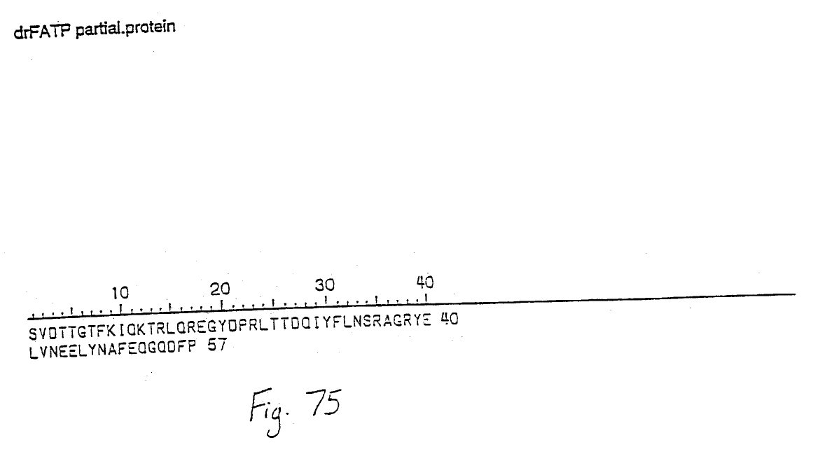

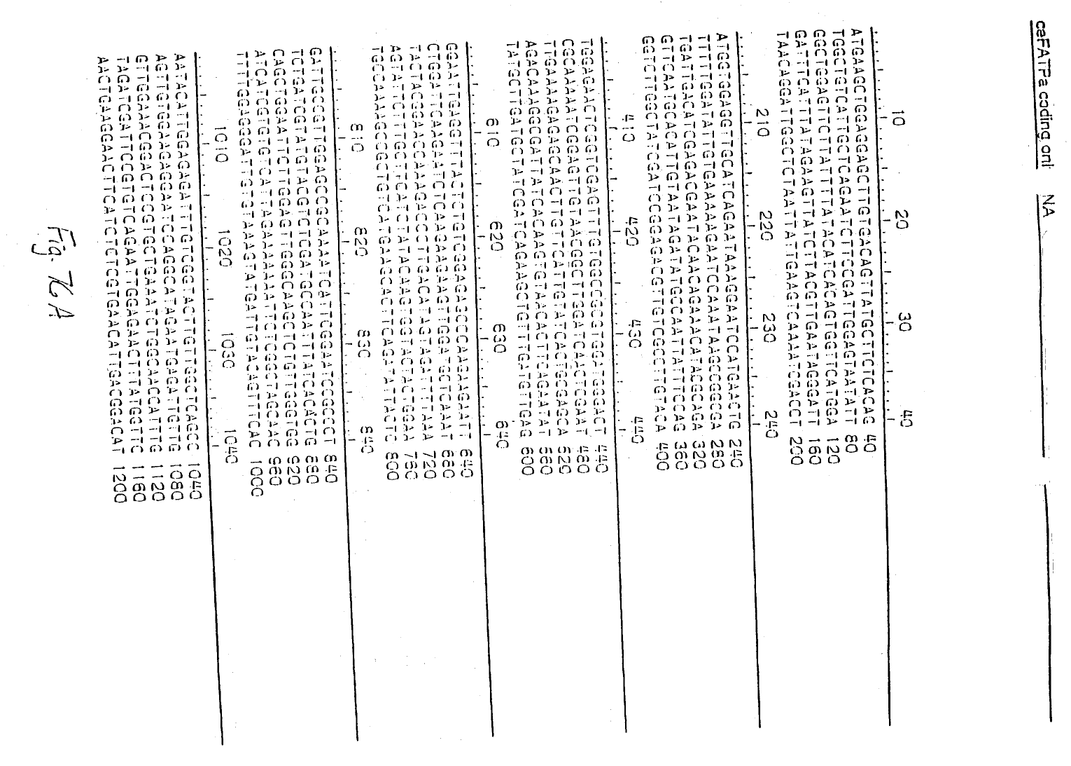

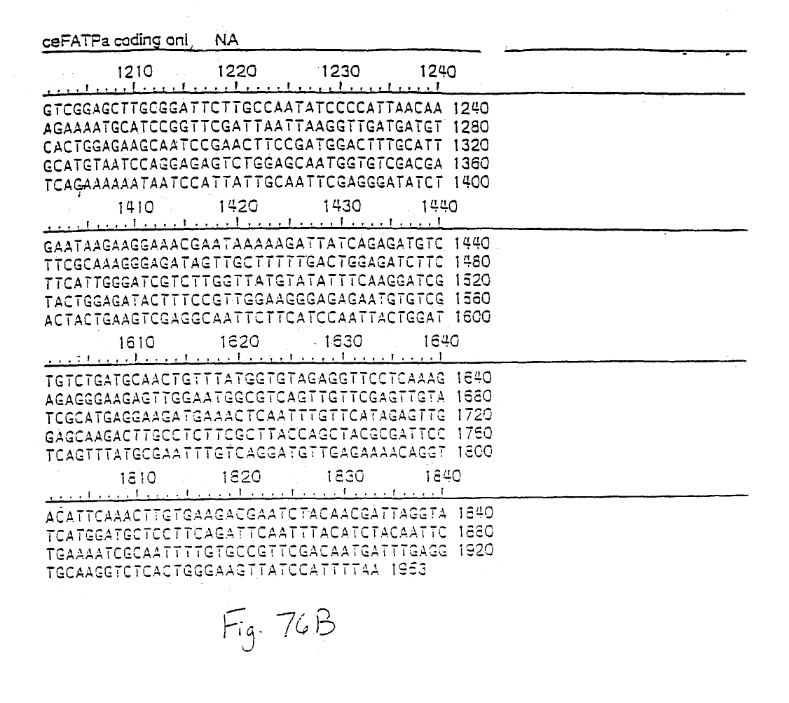

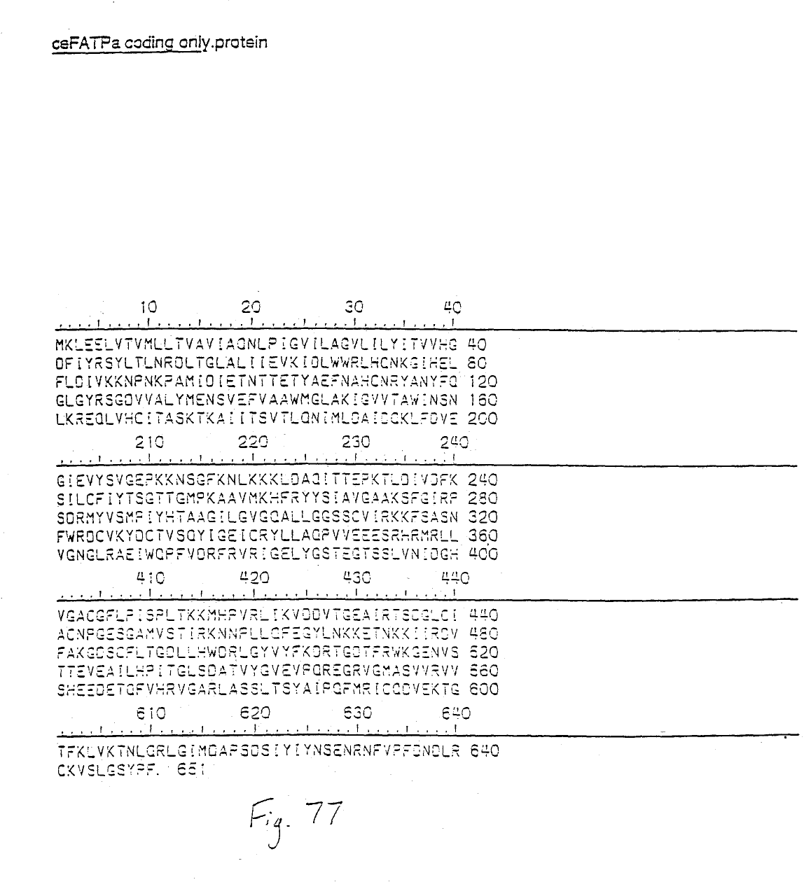

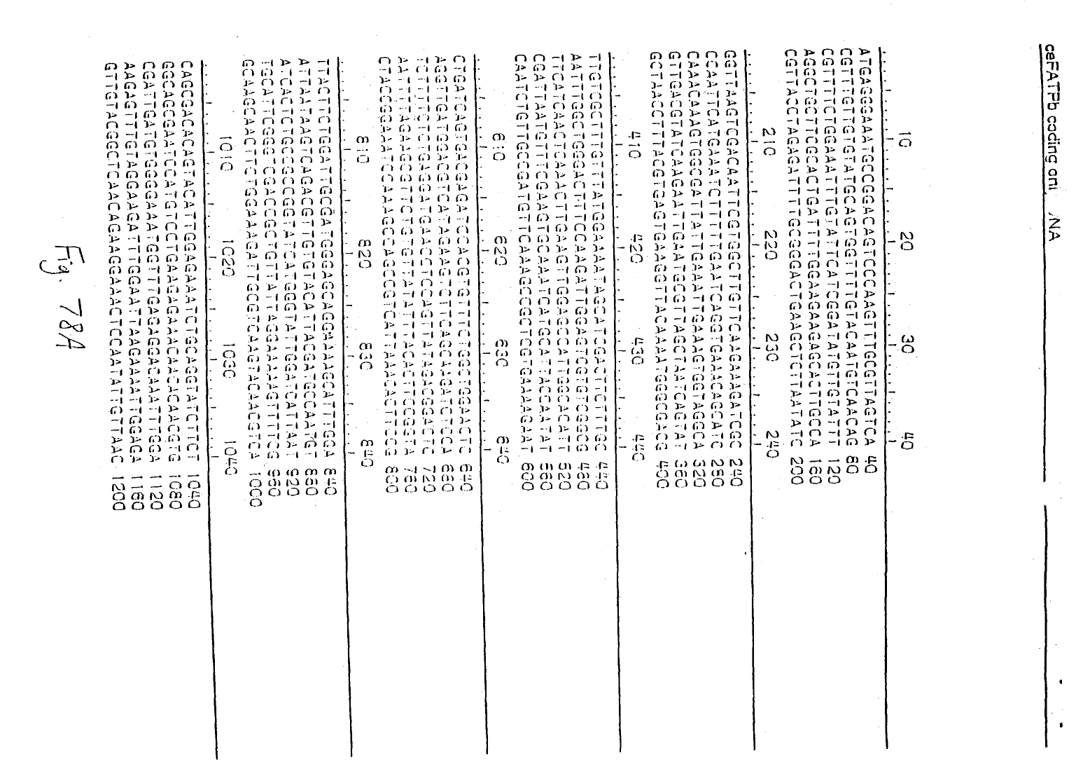

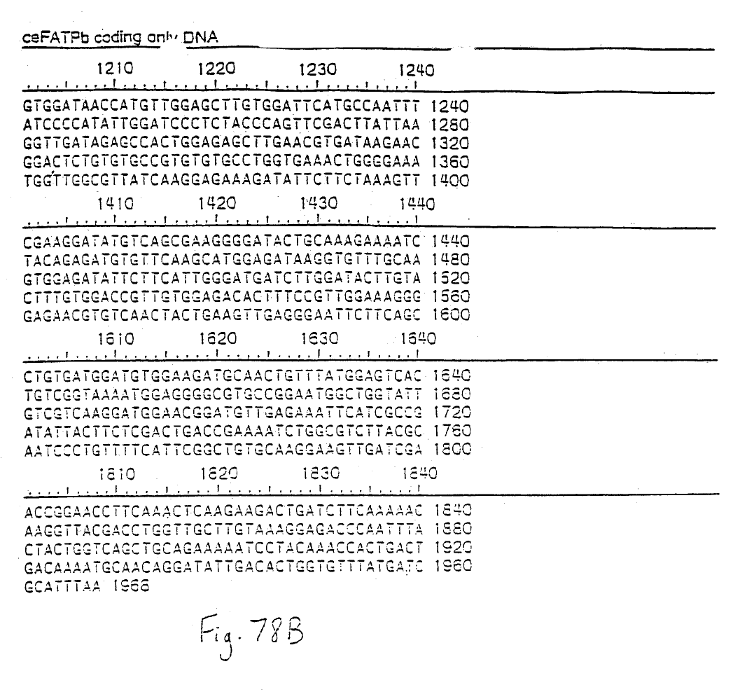

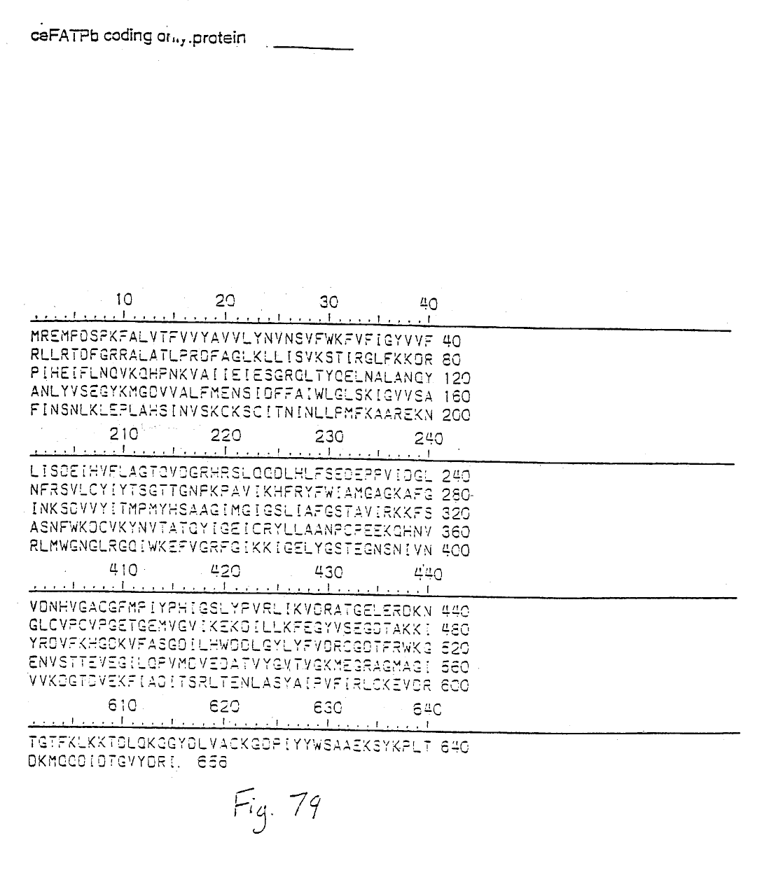

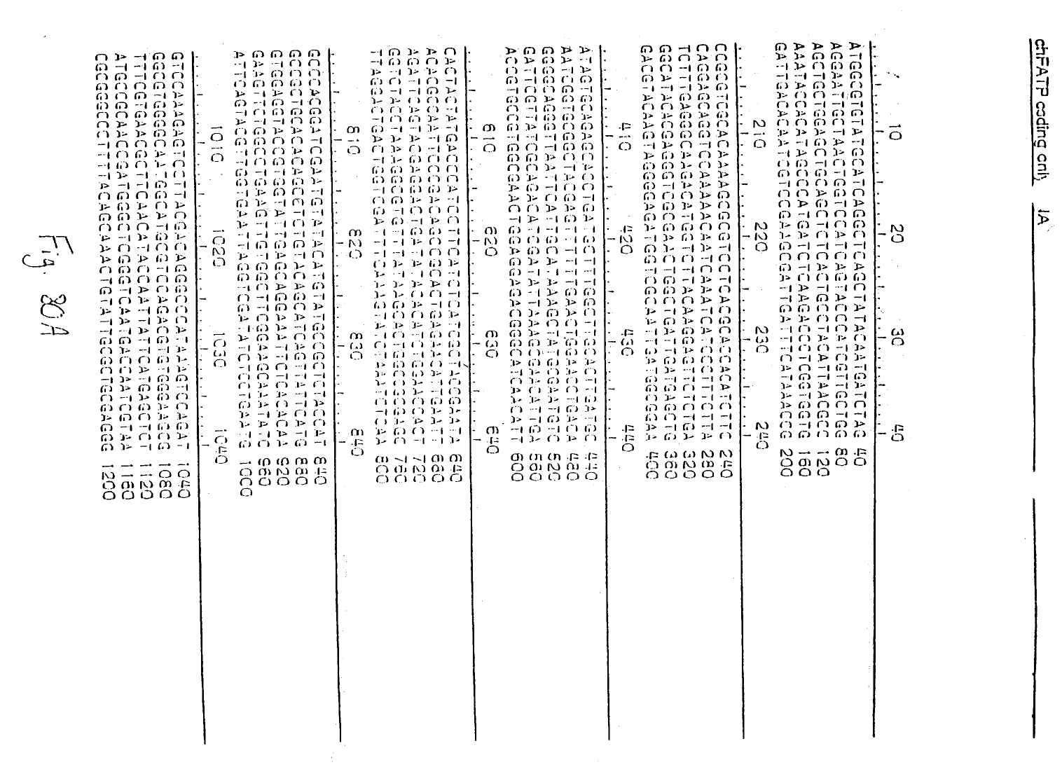

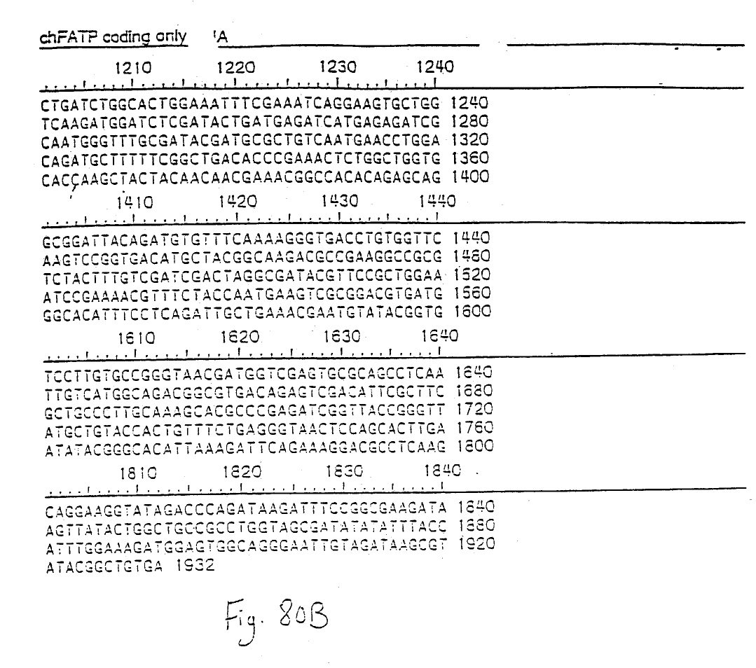

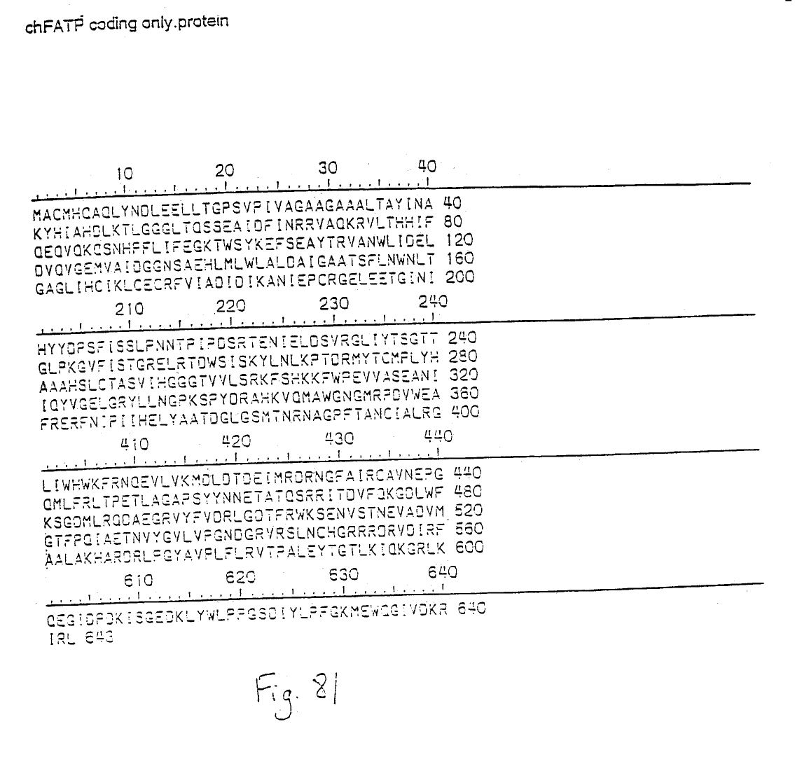

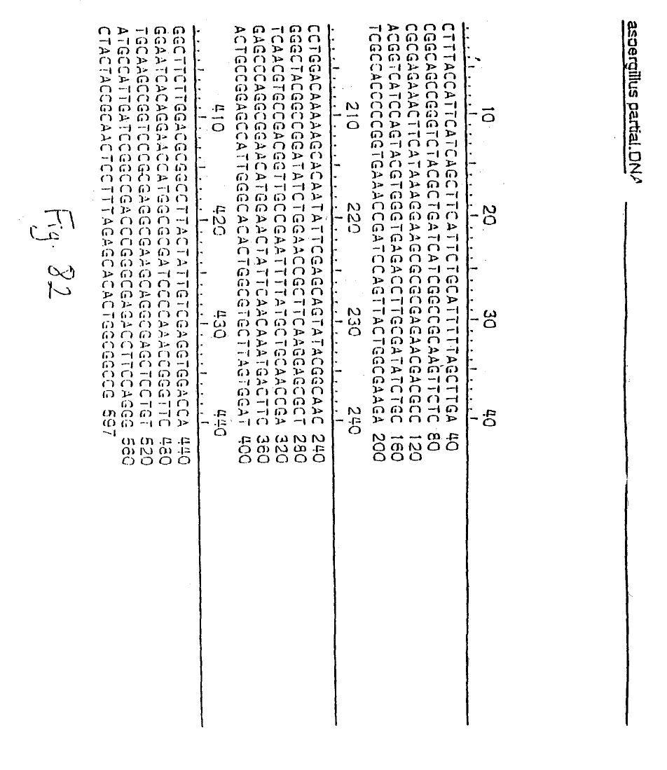

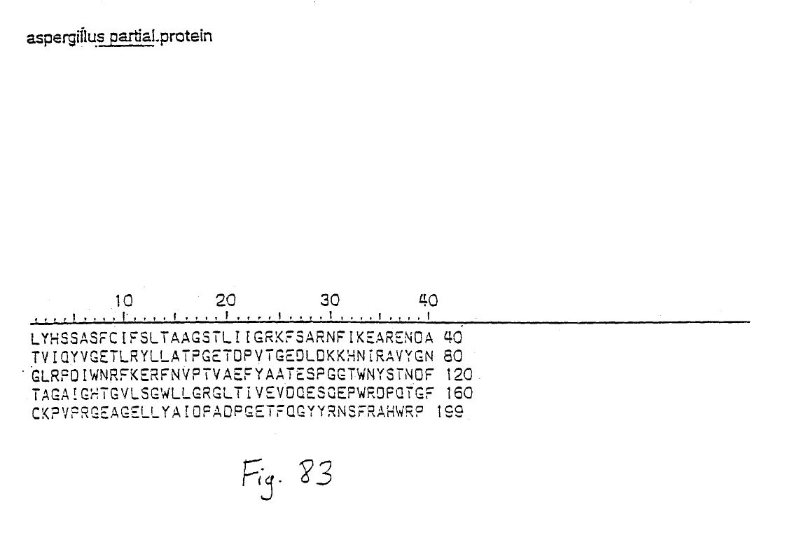

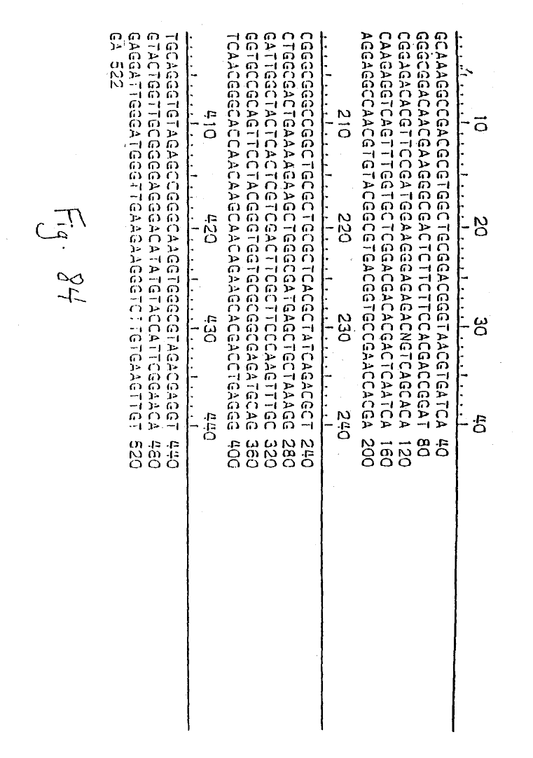

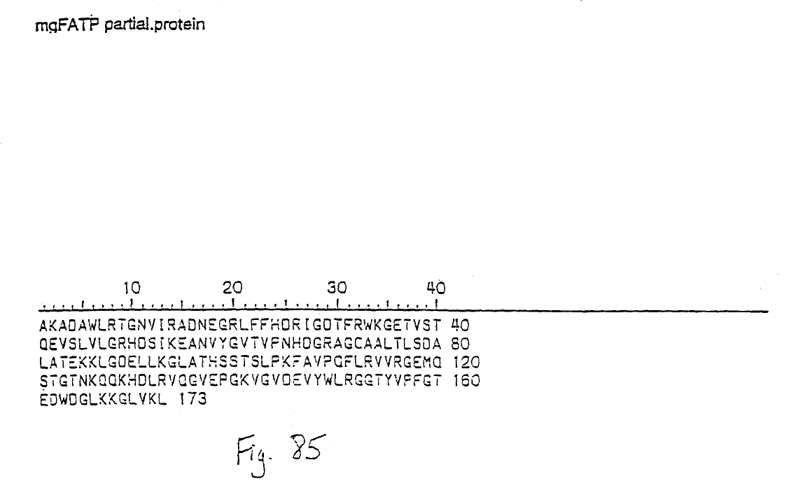

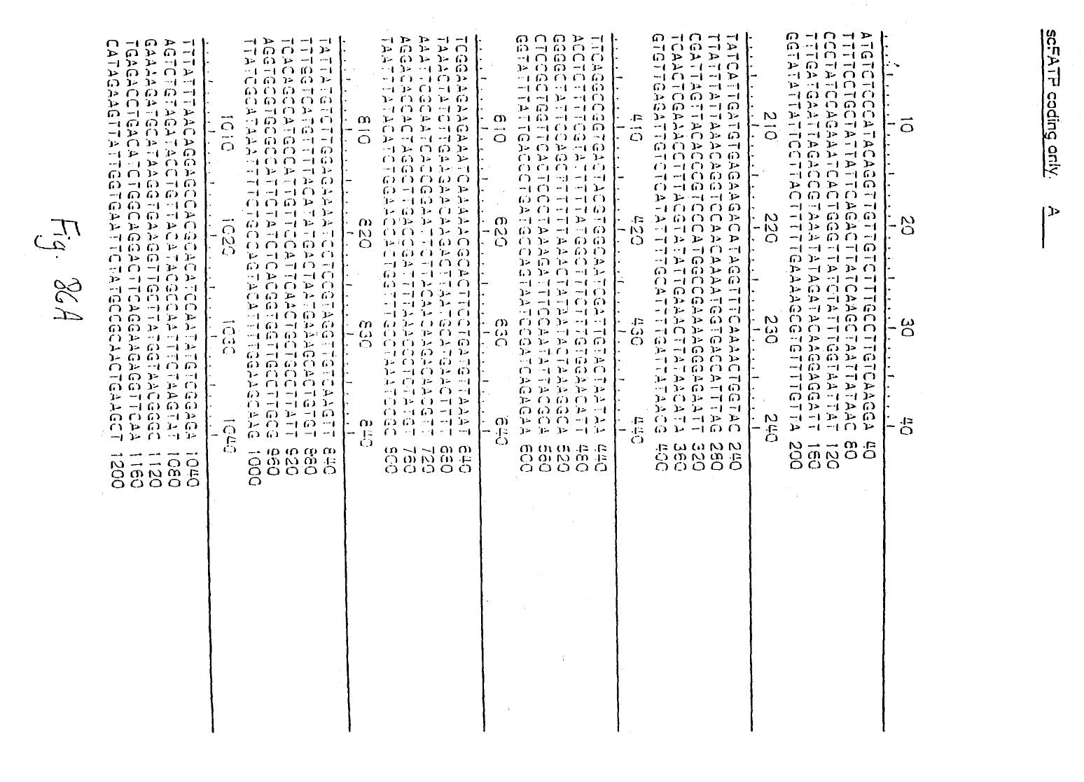

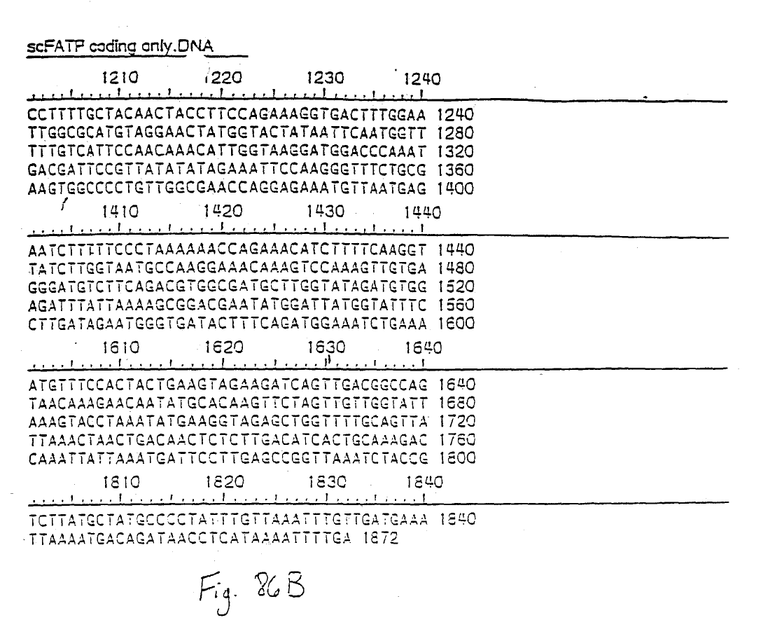

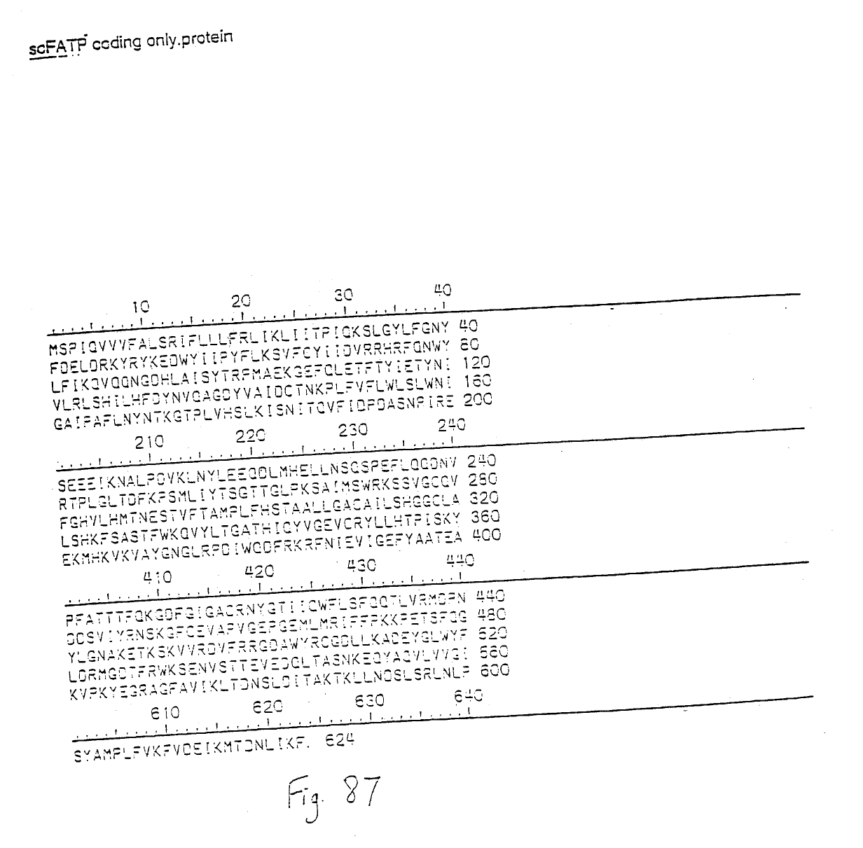

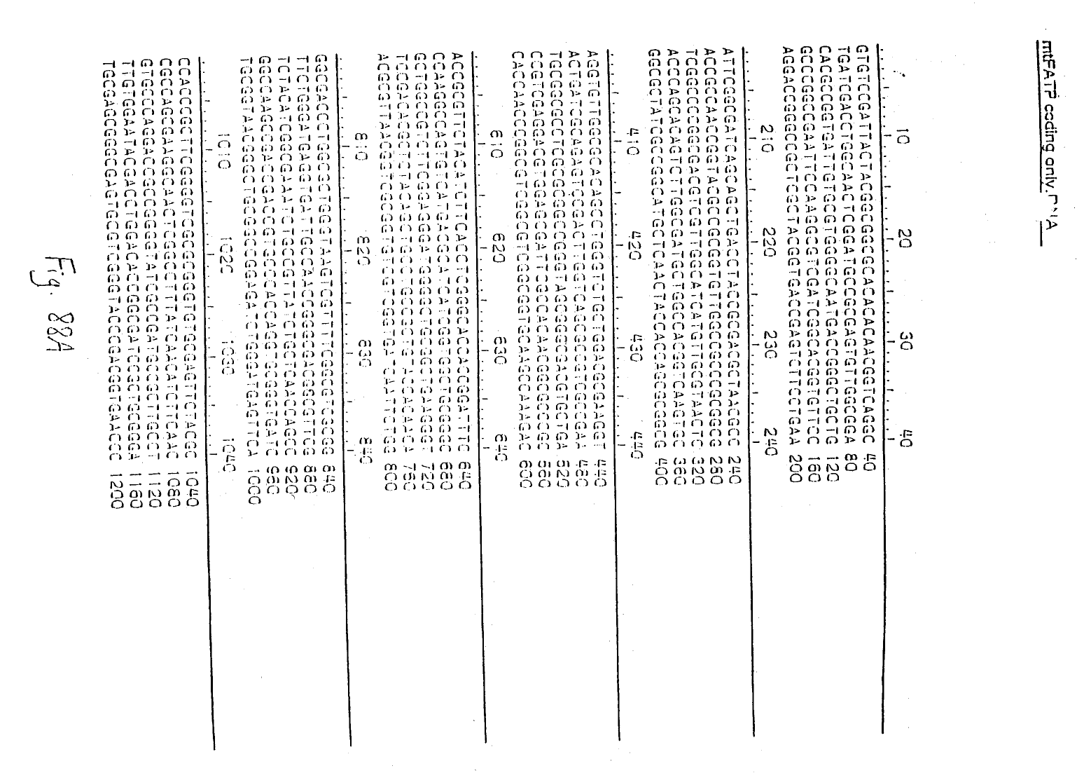

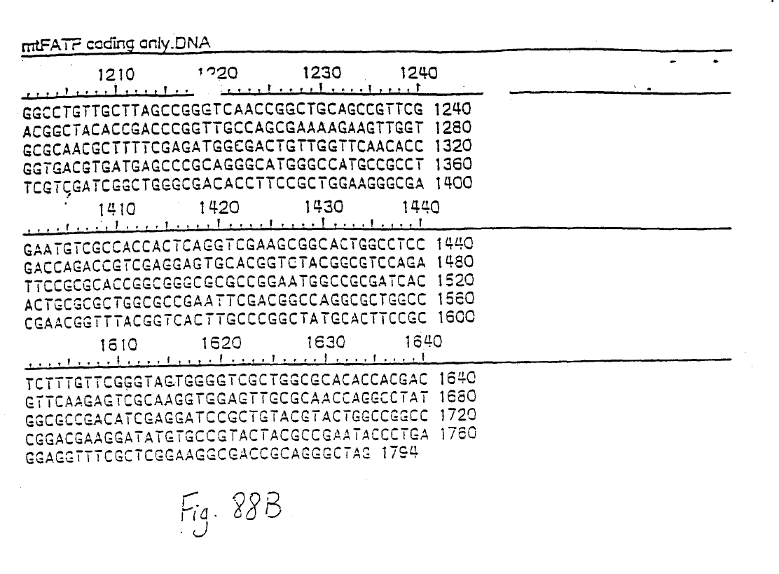

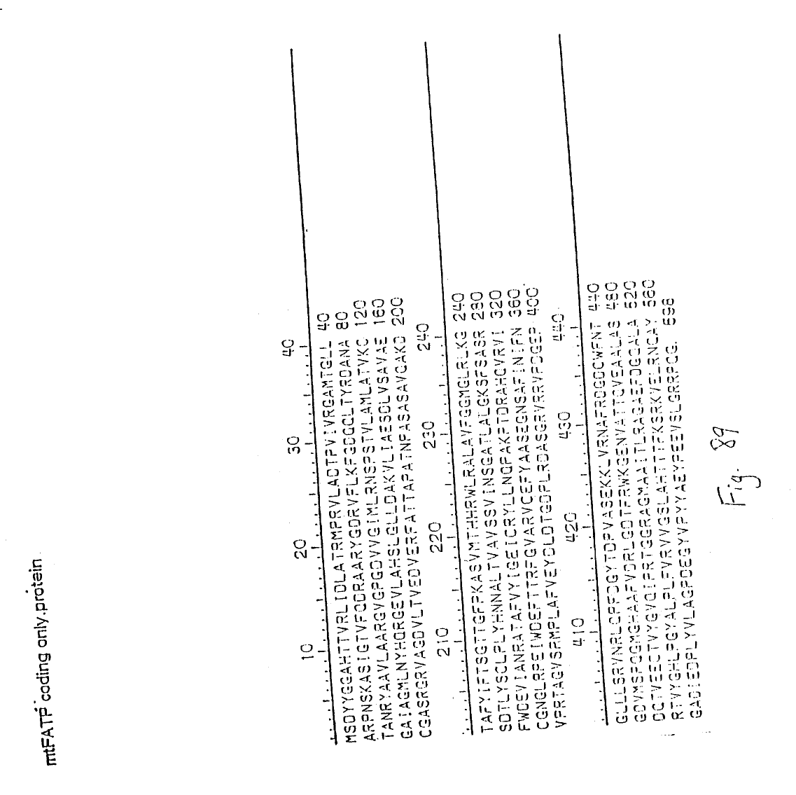

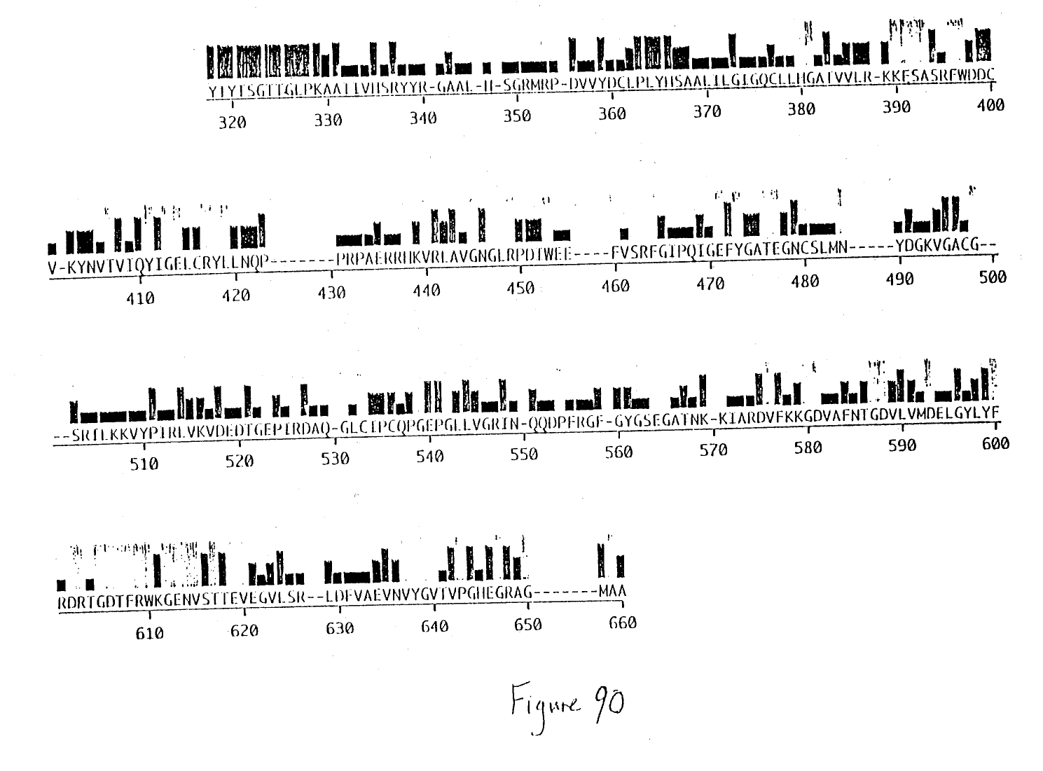

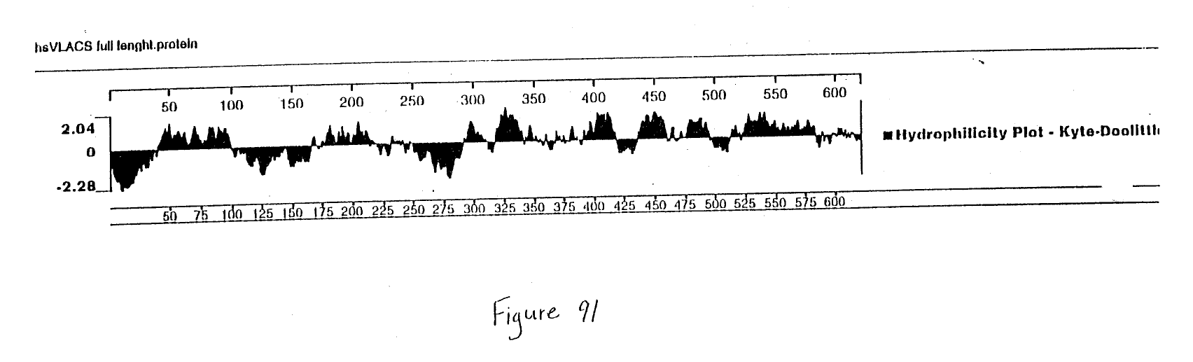

One aspect of the invention relates to isolated nucleic acids that encode a FATP as described herein, such as those FATPs having an amino acid sequence in FIG. 45 (SEQ ID NO:47), FIG. 47 (SEQ ID NO:49), FIG. 112 (SEQ ID NO: 117), FIG. 51 (SEQ ID NO:53), FIG. 53 (SEQ ID NO:55), and FIG. 55 (SEQ ID NO:57) and nucleic acids closely related thereto as described herein.

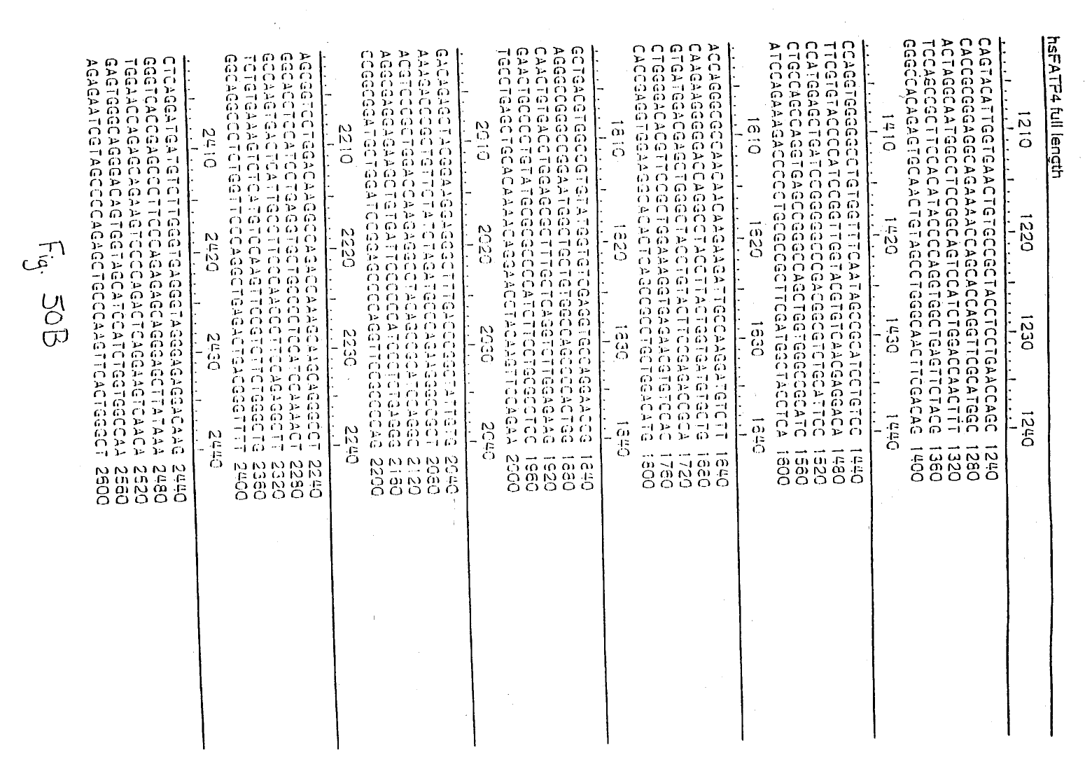

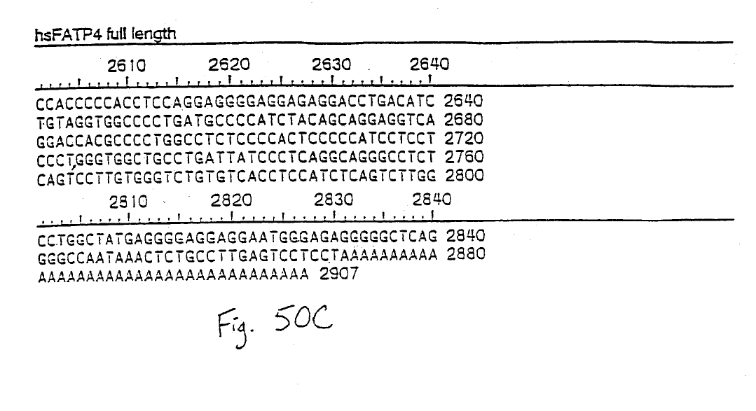

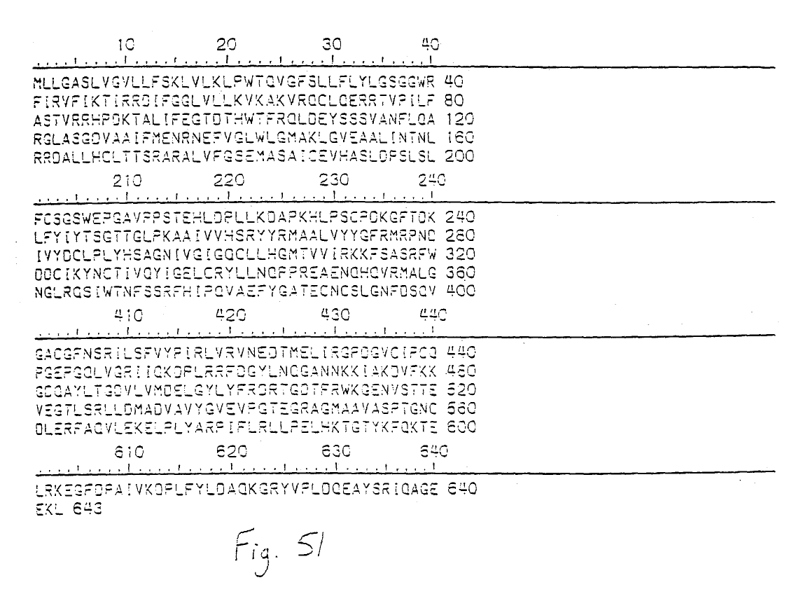

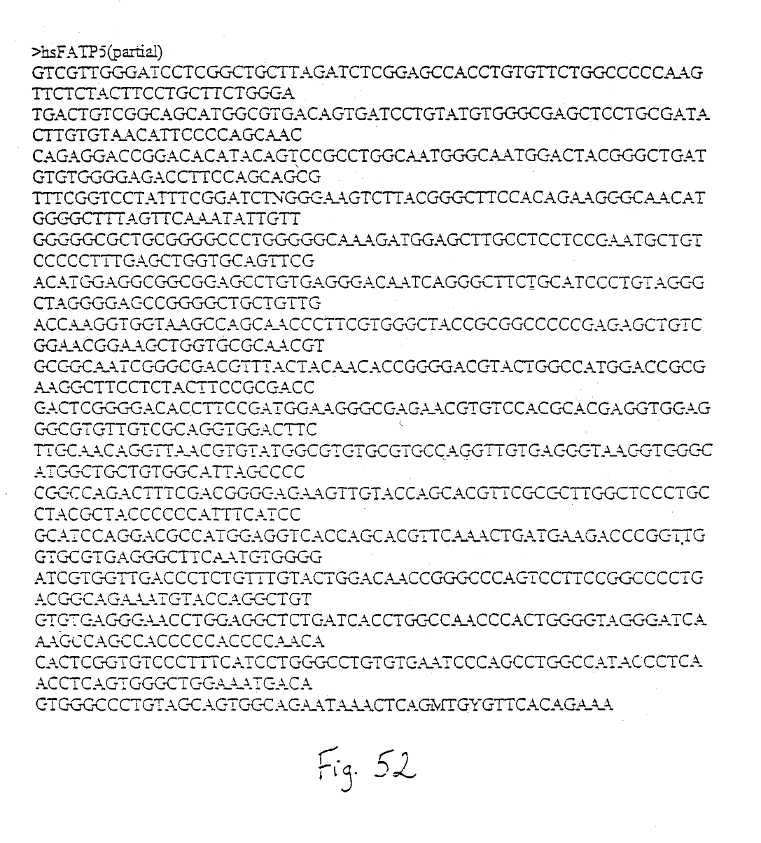

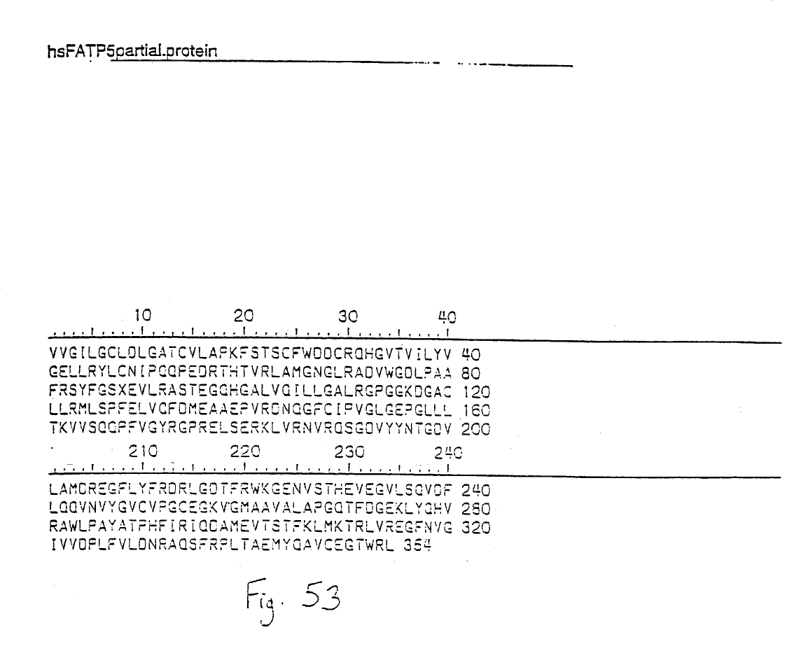

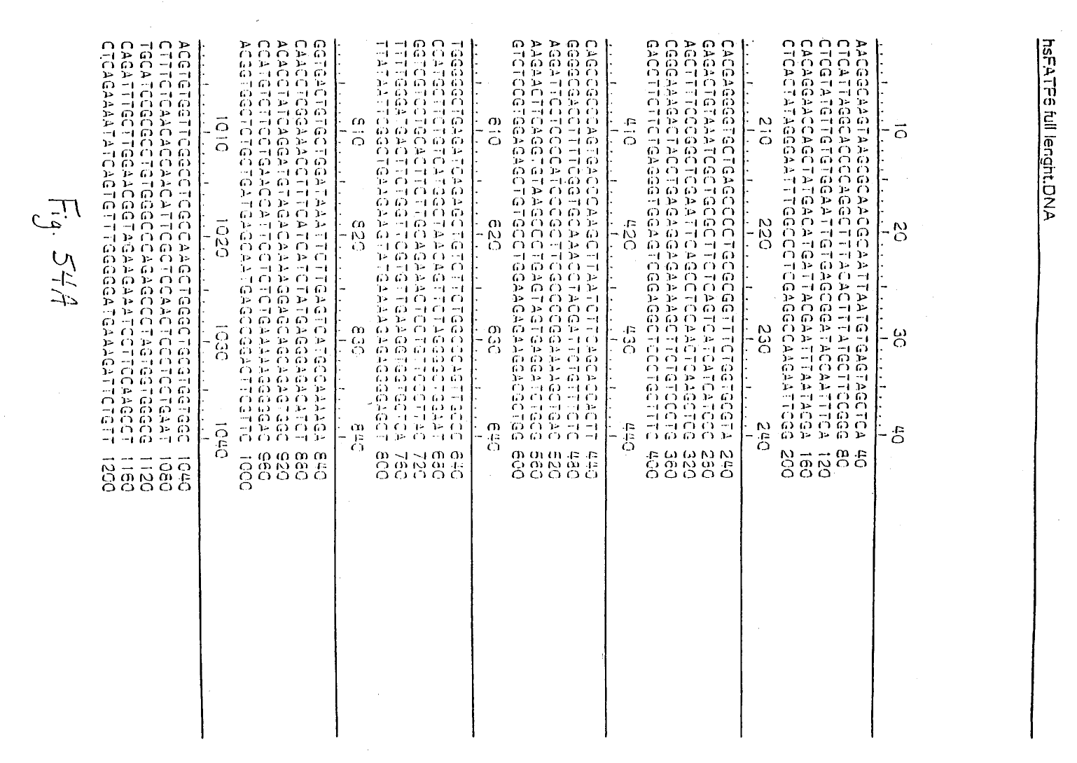

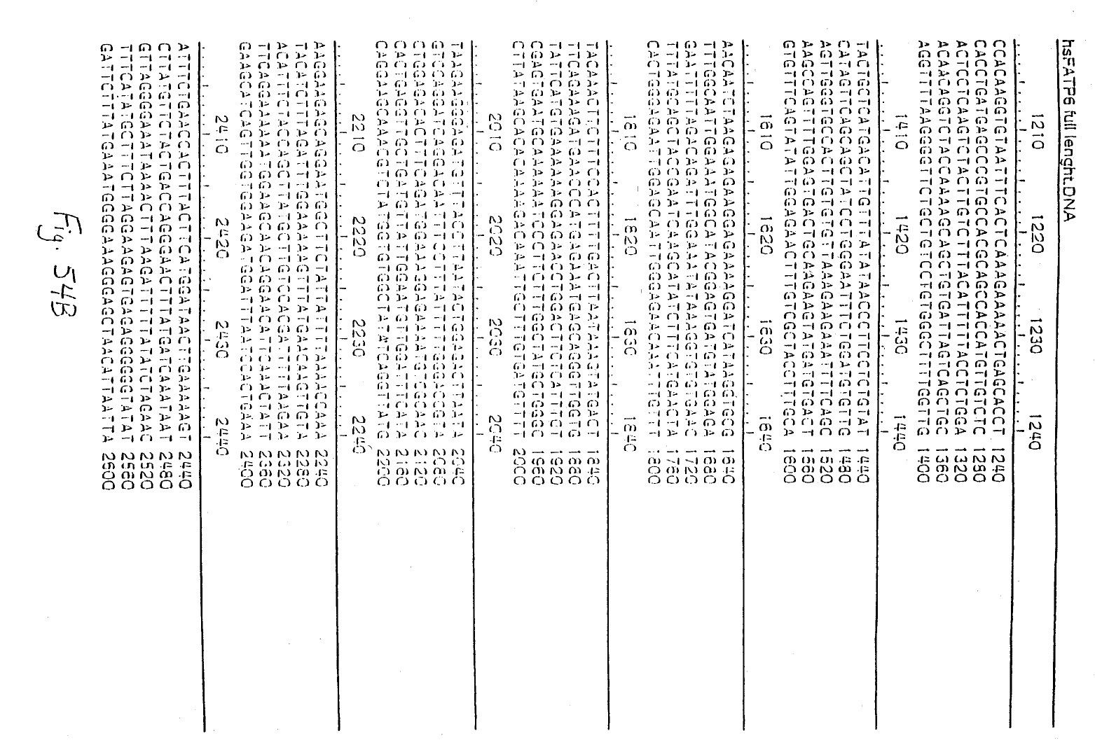

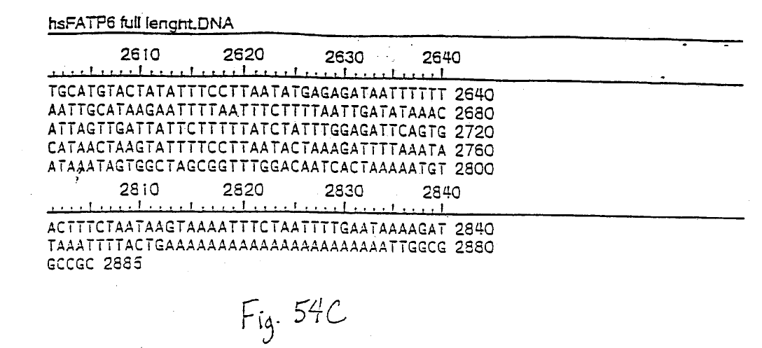

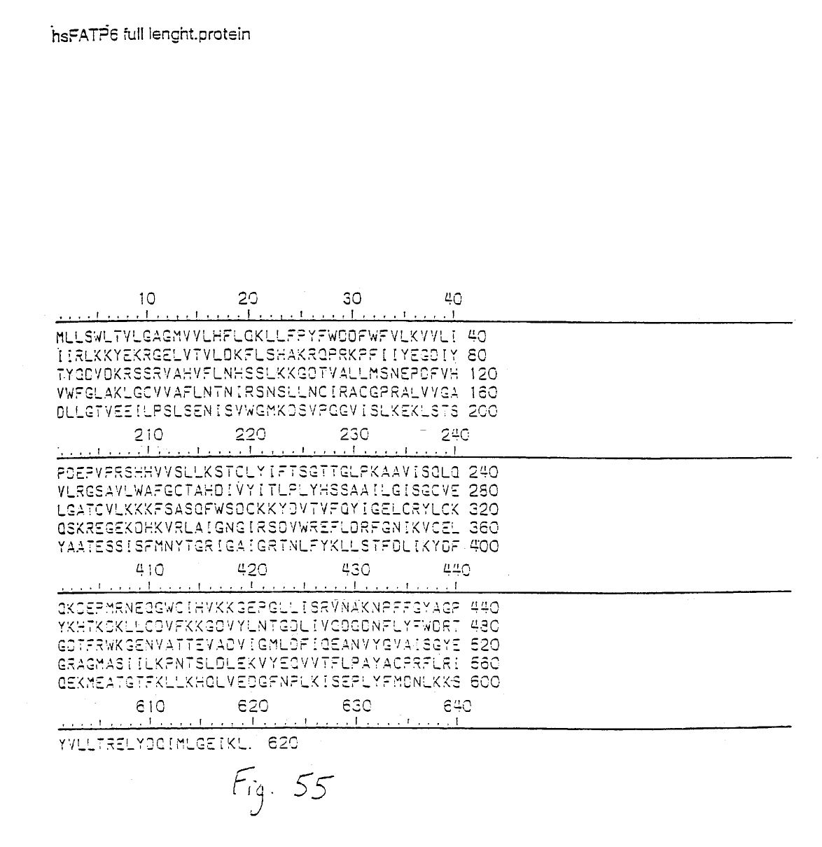

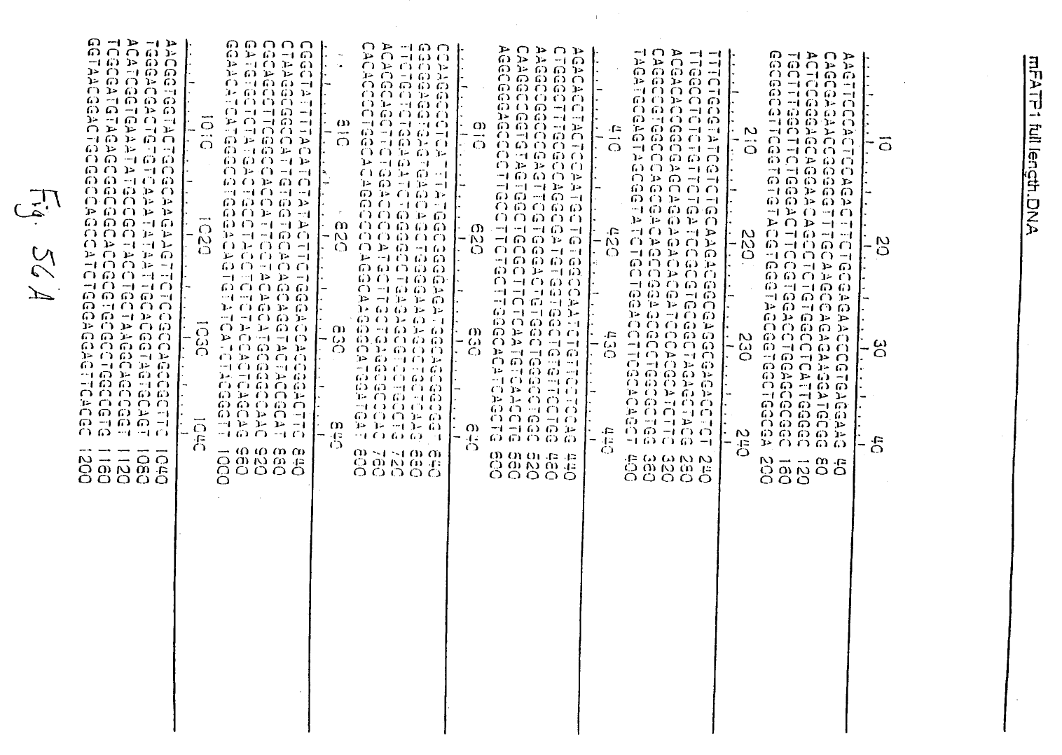

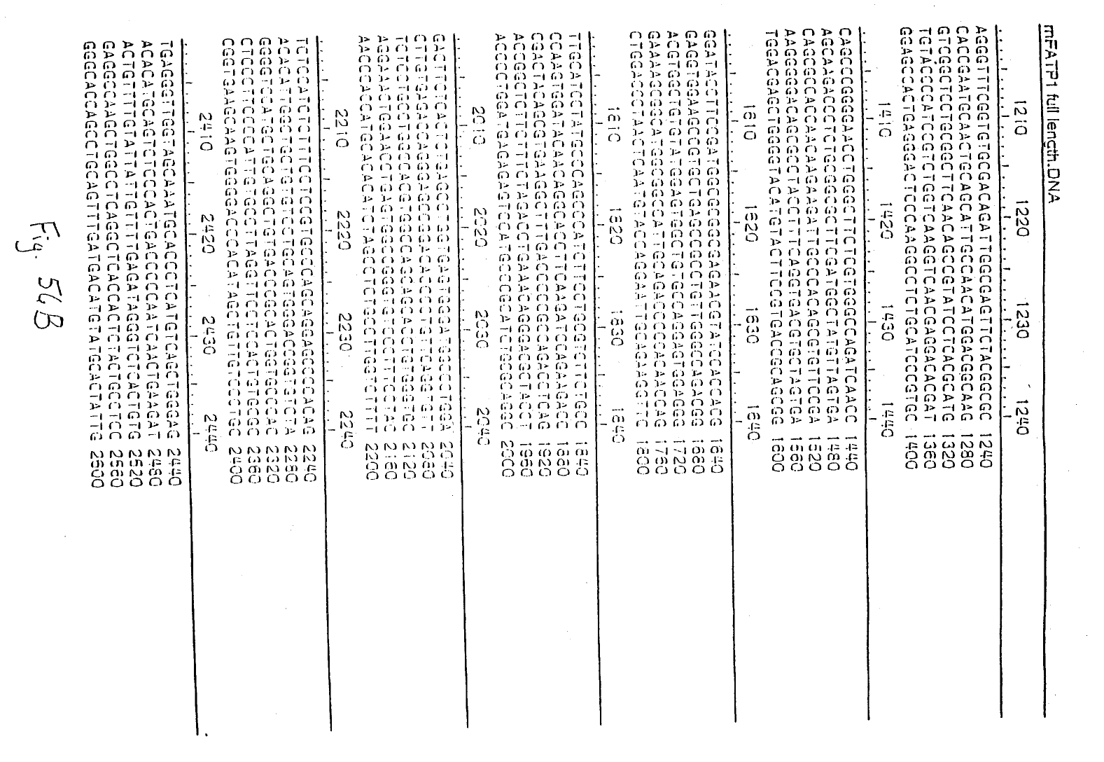

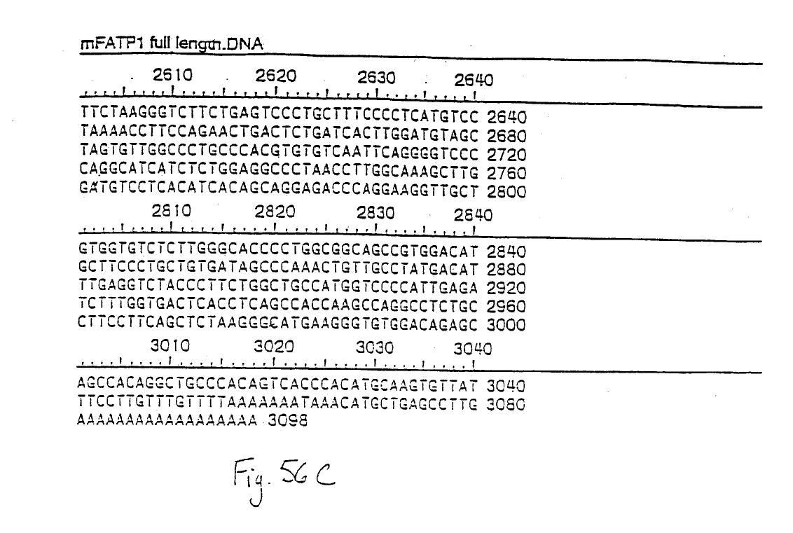

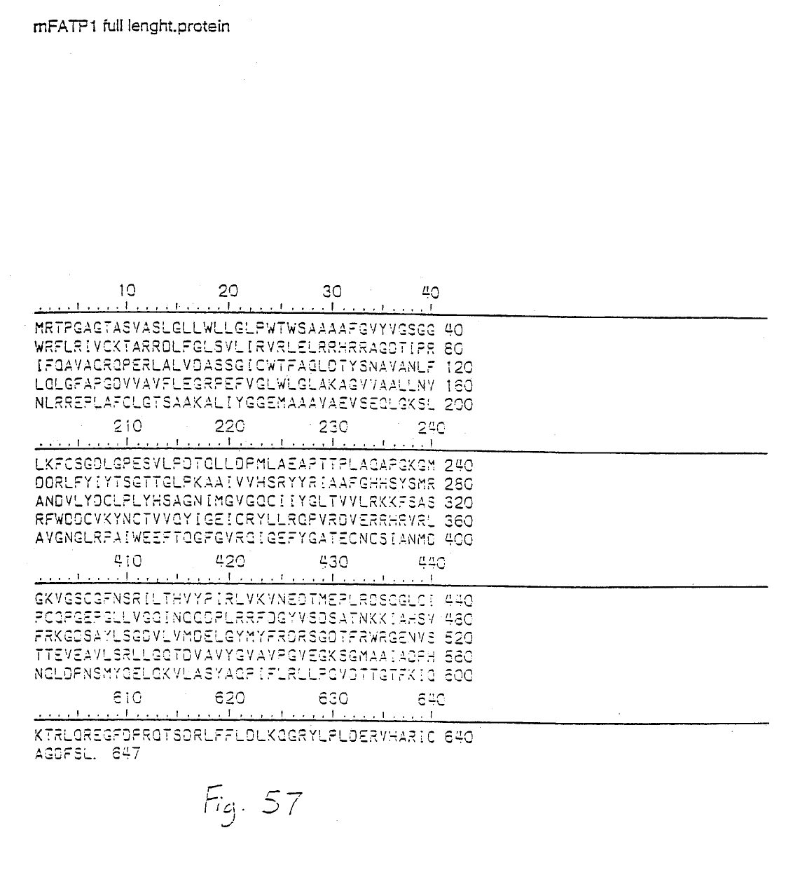

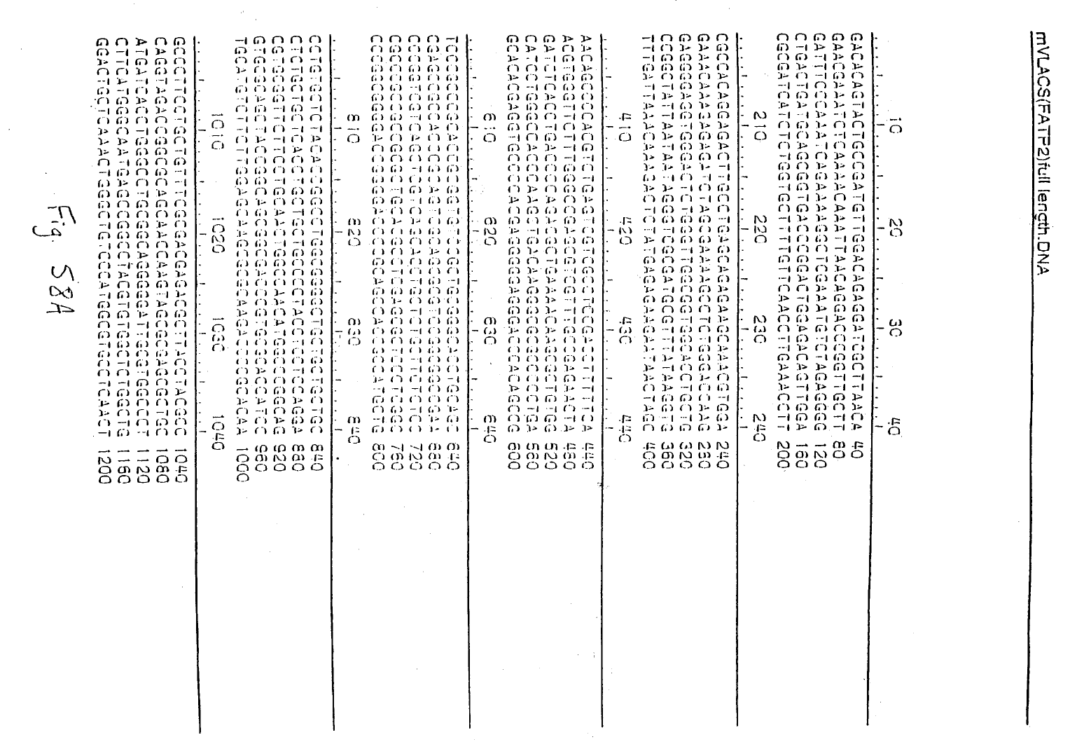

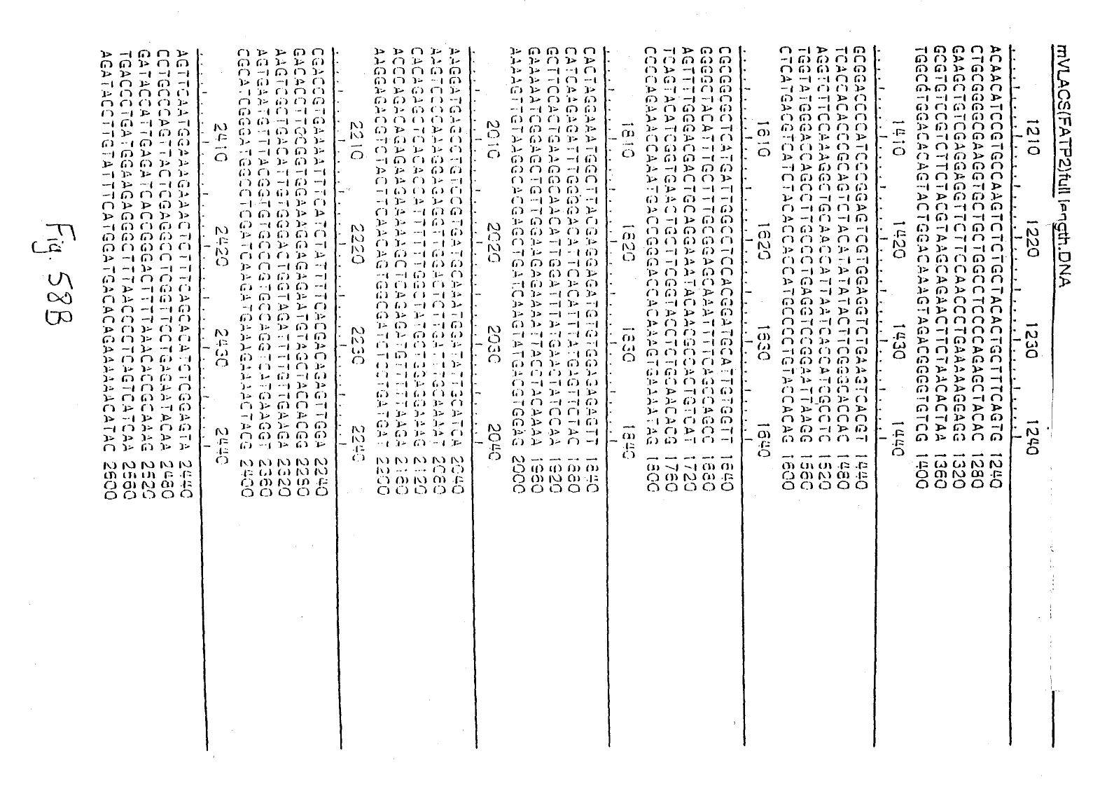

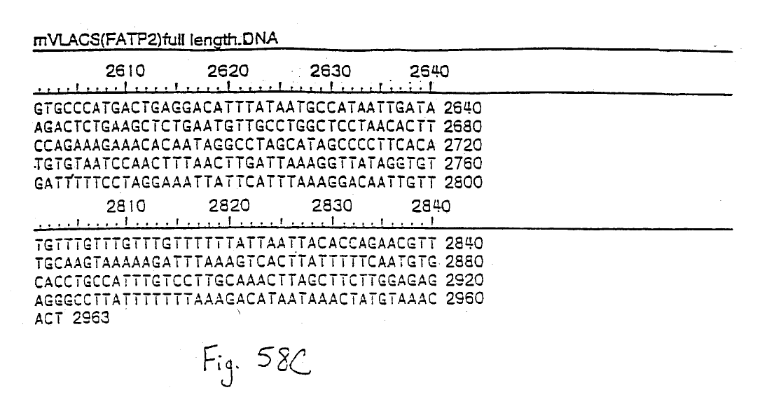

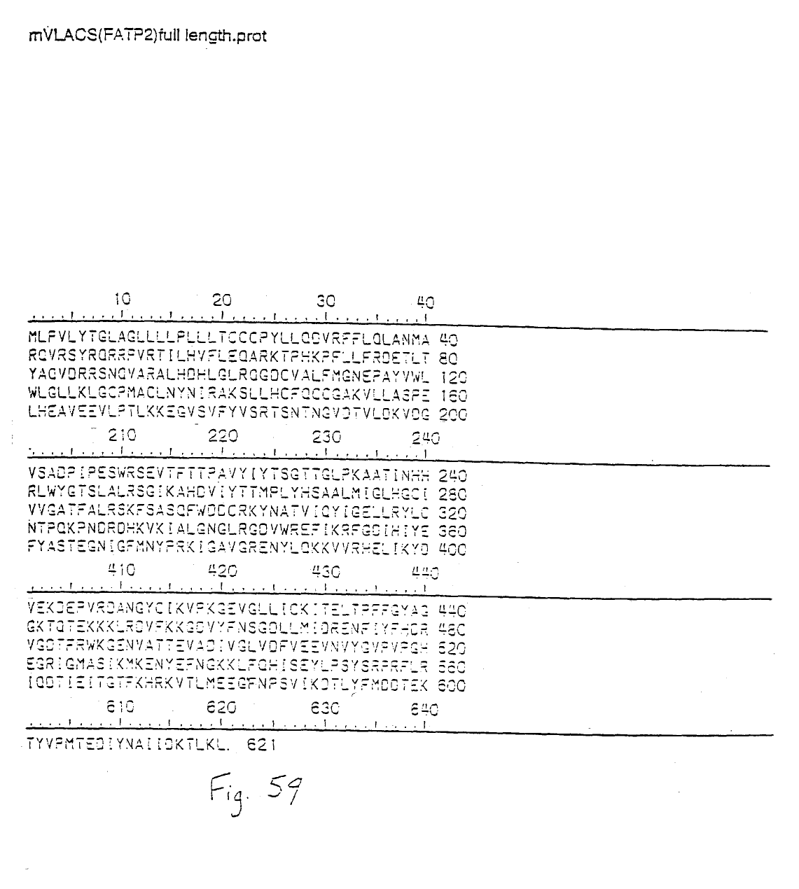

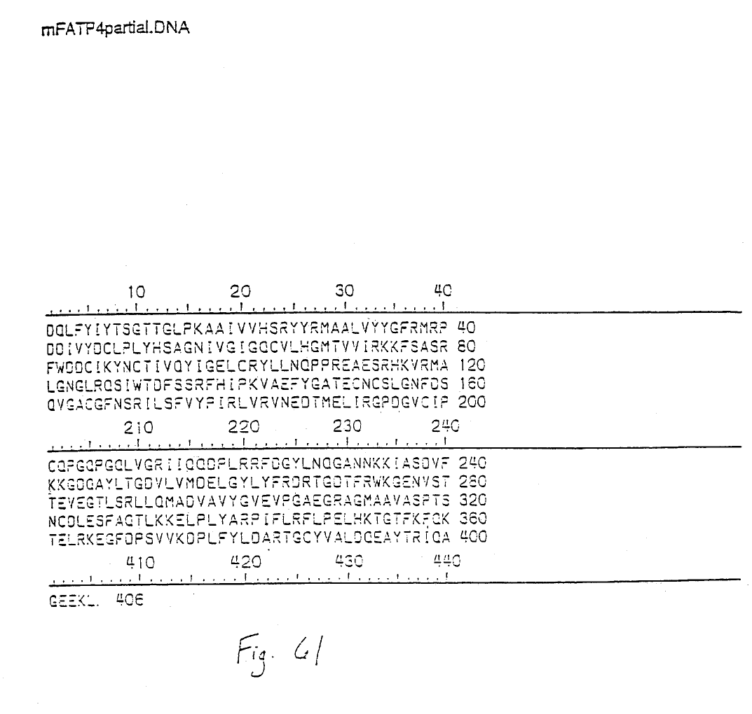

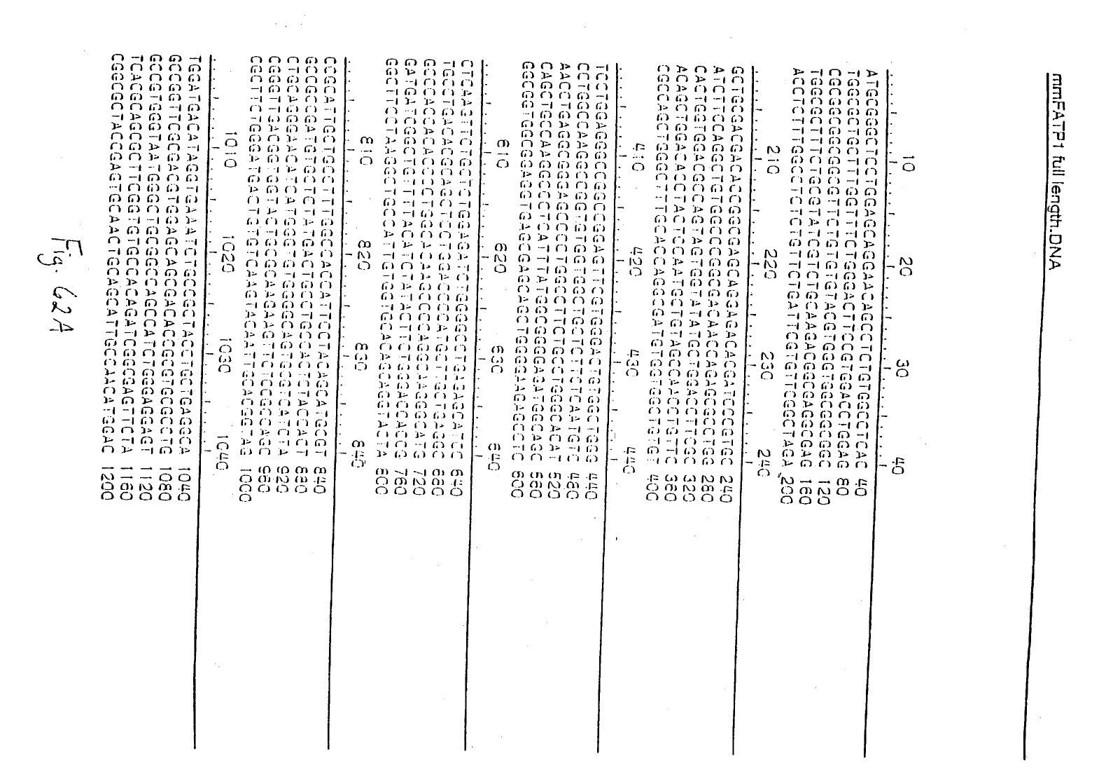

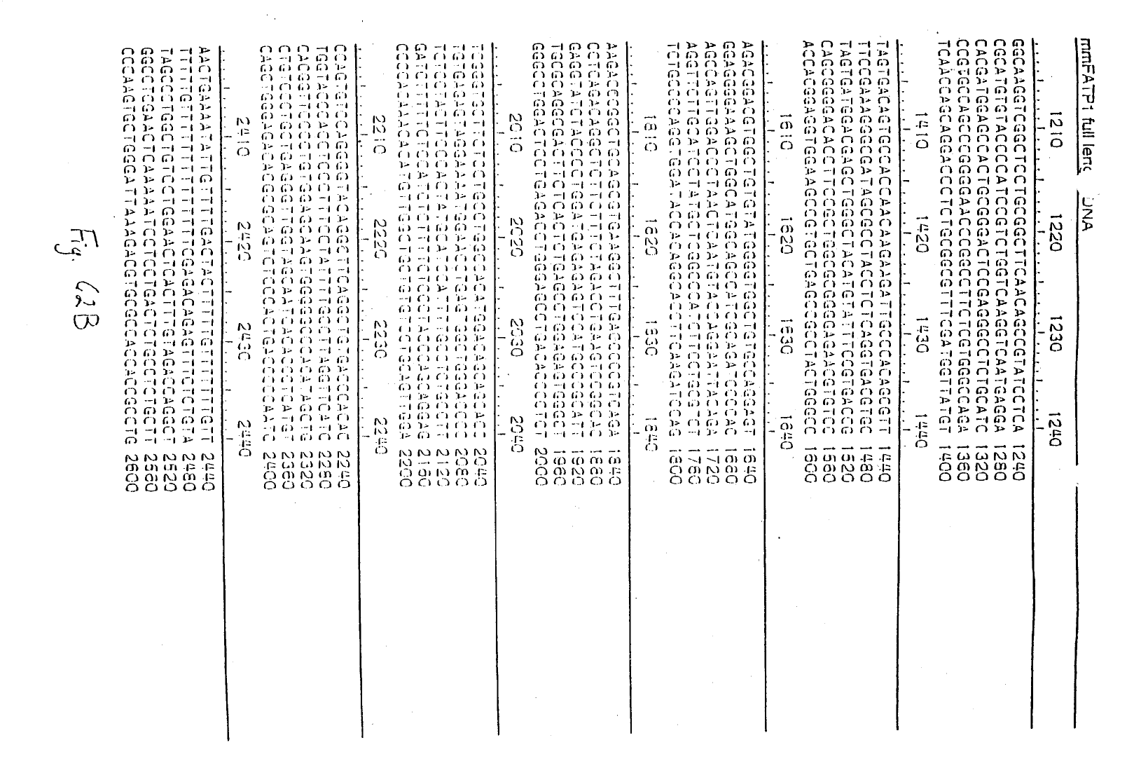

Using the information provided herein, such as a nucleic acid sequence set forth in FIGS. 44A-44C (SEQ ID NO:46), FIGS. 46A and 46B (SEQ ID NO:48), FIG. 112 (SEQ ID NO:116), FIGS. 50A-50C (SEQ ID NO:52), FIG. 52 (SEQ ID NO:54), and FIGS. 54A-54C (SEQ ID NO:56), a nucleic acid of the invention encoding a FATP polypeptide has been obtained using standard cloning and screening methods, such as those for cloning and sequencing cDNA library fragments, followed by obtaining a full length clone. For example, to obtain a nucleic acid of the invention, a library of clones of cDNA of human or other mammalian DNA can be probed with a labeled oligonucleotide, such as a radiolabeled oligonucleotide, preferably about 17 nucleotides or longer, derived from a partial sequence. Clones carrying DNA identical to that of the probe can then be distinguished using stringent (also, “high stringency”) hybridization conditions. By sequencing the individual clones thus identified with sequencing primers designed from the original sequence it is then possible to extend the sequence in both directions to determine the full length sequence. Suitable techniques are described, for example, in Current Protocols in Molecular Biology (F. M. Ausubel et al, eds), containing supplements through Supplement 42, 1998, John Wiley and Sons, Inc., especially chapters 5, 6 and 7.

Embodiments of the invention include isolated nucleic acid molecules comprising any of the following nucleotide sequences: 1.) a nucleotide sequence which encodes a protein comprising the amino acid sequence of hsFATP1 (SEQ ID NO:47), the amino acid sequence of hsFATP2 (SEQ ID NO:49), the amino acid sequence of hsFATP3 (SEQ ID NO:117), the amino acid sequence of hsFATP4 (SEQ ID NO: 53), the amino acid sequence of hsFATP5 (SEQ ID NO:55) or the amino acid sequence of hsFATP6 (SEQ ID NO:57); 2.) nucleotide sequences of hsFATP1, hsFATP2, hsFATP3, hsFATP4, hsFATP5, or hsFATP6 (SEQ ID NO:46, 48, 116, 52, 54, or 56, respectively); 3.) a nucleotide sequence which is complementary to the nucleotide sequence of hsFATP1 (SEQ ID NO:46), hsFATP2 (SEQ ID NO:48), hsFATP3 (SEQ ID NO:116), hsFATP4 (SEQ ID NO:52), hsFATP5 (SEQ ID NO:54) or hsFATP6 (SEQ ID NO:56); 4.) a nucleotide sequence which consists of the coding region of hsFATP1 (SEQ ID NO:46), the coding region of hsFATP2 (SEQ ID NO:48), the coding region of hsFATP3 (SEQ ID NO:116), the coding region of hsFATP4 (SEQ ID NO:52), the coding region of hsFATP5 (SEQ ID NO:54), or the coding region of hsFATP6 (SEQ ID NO:56).

The invention further relates to nucleic acids (nucleic acid molecules or polynucleotides) having nucleotide sequences identical over their entire length to those shown in the figures, for instance FIGS. 44A-44C (SEQ ID NO:46), FIGS. 46A and 46B (SEQ ID NO:48), FIGS. 111A-B (SEQ ID NO:116), FIGS. 50A-50C (SEQ ID NO:52), FIG. 52 (SEQ ID NO:54), and FIGS. 54A-54C (SEQ ID NO:56). It further relates to DNA, which due to the degeneracy of the genetic code, encodes a FATP encoded by one of the FATP-encoding DNAs, whose amino acid sequence is provided herein. Also provided by the invention are nucleic acids having the coding sequences for the mature polypeptides or fragments in reading frame with other coding sequences, such as those encoding a leader or secretory sequence, a pre-, or pro- or prepro-protein sequence. The nucleic acids of the invention encompass nucleic acids that include a single continuous region or discontinuous regions encoding the polypeptide, together with additional regions, that may also contain coding or non-coding sequences. The nucleic acids may also contain non-coding sequences, including, for example, but not limited to, non-coding 5′ and 3′ sequences, such as the transcribed, non-translated sequences, termination signals, ribosome binding sites, sequences that stabilize mRNA, introns, polyadenylation signals, and additional coding sequences which encode additional amino acids. For example, a marker sequence that facilitates purification of the fused polypeptide can be encoded. In certain embodiments of the invention, the marker sequence can be a hexa-histidine peptide, as provided in the pQE vector (Qiagen, Inc., Venlo, The Netherlands) and described in Gentz et al., Proc. Natl. Acad. Sci. USA 86: 821-824 (1989), or an HA tag (Wilson et al., Cell 37: 767 (1984)), or a sequence encoding glutathione S-transferase of Schistosoma japonicum (vectors available from Pharmacia; see Smith, D. B. and Johnson K. S., Gene 67:31 (1988) and Kaelin, W. G. et al., Cell 70:351 (1992)). Nucleic acids of the invention also include, but are not limited to, nucleic acids comprising a structural gene and its naturally associated sequences that control gene expression.

The invention further relates to nucleic acids (nucleic acid molecules or polynucleotides) that encode a FATP polypeptide. In a particular embodiment, a nucleic acid encodes a portion of a FATP which includes a motif or domain, for example, a lipocalin domain or an AMP-binding domain. Such a polypeptide portion can be a functional portion of a FATP protein. The term “lipocalin domain” is an art recognized term and as used herein refers to a particular domain present in FATP proteins. This domain is described as including regions of sequence homology as well as a common tertiary structure represented as an eight stranded antiparallel beta-barrel. (see Banaszak, L. et al., Advances in Protein Chemistry, 45: 89-151). Many lipocalin domains can be identified structurally as a sequence contained within the general formula: [DENG]-X-[DENQGSTARK]-X(0,2)-[DENQARK]-[LIVFY]-{CP}-G-{C}-W-[FYWLRH-X]-[LIVMTA], e.g., the lipocalin signature sequence or consensus pattern (SEQ ID NO: 125). One skilled in the art will recognize that a lipocalin domain for a particular FATP protein can vary in sequence from this general formula. A FATP lipocalin domain can be, for example, identical to the lipocalin signature sequence or can exhibit 60, 65, 70, 75, 80, 85, 90, 95 or greater per cent sequence identity compared to the general formula provided that it retains lipocalin binding function. For example, a lipocalin domain for each of the human FATPs, hsFATP1 (SEQ ID NO:126), hsFATP2 (SEQ ID NO:127), hsFATP3 (SEQ ID NO:128), hsFATP4 (SEQ ID NO:129), hsFATP5 (SEQ ID NO: 130), and hsFATP6 (SEQ ID NO:131) has been identified. The pattern of these lipocalin domains are highly conserved across the FATP family.

A nucleic acid encoding a portion of a FATP polypeptide can encode one or more domains, and also can include additional nucleotides. For example, the nucleic acid can also include nucleotide sequences that encode a portion of a FATP protein that is upstream from a lipocalin domain. As the term “upstream” or “upstream sequences” is used herein in relation to the lipocalin domain, it is intended to refer to the nucleotide sequence which encodes all or a portion of a FATP protein located between the signal peptide (when one is present) and the lipocalin domain. In the absence of a signal peptide, the term refers to the nucleotide sequence which encodes all or a portion of a FATP protein between the lipocalin domain and the amino terminus (see FIG. 115).

The invention further relates to variants, including naturally-occurring allelic variants, of those nucleic acids described specifically herein by DNA sequence, that encode variants of such polypeptides as those having the amino acid sequences shown in FIG. 45 (SEQ ID NO:47), FIG. 47 (SEQ ID NO:49), FIG. 112 (SEQ ID NO: 117), FIG. 51 (SEQ ID NO:53) FIG. 53 (SEQ ID NO:55), or FIG. 55 (SEQ ID NO:57). Such variants include nucleic acids encoding variants of the above-listed amino acid sequences, wherein those variants have several, such as 5 to 10, 1 to 5, or 3, 2 or 1 amino acids substituted, deleted, or added, in any combination. Variants include polynucleotides encoding polypeptides with at least 95% but less than 100% amino acid sequence identity to the polypeptides described herein by amino acid sequence. Variant polynucleotides hybridize, under low to high stringency conditions, to the alleles described herein by DNA sequence. In one embodiment, variants have silent substitutions, additions and deletions that do not alter the properties and activities of the FATP. Allelic variants of the polynucleotides encoding hsFATP1 (FIG. 45; SEQ ID NO:47), hsFATP2 (FIG. 47; SEQ ID NO:49), hsFATP3 (FIG. 112; SEQ ID NO:117), hsFATP4 (FIG. 51; SEQ ID NO:53), hsFATP5 (FIG. 53; SEQ ID NO:55) and hsFATP6 (FIG. 55; SEQ ID NO:57) will be identified as mapping to chromosomal locations listed for the corresponding wild type genes in Table 2 in Example 1.

Orthologous genes are gene loci in different species that are sufficiently similar to each other in their nucleotide sequences to suggest that they originated from a common ancestral gene. Orthologous genes arise when a lineage splits into two species, rather than when a gene is duplicated within a genome. Proteins that are orthologs are encoded by genes of two different species, wherein the genes are said to be orthologous.

The invention further relates to polynucleotides encoding polypeptides which are orthologous to those polypeptides having a specific amino acid sequence described herein, such as the amino acid sequences shown in FIG. 45 (SEQ ID NO:47), FIG. 47 (SEQ ID NO:49), FIG. 112 (SEQ ID NO: 117), FIG. 51 (SEQ ID NO:53), FIG. 53 (SEQ ID NO:55), or FIG. 55 (SEQ ID NO:57). These polynucleotides, which can be called ortholog polynucleotides, encode orthologous polypeptides that can range in amino acid sequence identity to a reference amino acid sequence described herein, from about 65% to less than 100%, but preferably 70% to 80%, more preferably 80% to 90%, and still more preferably 90% to less than 100%. Orthologous polypeptides can also be those polypeptides that range in amino acid sequence similarity to a reference amino acid sequence described herein from about 75% to 100%, within the signature sequence. The amino acid sequence similarity between the signature sequences of orthologous polypeptides is preferably 80%, more preferably 90%, and still more preferably, 95%. The ortholog polynucleotides encode polypeptides that have similar functional characteristics (e.g., fatty acid transport activity) and similar tissue distribution, as appropriate to the organism from which the ortholog polynucleotides can be isolated.

Ortholog polynucleotides can be isolated from (e.g., by cloning or nucleic acid amplification methods) a great number of species, as shown by the sample of FATPs from evolutionarily divergent species described herein (see, e.g., FIGS. 44A-C through FIG. 89). Ortholog polynucleotides corresponding to those in FIG. 45 (SEQ ID NO:47), FIG. 47 (SEQ ID NO:49), FIGS. 111A-B (SEQ ID NO:116), FIG. 51 (SEQ ID NO:53), FIG. 52 (SEQ ID NO:55) and FIG. 55 (SEQ ID NO:57) are those which can be isolated from mammals such as rat, dog, chimpanzee, monkey, baboon, pig, rabbit and guinea pig, for example.

Further variants that are fragments of the nucleic acids of the invention may be used to synthesize full-length nucleic acids of the invention, such as by use as primers in a polymerase chain reaction. As used herein, the term primer refers to a single-stranded oligonucleotide which acts as a point of initiation of template-directed DNA synthesis under appropriate conditions (e.g., in the presence of four different nucleoside triphosphates and an agent for polymerization, such as DNA or RNA polymerase or reverse transcriptase) in an appropriate buffer and at a suitable temperature. The appropriate length of a primer depends on the intended use of the primer, but typically ranges from 15 to 30 nucleotides. Short primer molecules generally require cooler temperatures to form sufficiently stable hybrid complexes with the template. A primer need not reflect the exact sequence of the template, but must be sufficiently complementary to hybridize with a template. The term primer site refers to the area of the target DNA to which a primer hybridizes. The term primer pair refers to a set of primers including a 5′ (upstream) primer that hybridizes with the 5′ end of the DNA sequence to be amplified and a 3′ (downstream) primer that hybridizes with the complement of the 3′ end of the sequence to be amplified.

Further embodiments of the invention are nucleic acids that are at least 80% identical over their entire length to a nucleic acid described herein, for example a nucleic acid having the nucleotide sequence in FIGS. 44A-44C (SEQ ID NO:46), FIGS. 46A-46B (SEQ ID NO:48), FIGS. 111A-B (SEQ ID NO:116), FIGS. 50A-50C (SEQ ID NO:52), FIG. 52 (SEQ ID NO:54), and FIGS. 54A-54C (SEQ ID NO:56). Additional embodiments are nucleic acids, and the complements of such nucleic acids, having at least 90% nucleotide sequence identity to the above-described sequences, and nucleic acids having at least 95% nucleotide sequence identity. In preferred embodiments, DNA of the present invention has 97% nucleotide sequence identity, 98% nucleotide sequence identity, or at least 99% nucleotide sequence identity with the DNA whose sequences are presented herein.

Other embodiments of the invention are nucleic acids that are at least 80% identical in nucleotide sequence to a nucleic acid encoding a polypeptide having an amino acid sequence as set forth in FIG. 45 (SEQ ID NO:47), FIG. 47 (SEQ ID NO:49), FIG. 112 (SEQ ID NO:1 17), FIG. 51 (SEQ ID NO:53), FIG. 53 (SEQ ID NO:55) or FIG. 55 (SEQ ID NO:57), or as such amino acid sequences are set forth elsewhere herein, and nucleic acids that are complementary to such nucleic acids. Specific embodiments are nucleic acids having at least 90% nucleotide sequence identity to a nucleic acid encoding a polypeptide having an amino acid sequence as described in the list above, nucleic acids having at least 95% sequence identity, and nucleic acids having at least 97% sequence identity.

The terms “complementary” or “complementarity” as used herein, refer to the natural binding of polynucleotides under permissive salt and temperature conditions by base-pairing. Complementarity between two single-stranded molecules may be “partial” in which only some of the nucleic acids bind, or it may be complete when total complementarity exists between the single-stranded molecules (that is, when A-T and G-C base pairing is 100% complete). The degree of complementarity between nucleic acid strands has significant effects on the efficiency and strength of hybridization between nucleic acid strands. This is of particular importance in amplification reactions, which depend on binding between nucleic acid strands.

The invention further includes nucleic acids that hybridize to the above-described nucleic acids, especially those nucleic acids that hybridize under stringent hybridization conditions. “Stringent hybridization conditions” or “high stringency conditions” generally occur within a range from about Tₘ minus 5° C. (5° C. below the strand dissociation temperature or melting temperature (Tₘ) of the probe nucleic acid molecule) to about 20° C. to 25° C. below Tₘ. As will be understood by those of skill in the art, the stringency of hybridization may be altered in order to identify or detect molecules having identical or related polynucleotide sequences. An example of high stringency hybridization follows. Hybridization solution is (6× SSC/10 mM EDTA/0.5% SDS/5× Denhardt's solution/100 μg/ml sheared and denatured salmon sperm DNA). Hybridization is at 64-65° C. for 16 hours. The hybridized blot is washed two times with 2× SSC/0.5% SDS solution at room temperature for 15 minutes each, and two times with 0.2× SSC/0.5% SDS at 65° C., for one hour each. Further examples of high stringency conditions can be found on pages 2.10.1-2.10.16 (see particularly 2.10.8-11) and pages 6.3.1-6 in Current Protocols in Molecular Biology (Ausubel, F. M. et al., eds., containing supplements up through Supplement 42, 1998). Examples of high, medium, and low stringency conditions can be found on pages 36 and 37 of WO 98/40404, which are incorporated herein by reference.

The invention further relates to nucleic acids obtainable by screening an appropriate library with a probe having a nucleotide sequence such as that set forth in FIGS. 44A-44C (SEQ ID NO:46), FIGS. 46A-46B (SEQ ID NO:48), FIG. 111 (SEQ ID NO:116), FIGS. 50A-50C (SEQ ID NO:52), FIG. 52 (SEQ ID NO:54) or FIGS. 54A-54C (SEQ ID NO:56), or a probe which is a sufficiently long fragment of any of the above; and isolating the nucleic acid. Such probes generally can comprise at least 15 nucleotides. Nucleic acids obtainable by such screenings may include RNAs, cDNAs and genomic DNA, for example, encoding FATPs of the FATP family described herein.

Further uses for the nucleic acid molecules of the invention, whether encoding a full-length FATP or whether comprising a contiguous portion of a nucleic acid molecule such as one given in SEQ ID NO:46, 48, 116, 52, 54, or 56, include use as markers for tissues in which the corresponding protein is preferentially expressed (to identify constitutively expressed proteins or proteins produced at a particular stage of tissue differentiation or stage of development of a disease state); as molecular weight markers on southern gels; as chromosome markers or tags (when labeled, for example with biotin, a radioactive label or a fluorescent label) to identify chromosomes or to map related gene positions; to compare with endogenous DNA sequences in a mammal to identify potential genetic disorders; as probes to hybridize and thus identify, related DNA sequences; as a source of information to derive PCR primers for genetic fingerprinting; as a probe to “subtract-out” known sequences in the process of discovering other novel nucleic acid molecules; for selecting and making oligomers for attachment to a “gene chip” or other support, to be used, for example, for examination of expression patterns; to raise anti-protein antibodies using DNA immunization techniques; and as an antigen to raise anti-DNA antibodies or to elicit another immune response.

In certain embodiments, a contiguous portion can be about 15, 25, 30, 40, 50, 75, 100, 200, 300, 400, 500, 750, 1000, 1100, 1250, 1500 or more nucleotides in length. In a particular embodiment, the contiguous portion encompasses the signature sequence of a FATP and is about 1080 nucleotides in length.

Further methods to obtain nucleic acids encoding FATPs of the FATP family include PCR and variations thereof (e.g., “RACE” PCR and semi-specific PCR methods). Portions of the nucleic acids having a nucleotide sequence set forth in FIGS. 44A-44C (SEQ ID NO:46), FIGS. 46A-46B (SEQ ID NO:48), FIGS. 111A-B (SEQ ID NO:116), FIGS. 50A-50C (SEQ ID NO:52), FIG. 52 (SEQ ID NO:54) or FIGS. 54A-54C (SEQ ID NO:56), (especially “flanking sequences” on either side of a coding region) can be used as primers in methods using the polymerase chain reaction, to produce DNA from an appropriate template nucleic acid.

Once a fragment of the FATP gene is generated by PCR, it can be sequenced, and the sequence of the product can be compared to other DNA sequences, for example, by using the BLAST Network Service at the National Center for Biotechnology Information. The boundaries of the open reading frame can then be identified using semi-specific PCR or other suitable methods such as library screening. Once the 5′ initiator methionine codon and the 3′ stop codon have been identified, a PCR product encoding the full-length gene can be generated using genomic DNA as a template, with primers complementary to the extreme 5′ and 3′ ends of the gene or to their flanking sequences. The full-length genes can then be cloned into expression vectors for the production of functional proteins.

The invention also relates to isolated proteins or polypeptides such as those encoded by nucleic acids of the present invention. Isolated proteins can be purified from a natural source or can be made recombinantly. Proteins or polypeptides referred to herein as “isolated” are proteins or polypeptides that exist in a state different from the state in which they exist in cells in which they are normally expressed in an organism, and include proteins or polypeptides obtained by methods described herein, similar methods or other suitable methods, and also include essentially pure proteins or polypeptides, proteins or polypeptides produced by chemical synthesis or by combinations of biological and chemical methods, and recombinant proteins or polypeptides which are isolated. Thus, the term “isolated” as used herein, indicates that the polypeptide in question exists in a physical milieu distinct from that in which it occurs in nature. Thus, “isolated” includes existing in membrane fragments and vesicles membrane fractions, liposomes, lipid bilayers and other artificial membrane systems. An isolated FATP may be substantially isolated with respect to the complex cellular milieu in which it naturally occurs, and may even be purified essentially to homogeneity, for example as determined by PAGE or column chromatography (for example, HPLC), but may also have further cofactors or molecular stabilizers, such as detergents, added to the purified protein to enhance activity. In one embodiment, proteins or polypeptides are isolated to a state at least about 75% pure; more preferably at least about 85% pure, and still more preferably at least about 95% pure, as determined by Coomassie blue staining of proteins on SDS-polyacrylamide gels. Proteins or polypeptides referred to herein as “recombinant” are proteins or polypeptides produced by the expression of recombinant nucleic acids.

In a preferred embodiment, an isolated polypeptide comprising a FATP, a functional portion thereof, or a functional equivalent of the FATP, has at least one function characteristic of a FATP, for example, transport activity, binding function (e.g., a domain which binds to AMP), or antigenic function (e.g., binding of antibodies that also bind to a naturally-occurring FATP, as that function is found in an antigenic determinant). Functional equivalents can have activities that are quantitatively similar to, greater than, or less than, the reference protein. These proteins include, for example, naturally occurring FATPs that can be purified from tissues in which they are produced (including polymorphic or allelic variants), variants (e.g., mutants) of those proteins and/or portions thereof. Such variants include mutants differing by the addition, deletion or substitution of one or more amino acid residues, or modified polypeptides in which one or more residues are modified, and mutants comprising one or more modified residues. Portions or fragments of a FATP can range in size from four amino acid residues to the entire amino acid sequence minus one amino acid and include contiguous portions or fragments about 4, 5, 6, 7, 8, 9, 10, 15, 25, 30, 40, 50, 75, 100, 150, 200, 300, 400, 500, 600 or more amino acid residues in length. In one particular embodiment, the portion or fragment includes the signature sequence of a FATP polypeptide and is about 360 amino acid residues in length.

The isolated proteins of the invention preferably include mammalian fatty acid transport proteins of the FATP family of homologous proteins. In one embodiment, the extent of amino acid sequence similarity between a polypeptide having one of the amino acid sequences shown in FIG. 45 (SEQ ID NO:47), FIG. 47 (SEQ ID NO:49), FIG. 112 (SEQ ID NO:117), FIG. 51 (SEQ ID NO:53), FIG. 53 (SEQ ID NO:55), or FIG. 55 (SEQ ID NO:57), and the respective functional equivalents of these polypeptides is at least about 88%. In other embodiments, the degree of amino acid sequence similarity between a FATP and its respective functional equivalent is at least about 91%, at least about 94%, or at least about 97%.

The polypeptides of the invention also include those FATPs encoded by polynucleotides which are orthologous to those polynucleotides, the sequences of which are described herein in whole or in part. FATPs which are orthologs to those described herein by amino acid sequence, in whole or in part, are, for example, fatty acid transport proteins 1-6 of dog, rat, chimpanzee, monkey, rabbit, guinea pig, baboon and pig, and are also embodiments of the invention.

To determine the percent identity or similarity of two amino acid sequences or of two nucleic acid sequences, the sequences are aligned for optimal comparison purposes (e.g., gaps can be introduced in one or both of a first and a second amino acid or nucleic acid sequence for optimal alignment, and non-homologous (dissimilar) sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein, amino acid or nucleic acid “identity” is equivalent to amino acid or nucleic acid “similarity”). The percent identity between the two sequences is a function of the number of identical positions shared by the sequences, taking into account the number of gaps, and the length of each gap, which need to be introduced for optimal alignment of the two sequences.

The invention also encompasses polypeptides having a lower degree of identity but having sufficient similarity so as to perform one or more of the same functions performed by the polypeptides described herein by amino acid sequence. Similarity for a polypeptide is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu, and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe and Tyr. Guidance concerning which amino acid changes are likely to be phenotypically silent is found in Bowie et al., Science 247:1306-1310 (1990).

TABLE 1
Conservative Amino Acid Substitutions

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

The comparison of sequences and determination of percent identity and similarity between two sequences can be accomplished using a mathematical algorithm. (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereaux, J., eds., M. Stockton Press, New York, 1991). In a preferred embodiment, the percent identity between two amino acid sequences is determined using the Needleman and Wunsch (J. Mol. Biol. (48):444-453 (1970)) algorithm which has been incorporated into the GAP program in the GCG software package (available on the worldwide web at gcg.com), using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (Devereux, J., et al., Nucleic Acids Res. 12(1):387 (1984)), (available on the worldwide web at gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against databases to, for example, identify other family members or related sequences. Such searches can be performed using the NBLAST and XBLAST programs (version 2.0) of Altschul, et al. (J. Mol. Biol. 215:403-10 (1990)). BLAST nucleotide searches can be performed with the NBLAST program, score=100, word length=12 to obtain nucleotide sequences homologous to (with calculatably significant similarity to) the nucleic acid molecules of the invention. BLAST protein searches can be performed with the XBLAST program, score=50, word length=3 to obtain amino acid sequences homologous to the proteins of the invention. To obtain gapped alignments for comparison purposes, Gapped BLAST can be utilized as described in Altschul et al., (Nucleic Acids Res. 25(17):3389-3402 (1997)). When utilizing BLAST and gapped BLAST programs, the default parameters of the respective programs (e.g., XBLAST and NBLAST) can be used. (see the worldwide web at ncbi.nlm.nih.gov)

Similarity for nucleotide and amino acid sequences can be defined in terms of the parameters set by the Advanced Blast search available from NCBI (the National Center for Biotechnology Information (see, for Advanced BLAST the worldwide web at ncbi.nlm.nih.gov/cgi-bin/BLAST/nph-newblast?Jform=1). These default parameters, recommended for a query molecule of length greater than 85 amino acid residues or nucleotides have been set as follows: gap existence cost, 11, per residue gap cost, 1; lambda ratio, 0.85. Further explanation of version 2.0 of BLAST can be found on related website pages and in Altschul, S. F. et al, Nucleic Acids Res. 25:3389-3402 (1997).

In certain embodiments, a contiguous portion can be about 4, 5, 6, 7, 8, 9, 10, 15, 25, 30, 40, 50, 75, 100, 150, 200, 300, 400, 500, 600 or more amino acid residues in length. In one particular embodiment, the portion or fragment includes the signature sequence of a FATP polypeptide and is about 360 amino acid residues in length.

The invention further relates to fusion proteins, comprising a FATP or functional portion thereof (as described above) as a first moiety, linked to a second moiety not occurring in the FATP as found in nature. Thus, the second moiety can be an amino acid, peptide or polypeptide. The first moiety can be in an N-terminal location, C-terminal location or internal to the fusion protein. In one embodiment, the fusion protein comprises a FATP as the first moiety, and a second moiety comprising a linker sequence and an affinity ligand. Fusion proteins can be produced by a variety of methods. For example, a fusion protein can be produced by the insertion of a FATP gene or portion thereof into a suitable expression vector, such as Bluescript SK +/− (Stratagene, La Jolla, Calif.), pGEX-4T-2 (Pharmacia, Peapack, N.J.), pET-24(+) (Novagen, Madison, Wis.), or vectors of similar construction. The resulting construct can be introduced into a suitable host cell for expression. Upon expression, fusion protein can be purified from cells by means of a suitable affinity matrix (See e.g., Current Protocols in Molecular Biology, Ausubel, F. M. et al., eds., Vol. 2, pp. 16.4.1-16.7.8, containing supplements up through Supplement 42, 1998).

The invention also relates to enzymatically produced, synthetically produced, or recombinantly produced portions of a fatty acid transport protein. Portions of a FATP can be made which have full or partial function on their own, or which when mixed together (though fully, partially, or nonfunctional alone), spontaneously assemble with one or more other polypeptides to reconstitute a functional protein having at least one function characteristic of a FATP.

Fragments of a FATP can be produced by direct peptide synthesis, for example those using solid-phase techniques (Roberge, J. Y. et al., Science 269:202-204 (1995); Merrifield, J., J. Am. Chem. Soc. 85:2149-2154 (1963)). Protein synthesis can be performed using manual techniques or by automation. Automated synthesis can be carried out using, for instance, an Applied Biosystems 431A Peptide Synthesizer (Perkin Elmer). Various fragments of a FATP can be synthesized separately and combined using chemical methods.

One aspect of the invention is a peptide or polypeptide having the amino acid sequence of a portion of a fatty acid transport protein which is hydrophilic rather than hydrophobic, and ordinarily can be detected as facing the outside of the cell membrane. Such a peptide or polypeptide can be thought of as being an extracellular domain of the FATP, or a mimetic of said extracellular domain. It is known, for example, that a portion of human FATP4 that includes a highly conserved motif is involved in AMP-CoA binding function (Stuhlsatz-Krouper, S. M. et al., J. Biol. Chem. 44:28642-28650 (1998)).

The term “mimetic” as used herein, refers to a molecule, the structure of which is developed from knowledge of the structure of the FATP of interest, or one or more portions thereof, and, as such, is able to effect some or all of the functions of a FATP.

Portions of a FATP can be prepared by enzymatic cleavage of the isolated protein, or can be made by chemical synthesis methods. Portions of a FATP can also be made by recombinant DNA methods in which restriction fragments, or fragments that may have undergone further enzymatic processing, or synthetically made DNAs are joined together to construct an altered FATP gene. The gene can be made such that it encodes one or more desired portions of a FATP. These portions of FATP can be entirely homologous to a known FATP, or can be altered in amino acid sequence relative to naturally occurring FATPs to enhance or introduce desired properties such as solubility, stability, or affinity to a ligand. A further feature of the gene can be a sequence encoding an N-terminal signal peptide directed to the plasma membrane.

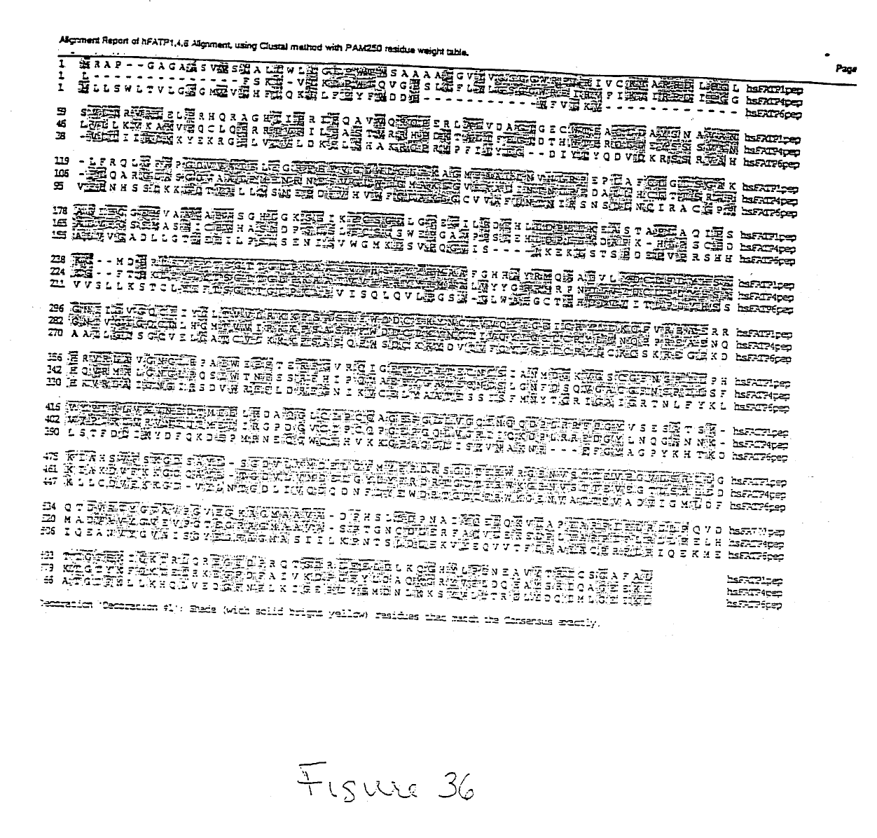

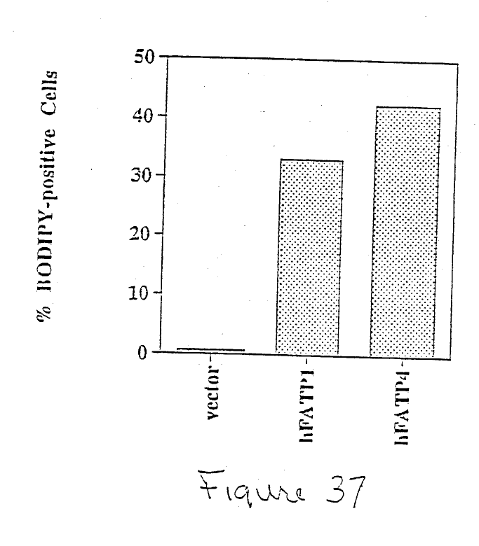

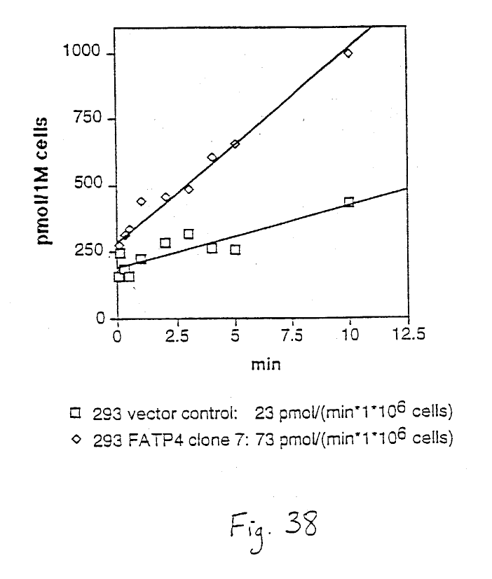

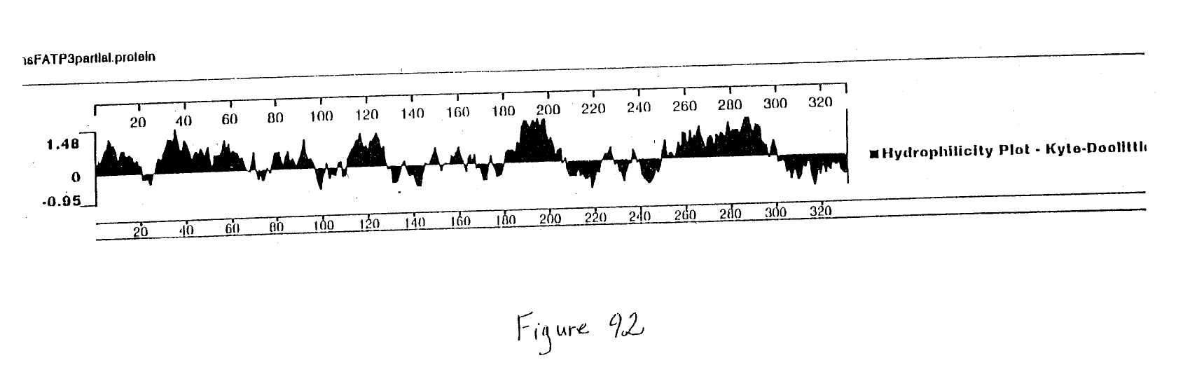

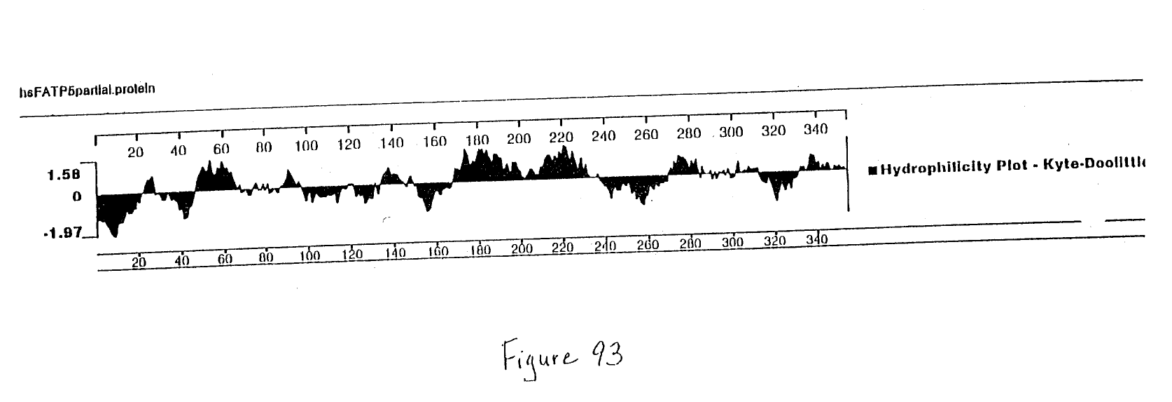

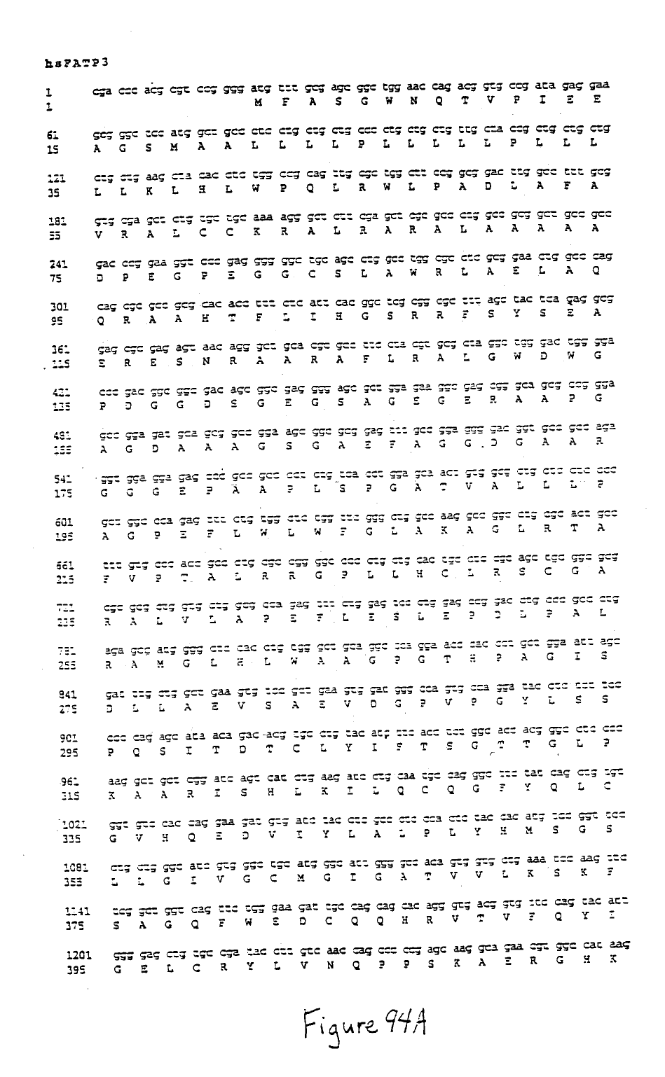

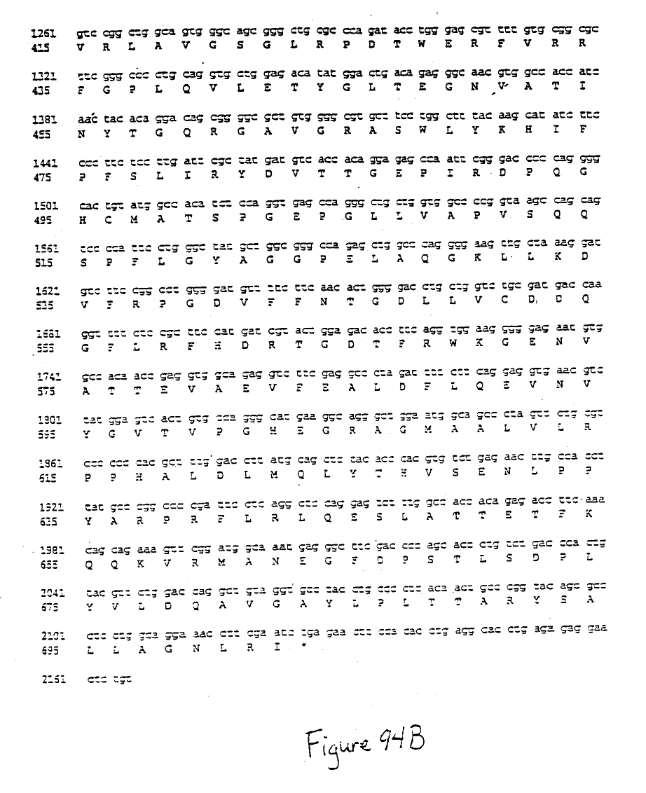

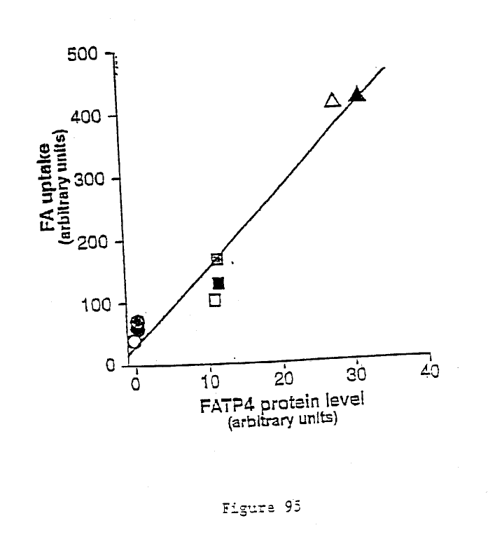

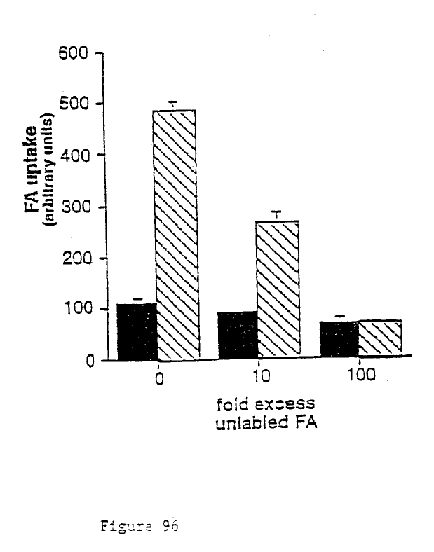

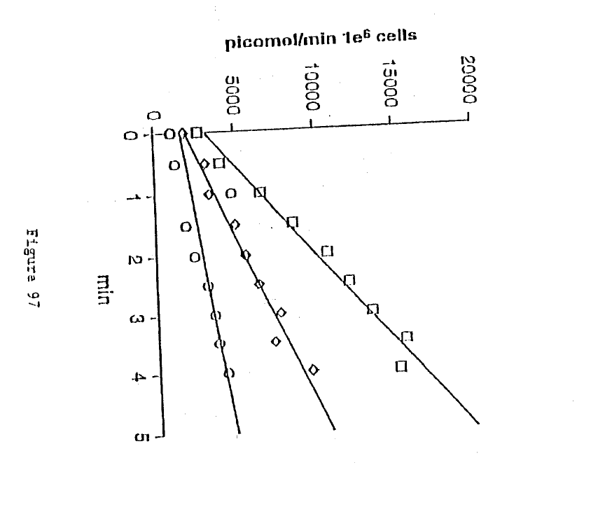

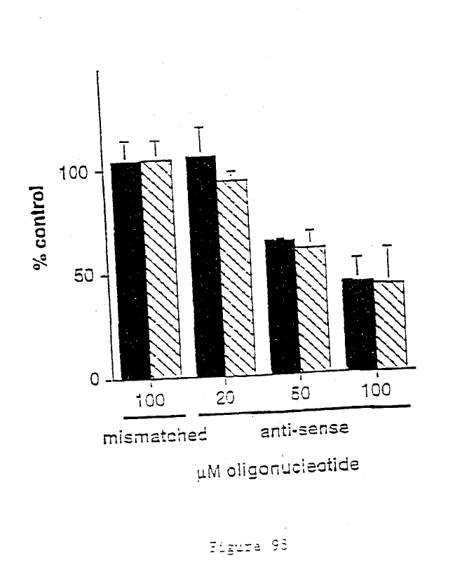

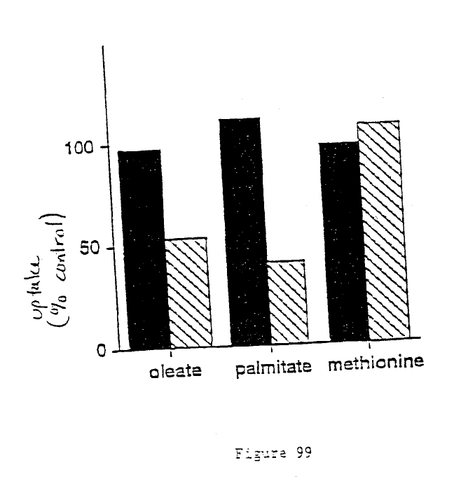

An extracellular domain can be determined by a hydrophobicity plot, such as those shown in FIGS. 28A, 29A, and 35A, or by a hydrophilicity plot such as those shown in FIGS. 28C, 29C, 35C, 91, 92 and 93. A polypeptide or peptide comprising all or a portion of a FATP extracellular domain can be used in a pharmaceutical composition. When administered to a mammal by an appropriate route, the polypeptide or peptide can bind to fatty acids and compete with the native FATPs in the membrane of cells, thereby making fewer fatty acid molecules available as substrates for transport into cells, and reducing the amount of fatty acids taken up by, for example, the heart, in the case of FATP6.

Another aspect of the invention relates to a method of producing a fatty acid transport protein, variants or portions thereof, and to expression systems and host cells containing a vector appropriate for expression of a fatty acid transport protein.

Cells that express a FATP, a variant or a portion thereof, or an ortholog of a FATP described herein by amino acid sequence, can be made and maintained in culture, under conditions suitable for expression, to produce protein in the cells for cell-based assays, or to produce protein for isolation. These cells can be procaryotic or eucaryotic. Examples of procaryotic cells that can be used for expression include Escherichia coli, Bacillus subtilis and other bacteria. Examples of eucaryotic cells that can be used for expression include yeasts such as Saccharomyces cerevisiae, Schizosaccharomyces pombe, Pichia pastoris and other lower eucaryotic cells, and cells of higher eucaryotes such as those from insects and mammals, such as primary cells and cell lines such as CHO, HeLa, 3T3 and BHK cells, preferably COS cells and human kidney 293 cells, and more preferably Jurkat cells. (See, e.g., Ausubel, F. M. et al., eds. Current Protocols in Molecular Biology, Greene Publishing Associates and John Wiley & Sons, Inc., containing Supplements up through Supplement 42, 1998)).

In one embodiment, host cells that produce a recombinant FATP, or a portion thereof, a variant, or an ortholog of a FATP described herein by amino acid sequence, can be made as follows. A gene encoding a FATP, variant or a portion thereof can be inserted into a nucleic acid vector, e.g., a DNA vector, such as a plasmid, phage, cosmid, phagemid, virus, virus-derived vector (e.g., SV40, vaccinia, adenovirus, fowl pox virus, pseudorabies viruses, retroviruses) or other suitable replicon, which can be present in a single copy or multiple copies, or the gene can be integrated in a host cell chromosome. A suitable replicon or integrated gene can contain all or part of the coding sequence for a FATP or variant, operably linked to one or more expression control regions whereby the coding sequence is under the control of transcription signals and linked to appropriate translation signals to permit translation. The vector can be introduced into cells by a method appropriate to the type of host cells (e.g., transfection, electroporation, infection). For expression from the FATP gene, the host cells can be maintained under appropriate conditions (e.g., in the presence of inducer, normal growth conditions, etc.). Proteins or polypeptides thus produced can be recovered (e.g., from the cells, as in a membrane fraction, from the periplasmic space of bacteria, from culture medium) using suitable techniques. Appropriate membrane targeting signals may be incorporated into the expressed polypeptide. These signals may be endogenous to the polypeptide or they may be heterologous signals.

Polypeptides of the invention can be recovered and purified from cell cultures (or from their primary cell source) by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and high performance liquid chromatography. Known methods for refolding protein can be used to regenerate active conformation if the polypeptide is denatured during isolation or purification.

In a further aspect of the invention are methods for assessing the transport function of any of the fatty acid transport proteins or polypeptides described herein, including orthologs, and in variations of these, methods for identifying an inhibitor (or an enhancer) of such function and methods for assessing the transport function in the presence of a candidate inhibitor or a known inhibitor.

A variety of systems comprising living cells can be used for these methods. Cells to be used in fatty acid transport assays, and further in methods for identifying an inhibitor or enhancer of this function, express one or more FATPs. See Examples 3, 6, 9, 12 and 14 for data on tissue distribution of expression of FATPs, and Examples 10 and 11 describing recombinant cells expressing FATP. Cells for use in cell-based assays described herein can be drawn from a variety of sources, such as isolated primary cells of various organs and tissues wherein one or more FATPs are naturally expressed. In some cases, the cells can be from adult organs, and in some cases, from embryonic or fetal organs, such as heart, lung, liver, intestine, skeletal muscle, kidney and the like. Cells for this purpose can also include cells cultured as fragments of organs or in conditions simulating the cell type and/or tissue organization of organs, in which artificial materials may be used as substrates for cell growth. Other types of cells suitable for this purpose include cells of a cell strain or cell line (ordinarily comprising cells considered to be “transformed”) transfected to express one or more FATPs.

A further embodiment of the invention is a method for detecting, in a sample of cells, a fatty acid transport protein, a portion or fragment thereof, a fusion protein comprising a FATP or a portion thereof, or an ortholog as described herein, wherein the cells can be, for instance, cells of a tissue, primary culture cells, or cells of a cell line, including cells into which nucleic acid has been introduced. The method comprises adding to the sample an agent that specifically binds to the protein, and detecting the agent specifically bound to the protein. Appropriate washing steps can be added to reduce nonspecific binding to the agent. The agent can be, for example, an antibody, a ligand or a substrate mimic. The agent can have incorporated into it, or have bound to it, covalently or by high affinity non-covalent interactions, for instance, a label that facilitates detection of the agent to which it is bound, wherein the label can be, but is not limited to, a phosphorescent label, a fluorescent label, a biotin or avidin label, or a radioactive label. The means of detection of a fatty acid transport protein can vary, as appropriate to the agent and label used. For example, for an antibody that binds to the fatty acid transport protein, the means of detection may call for binding a second antibody, which has been conjugated to an enzyme, to the antibody which binds the fatty acid transport protein, and detecting the presence of the second antibody by means of the enzymatic activity of the conjugated enzyme.

Similar principles can also be applied to a cell lysate or a more purified preparation of proteins from cells that may comprise a fatty acid transport protein of interest, for example in the methods of immunoprecipitation, immunoblotting, immunoaffinity methods, that in addition to detection of the particular FATP, can also be used in purification steps, and qualitative and quantitative immunoassays. See, for instance, chapters 11 through 14 in Antibodies: A Laboratory Manual, E. Harlow and D. Lane, eds., Cold Spring Harbor Laboratory, 1988.

Isolated fatty acid transport protein or, an antigenically similar portion thereof, especially a portion that is soluble, can be used in a method to select and identify molecules which bind specifically to the FATP. Fusion proteins comprising all of, or a portion of, the fatty acid transport protein linked to a second moiety not occurring in the FATP as found in nature, can be prepared for use in another embodiment of the method. Suitable fusion proteins for this purpose include those in which the second moiety comprises an affinity ligand (e.g., an enzyme, antigen, epitope). FATP fusion proteins can be produced by the insertion of a gene encoding the FATP or a variant thereof, or a suitable portion of such gene into a suitable expression vector, which encodes an affinity ligand (e.g., pGEX-4T-2 and pET-15b, encoding glutathione S-transferase and His-Tag affinity ligands, respectively). The expression vector can be introduced into a suitable host cell for expression. Host cells are lysed and the lysate, containing fusion protein, can be bound to a suitable affinity matrix by contacting the lysate with an affinity matrix.

In a particular embodiment, a nucleic acid encodes a portion of a FATP polypeptide which includes a motif or domain, for example, a lipocalin domain or an AMP-binding domain. Such a polypeptide portion can be a functional portion of a FATP protein. The term “lipocalin domain” is an art recognized term and as used herein refers to a particular domain present in FATP proteins. This domain is described as including regions of sequence homology as well as a common tertiary structure represented as an eight stranded antiparallel beta-barrel. (see Banaszak, L. et al., Advances in Protein Chemistry, 45: 89-151). Many lipocalin domains can be identified structurally as a sequence contained within the general formula: [DENG]-X-[DENQGSTARK]-X(0,2)-[DENQARK]-[LIVFY]-{CP}-G-{C}-W-[FYWLRH-X]-[LIVMTA], e.g., the lipocalin signature sequence or consensus pattern (SEQ ID NO: 125). One skilled in the art will recognize that a lipocalin domain for a particular FATP protein can vary in sequence from this general formula. A FATP lipocalin domain can be, for example, identical to the lipocalin signature sequence, or, can exhibit 60, 65, 70, 75, 80, 85, 90, 95 or greater sequence percent identity in comparison to the general formula provided that it still retains the necessary lipocalin binding function.

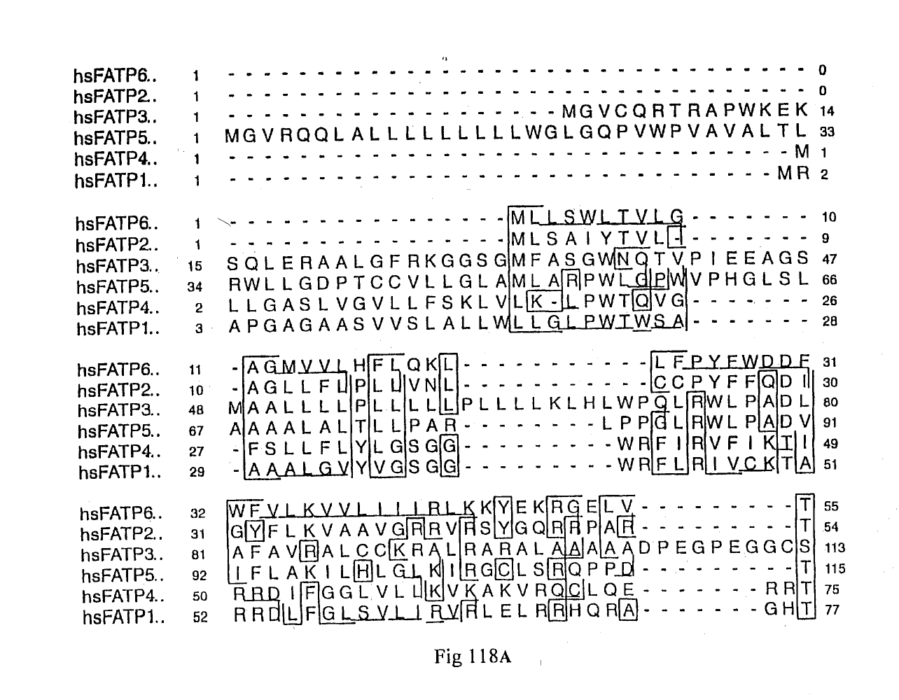

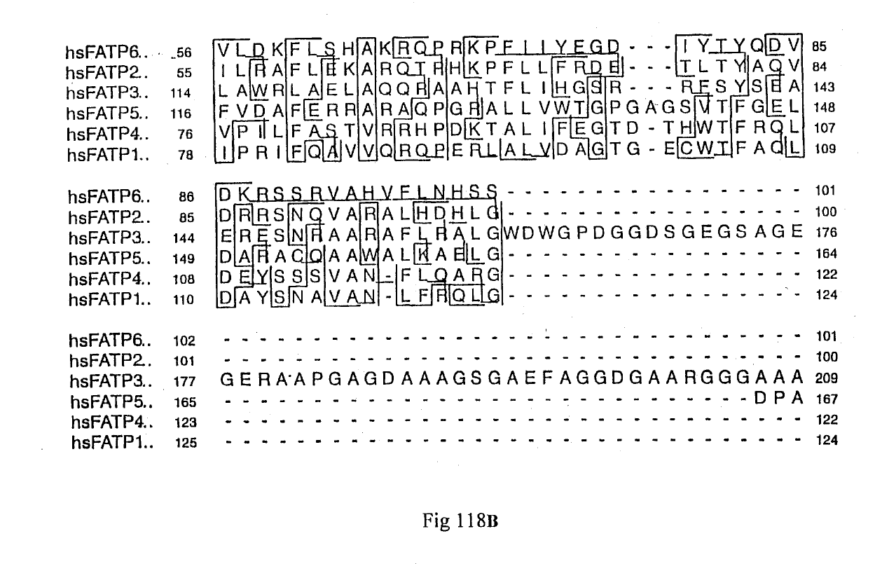

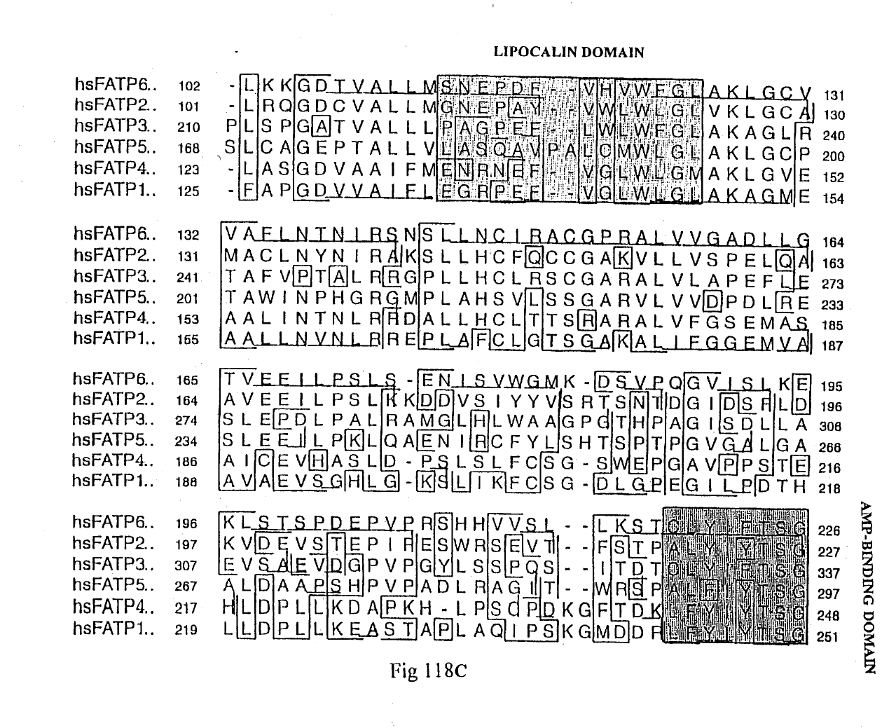

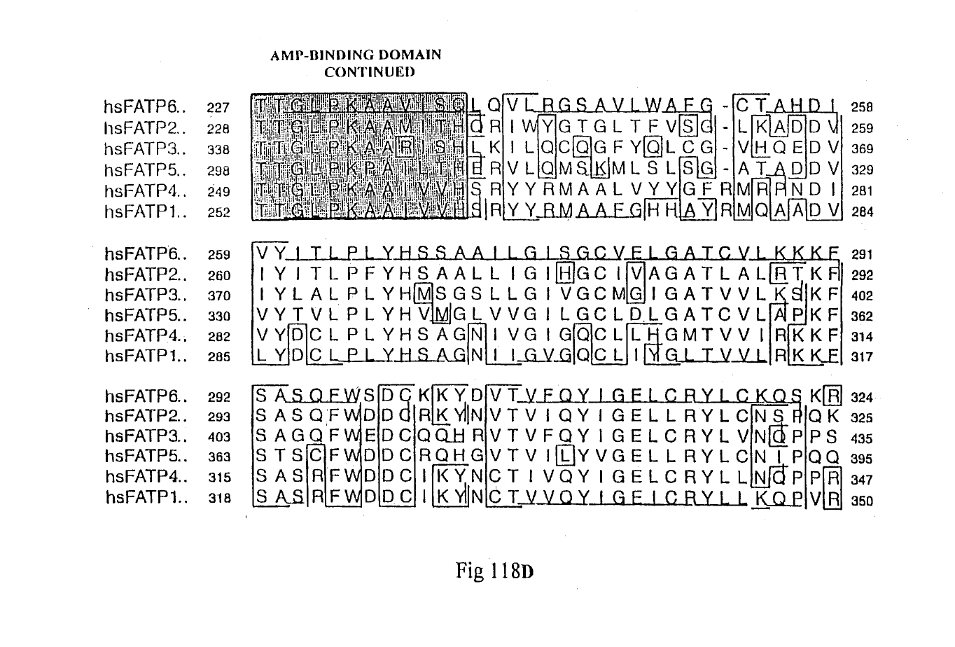

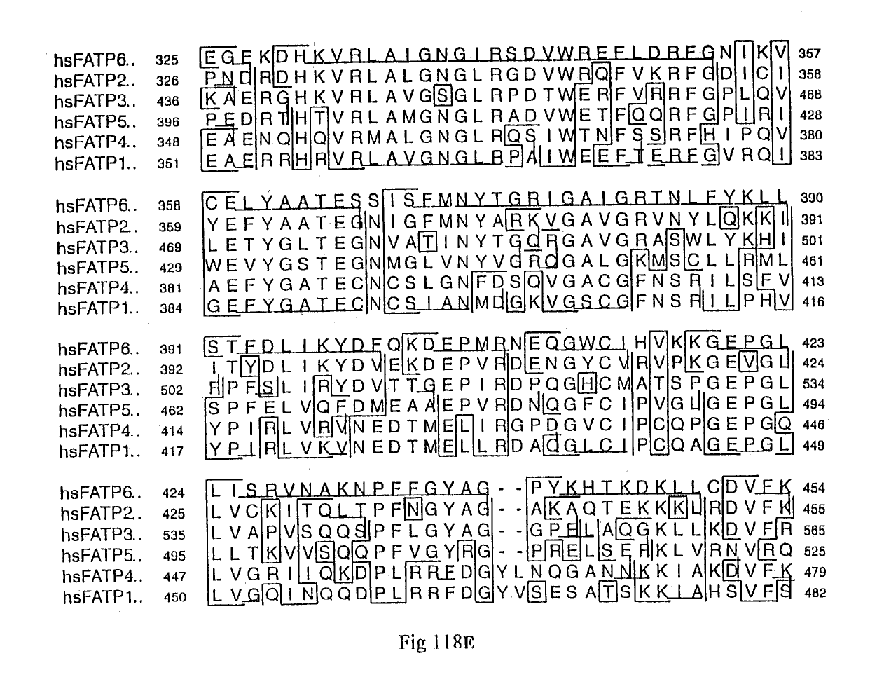

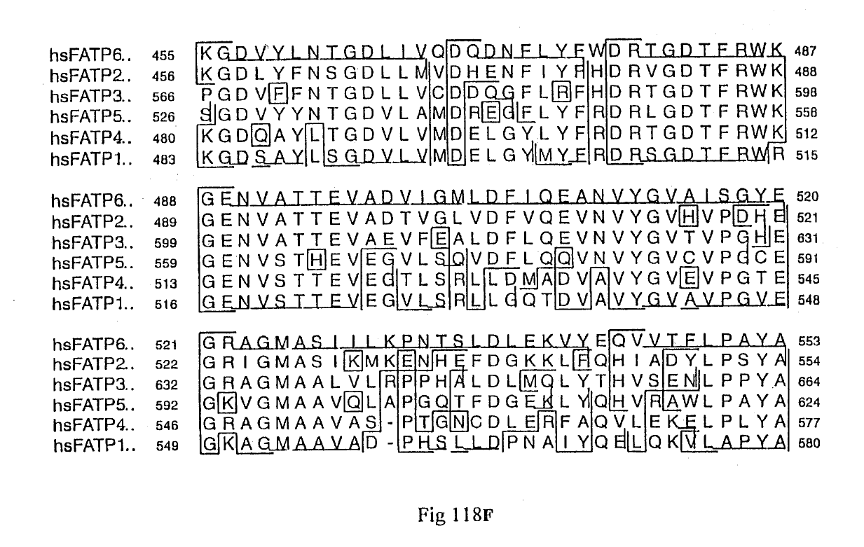

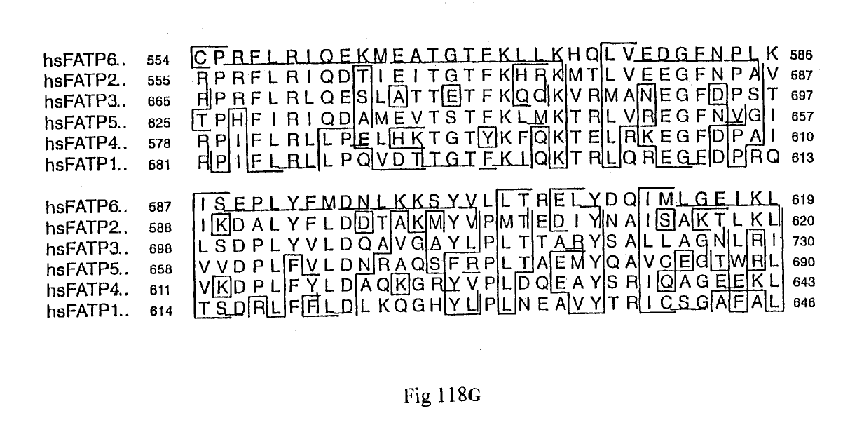

For example, a lipocalin domain for each of the human FATPs, hsFATP1 (SEQ ID NO: 126), hsFATP2 (SEQ ID NO: 127), hsFATP3 (SEQ ID NO: 128), hsFATP4 (SEQ ID NO: 129), hsFATP5 (SEQ ID NO: 130), and hsFATP6 (SEQ ID NO: 131) has been identified. These particular lipocalin domains are located near the N-terminal portion of the specified proteins (see FIG. 118). The sequences of these lipocalin domains are highly conserved across the FATP family. A search using the lipocalin signature sequence conducted on a public database (worldwide web at ebi.ac.uk/interpro/), indicated that the lipocalin domains of hsFATP1 and hsFATP4 share identity with signature sequence. In addition, a search directed to identifying sequences having at least 80% identity to the lipocalin signature sequence identified three additional human FATPs, hsFATP3, hsFATP5 and hsFATP6.

A lipocalin domain can also be identified functionally since, for example, it has been identified as a binding motif capable of binding fatty acids. In particular, the studies described in Experiment 20 demonstrated that fusion proteins including the lipocalin domains from hsFATP4 bound long chain fatty acids such as oleates and palmitates with great specificity. Other fatty acids can also be used to assess binding in FATP4 and other members of the FATP family.

Polypeptides, including fusion polypeptides, which contain a lipocalin domain can also include additional components. For example, fusion polypeptides containing a lipocalin domain can include amino acid residues from the portion of the protein which is located upstream, i. e., in the direction of the N-terminal end of a FATP protein, from the lipocalin domain. As the term “upstream sequences” is used herein in relation to the lipocalin domain, it is intended to refer to the amino acid residues of a FATP protein which are located between the signal peptide (when one is present) and the lipocalin domain. In the absence of a signal peptide, the term refers to the portion of a FATP protein between the lipocalin domain and the amino terminus (see FIG. 115).

Fusion polypeptides which contain a lipocalin domain can also include additional domains or motifs, for example, an AMP binding domain can be included. For example, an AMP binding domain for each of the human FATPs, hsFATP1 (SEQ ID NO: 132), hsFATP2 (SEQ ID NO: 133), hsFATP3 (SEQ ID NO: 134), hsFATP4 (SEQ ID NO: 135), hsFATP5 (SEQ ID NO: 136) and hsFATP6 (SEQ ID NO: 137) has been identified (see FIG. 118).

In one embodiment, the fusion protein can be immobilized on a suitable affinity matrix under conditions sufficient to bind the affinity ligand portion of the fusion protein to the matrix, and is contacted with one or more candidate binding agents (e.g., a mixture of peptides) to be tested, under conditions suitable for binding of the binding agents to the FATP portion of the bound fusion protein. Next, the affinity matrix with bound fusion protein can be washed with a suitable wash buffer to remove unbound candidate binding agents and non-specifically bound candidate binding agents. Those agents which remain bound can be released by contacting the affinity matrix with fusion protein bound thereto with a suitable elution buffer. Wash buffer can be formulated to permit binding of the fusion protein to the affinity matrix, without significantly disrupting binding of specifically bound binding agents. In this aspect, elution buffer can be formulated to permit retention of the fusion protein by the affinity matrix, but can be formulated to interfere with binding of the candidate binding agents to the target portion of the fusion protein. For example, a change in the ionic strength or pH of the elution buffer can lead to release of specifically bound agent, or the elution buffer can comprise a release component or components designed to disrupt binding of specifically bound agent to the target portion of the fusion protein.

Immobilization can be performed prior to, simultaneous with, or after, contacting the fusion protein with candidate binding agent, as appropriate. Various permutations of the method are possible, depending upon factors such as the candidate molecules tested, the affinity matrix-ligand pair selected, and elution buffer formulation. For example, after the wash step, fusion protein with binding agent molecules bound thereto can be eluted from the affinity matrix with a suitable elution buffer (a matrix elution buffer, such as glutathione for a GST fusion). Where the fusion protein comprises a cleavable linker, such as a thrombin cleavage site, cleavage from the affinity ligand can release a portion of the fusion with the candidate agent bound thereto. Bound agent molecules can then be released from the fusion protein or its cleavage product by an appropriate method, such as extraction.

One or more candidate binding agents can be tested simultaneously. Where a mixture of candidate binding agents is tested, those found to bind by the foregoing processes can be separated (as appropriate) and identified by suitable methods (e.g., PCR, sequencing, chromatography). Large libraries of candidate binding agents (e.g., peptides, RNA oligonucleotides) produced by combinatorial chemical synthesis or by other methods can be tested (see e.g., Ohlmeyer, M. H. J. et al., Proc. Natl. Acad. Sci. USA 90:10922-10926 (1993) and DeWitt, S. H. et al., Proc. Natl. Acad. Sci. USA 90:6909-6913 (1993), relating to tagged compounds; see also Rutter, W. J. et al. U.S. Pat. No. 5,010,175; Huebner, V. D. et al., U.S. Pat. No. 5,182,366; and Geysen, H. M., U.S. Pat. No. 4,833,092). Random sequence RNA libraries (see Ellington, A. D. et al., Nature 346:818-822 (1990); Bock, L. C. et al., Nature 355:584-566 (1992); and Szostak, J. W., Trends in Biochem. Sci. 17:89-93 (March, 1992)) can also be screened according to the present method to select RNA molecules which bind to a target FATP or FATP fusion protein. Where binding agents selected from a combinatorial library by the present method carry unique tags, identification of individual biomolecules by chromatographic methods is possible. Where binding agents do not carry tags, chromatographic separation, followed by mass spectrometry to ascertain structure, can be used to identify binding agents selected by the method, for example.

The invention also comprises a method for identifying an agent which inhibits interaction between a fatty acid transport protein (e.g., one comprising the amino acid sequence in SEQ ID NO:47, SEQ ID NO:49, SEQ ID NO:117, SEQ ID NO:53, SEQ ID NO:55, or SEQ ID NO:57), and a ligand of said protein. The FATP can be one described by an amino acid sequence herein, a portion or fragment thereof, a variant thereof, or an ortholog thereof, or a FATP fusion protein. Here, a ligand can be, for instance, a substrate, or a substrate mimic, an antibody, or a compound, such as a peptide, that binds with specificity to a site on the protein. The method comprises combining, not limited to a particular order, the fatty acid protein, the ligand of the protein, and a candidate agent to be assessed for its ability to inhibit interaction between the protein and the ligand, under conditions appropriate for interaction between the protein and the ligand (e.g., pH, salt, temperature conditions conducive to appropriate conformation and molecular interactions); determining the extent to which the protein and ligand interact; and comparing (1) the extent of protein-ligand interaction in the presence of candidate agent with (2) the extent of protein-ligand interaction in the absence of candidate agent, wherein if (1) is less than (2), then the candidate agent is one which inhibits interaction between the protein and the ligand.

The method can be facilitated, for example, by using an experimental system which employs a solid support (column chromatography matrix, wall of a plate, microtiter wells, column pore glass, pins to be submerged in a solution, beads, etc.) to which the protein can be attached. Accordingly, in one embodiment, the protein can be fixed to a solid phase directly or indirectly, by a linker. The candidate agent to be tested is added under conditions conducive for interaction and binding to the protein. The ligand is added to the solid phase system under conditions appropriate for binding. Excess ligand is removed, as by a series of washes done under conditions that do not disrupt protein-ligand interactions. Detection of bound ligand can be facilitated by using a ligand that carries a label (e.g., fluorescent, chemiluminescent, radioactive). In a control experiment, protein and ligand are allowed to interact in the absence of any candidate agent, under conditions otherwise identical to those used for the “test” conditions where candidate inhibiting agent is present, and any washes used in the test conditions are also used in the control. The extent to which ligand binds to the protein in the presence of candidate agent is compared to the extent to which ligand binds to the protein in the absence of the candidate agent. If the extent to which interaction of the protein and the ligand occurs is less in the presence of the candidate agent than in the absence of the candidate agent, the candidate agent is an agent which inhibits interaction between the protein and the ligand of the protein.

In a further embodiment, an inhibitor (or an enhancer) of a fatty acid transport protein can be identified. The method comprises steps which are, or are variations of the following: contacting the cells with fatty acid, wherein the fatty acid can be labeled for convenience of detection; contacting a first aliquot of the cells with an agent being tested as an inhibitor (or enhancer) of fatty acid uptake while maintaining a second aliquot of cells under the same conditions but without contact with the agent; and measuring (e.g., quantitating) fatty acid in the first and second aliquots of cells; wherein a lesser quantity of fatty acid in the first aliquot compared to that in the second aliquot is indicative that the agent is an inhibitor of fatty acid uptake by a fatty acid transport protein. A greater quantity of fatty acid in the first aliquot compared to that in the second aliquot is indicative that the agent is an enhancer of fatty acid uptake by a fatty acid transport protein.

A particular embodiment of identifying an inhibitor or enhancer of fatty acid transport function employs the above steps, but also employs additional steps preceding those given above: introducing into cells of a cell strain or cell line (“host cells” for the intended introduction of, or after the introduction of, a vector) a vector comprising a fatty acid transport protein gene, wherein expression of the gene can be regulatable or constitutive, and providing conditions to the host cells under which expression of the gene can occur.

The terms “contacting” and “combining” as used herein in the context of bringing molecules into close proximity to each other, can be accomplished by conventional means. For example, when referring to molecules that are soluble, contacting is achieved by adding the molecules together in a solution. “Contacting” can also be adding an agent to a test system, such as a vessel containing cells in tissue culture.

The term “inhibitor” or “antagonist”, as used herein, refers to an agent which blocks, diminishes, inhibits, hinders, limits, decreases, reduces, restricts or interferes with fatty acid transport into the cytoplasm of a cell, or alternatively and additionally, prevents or impedes the cellular effects associated with fatty acid transport. The term “enhancer” or “agonist”, as used herein, refers to an agent which augments, enhances, or increases fatty acid transport into the cytoplasm of a cell. An antagonist will decrease fatty acid concentration, fatty acid metabolism and byproduct levels in the cell, leading to phenotypic and molecular changes.

In order to produce a “host cell” type suitable for fatty acid uptake assays and for assays derived therefrom for identifying inhibitors or enhancers thereof, a nucleic acid vector can be constructed to comprise a gene encoding a fatty acid transport protein, for example, human FATP1, FATP2, FATP3, FATP4, FATP5, FATP6, a mutant or variant thereof, an ortholog of the human proteins, such as mouse orthologs or orthologs found in other mammals, or a FATP family protein of origin in an organism other than a mammal. The gene of the vector can be regulatable, such as by the placement of the gene under the control of an inducible or repressible promoter in the vector (e.g., inducible or repressible by a change in growth conditions of the host cell harboring the vector, such as addition of inducer, binding or functional removal of repressor from the cell millieu, or change in temperature) such that expression of the FATP gene can be turned on or initiated by causing a change in growth conditions, thereby causing the protein encoded by the gene to be produced, in host cells comprising the vector, as a plasma membrane protein. Alternatively, the FATP gene can be constitutively expressed.

A vector comprising a FATP gene, such as a vector described herein, can be introduced into host cells by a means appropriate to the vector and to the host cell type. For example, commonly used methods such as electroporation, transfection, for instance, transfection using CaCl₂, and transduction (as for a virus or bacteriophage) can be used. Host cells can be, for example, mammalian cells such as primary culture cells or cells of cell lines such as COS cells, 293 cells or Jurkat cells. Host cells can also be, in some cases, cells derived from insects, cells of insect cell lines, bacterial cells, such as E. coli, or yeast cells, such as S. cerevisiae. It is preferred that the fatty acid transport protein whose function is to be assessed, with or without a candidate inhibitor or enhancer, be produced in host cells whose ancestor cells originated in a species related to the species of origin of the FATP gene encoding the fatty acid transport protein. For example, it is preferable that tests of function or of inhibition or enhancement of a mammalian FATP be carried out in host mammalian cells producing the FATP, rather than bacterial cells or yeast cells.

Host cells comprising a vector comprising a regulatable FATP gene can be treated so as to allow expression of the FATP gene and production of the encoded protein (e.g., by contacting the cells with an inducer compound that effects transcription from an inducible promoter operably linked to the FATP gene).

Alternatively, host cells containing an endogenous FATP gene can be engineered to activate or deactivate expression of the FATP gene and production of the encoded protein. For example, homologous recombination, often referred to as targeting, can be utilized to alter the regulatory region associated with the FATP gene to increase or decrease the level of expression. Alteration of the regulatory region can include disablement of the regulatory region associated with the FATP gene and/or replacement of the region or a portion of the region. A variety of regulatory regions are known which can be transfected into cells to cause an endogenous gene to display a pattern of induction or expression that differs from that of the cell prior to transfection.

The test agent (e.g., an agonist or antagonist) is added to the cells to be used in a fatty acid transport assay, in the presence or absence of test agent, under conditions suitable for production and/or maintenance of the expressed FATP in a conformation appropriate for association of the FATP with test agent and substrate. For example, conditions under which an agent is assessed, such as media and temperature requirements, can, initially, be similar to those necessary for transport of typical fatty acid substrates across the plasma membrane. One of ordinary skill in the art will know how to vary experimental conditions depending upon the biochemical nature of the test agent. The test agent can be added to the cells in the presence of fatty acid, or in the absence of fatty acid substrate, with the fatty acid substrate being added following the addition of the test agent. The concentration at which the test agent can be evaluated can be varied, as appropriate, to test for an increased effect with increasing concentrations.

Test agents to be assessed for their effects on fatty acid transport can be any chemical (element, molecule, compound), made synthetically, made by recombinant techniques or isolated from a natural source. For example, test agents can be peptides, polypeptides, peptoids, sugars, hormones, or nucleic acid molecules, such as antisense nucleic acid molecules. In addition, test agents can be small molecules or molecules of greater complexity made by combinatorial chemistry, for example, and compiled into libraries. These libraries can comprise, for example, alcohols, alkyl halides, amines, amides, esters, aldehydes, ethers and other classes of organic compounds. Test agents can also be natural or genetically engineered products isolated from lysates of cells, bacterial, animal or plant, or can be the cell lysates themselves. Presentation of test compounds to the test system can be in either an isolated form or as mixtures of compounds, especially in initial screening steps.

Thus, the invention relates to a method for identifying agents which alter fatty acid transport, the method comprising providing the test agent to the cell (wherein “cell” includes the plural, and can include cells of a cell strain, cell line or culture of primary cells or organ culture, for example), under conditions suitable for binding to its target, whether to the FATP itself or to another target on or in the cell, wherein the transformed cell comprises a FATP.

In greater detail, to test one or more agents or compounds (e.g., a mixture of compounds can conveniently be screened initially) for inhibition of the transport function of a fatty acid transport protein, the agent(s) can be contacted with the cells. The cells can be contacted with a labeled fatty acid. The fatty acid can be, for example, a known substrate of the fatty acid transport protein such as oleate or palmitate. The fatty acid can itself be labeled with a radioactive isotope, (e.g., ³H or 14C) or can have a radioactively labeled adduct attached. In other variations, the fatty acid can have chemically attached to it a fluorescent label, or a substrate for an enzyme occurring within the cells, wherein the substrate yields a detectable product, such as a highly colored or fluorescent product. Addition of candidate inhibitors and labeled substrate to the cells comprising fatty acid transport protein can be in either order or can be simultaneous.

A second aliquot of cells, which can be called “control” cells (a “first” aliquot of cells can be called “test” cells), is treated, if necessary (as in the case of transformed “host” cells), so as to allow expression of the FATP gene, and is contacted with the labeled substrate of the fatty acid transport protein. The second aliquot of cells is not contacted with one or more agents to be tested for inhibition of the transport function of the protein produced in the cells, but is otherwise kept under the same culture conditions as the first aliquot of cells.

In a further step of a method to identify inhibitors of a fatty acid transport protein, the labeled fatty acid is measured in the first and second aliquots of cells. A preliminary step of this measurement process can be to separate the external medium from the cells so as to be able to distinguish the labeled fatty acid external to the cells from that which has been transported inside the cells. This can be accomplished, for instance, by removing the cells from their growth container, centrifuging the cell suspension, removing the supernatant and performing one or more wash steps to extensively dilute the remaining medium which may contain labeled fatty acid. Detection of the labeled fatty acid can be by a means appropriate to the label used. For example, for a radioactive label, detection can be by scintillation counting of appropriately prepared samples of cells (e.g., lysates or protein extracts); for a fluorescent label, by measuring fluorescence in the cells by appropriate instrumentation.

If a compound tested as a candidate inhibitor of transport function causes the test cells to have less labeled fatty acid detected in the cells than that detected in the control cells, then the compound is an inhibitor of the fatty acid transport protein. Procedures analogous to those above can be devised for identifying enhancers (agonists of FATPs) of fatty acid transport function wherein if the test cells contain more labeled fatty acid than that detected in the control cells, or if the fatty acid is taken up at a higher rate, then the compound being tested can be concluded to be an enhancer of the fatty acid transport protein.

Example 13 describes use of an assay of this type to identify an inhibitor of a FATP. In Example 13, an antisense oligonucleotide which specifically inhibits biosynthesis of mmFATP4 was demonstrated to inhibit fatty acid uptake into mouse enterocytes. Similarly, antisense oligonucleotides directed towards specifically inhibiting the biosynthesis of FATP6 in heart cells, FATP5 in liver cells, FATP3 in lung cells, and FATP2 in colon cells, can be demonstrated as examples of “test agents” that inhibit fatty acid transport.

Another assay to determine whether an agent is an inhibitor (or enhancer) of fatty acid transport employs animals, one or more of which are administered the agent, and one or more of which are maintained under similar conditions, but are not administered the agent. Both groups of animals are given fatty acids (e.g., orally, intravenously, by tube inserted into stomach or intestine), and the fatty acids taken up into a bodily fluid (e.g., serum) or into an organ or tissue of interest are measured from comparable samples taken from each group of animals. The fatty acids may carry a label (e.g., radioactive) to facilitate detection and quantitation of fatty acids taken up into the fluid or tissue being sampled. This type of assay can be used alone or can be used in addition to in vitro assays of a candidate inhibitor or enhancer.

An agent determined to be an inhibitor (or enhancer) of FATP function, such as fatty acid binding and/or fatty acid uptake, can be administered to cells in culture, or in vivo, to a mammal (e.g. human) to inhibit (or enhance) FATP function. Such an agent may be one that acts directly on the FATP (for example, by binding) or can act on an intermediate in a biosynthetic pathway to produce FATP, such as transcription of the FATP gene, processing of the mRNA, or translation of the mRNA. An example of such an agent is antisense oligonucleotide.

Antisense methods similar to those illustrated in Example 13 can be used to determine the target FATP of a compound or agent that has an inhibitory or enhancing effect on fatty acid uptake. For example, antisense oligonucleotide directed to the inhibition of FATP4 biosynthesis can be added to lung cells or cell lines derived from lung cells. In addition, antisense oligonucleotides directed to the inhibition of other FATPs, except for FATP3, can also be added to the lung cells. The administration of antisense oligonucleotides in this manner ensures that the predominant FATP activity remaining in the cells comes from FATP3. After a period of incubation of the cells with the antisense oligonucleotides sufficient to deplete the plasma membrane of the FATPs whose biosynthesis has been inhibited, a test agent, preferably one that has been shown by some preliminary test to have an inhibitory or enhancing activity on fatty acid transport, can be added to the lung cells. If the test agent is now demonstrated, after treatment of the cells with antisense oligonucleotides, to have an inhibitory or enhancing activity on fatty acid transport in the lung cells, it can be concluded that the target of the test agent is FATP3, or a molecule involved in the biosynthesis or activity of FATP3.

In another type of cell-based assay for uptake of fatty acids, a change of intracellular pH resulting from the uptake of fatty acids can be followed by an indicator fluorophore. The fluorophore can be taken up by the cells in a preincubation step. Fatty acids can be added to the cell medium, and after some period of incubation to allow FATP-mediated uptake of fatty acids, the change in λₘₐₓ of fluorescence can be measured, as an indicator of a change in intracellular pH, as the λₘₐₓ of fluorescence of the fluorophore changes with the pH of its environment, thereby indicating uptake of fatty acids. One such fluorophore is BCECF (2′, 7′-bis(2-carboxyethyl)-5(6)-carboxyfluorescein; Rink, T. J. et al., J Cell. Biol. 95: 189 (1982)).

In assays similar to those described above, a candidate inhibitor or enhancer of fatty acid transport function can be added (or mock-added, for control cultures) to cultures of cells engineered to express a desired FATP to which fatty acid substrate is also added. Inhibition of fatty acid uptake is indicated by a lack of the drop in pH, indicating fatty acid uptake, that is seen in control cells. Enhancement of fatty acid uptake is indicated by a decrease in intracellular pH, as compared to control cells not receiving the candidate enhancer of fatty acid transport function.

Yeast cells can be used in a similar cell-based assay for the uptake of fatty acids mediated by a FATP, and such an assay can be adapted to a screening assay for the identification of agents that inhibit or enhance fatty acid uptake by an FATP. Yeast cells lacking an endogenous FATP activity (mutated, disrupted or deleted for FAT1; Faergeman, N. J. et al., J. Biol. Chem. 272(13):8531-8538 (1997); Watkins, P. A. et al., J. Biol. Chem. 273(29):18210-18219 (1998)) can be engineered to harbor a related gene of the family of FATP-encoding genes, such as a mammalian FATP (e.g., human FATP4).

Examples of expression vectors include pEG (Mitchell, D. A., et al., Yeast 9:715-723 (1993)) and pDAD1 and pDAD2, which contain a GAL1 promoter (Davis, L. I. and Fink, G. R., Cell 61:965-978 (1990)). A variety of promoters are suitable for expression. Available yeast vectors offer a choice of promoters. In one embodiment, the inducible GAL1 promoter is used. In another embodiment, the constitutive ADH1 promoter (alcohol dehydrogenase; Bennetzen, J. L. and Hall, B. D., J. Biol. Chem. 257:3026-3031 (1982)) can be used to express an inserted gene on glucose-containing media. An example of a vector suitable for expression of a heterologous FATP gene in yeast is pQB169.

With the introduced FATP gene providing the only fatty acid transport protein function for the yeast cells, it is possible to study effect of the heterologous FATP on fatty acid transport into the yeast cells in isolation. Assays for the uptake of fatty acids into the yeast cells can be devised that are similar to those described above and/or those assays that have been illustrated in the Examples. Tests for candidate inhibitors or enhancers of the heterologous FATP can be done in cultures of yeast cells, wherein the yeast cells are incubated with fatty acid substrate and an agent to be tested as an inhibitor or enhancer of FATP function. FATP uptake after a period of time can be measured by analyzing the contents of the yeast cells for fatty acid substrate, as compared with control yeast cells incubated with the fatty acid, but not with the test agent. Yeast cells have the additional advantage, over mammalian cells in culture, for example, that yeast cells can be forced to rely upon fatty acids as their only source of carbon, if the growth medium supplied to the yeast cells is formulated to contain no other source of carbon. Thus, the effect of the heterologous FATP on fatty acid uptake and metabolism in the engineered yeast cells can be amplified. An agent that efficiently blocks transport function of the heterologous FATP could result in death of the yeast cells. Thus, in this case, inhibition of function of the heterologous FATP can result in loss of viability. A simple measure of viability is turbidity of the yeast suspension culture, which can be adapted to a high throughput screening assay for effects of various agents to be tested, using microtiter plates or similar devices for small-volume cultures of the engineered yeast cells.

Cell-free assays can also be used to measure the transport of fatty acids across a membrane, and therefor also to assess a test treatment or test agent for its effect on the rate or extent of fatty acid transport. An isolated FATP, for example in the presence of a detergent that preserves the native 3-dimensional structure of the FATP, or partially purified FATP, can be used in an artificial membrane system typically used to preserve the native conformation and activity of membrane proteins. Such systems include liposomes, artificial bilayers of phospholipids, isolated plasma membrane such as cell membrane fragments, cell membrane fractions, or cell membrane vesicles, and other systems in which the FATP can be properly oriented within the membrane to have transport activity. Assays for transport activity can be performed using methods analogous to those that can be used in cells engineered to predominantly express one FATP whose function is to be measured. A labeled (e.g., radioactively labeled) fatty acid substrate can be incubated with one side of a bilayer or in a suspension of liposomes constructed to integrate a properly oriented FATP. The accumulation of fatty acids with time can be measured, using appropriate means to detect the label (e.g., scintillation counting of medium on each side of the bilayer, or of the contents of liposomes isolated from the surrounding medium). Assays such as these can be adapted to use for the testing of agents which might interact with the FATP to produce an inhibitory or an enhancing effect on the rate or extent of fatty acid transport. That is, the above-described assay can be done in the presence or absence of the agent to be tested, and the results compared.

For examples of isolation of membrane proteins (ADP/ATP carrier and uncoupling protein), reconstitution into phospholipid vesicles, and assays of transport, see Klingenberg, M. et al., Methods Enzymol. 260:369-389 (1995). For an example of a membrane protein (phosphate carrier of Saccharomyces cerevisiae) that was purified and solubilized from E. coli inclusion bodies, see Schroer, A. et al., J. Biol. Chem. 273: 14269-14276 (1998). The Glut1 glucose transporter of rat has been expressed in yeast. A crude membrane fraction of the yeast was prepared and reconstituted with soybean phospholipids into liposomes. Glucose transport activity could be measured in the liposomes (Kasahara, T. and Kasahara, M., J. Biol. Chem. 273: 29113-29117 (1998)). Similar methods can be applied to the proteins and polypeptides of the invention.

Another embodiment of the invention is a method for inhibiting fatty acid uptake in a mammal (e.g., a human), comprising administering to the mammal a therapeutically effective amount of an inhibitor of the transport function of one or more of the fatty acid transport proteins, thereby decreasing fatty acid uptake by cells comprising the fatty acid protein(s). Where it is desirable to reduce the uptake of fatty acids, for example, in the treatment of chronic obesity or as a part of a program of weight control or hyperlipidemia control in a human, one or more inhibitors of one or more of the fatty acid transport proteins can be administered in an effective dose, and by an effective route, for example, orally, or by an indwelling device that can deliver doses to the small intestine. The inhibitor can be one identified by methods described herein, or can be one that is, for instance, structurally related to an inhibitor identified by methods described herein (e.g., having chemical adducts to better stabilize or solubilize the inhibitor). The invention further relates to compositions comprising inhibitors of fatty acid uptake in a mammal, which may further comprise pharmaceutical carriers suitable for administration to a subject mammal, such as sterile solubilizing or emulsifying agents.

A further embodiment of the present invention is a method of enhancing or increasing fatty acid uptake, such as enhancing or increasing LCFA uptake in the small intestine (e.g., to treat or prevent a malabsorption syndrome or other wasting condition) or in the liver (e.g., by an enhancer of FATP5 transport activity to treat acute liver failure) or in the kidney (e.g., by an enhancer of FATP2 transport activity to treat kidney failure). In this embodiment, a therapeutically effective amount of an enhancer of the transport function of one or more of the fatty acid transport proteins can be administered to a mammalian subject, with the result that fatty acid uptake in the small intestine is enhanced. In this embodiment, one or more enhancers of one or more of fatty acid transport proteins is administered in an effective dose and by a route (e.g., orally or by a device, such as an indwelling catheter or other device) which can deliver doses to the gut. The enhancer of FATP function (e.g., an enhancer of FATP4 function) can be identified by methods described herein or can be one that is structurally similar to an enhancer identified by methods described herein.

Aerobic reperfusion of ischemic myocardium is a common clinical event which can occur during such treatments as cardiac surgery, angioplasty, and thrombolytic therapy after a myocardial infarction. During reperfusion, a rapid recovery of myocardial energy production is essential for the complete recovery of contractile function. Not only the extent of recovery of myocardial energy metabolism but also the type of energy substrate used by the heart during reperfusion are important determinants of functional recovery. Circulating fatty acid levels increase following acute myocardial infarction or during cardiac surgery, such that during and following ischemia the heart muscle can be exposed to very high concentrations of fatty acids (Lopaschuk, G. D. and W. C. Stanley, Science and Medicine (November/December 1997)). High plasma fatty acid concentrations increase the severity of ischemic damage in a number of experimental models of cardiac ischemia and have been linked to depression of mechanical function during aerobic reperfusion of previously ischemic hearts. Further data show that modifying fatty acid utilization can be beneficial for heart function in ischemia and can be a useful approach for the treatment of angina. See, e.g., Desideri and Celegon, Am. J. Cardiol. 82(5A):50K-53K; Lopaschuk, Am. J. Cardiol. 82(5A):14K-1 7K. Plasma fatty acid concentrations can be reduced by administering to a human subject or other mammal an effective amount of an inhibitor of a FATP such as FATP2 or FATP4, thereby providing a way of reducing fatty acid utilization by the heart.

In a further embodiment of the invention, a therapeutically effective amount of an inhibitor of hsFATP6 can be administered to a human patient by a suitable route, to reduce the uptake of fatty acids by cardiac muscle. This treatment is desirable in patients who are diagnosed as having, or who are at risk of, abnormal accumulations of fatty acids in the heart or a detrimentally high rate of uptake of fatty acids into the heart, because of ischemic heart disease, or following ischemia or trauma to the heart.

The invention further relates to antibodies that bind to an isolated or recombinant fatty acid transport protein of the FATP family, including portions of antibodies, which can specifically recognize and bind to one or more FATPs. The antibodies and portions thereof of the invention include those which bind to one or more FATPs of mouse or other mammalian species. In a preferred embodiment, the antibodies specifically bind to a naturally occurring FATP of humans. The antibodies can be used in methods to detect or to purify a protein of the present invention or a portion thereof by various methods of immunoaffinity chromatography, to inhibit the function of a protein in a method of therapy, or to selectively inactivate an active site, or to study other aspects of the structure of these proteins, for example.

The antibodies of the present invention can be polyclonal or monoclonal. The term antibody is intended to encompass both polyclonal and monoclonal antibodies. Antibodies of the present invention can be raised against an appropriate immunogen, including proteins or polypeptides of the present invention, such as an isolated or recombinant FATP1, FATP2, FATP3, FATP4, FATP5, FATP6, mtFATP, ceFATPa, ceFATPb, scFATP or portions thereof, or synthetic molecules, such as synthetic peptides (e.g., conjugated to a suitable carrier). Preferred embodiments are antibodies that bind to any of the following: hsFATP 1, hsFATP2, hsFATP3, hsFATP4, hsFATP5 or hsFATP6. The immunogen can be a polypeptide comprising a portion of a FATP and having at least one function of a fatty acid transport protein, as described herein.

The term antibody is also intended to encompass single chain antibodies, chimeric, humanized or primatized (CDR-grafted) antibodies and the like, as well as chimeric or CDR-grafted single chain antibodies, comprising portions from more than one species. For example, the chimeric antibodies can comprise portions of proteins derived from two different species, joined together chemically by conventional techniques or prepared as a single contiguous protein using, genetic engineering techniques (e.g., DNA encoding the protein portions of the chimeric antibody can be expressed to produce a contiguous protein chain. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., U.S. Pat. No. 5,585,089; and Queen et al., European Patent No. EP 0 451 216 B1. See also, Newman, R. et al., BioTechnology, 10: 1455-1460 (1992), regarding primatized antibody, and Ladner et al., U.S. Pat. No. 4,946,778 and Bird, R. E. et al., Science, 242:423-426 (1988) regarding single chain antibodies.)

Whole antibodies and biologically functional fragments thereof are also encompassed by the term antibody. Biologically functional antibody fragments which can be used include those fragments sufficient for binding of the antibody fragment to a FATP to occur, such as Fv, Fab, Fab′ and F(ab′)₂ fragments. Such fragments can be produced by enzymatic cleavage or by recombinant techniques. For instance, papain or pepsin cleavage can generate Fab or F(ab′)₂ fragments, respectively. Antibodies can also be produced in a variety of truncated forms using antibody genes in which one or more stop codons have been introduced upstream of the natural stop site. For example, a chimeric gene encoding a F(ab′)₂ heavy chain portion can be designed to include DNA sequences encoding the CH₁ domain and hinge region of the heavy chain.

Preparation of immunizing antigen (whole cells comprising FATP on the cell surface or purified FATP), and polyclonal and monoclonal antibody production can be performed using any suitable technique. A variety of methods have been described (See e.g., Kohler et al., Nature, 256: 495-497 (1975) and Eur. J. Immunol. 6: 511-519 (1976); Milstein et al., Nature 266: 550-552 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Chapter 11 In Current Protocols In Molecular Biology, Vol. 2 (containing supplements up through Supplement 42, 1998), Ausubel, F. M. et al., eds., (John Wiley & Sons: New York, N.Y.)). Generally, a hybridoma can be produced by fusing a suitable immortal cell line (e.g., a myeloma cell line such as SP2/0) with antibody producing cells. The antibody producing cells, preferably those obtained from the spleen or lymph nodes, can be obtained from animals immunized with the antigen of interest. Immunization of animals can be by introduction of whole cells comprising fatty acid transport protein on the cell surface. The fused cells (hybridomas) can be isolated using selective culture conditions, and cloned by limiting dilution. Cells which produce antibodies with the desired specificity can be selected by a suitable assay (e.g., ELISA).

Other suitable methods of producing or isolating antibodies (including human antibodies) of the requisite specificity can used, including, for example, methods which select recombinant antibody from a library (e.g., Hoogenboom et al., WO 93/06213; Hoogenboom et al., U.S. Pat. No. 5,565,332; WO 94/13804, published Jun. 23, 1994; and Dower, W. J. et al., U.S. Pat. No. 5,427,908), or which rely upon immunization of transgenic animals (e.g., mice) capable of producing a full repertoire of human antibodies (see e.g., Jakobovits et al., Proc. Natl. Acad. Sci. USA, 90: 2551-2555 (1993); Jakobovits et al., Nature, 362:255-258 (1993); Lonberg et al., U.S. Pat. No. 5,569,825; Lonberg et al., U.S. Pat. No. 5,545,806; Surani et al., U.S. Pat. No. 5,545,807; and Kucherlapati, R. et al., European Patent No. EP 0 463 151 B1).

Another aspect of the invention is a method for directing an agent to cardiac muscle. The differential expression of FATP6 in cardiac muscle but not in other tissue types allows for the specific targeting of drugs, diagnostic agents, tagging labels, histological stains or other substances specifically to cardiac muscle. A targeting vehicle can be used for the delivery of such a substance. Targeting vehicles which bind specifically to FATP6 can be linked to a substance to be delivered to the cells of cardiac muscle. The linkage can be, for instance, via one or more covalent bonds, or by high affinity non-covalent bonds. A targeting vehicle can be an antibody, for instance, or other compound (e.g., a fatty acid or fatty acid analog) which binds to FATP6 with high specificity.

Targeting vehicles specific to the heart-specific protein FATP6 have in vivo (e.g., therapeutic and diagnostic) applications. For example, an antibody which specifically binds to FATP6 can be conjugated to a drug to be targeted to the heart (e.g., a cardiac glycoside to treat congestive heart failure, or β-adrenergic agents, sodium channel blockers or calcium channel blockers to treat arrhythmias). A substance (e.g., a radioactive substance) which can be detected (e.g., a label) in vivo can also be linked to a targeting vehicle which specifically binds to a heart-specific protein such as FATP6, and the conjugate can be used as a labeling agent to identify cardiac muscle cells.

Targeting vehicles specific to FATP6 find further applications in vitro. For example, an FATP6-specific targeting vehicle, such as an antibody (a polyclonal preparation or monoclonal) which specifically binds to FATP6, can be linked to a substance which can be used as a stain for a tissue sample (e.g., horseradish peroxidase) to provide a method for the identification of cardiac muscle in a sample, as can be used in embryology studies, for example.

In a similar manner, an agent can be directed to the liver of a mammal, as FATP5 is expressed in liver but not in other tissue types. A targeting vehicle which specifically binds to FATP5 can be conjugated to a drug for delivery of the drug to the liver, such as a drug to treat hepatitis, Wilson's disease, lipid storage diseases and liver cancer. As with targeting vehicles specific to FATP6, targeting vehicles specific to FATP5 can be used in studying tissue samples in vitro.

The invention also relates to compositions comprising a modulator of FATP function. The term “modulate” as used herein refers to the ability of a molecule to alter the function of another molecule. Thus, modulate could mean, for example, inhibit, antagonize, agonize, upregulate, downregulate, induce, or suppress. A modulator has the capability of altering function of its target. Such alteration can be accomplished at any stage of the transcription, translation, expression or function of the protein, so that, for example, modulation of a target gene can be accomplished by modulation of the DNA or RNA encoding the protein, and the protein itself.

Antagonists or agonists (inhibitors or enhancers) of the FATPs of the invention, antibodies that bind a FATP, or mimetics of a FATP can be employed in combination with a non-sterile or sterile carrier or carriers for use with cells, tissues or organisms, such as a pharmaceutical carrier suitable for administration to a mammalian subject. Such compositions comprise, for instance, a media additive or a therapeutically effective amount of an inhibitor or enhancer compound to be identified by an assay of the invention and a pharmaceutically acceptable carrier or excipient. Such carriers may include, but are not limited to, saline, buffered saline, dextrose, water, ethanol, surfactants, such as glycerol, excipients such as lactose and combinations thereof. The formulation can be chosen by one of ordinary skill in the art to suit the mode of administration. The chosen route of administration will be influenced by the predominant tissue or organ location of the FATP whose function is to be inhibited or enhanced. For example, for affecting the function of FATP4, a preferred administration can be oral or through a tube inserted into the stomach (e.g., direct stomach tube or nasopharyngeal tube), or through other means to accomplish delivery to the small intestine. The invention further relates to diagnostic and pharmaceutical packs and kits comprising one or more containers filled with one or more of the ingredients of the aforementioned compositions of the invention.

Compounds of the invention which are FATPs, FATP fusion proteins, FATP mimetics, FATP gene-specific antisense poly- or oligonucleotides, inhibitors or enhancers of a FATP may be employed alone or in conjunction with other compounds, such as therapeutic compounds. The pharmaceutical compositions may be administered in any effective, convenient manner, including administration by topical, oral, anal, vaginal, intravenous, intraperitoneal, intramuscular, subcutaneous, intranasal, transdermal or intradermal routes, among others. In therapy or as a prophylactic, the active agent may be administered to an individual as an injectable composition, for example as a sterile aqueous dispersion, preferably isotonic.

Alternatively, the composition may be formulated for topical application, for example, in the form of ointments, creams, lotions, eye ointments, eye drops, ear drops, mouthwash, impregnated dressings and sutures and aerosols, and may contain appropriate conventional additives, including, for example, preservatives, solvents to assist drug penetration, and emollients in ointments and creams. Such topical formulations may also contain compatible conventional carriers, for example cream or ointment bases, and ethanol or oleyl alcohol for lotions.

In addition, the amount of the compound will vary depending on the size, age, body weight, general health, sex, and diet of the host, and the time of administration, the biological half-life of the compound, and the particular characteristics and symptoms of the disorder to be treated. Adjustment and manipulation of established dose ranges are well within the ability of those of skill in the art.

A further aspect of the invention is a method to identify a polymorphism, or the presence of an alternative or variant allele of a gene in the genome of an organism (of interest here, genes encoding FATPs). As used herein, polymorphism refers to the occurrence of two or more genetically determined alternative sequences or alleles in a population. A polymorphic locus may be as small as a base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified alleleic form, or the most frequently occurring form can be arbitrarily designated as the reference (usually, “wildtype”) form, and other allelic forms are designated as alternative (sometimes, “mutant” or “variant”). Dipolid organisms may be homozygous or heterozygous for allelic forms.

An “allele” or “allelic sequence” is an alternative form of a gene which may result from at least one mutation in the nucleotide sequence. Alleles may result in altered mRNAs or polypeptides whose structure or function may or may not be altered. Any given gene may have none, one, or many allelic forms (polymorphism). Common mutational changes which give rise to alleles are generally ascribed to natural deletions, additions, or substitutions of nucleotides. Each of these types of changes may occur alone, or in combination with the others, one or more times in a given sequence.

Several different types of polymorphisms have been reported. A restriction fragment length polymorphism (RFLP) is a variation in DNA sequence that alters the length of a restriction fragment (Botstein et al., Am. J. Hum. Genet. 32:314-331 (1980)). The restriction fragment length polymorphism may create or delete a restriction site, thus changing the length of the restriction fragment. RFLPs have been widely used in, human and animal genetic analyses (see WO 90/13668; WO 90/11369; Donis-Keller, Cell 51:319-337 (1987); Lander et al., Genetics 121:85-99 (1989)). When a heritable trait can be linked to a particular RFLP, the presence of the RFLP in an individual can be used to predict the likelihood that the individual will also exhibit the trait.

Other polymorphisms take the form of short tandem repeats (STRs) that include tandem di-, tri- and tetra-nucleotide repeated motifs. These tandem repeats are also referred to as variable number tandem repeat (VNTR) polymorphisms. VNTRs have been used in identity and paternity analysis (U.S. Pat. No. 5,075,217; Armour et al., FEBS Lett. 307:113-115 (1992); Horn et al., WO 91/14003; Jeffreys, EP 370,719), and in a large number of genetic mapping studies.

Other polymorphisms take the form of single nucleotide variations between individuals of the same species. Such polymorphisms are far more frequent than RFLPs, STRs (short tandem repeats) and VNTRs (variable number tandem repeats). Some single nucleotide polymorphisms occur in protein-coding sequences, in which case, one of the polymorphic forms may give rise to the expression of a defective or other variant protein and, potentially, a genetic disease. Other single nucleotide polymorphisms occur in noncoding regions. Some of these polymorphisms may also result in defective protein expression (e.g., as a result of defective splicing). Other single nucleotide polymorphisms have no phenotypic effects.

Many of the methods described below require amplification of DNA from target samples and purification of the amplified products. This can be accomplished by PCR, for instance. See generally, PCR Technology, Principles and Applications for DNA Amplification (ed. H. A. Erlich), Freeman Press, New York, N.Y., 1992; PCR Protocols. A Guide to Methods and Applications (eds. Innis, et al.), Academic Press, San Diego, Calif., 1990; Mattila et al., Nucleic Acids Res. 19:4967 (1991); Eckert et al., PCR Methods and Applications 1:17 (1991); PCR (eds. McPherson et al., IRS Press, Oxford); and U.S. Pat. No. 4,683,202.

Other suitable amplification methods include the ligase chain reaction (LCR) (see Wu and Wallace, Genomics 4:560 (1989); Landegren et al., Science 241:1077 (1988)), transcription amplification (Kwoh et al., Proc. Natl. Acad. Sci. USA 86:1173 (1989), self-sustained sequence replication (Guatelli et al., Proc. Natl. Acad. Sci. USA 87:1874 (1990), and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

Another aspect of the invention is a method for detecting a variant allele of a human FATP gene, comprising preparing amplified, purified FATP DNA from a reference human and amplified, purified, FATP DNA from a “test” human to be compared to the reference as having a variant allele, using the same or comparable amplification procedures, and determining whether the reference DNA and test DNA differ in DNA sequence in the FATP gene, whether in a coding or a noncoding region, wherein, if the test DNA differs in sequence from the reference DNA, the test DNA comprises a variant allele of a human FATP gene. The following is a discussion of some of the methods by which it can be determined whether the reference FATP DNA and test FATP DNA differ in sequence.

Direct Sequencing.

The direct analysis of the sequence of variant alleles of the present invention can be accomplished using either the dideoxy chain termination method or the Maxam and Gilbert method (see Sambrook et al., Molecular Cloning: A Laboratory Manual, 2nd ed., Cold Spring Harbor Press, New York 1989; Zyskind et al., Recombinant DNA Laboratory Manual, Acad. Press, 1988)).

Denaturing Gradient Gel Electrophoresis.

Amplification products generated using the polymerase chain reaction can be analyzed by the use of denaturing gradient gel eletrophoresis. Different alleles can be identified based on the different sequence-dependent strand dissociation properties and electrophoretic migration of DNA in solution (chapter 7 in Erlich, ed. PCR Technology, Principles and Applications for DNA Amplification, W. H. Freeman and Co., New York, 1992).

Single-Strand Conformation Polymorphism Analysis.

Alleles of target sequences can be differentiated using single-strand conformation polymorphism analysis, which identifies base differences by alteration in electrophoretic migration of single stranded PCR products, as described in Orita et al., Proc. Natl. Acad. Sci. USA 86:2766-2770 (1989). Amplified PCR products can be generated as described above, and heated or otherwise denatured, to form single-stranded amplification products. Single-stranded nucleic acids may refold or form secondary structures which are partially dependent on the base sequence. The different electrophoretic mobilities of single-stranded amplification products can be related to base-sequence differences between alleles of target sequences.

Detection of Binding by Protein that Binds to Mismatches.

Amplified DNA comprising the FATP gene or portion of the gene of interest from genomic DNA, for example, of a normal individual is prepared, using primers designed on the basis of the DNA sequences provided herein. Amplified DNA is also prepared, in a similar manner, from genomic DNA of an individual to be tested for bearing a distinguishable allele. The primers used in PCR carry different labels, for example, primer 1 with biotin, and primer 2 with ³²P. Unused primers are separated from the PCR products, and the products are quantitated. The heteroduplexes are used in a mismatch detection assay using immobilized mismatch binding protein (MutS) bound to nitrocellulose. The presence of biotin-labeled DNA wherein mismatched regions are bound to the nitrocellulose via MutS protein, is detected by visualizing the binding of streptavidin to biotin. See WO 95/12689. MutS protein has also been used in the detection of point mutations in a gel-mobility-shift assay (Lishanski, A. et al., Proc. Natl. Acad. Sci. USA 91:2674-2678 (1994)).

Other methods, such as those described below, can be used to distinguish a FATP allele from a reference allele, once a particular allele has been characterized as to DNA sequence.

Allele-Specific Probes.

The design and use of allele-specific probes for analyzing polymorphims is described by e.g., Saiki et al., Nature 324:163-166 (1986); Dattagupta, EP 235,726, Saiki, WO 89/11548. Allele-specific probes can be designed so that they hybridize to a segment of a target DNA from one individual but do not hybridize to the corresponding segment from another individual due to the presence of different polymorphic forms in the respective segments from the two individuals. Hybridization conditions should be sufficiently stringent that there is a significant difference in hybridization intensity between alleles, and preferably an essentially binary response, whereby a probe hybridizes to only one of the alleles. Some probes are designed to hybridize to a segment of target DNA such that the polymorphic site aligns with a central position (e.g., in a 15-mer at the 7 position; in a 16-mer, at either the 8 or 9 position) of the probe. This design of probe achieves good discrimination in hybridization between different allelic forms.

Allele-specific probes are often used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence.

Allele-Specific Primers.

An allele-specific primer hybridizes to a site on target DNA overlapping a polymorphism, and only primes amplification of an allelic form to which the primer exhibits perfect complementarity. See Gibbs, Nucleic Acid Res. 17:2427-2448 (1989). This primer is used in conjunction with a second primer which hybridizes at a distal site. Amplification proceeds from the two primers, resulting in a detectable product which indicates the particular allelic form is present. A control is usually performed with a second pair of primers, one of which shows a single base mismatch at the polymorphic site and the other of which exhibits perfect complementarity to a distal site. The single-base mismatch prevents amplification and no detectable product is formed. The method works best when the mismatch is included in the 3′-most position of the oligonucleotide aligned with the polymorphism because this position is most destabilizing to elongation from the primer (see, e.g., WO 93/22456).

Gene Chips.

Allelic variants can also be identified by hybridization to nucleic acids immobilized on solid supports (gene chips), as described, for example, in WO 95/11995 and U.S. Pat. No. 5,143,854, both of which are incorporated herein by reference. WO 95/11995 describes subarrays that are optimized for detection of a characterized variant allele. Such a subarray contains probes designed to be complementary to a second reference sequence, which is an allelic variant of the first reference sequence.

The present method is illustrated by the following examples, which are not intended to be limiting in any way.

### EXAMPLES

### Materials and Methods

The following Materials and Methods were used in the work described in Examples 1-5.

Sequence Alignment of FATP Clones.

The DNA sequence for mouse FATP1 was obtained from the National Center for Biotechnology Information nonredundant database. cDNAs for mmFATP2, 3, 4, and 5 were obtained by screening mouse expression libraries (purchased from GIBCO/BRL, Rockville, Md.) with probes derived from the cloned expressed sequence tags (ESTs) (Research Genetics, Huntsville, Ala.). Full-length clones were obtained for mmFATP2 and 5 and partial sequences for mmFATP3 and 4. The sequences described herein have been deposited in the GenBank database (Accession Nos. FATP2, AF072760; FATP3, AF072759; FATP4, AF072758; FATP5, AF072757).

Neither FATP2 nor FATP5 contains an in-frame stop codon upstream of the putative initiator methionine; initiator methionines were assigned by homology with that in mmFATP 1 and by the presence of a signal sequence immediately after it. The Mycobacterium tuberculosis, Caenorhabditis elegans, and Saccharomyces cerevisiae sequences were present in the dbEST database as part of the sequencing projects for these organisms. Sequences were aligned utilizing a ClustalX algorithm and the resulting alignment exported to SeqVu. Homologous amino acid substitutions are boxed in FIG. 1 and were determined using the Dayhoff 250 method with a 50% homology cutoff.

Cell Transfection and LCFA Uptake.

COS cells were cotransfected using the DEAE-dextran method with the mammalian expression vector pCDNA 3.1 (Invitrogen, Carlsbad, Calif.) expressing the gene for CD2 (pCDNA-CD2) in combination with either a pCDNA 3.1 or pCMVSPORT2 (GIBCO/BRL, Rockville, Md.) expression vector containing one of the murine or nematode FATP genes (pCDNA-mmFATP1, pCDNA-FATP2, pCMVSPORT-FATP5, pCDNA-ceFATPb). Two days after transfection, cells were assayed for CD2 expression with a phycoerythrin-coupled anti-CD2(PE-CD2) monoclonal antibody (PharMingen, Franklin Lakes, N.J.), and fatty acid uptake was assayed with a BODIPY-labeled fatty acid analogue (Molecular Probes). Briefly, cells were washed twice with PBS (phosphate buffered saline) and stained with PE-CD2 at 4° C. for 30 min in PBS containing 10% fetal calf serum. They were then washed three times with PBS/fetal calf serum for 5 min followed by an incubation for 2 min at 37° C. in fatty acid uptake solution, which contained 0.1 μM BODIPY-FA and 0.1% fatty acid-free BSA (bovine serum albumin) in PBS (Schaffer, J. E. & Lodish, H. F. (1994) Cell 79:427-436). After 2 min, the cells were washed four times with ice-cold PBS/0.1% BSA. The cells were then removed from the plates with PBS containing 5 mM EDTA and resuspended in PBS containing 10% fetal calf serum and 10 mM EDTA. PE-CD2 and BODIPY-FA fluorescence were measured using a FACScan (Becton Dickinson, Franklin Lakes, N.J.). COS cells were gated on forward scatter (FSC) and side scatter (SS). Cells exhibiting more than 300 CD2 fluorescence units (dsim) representing 15% of all cells were deemed CD2 positive and their BODIPY-FA fluorescence was quantitated.

E. coli-Based LCFA Uptake Assay.

The full-length coding region of mtFATP and a control protein, the mammalian transcription factor TFE3, were subcloned into the inducible, prokaryotic expression vector pET (Novagen, Madison, Wis.). Expression was induced with 1 mM isopropyl β-D-thiogalactoside (IPTG) for 1 hour, or cells were left uninduced. Cells were washed in PBS/0.1% BSA and resuspended in 1 ml PBS/0. 1% BSA containing 0.1 μM [³H]palmitate (NEN) at 37° C. Uptake was stopped after the indicated incubation time by transferring the cells onto filter paper using a cell harvester (Brandel, Bethesda, Md.). Filters were washed extensively with ice-cold PBS/0.1% BSA, and [³H]palmitate was quantitated by scintillation counting.

Northern Blots.

Northern blot analysis of murine FATP expression was done using poly(A) mRNA blots (Clontech, Palo Alto, Calif.). Probes of each of the FATPs were derived from the 3′ untranslated regions of each gene and were <60% identical in sequence. Probes were labeled by random priming (Boehringer Mannheim, Indianapolis, Ind.) and hybridized at 65° C. Blots were extensively washed in 0.2% SSC/0.1% SDS at 65° C.

Generation of Phylogenetic Trees.

Complete and partial sequences for FATP genes from human, rat, mouse, puffer fish, Drosophila melanogaster, C. elegans, S. cerevisiae, and M. tuberculosis were aligned using ClustalX. A homologous region of 48 amino acids (residues 472-519 in mmFATP1) from all of the genes was used to determine phylogenetic relationship within ClustalX. Based on these data a phylogenetic tree was generated using Tree View PPC (FIG. 5).

Nomenclature.

It is proposed that the FATP genes be given a species specific prefix (mm, Mus musculus; hs, Homo sapiens; mt, M tuberculosis; dm, D. melanogaster; ce, C. elegans, sc, S. cerevisiae) and numbered such that mammalian homologues in different species share the same number but differ in their prefix. Since the two C. elegans genes cannot be paired with a specific human or mouse FATP, they have been designated ceFATPa and ceFATPb.

### Example 1

### Identification of Novel Mammalian FATPs

The National Center for Biotechnology Information EST database was screened, using the mouse FATP protein sequence (mmFATP1), to identify novel FATPs. This strategy led to the identification of more than 50 murine EST sequences which could be assembled into five distinct contiguous DNA sequences (contigs). One contig was identical to the previously cloned FATP, which has been renamed FATP1. Another, which has been renamed FATP2, is the murine homologue of a rat gene previously identified by others as a very long chain acyl-CoA synthase (Uchiyama, A., Aoyama, T., Kamijo, K., Uchida, Y., Kondo, N., Orii, T. & Hashimoto, T. (1996) J. Biol. Chem. 271:30360-30365). The other three contigs represented novel genes (FATP3, 4, and 5). Full-length clones for FATP2 and FATP5 and nearly complete sequences for FATP3 and 4 (FIG. 1) were obtained by screening cDNA libraries made from mouse day 10.5 embryos and adult liver. Also identified were human homologues for each of the murine genes in the EST database. A sixth human gene was also identified; whether this gene is also present in the mouse will require additional studies. Map positions are given in Tables 2 and 3.

The genetic loci for all of the human genes, with the exception of FATP5 which was already mapped as an unknown EST, were determined using the radiation hybrid panels. The map positions given below show the distance (in centiRays) from the closest framework marker. As a guideline, there are approximately 300 kb/cR.

TABLE 2
Mapping Data for Human Genes

| | |
|---|---|
| hsFATP1 | Chromosome Chr19 |
| | places 13.35 cR from WI-6344 (lod > 3.0) |
| hsFATP2 | Chromosome Chr15 |
| | places 4.92 cR from D15S126 (lod > 3.0) |
| hsFATP3 | Chromosome Chr1 |
| | places 13.24 cR from WI-2862 (lod > 3.0) |
| hsFATP4 | Chromosome Chr9 |
| | places 7.80 cR from WI-9685 (lod > 3.0) |
| hsFATP5 | unknown EST previously mapped to near D19S418 |
| hsFATP6 | Chromosome Chr5 |
| | places 1.41 cR from WI-4907 (lod > 3.0) |

The mouse map is an internal backcross panel consisting of 188 mouse backcross DNA's plus 4 controls (B6, Spretus, F1, Water). The backcross was constructed by crossing B6 by Spretus animals and then crossing those F1's back to B6. Mapping is accomplished by taking advantage of recombinational events during meiosis, and the use of PCR primers to detect the differences (by size or re-annealing events) at any given locus between the B6 and Spretus allele.

For the purposes of mapping, a novel set of primers (gene of interest) is used to amplify from all 188 DNA's and then typed as being a B6 (“B”) or a Spretus (“S”). This string of B's and S's is entered into the Map Manager program, which does a best fit calculation by comparing the string of 188 typings from the gene of interest to all loci already extant in the panel, for all 20 chromosomes. The gene of interest is then assigned to a particular area on a particular chromosome according to a number of parameters, including the minimalization of double cross-overs, and the highest LOD scores. Indicated in Table 3 are distances to the closest markers on either side of the FATP locus.

TABLE 3
Mapping Data for Mouse Genes

| | |
|---|---|
| mmFATP1 | Chromosome 8 |
| | places 2.82 cM from D8Mit132 (lod 43.4) and 1.81 cM |
| | from D8Mit74 (lod 43.5) |
| mmFATP2 | Chromosome 2 |
| | places 1.29 cM from D2Mit258 (lod 47.9) and 1.75 cM |
| | from D2NDS3 (lod 44.9) |
| mmFATP3 | Chromosome 3 |
| | places 2.54 cM from D3Mit22 (lod 29.5) and 19.62 cM |
| | from D3Mit42 (lod 13.6) |
| mmFATP4 | Chromosome 2 |
| | places 13.78 cM from D2Mit1 (lod 22.9) and 3.85 cM |
| | from D2Mit65 (lod 41.9) |
| mmFATP5 | Chromosome 7 |
| | places 7.28 cM proximal of D7Mit21 (lod 28.3) |

### Example 2

### Assessment of Function

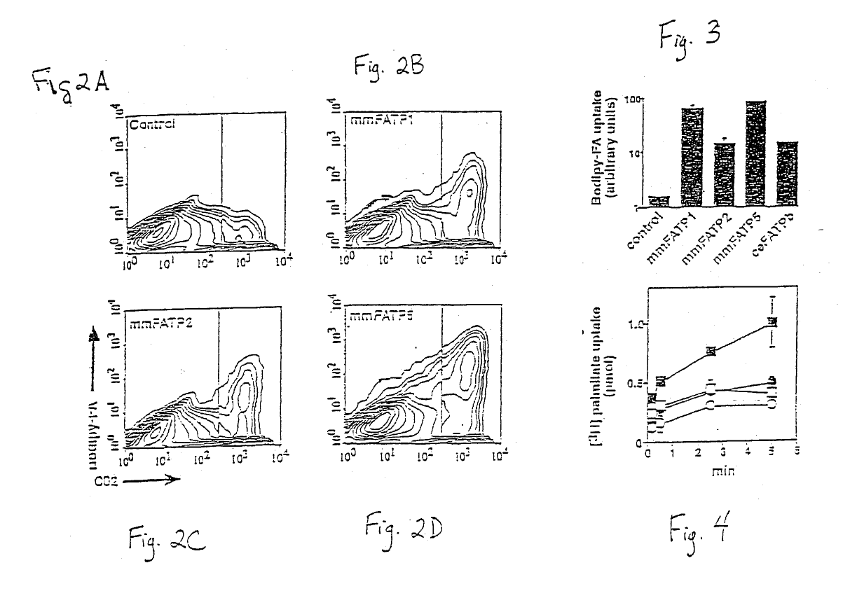

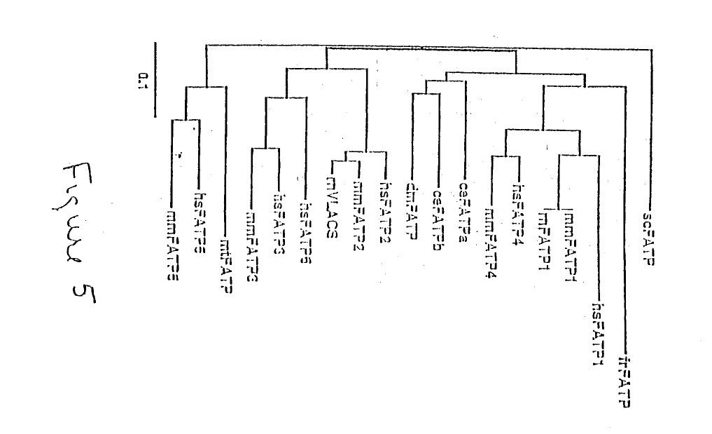

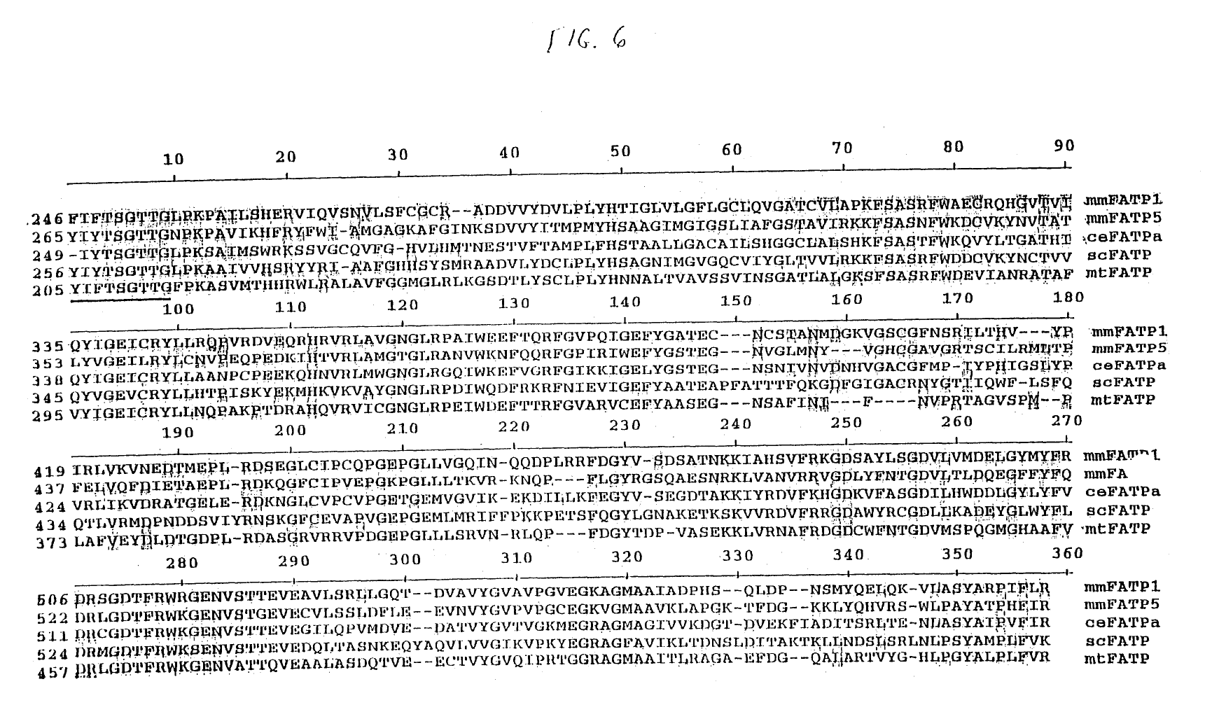

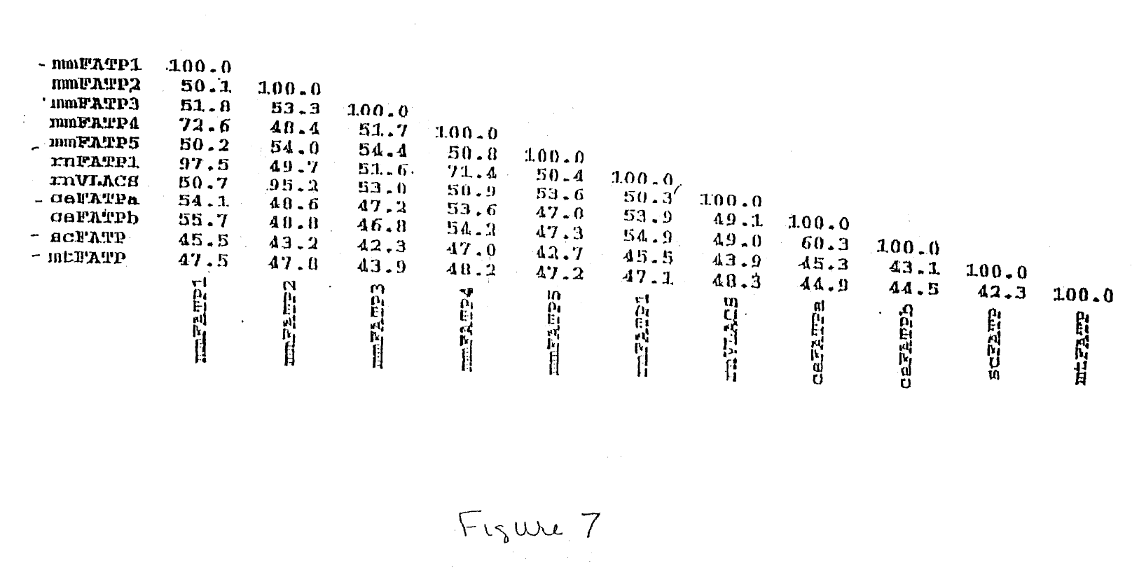

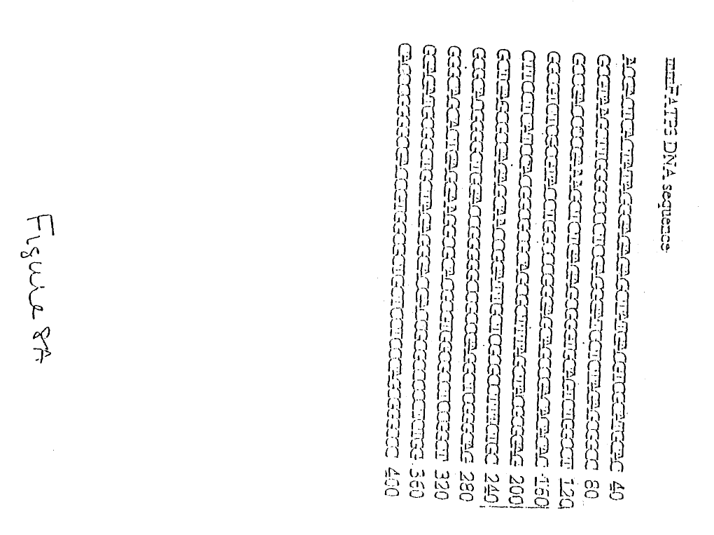

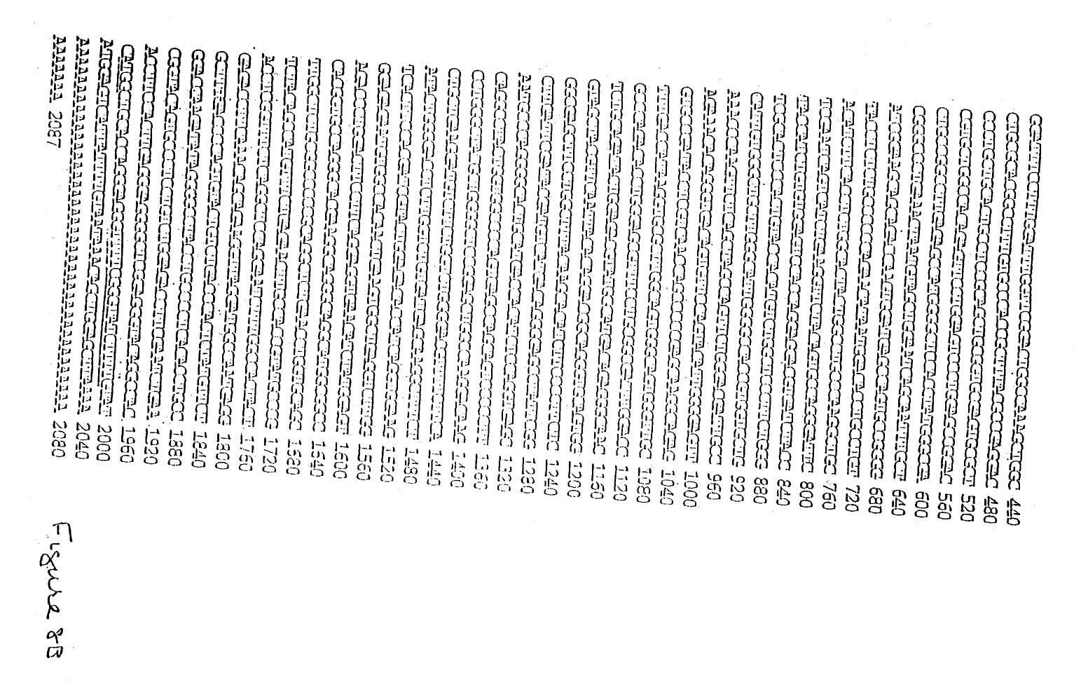

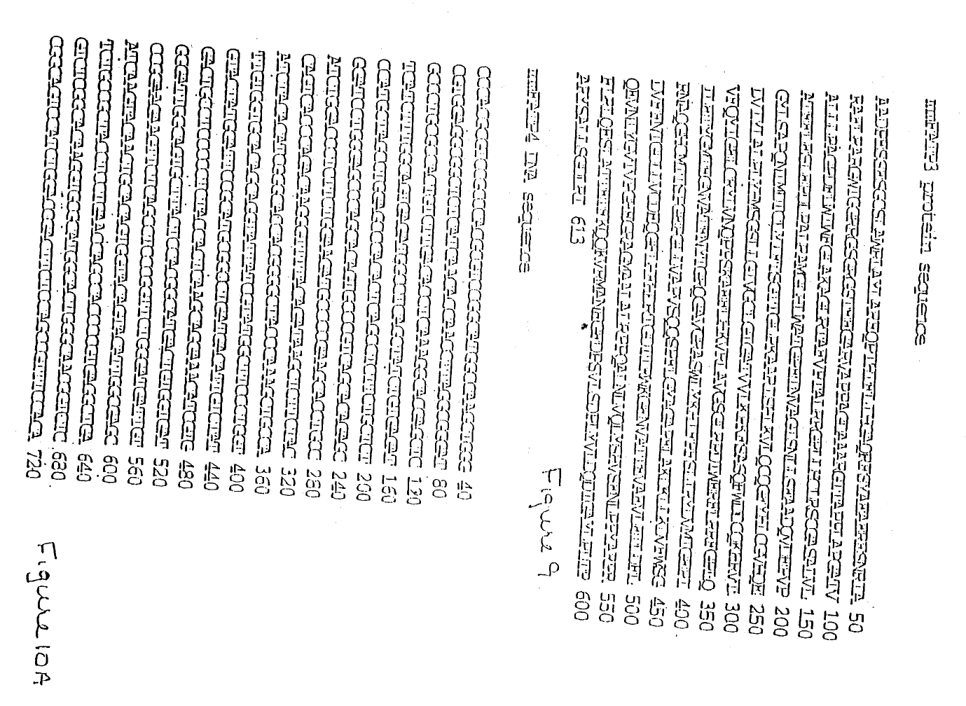

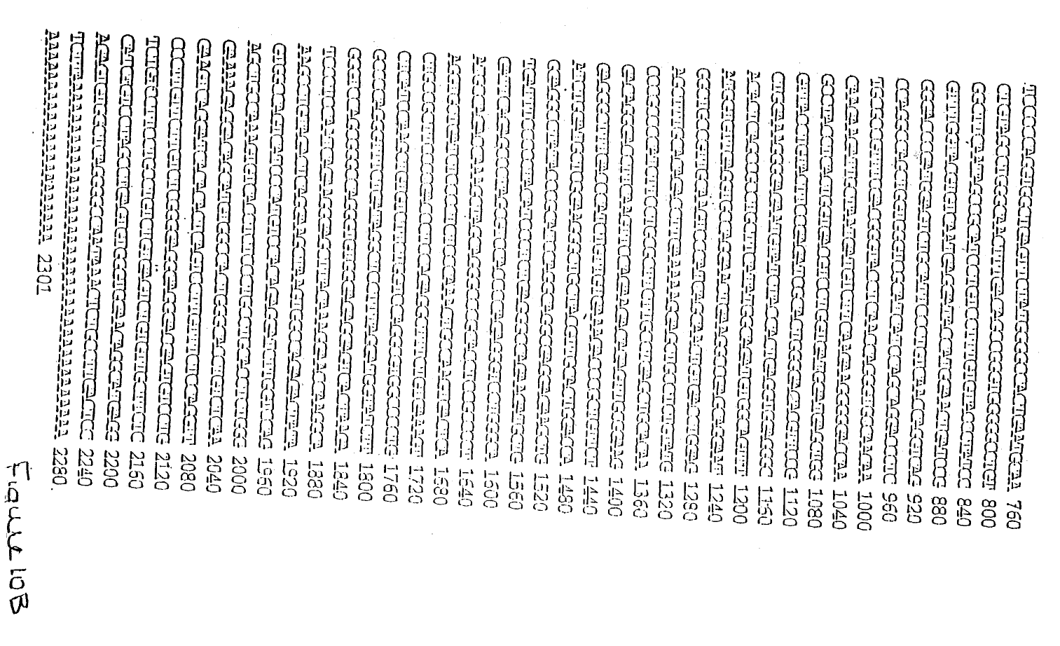

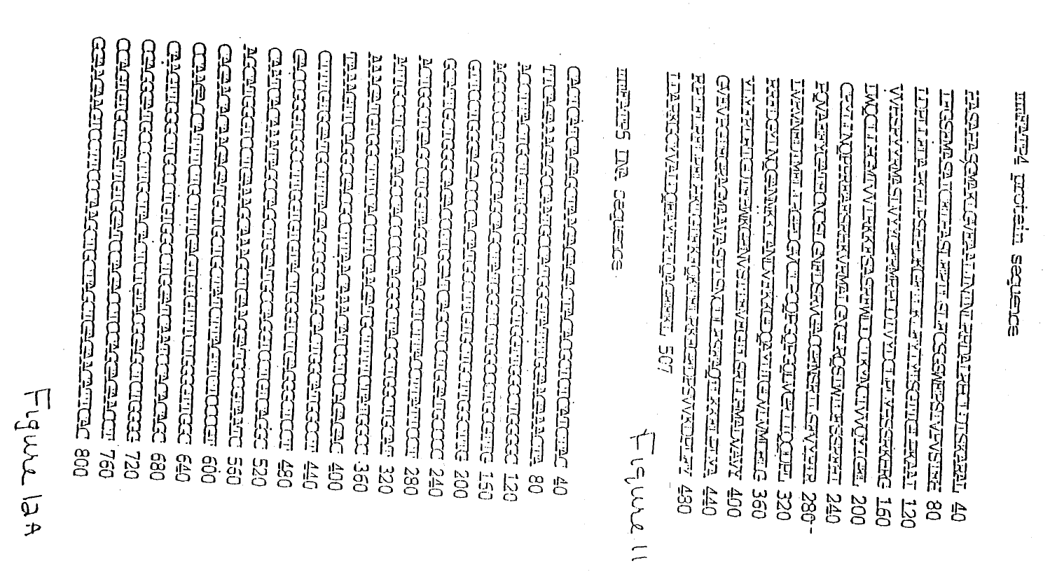

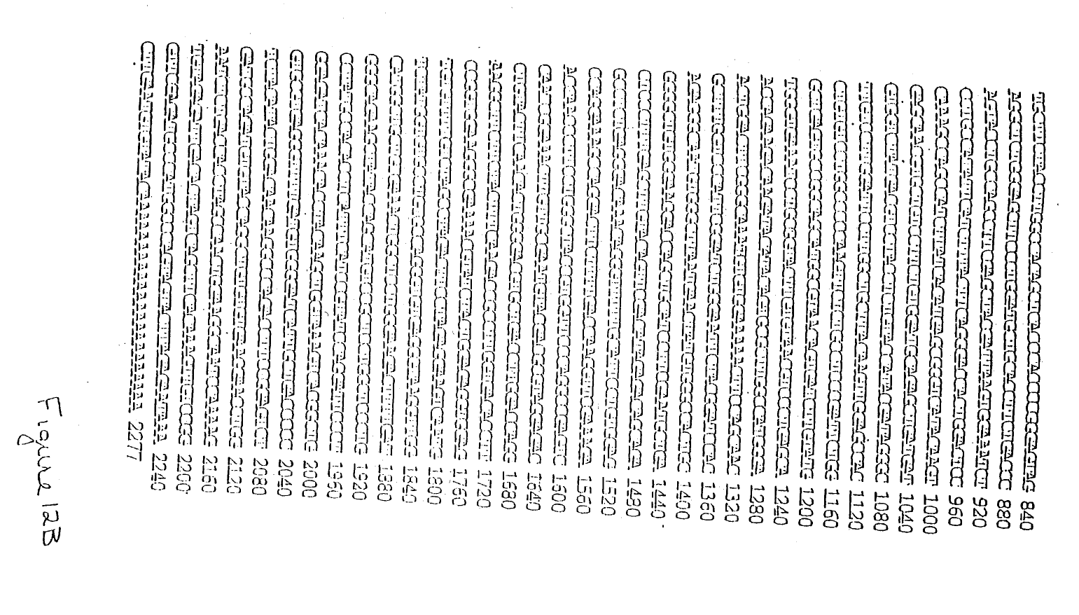

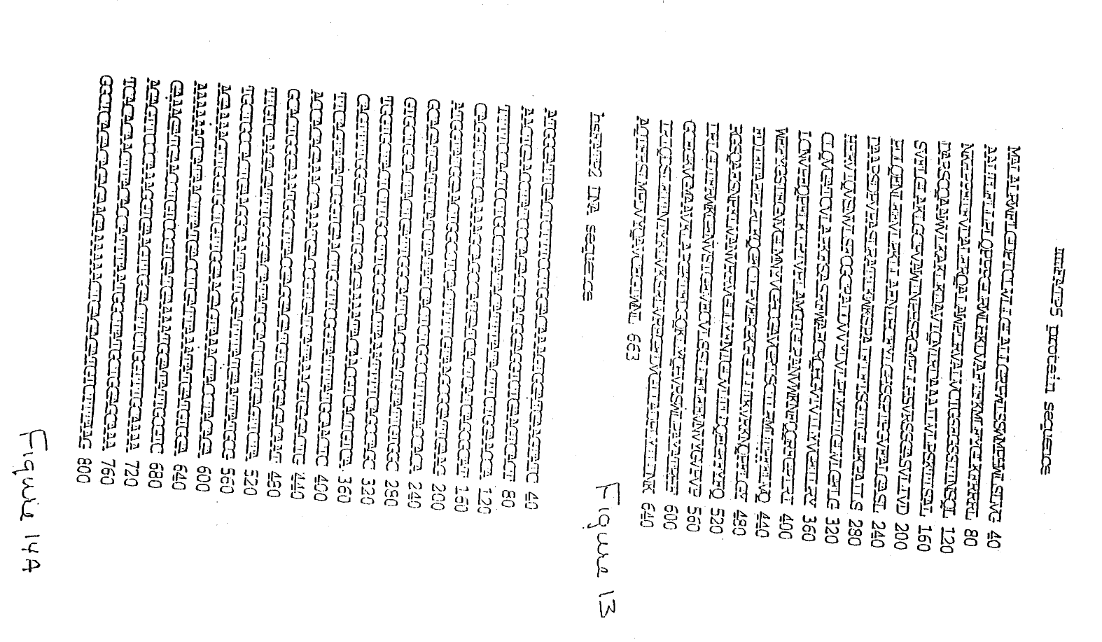

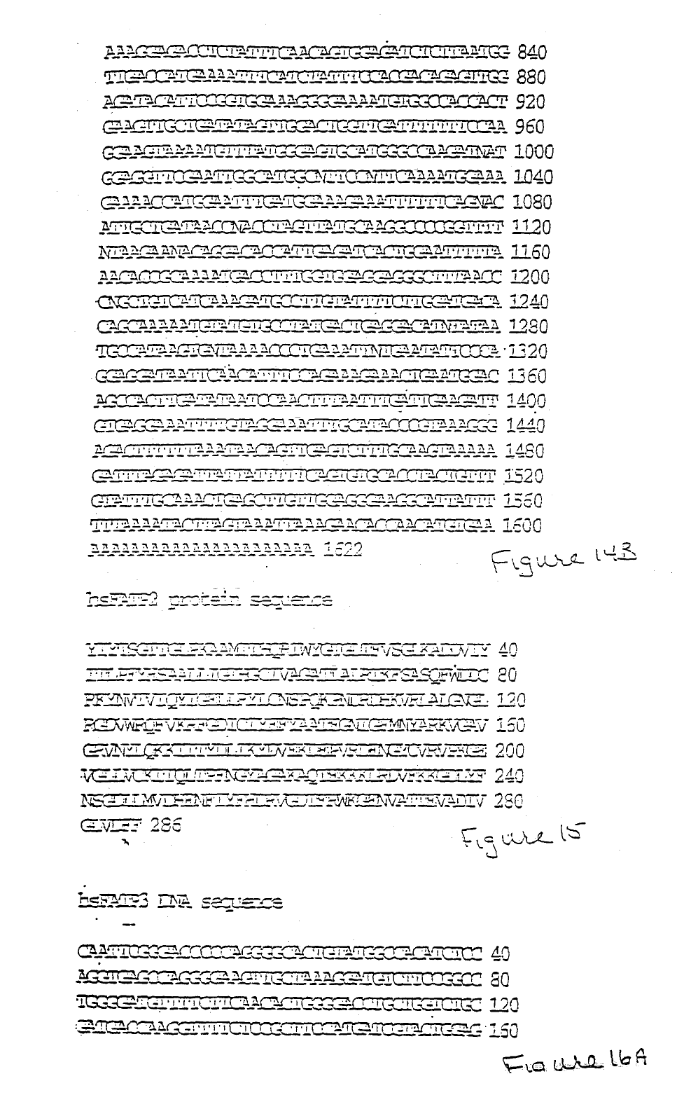

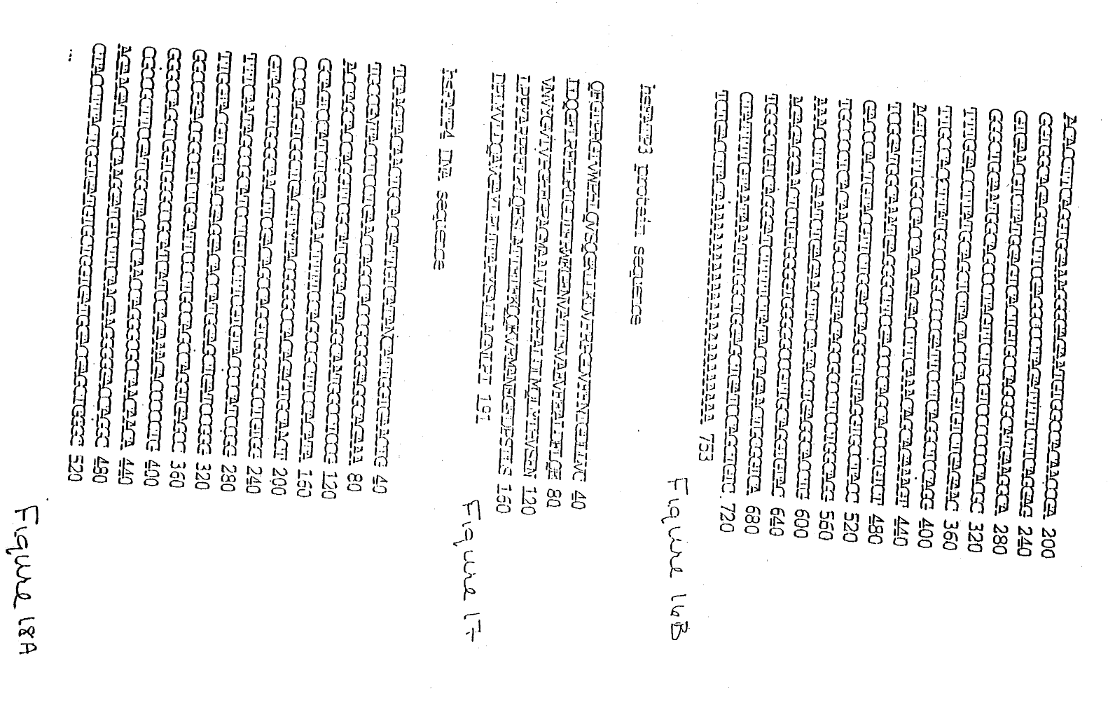

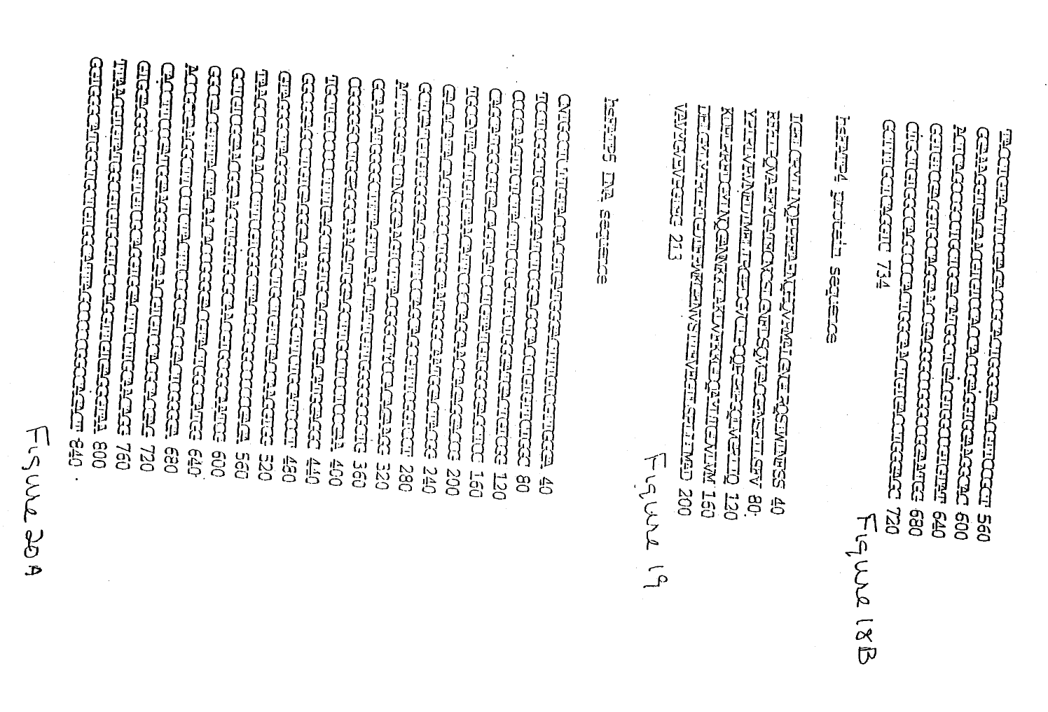

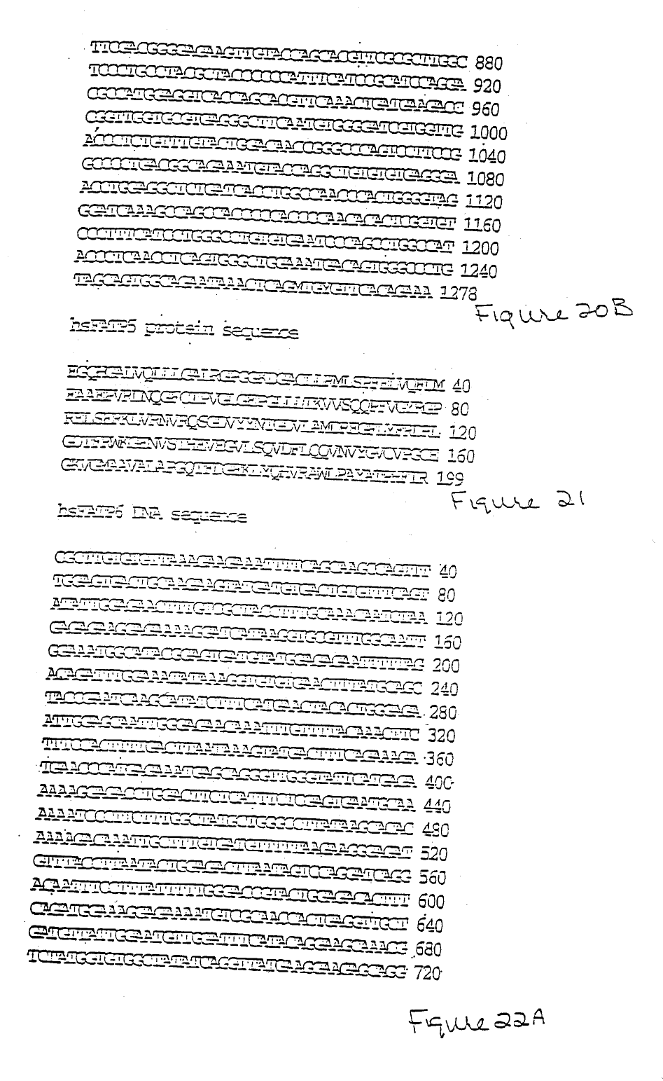

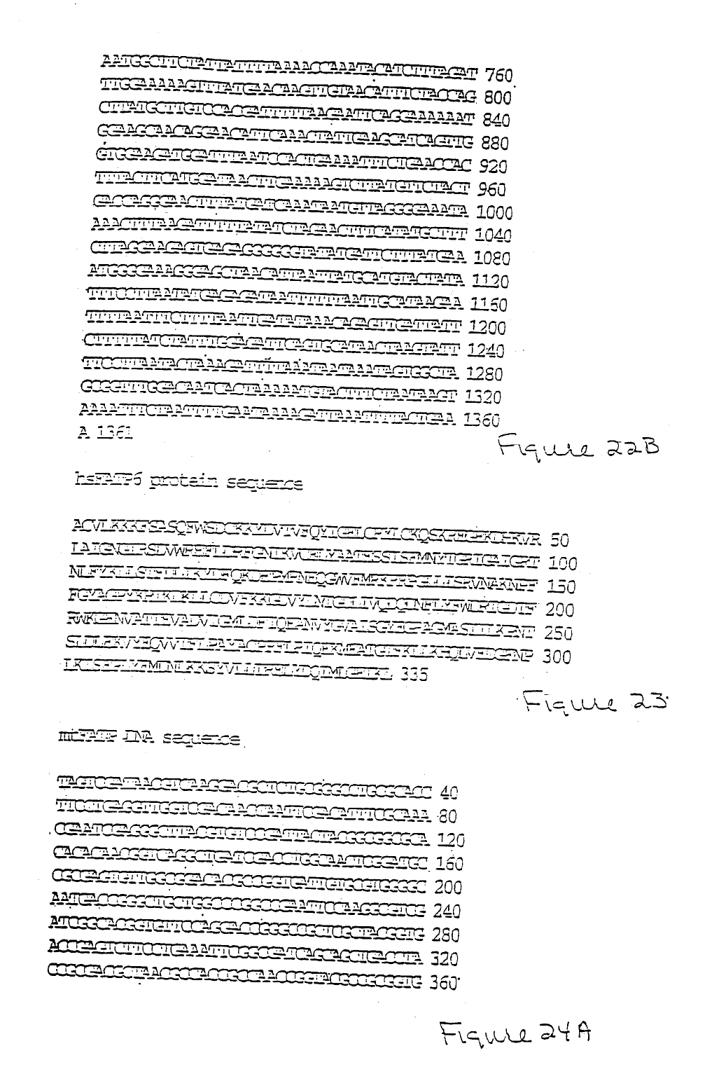

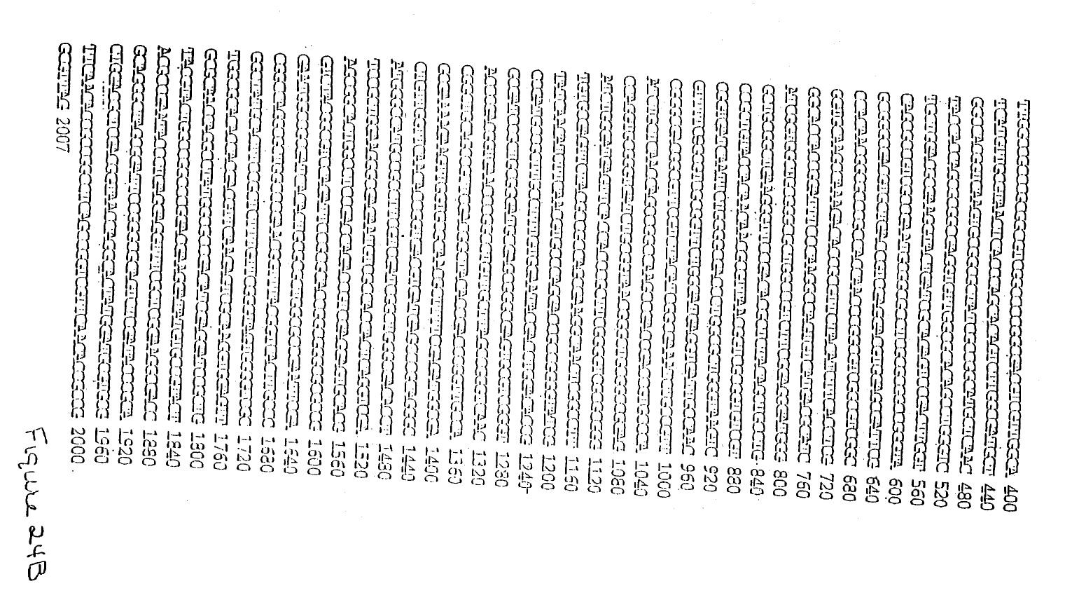

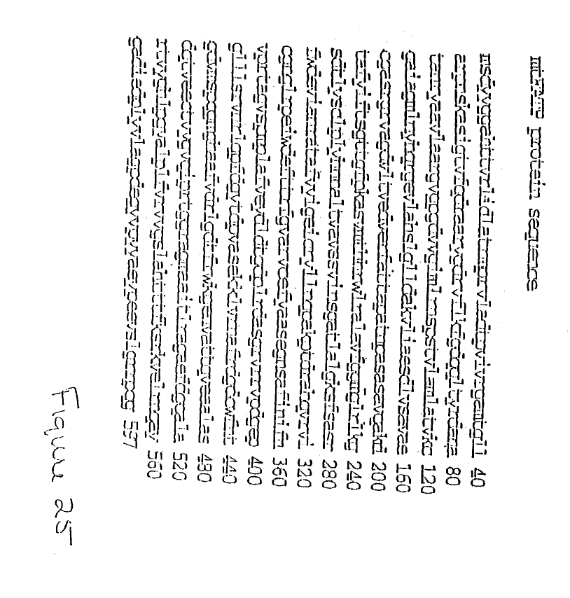

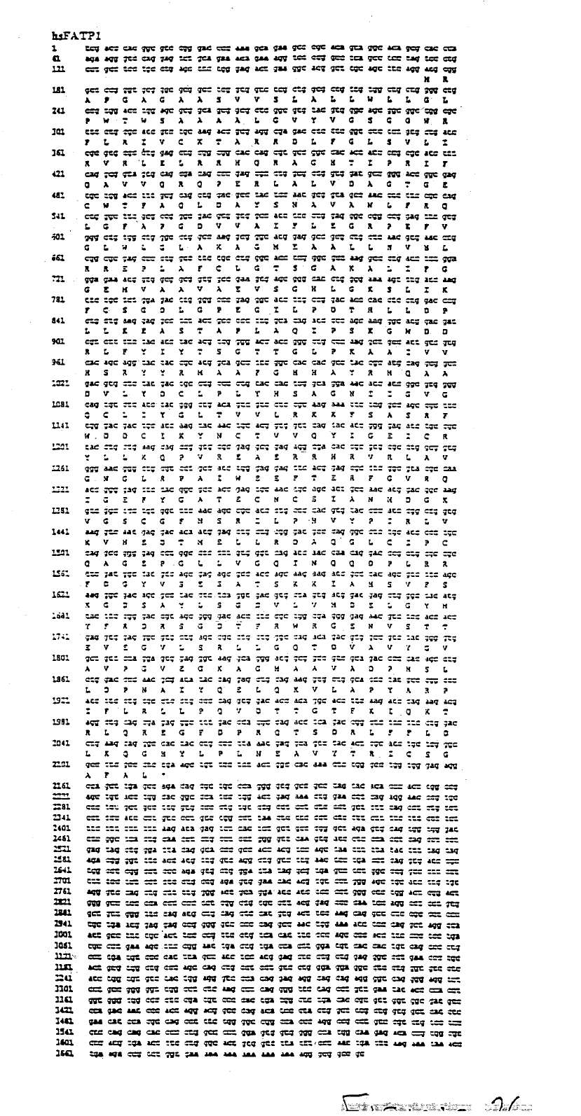

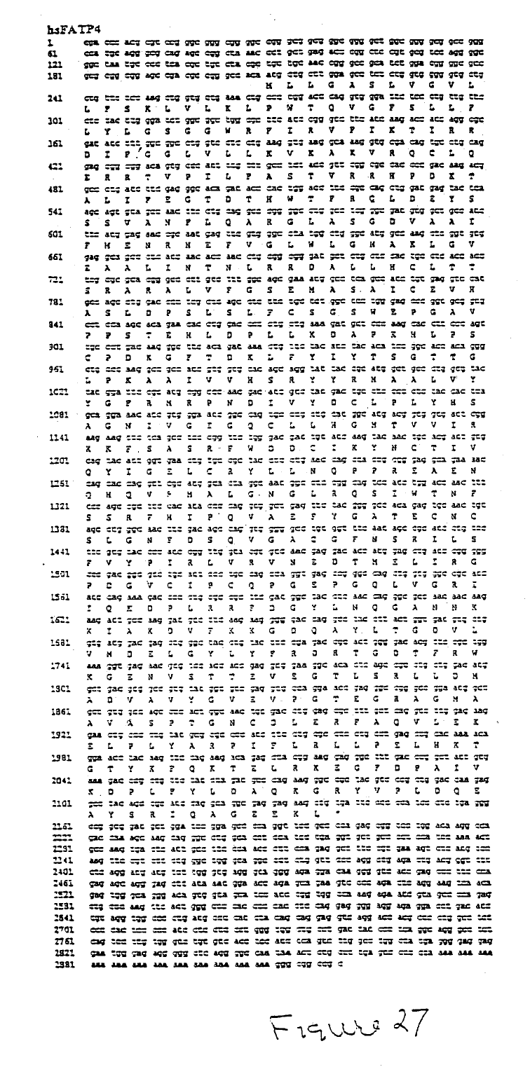

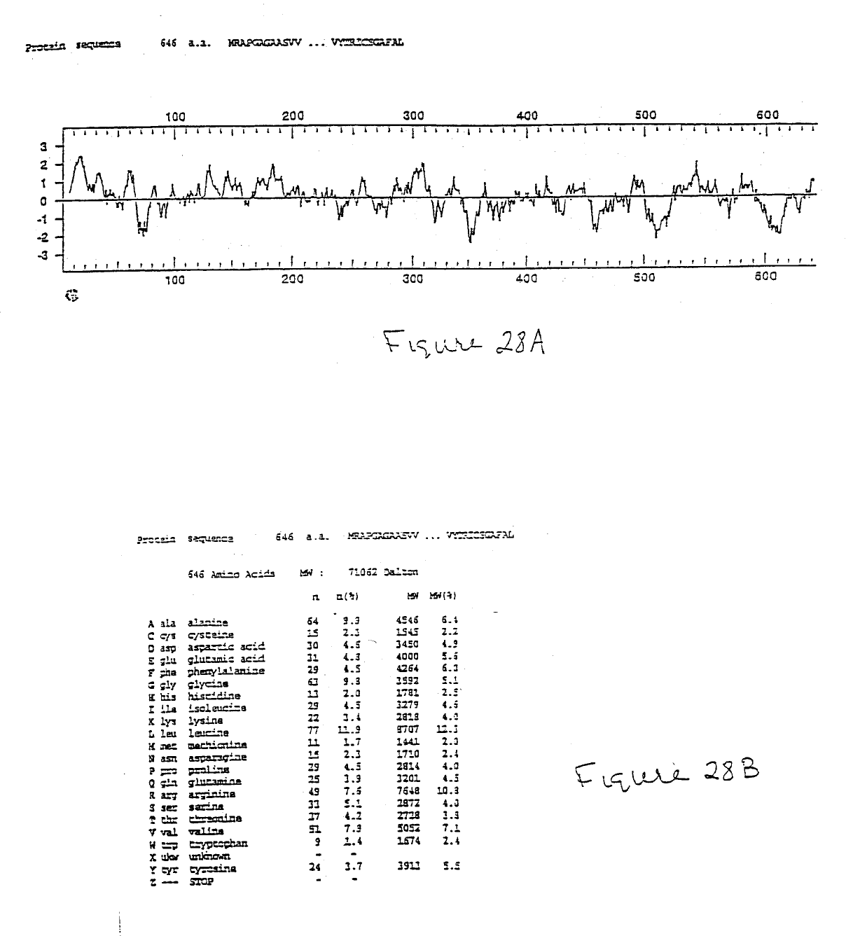

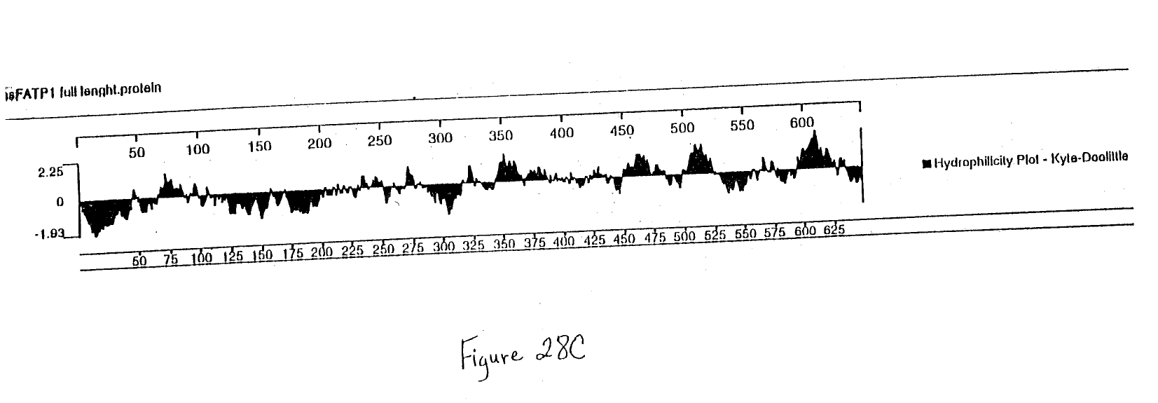

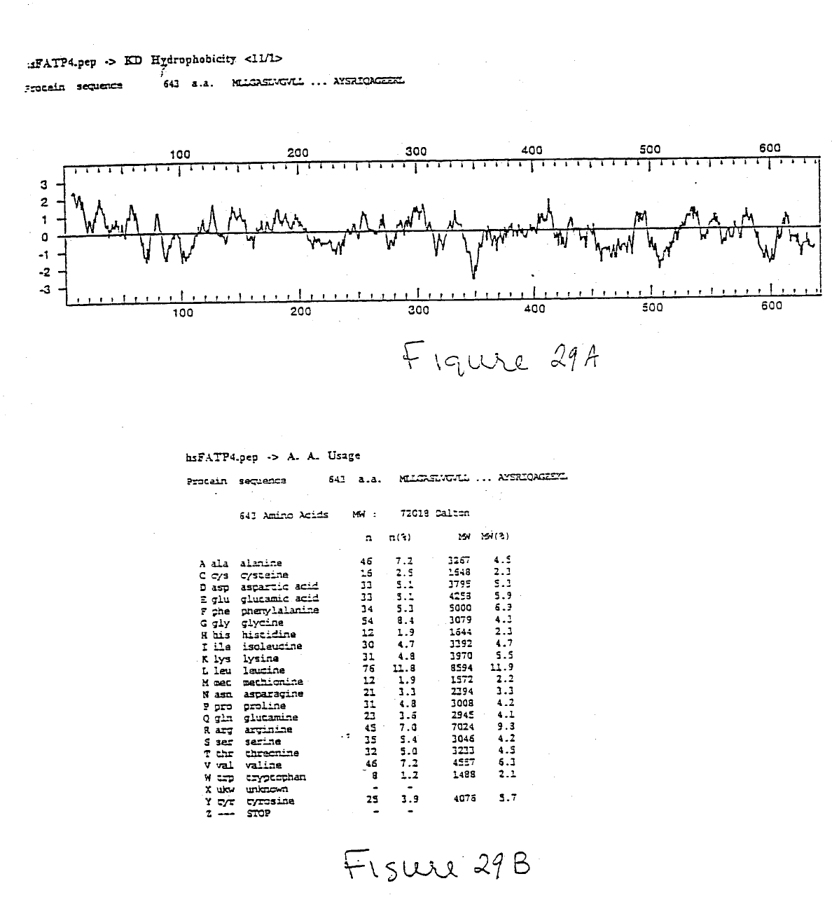

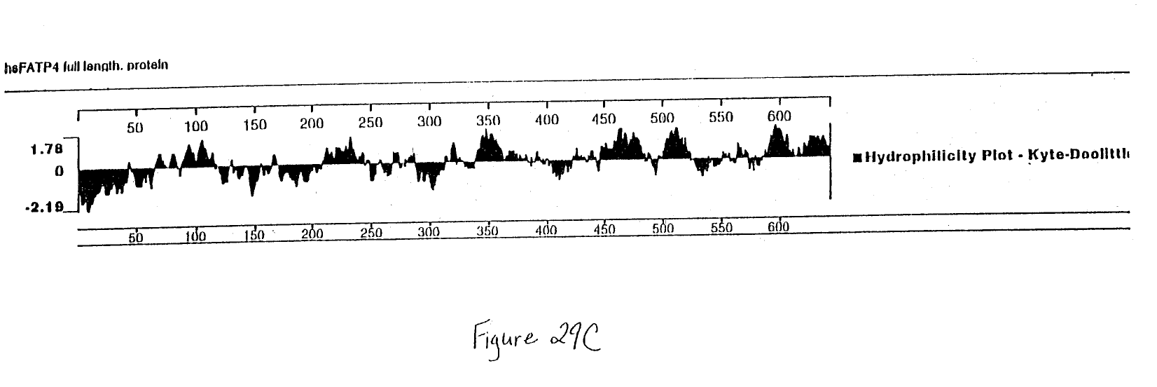

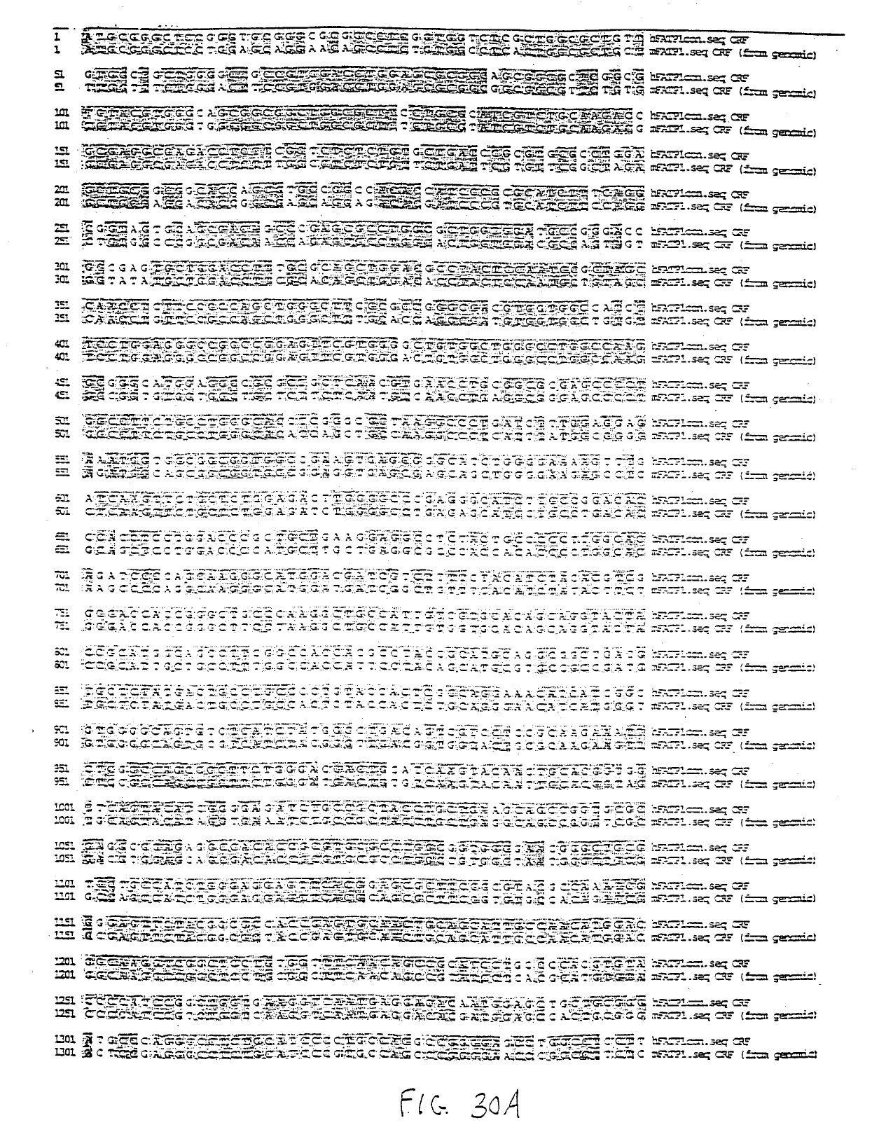

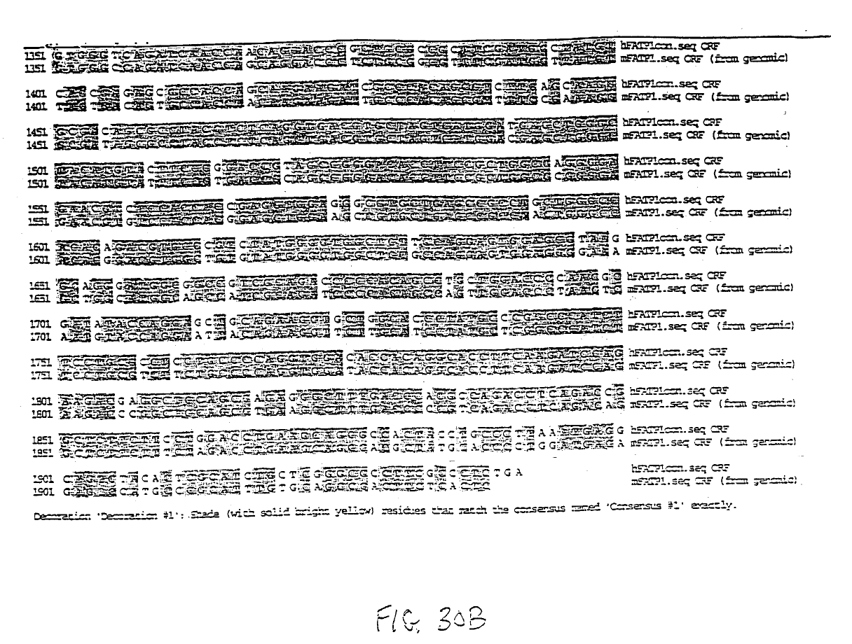

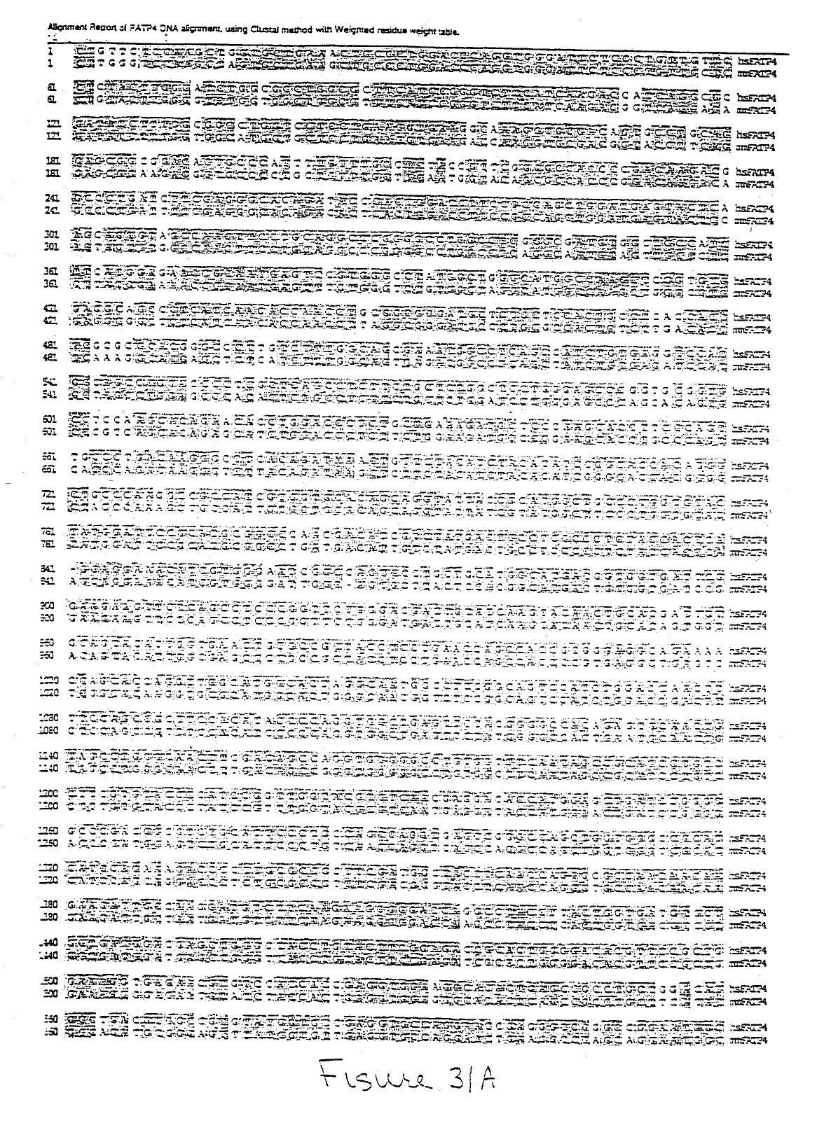

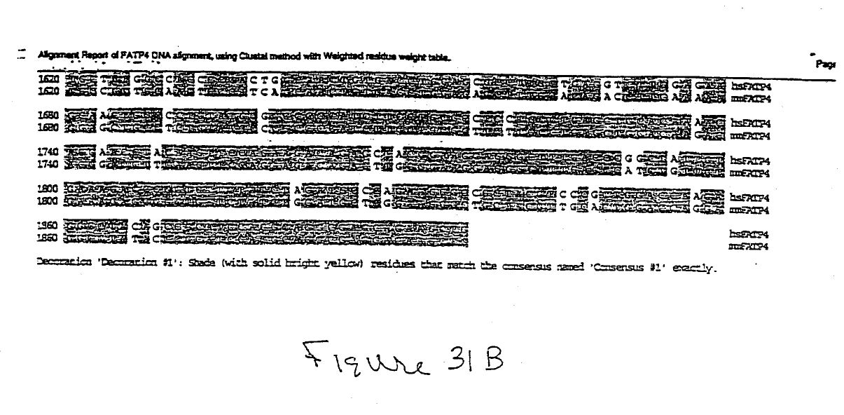

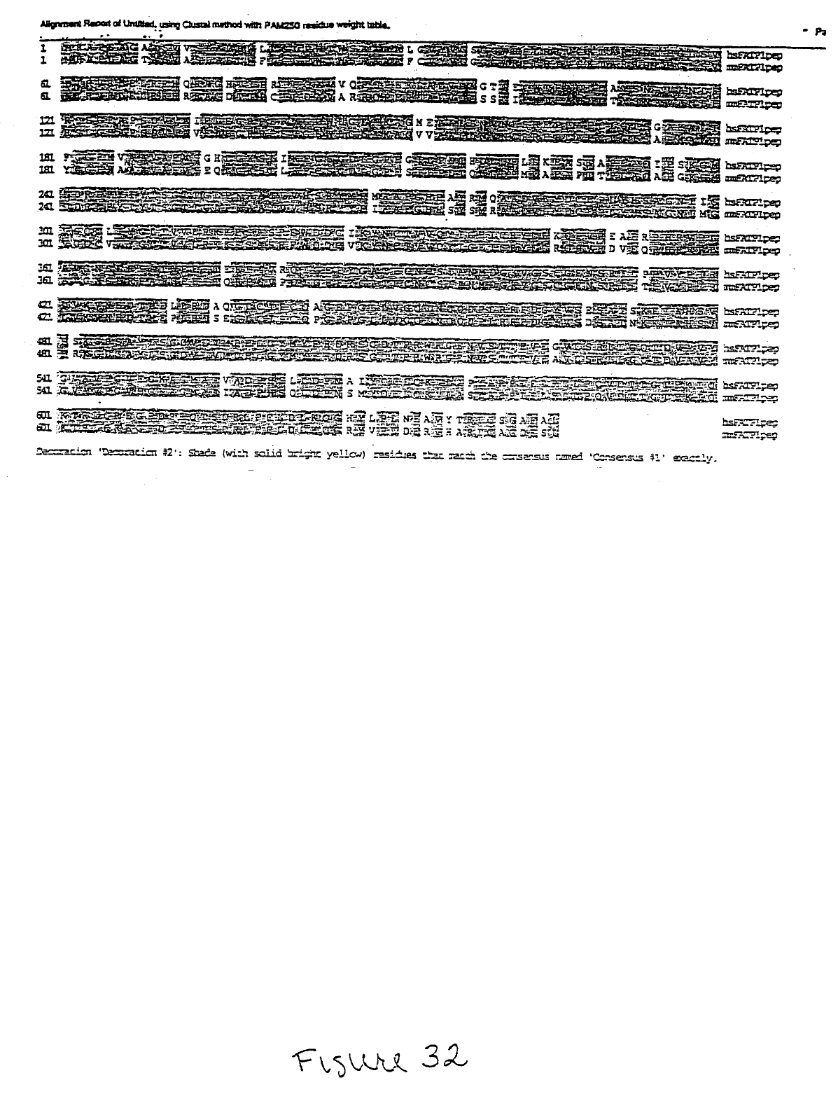

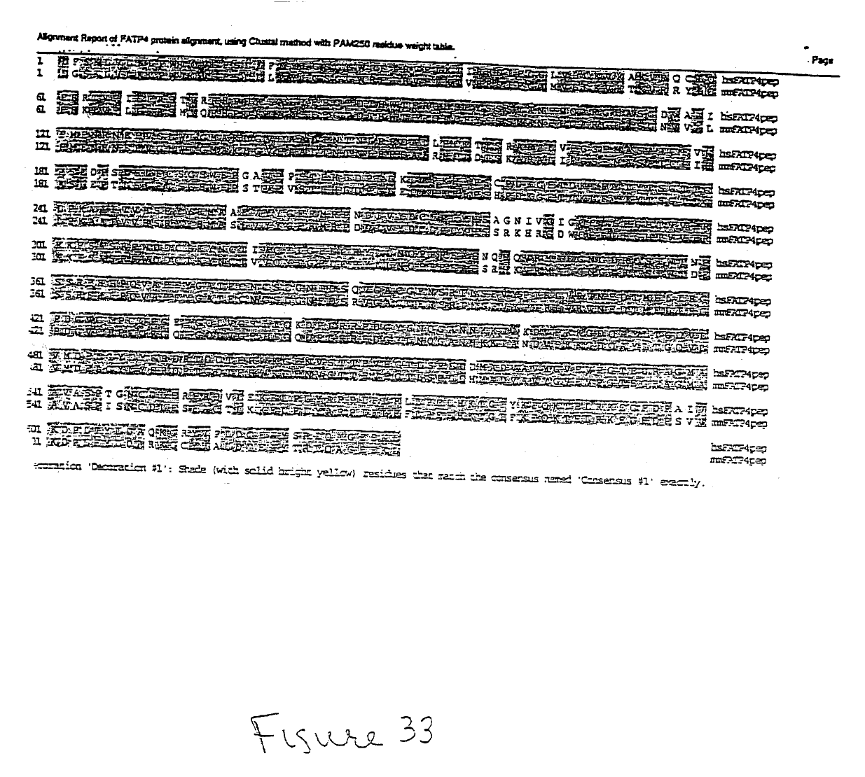

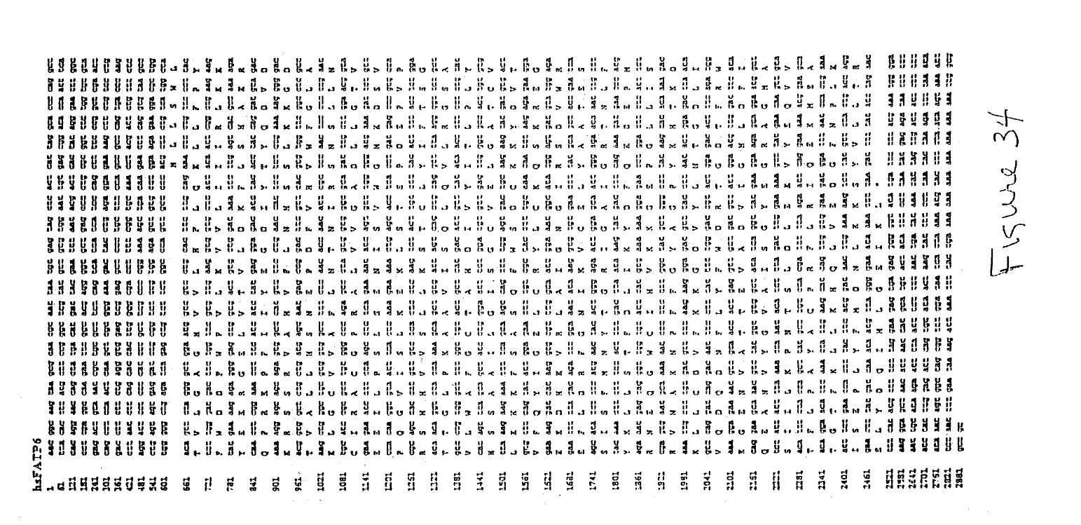

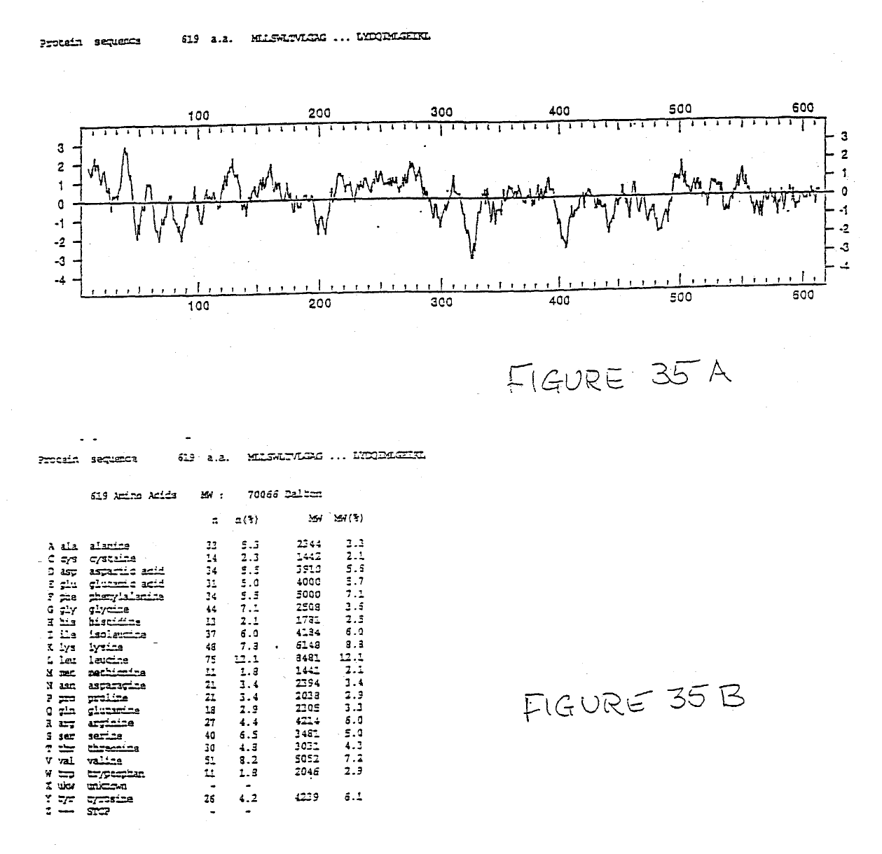

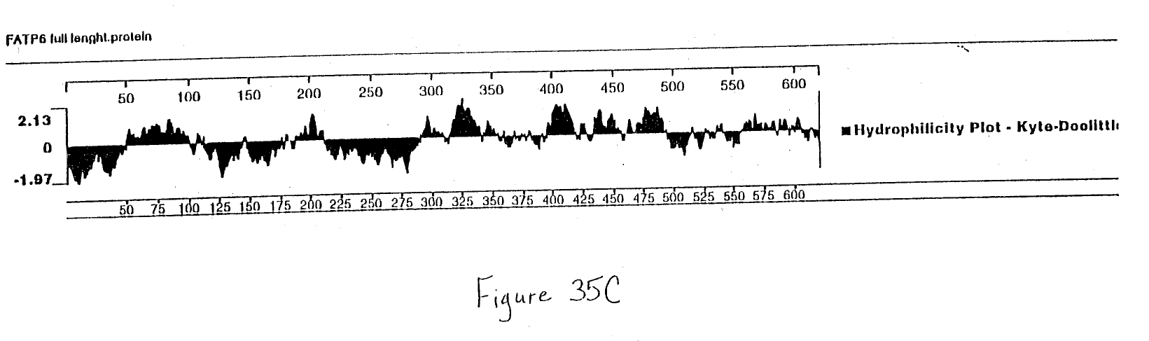

The ability of the newly identified mouse genes to function as fatty acid transporters was assessed using a fluorescence-activated cell sorting-based assay. COS cells were transiently cotransfected with expression vectors encoding the cell surface protein CD2 and either mmFATP1, mmFATP2, or mmFATP5, respectively. Two days after transfection, COS cells were stained with an antibody to CD2 and then incubated with a BODIPY-labeled fatty acid [BODIPY-FA, (Schaffer, J. E. & Lodish, H. F. (1994) Cell 79:427-436)]. The cells were then washed extensively, lifted off the dish, and analyzed by fluorescence-activated cell sorting. As judged by the number of CD2-positive cells, the transfection efficiency was approximately 20-30%. Fatty acid uptake was quantitated in the transiently transfected COS cells by measuring the BODIPY-FA fluorescence of the CD2-positive cells. Expression of CD2 had no effect on fatty acid uptake as shown by the finding that COS cells expressing only the transfected CD2 cDNA (CD2-positive) had the same low level of BODIPY-FA uptake as did untransfected (CD2-negative) control cells (FIG. 2A, control). In COS cells cotransfected with CD2 and mmFATP1, mmFATP2, or mmFATP5, uptake of BODIPY-FA by the transfected (CD2-positive) cells was increased between 15- to 90-fold over control (CD2 cDNA only) cells (FIGS. 2A-2D).

### Example 3

### Expression Patterns of Murine FATPs

Expression patterns of members of the murine FATP gene family were characterized by Northern blot analysis; to avoid cross-hybridization, the probes used were from the 3′ untranslated region of these genes, which are less than 60% identical in sequence. The expression pattern of FATP1 agrees with that previously found (Schaffer, J. E. & Lodish, H. F. (1994) Cell 79:427-436). Here, expression was seen primarily in heart and kidney. FATP2 is expressed almost exclusively in liver and kidney, which corresponds to the reported tissue distribution of the rat homologue [very long chain acyl-CoA (VLACS)] as assessed by Western blotting (Uchiyama, A., Aoyama, T., Kamijo, K., Uchida, Y., Kondo, N., Orii, T. & Hashimoto, T. (1996) J. Biol. Chem. 271:30360-30365). FATP3 is present in lung, liver, and testis. FATP5 is expressed only in liver and cannot be detected in other tissues even when the blot is overexposed. The human homologue of FATP5 is also liver specific and is not expressed in a wide array of other tissues tested, including fetal liver.

### Example 4

### FATPs Are Evolutionarily Conserved

The EST database was searched, using sequences conserved among the five murine FATP genes, for FATP genes in other organisms. Two homologues were found in C. elegans and one in M. tuberculosis. One of the C. elegans genes was cloned from a cDNA library and expressed in COS cells, as described for the murine FATPs. Overexpression of the nematode FATP resulted in a 15-fold increase of BODIPY-FA uptake compared with control cells (FIG. 3). The mycobacterial FATP gene was isolated from a phage library and assessed for its ability to facilitate fatty acid uptake. E. coli transformed with a prokaryotic, isopropyl P-D-thiogalactoside-inducible expression vector containing the mycobacterial FATP gene demonstrated a significant increase in the rate of [³H]palmitate uptake after induction, compared with uninduced bacteria or E. coli transformed with a control protein (FIG. 4). Novel FATP genes were also identified in F. rubripes (puffer fish) and D. melanogaster.

### Example 5

### Phylogenetic Tree of FATPs

Faergeman et al. (Faergeman, N. J., DiRusso. C. C., Elberger, A., Knudsen, J. & Black, P. N. (1997) J. Biol. Chem. 272:8531-8538) identified three regions of very strong conservation between the scFATP and mmFATP1 genes. The sequences of the FATPS were compared over a 311-amino acid FATP “signature sequence” which includes these conserved regions corresponding to amino acids 246-557 in mmFATP1 (underlined in FIG. 1). When compared with the National Center for Biotechnology Information nonredundant database, only one region of the “FATP signature sequence” shows significant homology to other proteins. This small stretch of amino acids (underlined in FIG. 1) is an AMP-binding motif found in a multitude of other proteins, such as acyl-CoA synthase, several CoA lipases, and gramicidin S synthetase component II (Schaffer, J. E. & Lodish, H. F. (1994) Cell 79:427-436). The relevance of this motif to fatty acid transport is unclear. Other highly conserved regions among the FATPs, including long stretches of amino acids >90% identical from mycobacteria to humans, are not found in any other class of proteins. A 48-amino acid segment of the FATP signature sequence was used to construct a phylogenetic tree (FIG. 5). Each of the human and mouse genes form their own branch; hsFATP6, which as yet has no murine homologue, is most closely related to hsFATP3 and mmFATP3. As expected, mVLACS is closer in sequence to mmFATP2 than to hsFATP2. The FATP genes of invertebrates i.e., C. elegans and D. melanogaster, are most closely related to each other. Surprisingly, the mycobacteral gene is more closely related to the human and mouse FATP5 genes than to the FATPs of any of the lower organisms. Whether this reflects coevolution of the mycobacterial and human genes awaits further study.

Materials and Methods

The following materials and methods were used in the work described in Examples 6-10.

Isolation of full-length human FATP 1 and 4

Full-length clones encoding human FATP1 and human FATP4 were identified by searching databases for sequences similar to murine FATP1-5 coding regions using the BlastX algorithm (Altschul et al., J. Mol. Biol. 215: 403-410, 1990).

A concatamer of nucleotide sequences comprising the coding sequences of mmFATP1 (Genbank Accession U15976), mmFATP2, mmFATP3 (SEQ ID NO:6), mmFATP4 (SEQ ID NO:8) and mmFATP5 (SEQ ID NO:10) was used to search the Millennium database using the BLASTX algorithm. Sequences with a score >150 were evaluated for whether they represented known FATP coding sequences.

Human clones with similarity to the 5′ end of murine FATP sequences were sequenced completely. Clones encoding full-length human FATP1 were obtained from a heart cDNA library constructed in the mammalian expression vector pMET7 (Tartaglia et al., Cell, 83: 1263-1271, 1995). Clones encoding full-length human FATP4 were obtained from a spleen cDNA library constructed in the mammalian expression vector pMET7.

Isolation of Full-Length Human FATP6

Several clones encoding human FATP6 were identified by searching public databases as described above. Five clones were analyzed further by restriction digestion and DNA sequencing. One of these clones (Genbank Accession # AA412064) appeared to be full-length and its entire insert was sequenced.

DNA Sequence Analysis

Sequences were aligned with the DNAStar program using the Clustal method. Hydrophobicity plots were generated with DNA Strider using the Kyte Doolittle method.

In situ Hybridization

Tissues were collected from 8 week old C57/B16 mice. Tissues were fresh frozen, cut on a cryostat at 10 μm thickness and mounted on Superfrost Plus slides (VWR). Sections were air dried for 20 minutes and then incubated with ice cold 4% paraformaldehyde (PFA)/phosphate buffered saline (PBS) for 10 minutes. Slides were washed 2 times 5 minutes with PBS, incubated with 0.25% acetic anhydride/1 M triethanolamine for 10 minutes, washed with PBS for 5 minutes and dehydrated with 70%, 80%, 95% and 100% ethanol for 1 minute each. Sections were incubated with chloroform for 5 minutes. Hybridizations were performed with ³⁵S-radiolabeled (5×10⁷ cpm/ml) cRNA probes generated from the 3′ untranslated regions of mouse FATPs by PCR followed by in vitro transcription in the presence of 50% formamide, 10% dextran sulfate, 1× Denhardt's solution, 600 mM NaCl, 10 mM DTT, 0.25% SDS and 10 μg/ml tRNA for 18 hours at 55° C. After hybridization, slides were washed with 10 mM Tris-HC1 pH 7.6, 500 mM NaCl, 1 mM EDTA (TNE) for 10 minutes, incubated in 40 μg/ml RNase A in TNE at 37° C. for 30 minutes, washed in TNE for 10 minutes, incubated once in 2× SSC at 60° C. for 1 hour, once in 0.2× SSC at 60° C. for 1 hour, once in 0.2× SSC at 65° C. for 1 hour and dehydrated with 50%, 70%, 80%, 90% and 100% ethanol. Localization of mRNA transcripts was detected by dipping slides in Kodak NBT-2 photoemulsion and exposing for 7 days at 4° C., followed by development with Kodak Dektol developer. Slides were counter stained with haematoxylon and eosin and photographed. Controls for the in situ hybridization experiments include the use of a sense probe which showed no signal above background in all cases.

Northern Blotting

Human mRNA blots were obtained from Invitrogen or Clontech. PCR fragments from the 3′ untranslated regions of human FATPs were used as probes. Blots were probed with ³²P-labeled DNA probes using the Rapid-Hyb buffer (Amersham, Buckinghamshire, UK) according to the manufacturer's instructions.

Cell transfection and LCFA uptake. COS cells were cotransfected, using lipofectamine (GIBCO BRL, Rockville, Md.) according to the manufacturer's instructions, with the mammalian expression vector pCDNA3.1 (Invitrogen, Carlsbad, Calif.) expressing the gene for CD2 in combination with a pMET7 expression vector (Tartaglia et al., Cell, 83:1263-1271, 1995) containing hsFATP1 (pMET7-hsFATP1) or hsFATP4 (pMET7-hsFATP4) or pMET7 alone. Two days after transfection, cells were assayed for CD2 expression with a phycoerythrin-coupled anti-CD2 (PE-CD2) monoclonal antibody (PharMingen, Franklin Lakes, N.J.), and fatty acid uptake was assayed with a BODIPY-labeled fatty acid analog (Molecular Probes) as described above.

### Example 6

### Determination of Expression of mmFATPs

mmFATP4, and to lesser extent mmFATP2, are expressed at high levels in the brush border layer of the small intestine.

Cell transfection and LCFA uptake. COS cells were cotransfected, using lipofectamine (GIBCO BRL, Rockville, Md.) according to the manufacturer's instructions, with the mammalian expression vector pCDNA3.1 (Invitrogen, Carlsbad, Calif.) expressing the gene for CD2 in combination with a pMET7 expression vector (Tartaglia et al., Cell, 83:1263-1271, 1995) containing hsFATP1 (pMET7-hsFATP1) or hsFATP4 (pMET7-hsFATP4) or pMET7 alone. Two days after transfection, cells were assayed for CD2 expression with a phycoerythrin-coupled anti-CD2 (PE-CD2) monoclonal antibody (PharMingen, Franklin Lakes, N.J.), and fatty acid uptake was assayed with a BODIPY-labeled fatty acid analog (Molecular Probes) as described above.

Absorption of dietary fat requires transport of free fatty acids across the apical membrane of epithelial cells in the small intestine. Previous studies suggested that this transport is protein-mediated; however, the transport protein had not yet been identified. In situ hybridization was performed on each of the three regions of the small intestine—duodenum, jejunum and ileum—as well as the colon, using probes from the 3′ untranslated regions of mmFATP1, mmFATP2, mmFATP3, mmFATP4 and mmFATP5, to determine whether any of the mouse FATPs are expressed in the small intestine. It was expected that a protein involved in fatty acid absorption would be expressed in the epithelial cells of the small intestine, but absent from the colon.

Expression of mmFATPs in the jejunum was identical to that in the ileum in all cases. High levels of mmFATP4 mRNA were present in the epithelial cells of the jejunum and ileum, and lower, but significant, amounts were detected in the epithelial cells of the duodenum. Significantly, FATP4 mRNA was absent from other cell types of the small intestine and no FATP4 mRNA could be detected in any of the cells of the colon. FATP2 mRNA was present in the epithelial cells of the duodenum at a level similar to that of FATP4, but was present at lower levels in the jejunum and ileum. No signals above background were detected for mmFATP1, mmFATP3 and mmFATP5 in any of the intestinal tissues. mmFATP3 and FATP5 were clearly detectable by in situ hybridization in adult liver and mmFATP1 could be detected in a variety of tissues on a whole embryo in situ, indicating that the FATP1, 3, and 5 probes were working.

mmFATP4 expression is predominant in the small intestine compared to the other organs of the mouse embryo. In the small intestine, FATP4 expression is limited to differentiated enterocytes, while no signal is detected in the connective tissue or the undifferentiated epithelial cells in the crypts. Differentiated enterocytes are known to be the cells that mediate the uptake of fatty acids. FATP4 is specifically and strongly expressed in the epithelial cells of adult murine duodenum and ileum but not colon. Other FATPs, such as FATP5, are not expressed in the small intestine. Thus, FATP4 is the major FATP in the mouse small intestine. Given its high level of expression, it is likely that FATP4, and to a lesser extent FATP2, play an important role in the absorption of fatty acids.

mmFATP2, and mmFATP5 are Expressed in Hepatocytes

Northern analysis of mmFATP2, mmFATP3, mmFATP4 and mmFATP5 showed expression in the liver. To determine whether these proteins are present in hepatocytes or other cells types present in liver homogenates, in situ hybridizations were performed. mmFATP2, and mmFATP5 mRNA was clearly present in hepatocytes, and was not concentrated in other cell types such as endothelial cells or macrophages. No signal above background was detected for mmFATP 1 in any of the cell types in the liver, consistent with the results of the Northern blotting.

### Example 7

### Isolation and Sequence Analysis of Full-Length Human FATP 1 and Full-Length Human FATP4

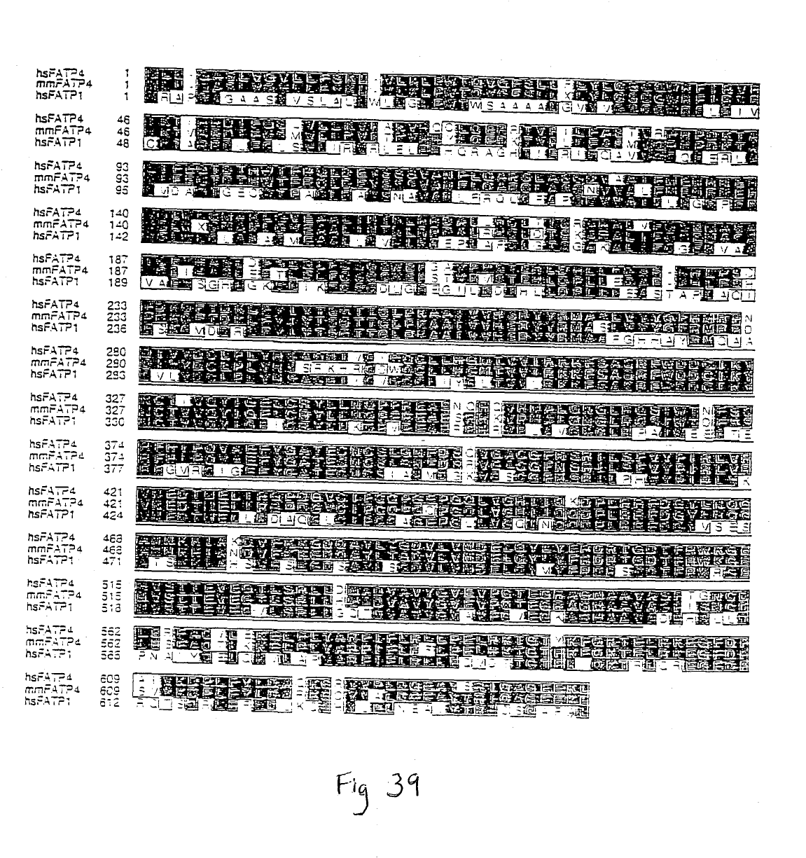

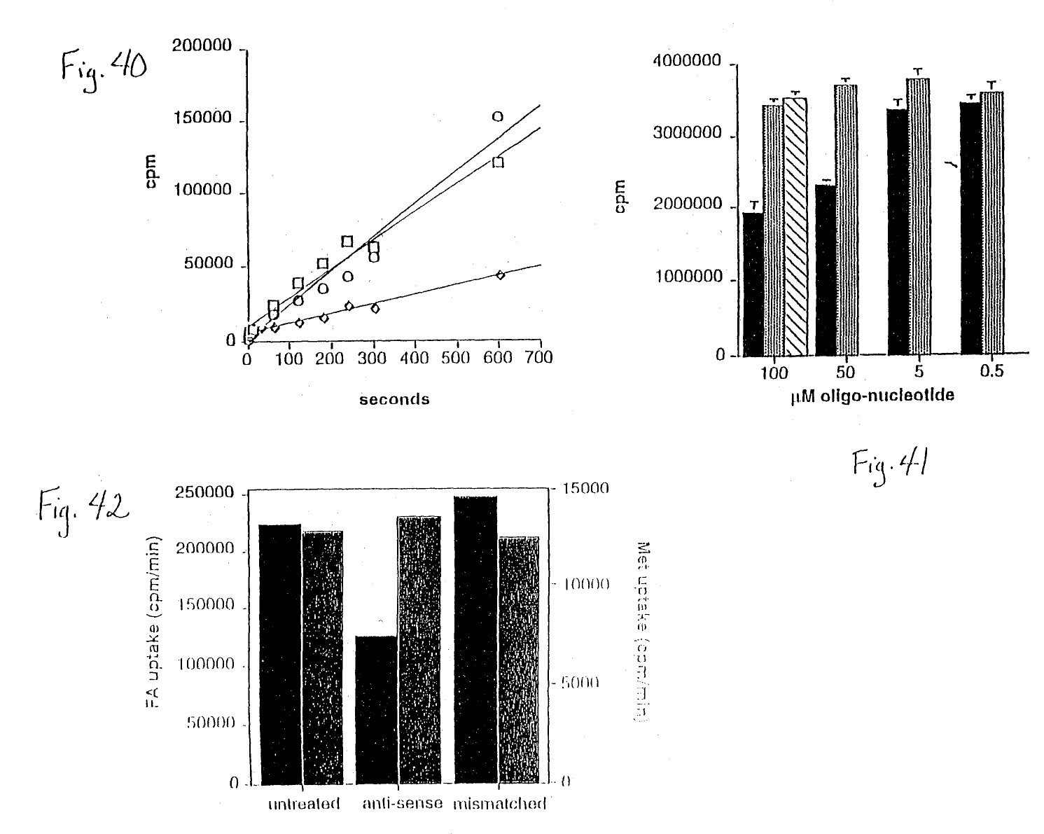

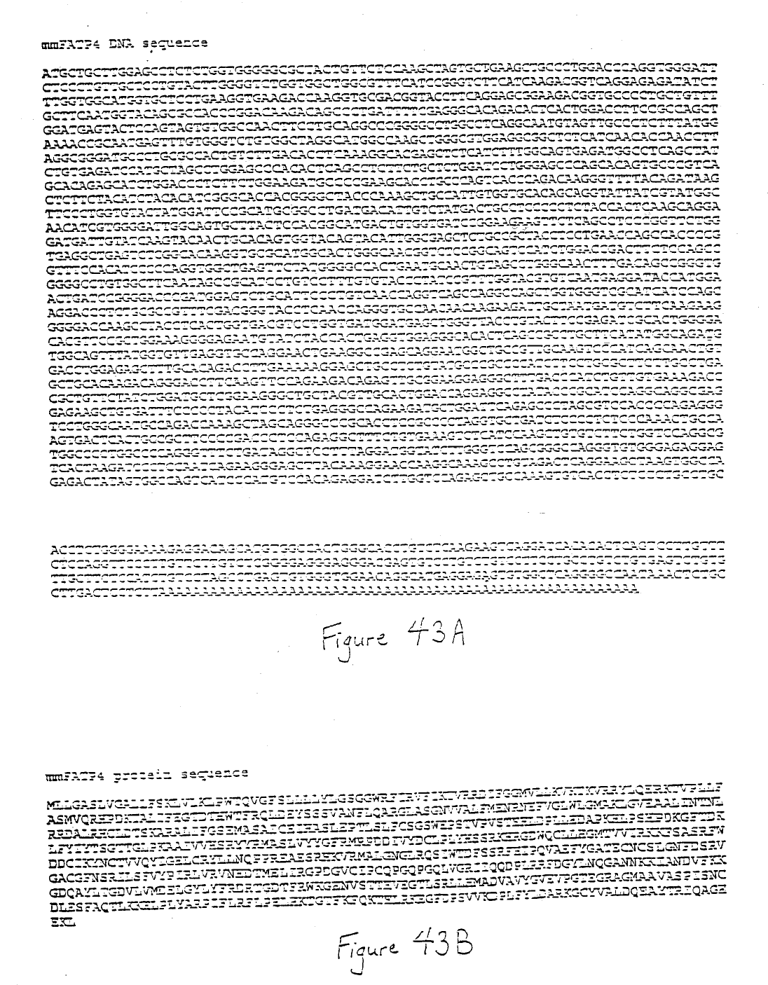

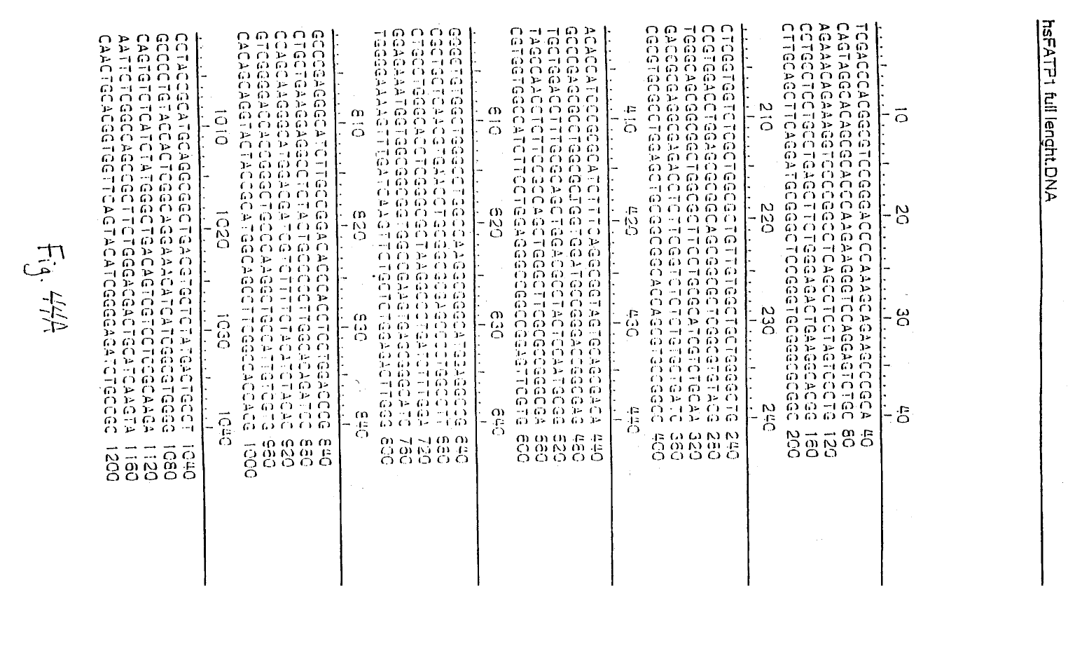

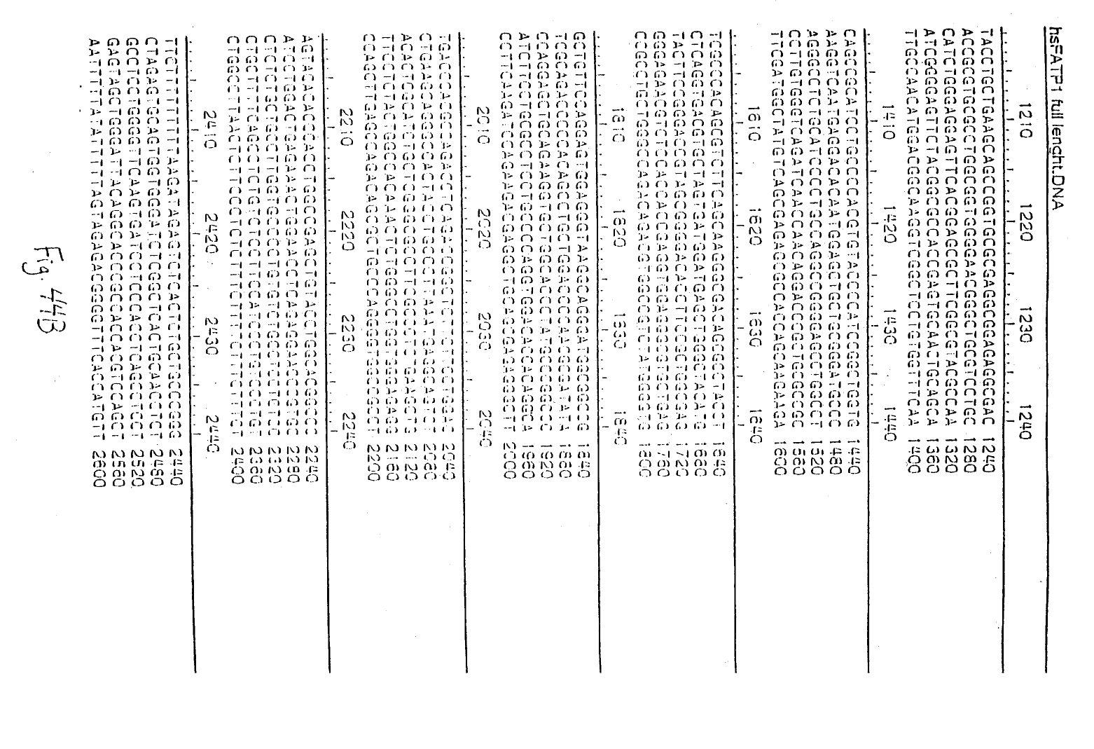

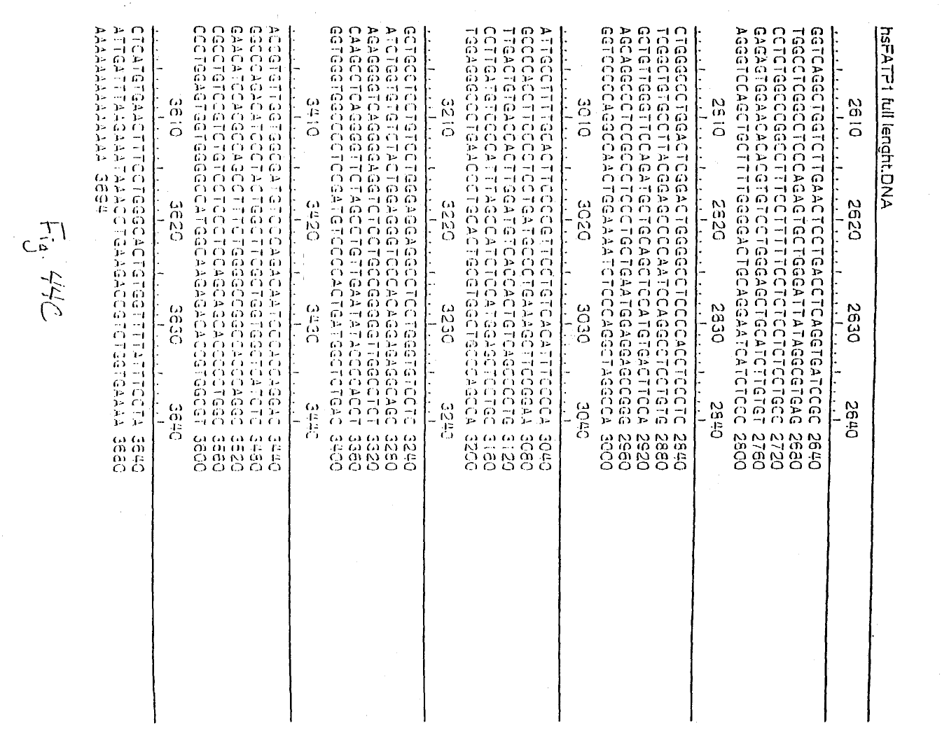

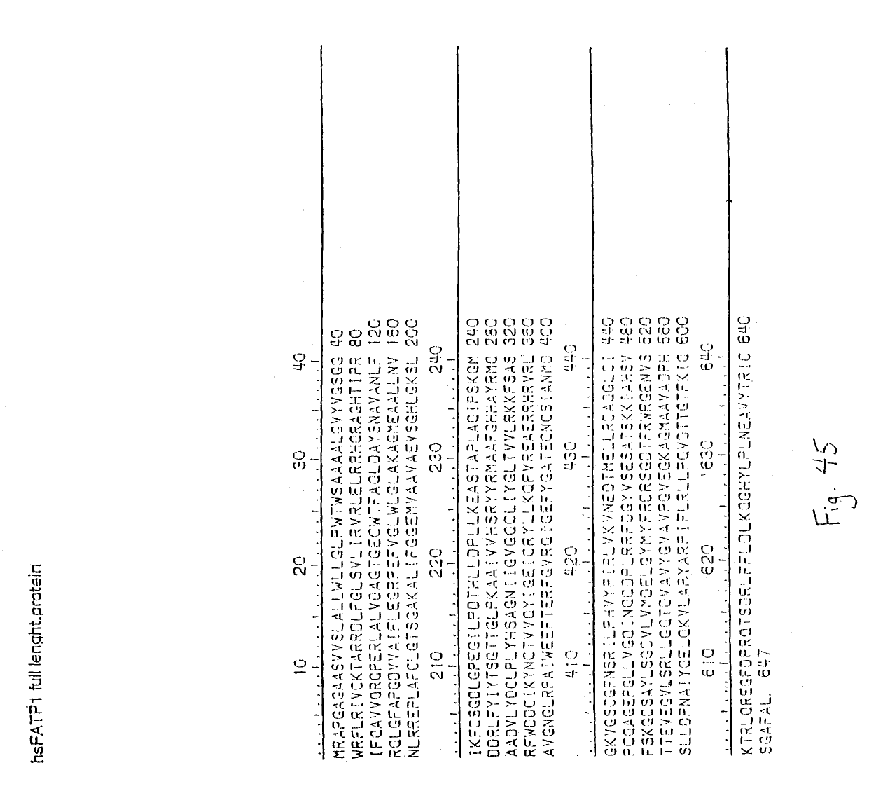

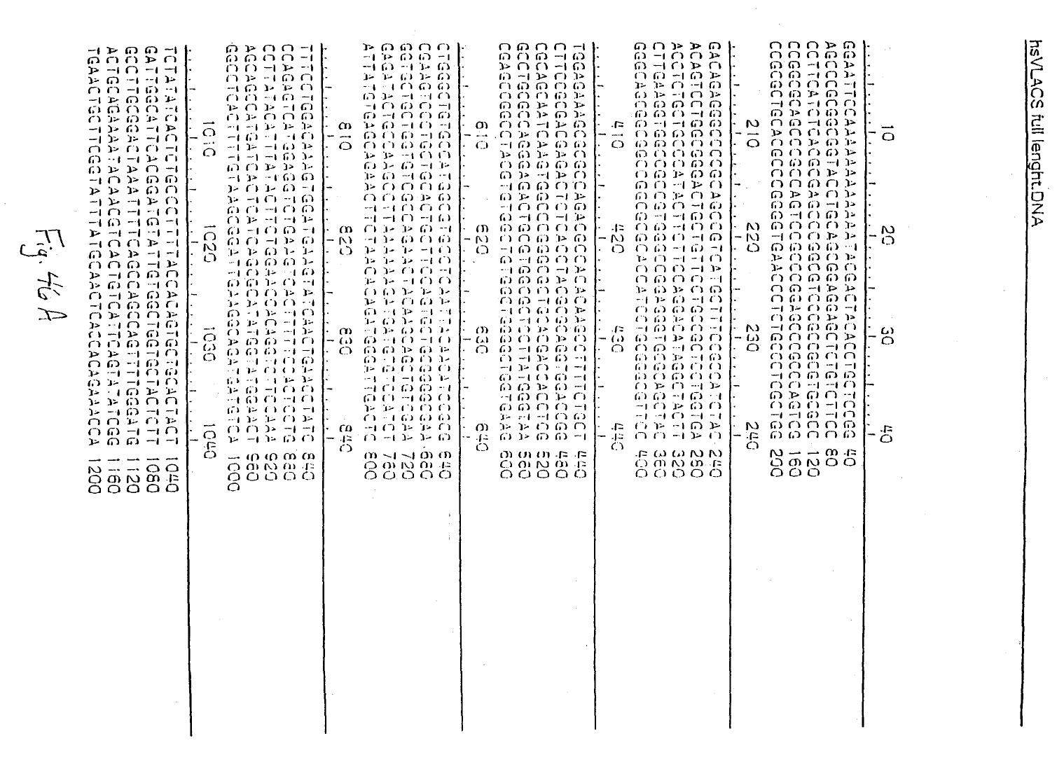

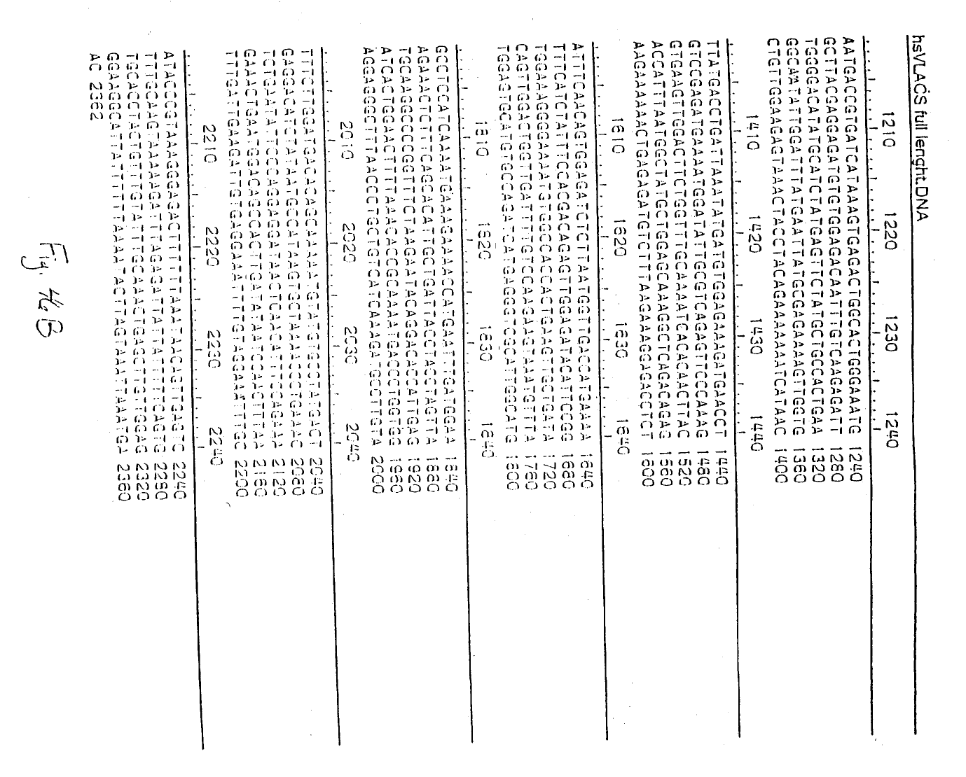

To identify human cDNA clones encoding FATP family members, Millennium databases were searched for sequences similar to murine FATP1-5 coding regions. Two clones were. analyzed in detail; inspection of the entire DNA sequence of these two clones showed that they encode the human orthologs of mmFATP1 and mm FATP4, respectively. These two clones were designated hsFATP1 and hsFATP4, and their DNA and predicted protein sequences are shown in FIGS. 44A-44C and 45, and 50A-50C and 51. hsFATP1 is predicted to encode a 646 amino acid, 71 kD protein with multiple membrane-spanning domains (FIG. 28A). HsFATP4 is predicted to encode a 643 amino acid, 72 kD protein with multiple membrane spanning domains (See FIG. 29A). A comparison of the DNA sequences of mouse and human FATP1 and mouse and human FATP4 (FIGS. 30A-30B and 31A-31B) shows that the mouse and human orthologs are 85% (FATP1) and 87% (FATP4) identical to each other within the coding sequences given in these figures. At the amino acid level, hsFATP1 and hsFATP4 are ˜90% identical to their respective mouse orthologs within the coding region shown in these figures (FIGS. 32 and 33). The sequence identities between mouse and human FATP1 and FATP4 are considerably higher than the ones observed between different FATP family members within one species (˜40%-60%) and are present in the N-terminal part of the protein, a region that is poorly conserved between different FATP family members. This high degree of sequence conservation clearly demonstrates that the newly identified human FATPs are orthologs of mouse FATP1 and FATP4 rather than novel FATP family members.

Table 4 is an identity/similarity matrix comparing the amino acid sequences of FATP1 and 4 from human and mouse. This shows that the gene whose sequence is shown in FIG. 43A is indeed human FATP4, since it is 91% identical with the murine FATP4 but only 62% identical with the closest related human FATP, which is FATP1.

TABLE 4
Identity/Similarity Matrix

| | hsFATP4 | mmFATP4 | hsFATP1 | mmFATP1 |
|---|---|---|---|---|
| hsFATP4 | — | 93.2 | 72.3 | 72.0 |
| mmFATP4 | 91.0 | — | 71.2 | 71.1 |
| hsFATP1 | 61.9 | 61.0 | — | 92.4 |
| mmFATP1 | 60.7 | 59.6 | 89.5 | — |

### Example 8

### Isolation and Sequence Analysis of Full-Length Human FATP6

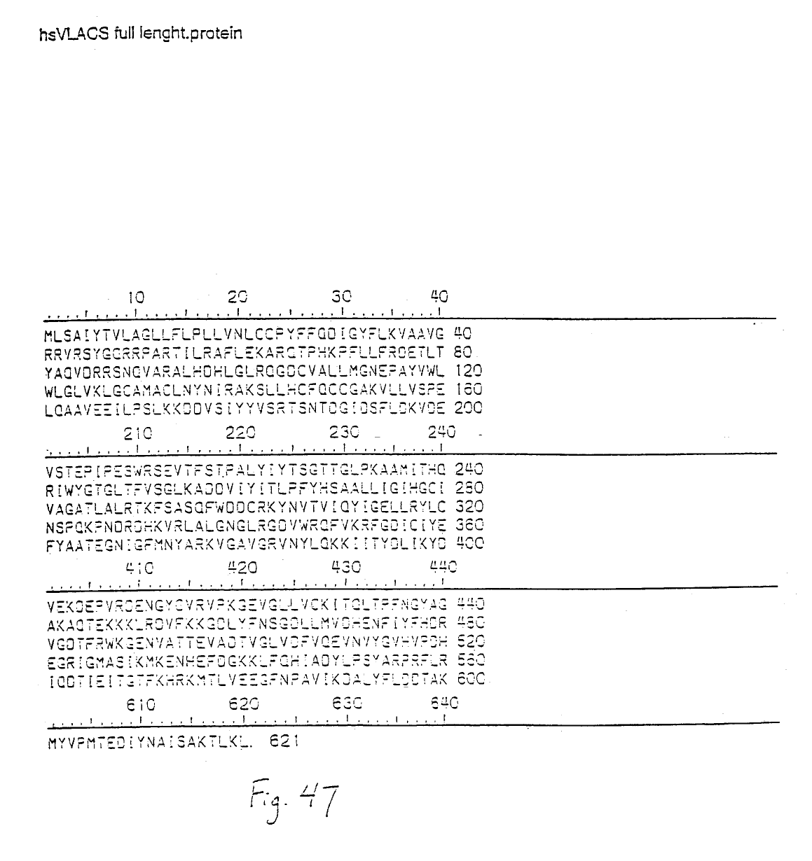

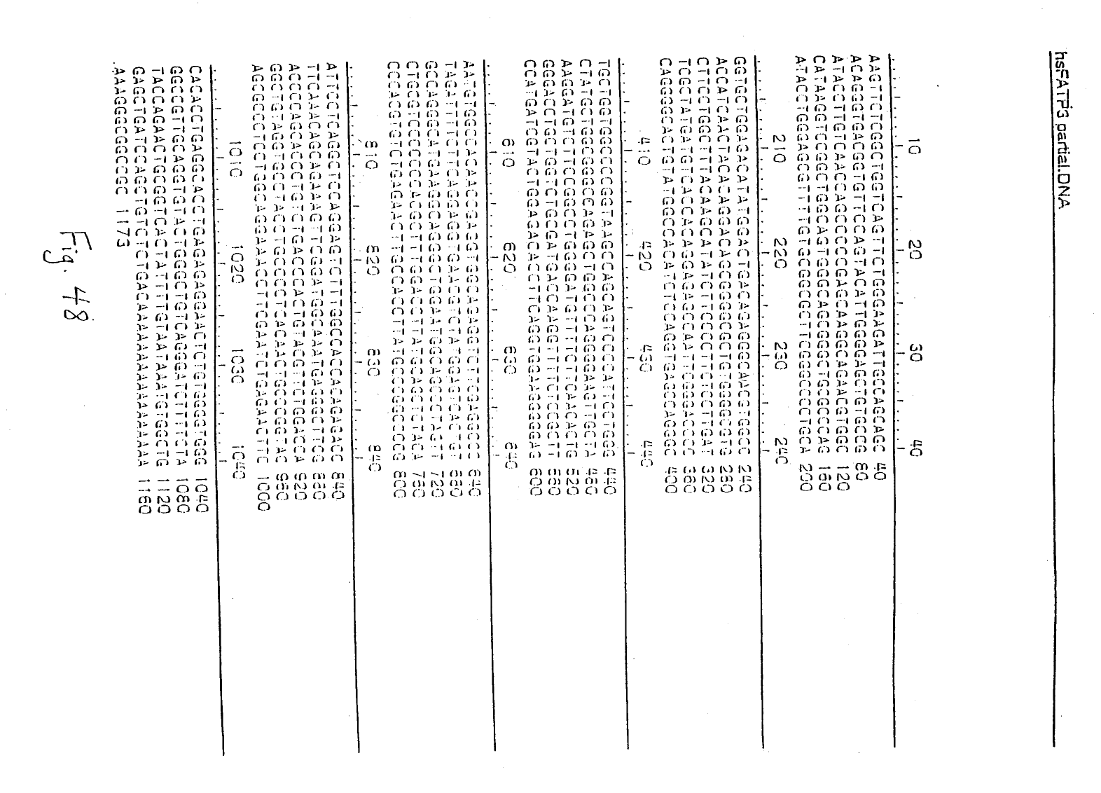

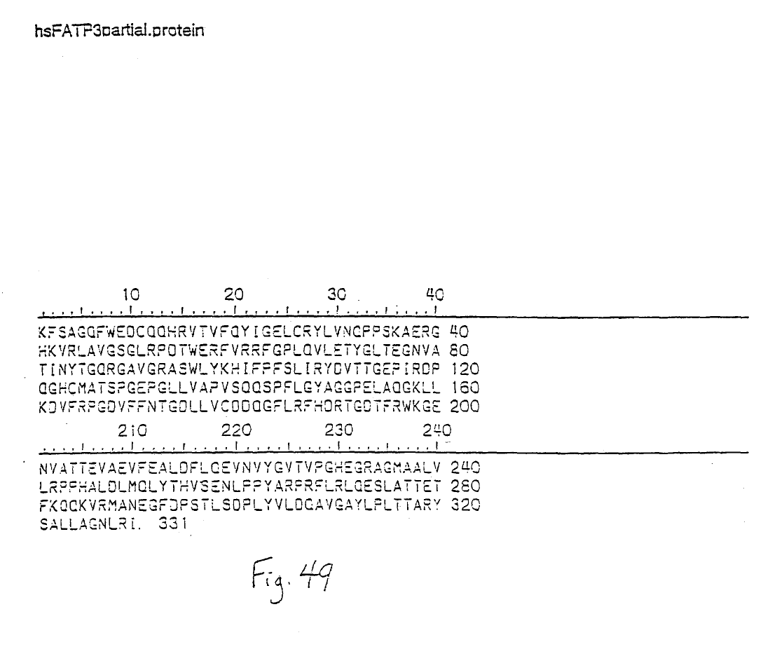

A search of EST databases identified a set of overlapping human sequences that were similar to FATPs, but did not have a clear mouse ortholog. One of these EST clones was found to encode a full-length cDNA. The entire insert of this clone was sequenced and designated hsFATP6. The DNA and predicted protein sequences of hsFATP6 are shown in FIGS. 54A-54C and 55. HsFATP6 is predicted to encode a 619 amino acid, 70 kD protein with multiple membrane-spanning domains (FIG. 35A). A comparison of the amino acid sequences of hsFATP6 with other human FATPs shows about 37% identity to either hsFATP1 or hsFATP4 (FIG. 36). This degree of sequence identity is similar to what is observed between different mouse FATPs. The phylogenetic analysis described above clearly demonstrates that hsFATP6 is a member of the FATP family, but not an ortholog of any of the mouse FATPs. Comparisons were done with “ALIGN” (E. Myers and W. Miller, “Optimal Alignments in Linear Space,” CABIOS 4:11-17 (1988) using standard settings.

### Example 9

### Tissue Distribution of Human FATPs

The tissue distribution of human FATPs was assessed by Northern blotting. Human FATP3 was expressed in a large variety of tissues. In contrast, human FATP5 was present at high levels in the liver, but was undetectable in all other tissues examined. Thus, both hsFATP3 and hsFATP5 recapitulate the expression pattern of their mouse orthologs (see above). HsFATP6 is a novel FATP with no mouse ortholog as yet. Northern blotting shows that hsFATP6 is expressed at high levels in the heart, but is undetectable in other tissues, including skeletal and smooth muscle. This tissue distribution suggests that human FATP6 performs an important role in energy metabolism in the heart; blocking FATP6-mediated fatty acid transport may therefore be beneficial for a number of heart diseases, e.g., ischemic heart disease.

To identify the major FATP expressed in the human small intestine, Northern blotting was performed on a blot containing mRNA from human stomach, jejunum, ileum, colon, rectum and lung. hsFATP5 and hsFATP6 were undetectable in any of these tissues. FATP5 is only expressed in liver and FATP6 only in heart. hsFATP2 was weakly expressed in the colon, and an even weaker signal was detectable in jejunum, ileum and lung lanes. hsFATP3 was expressed well in the lung, but was only weakly expressed in the other tissues tested. Importantly, no difference was seen in the expression of hsFATP3 between small intestine and stomach or colon, suggesting that the expression observed is not related to fatty acid absorption in the small intestine. hsFATP4 was clearly expressed in both jejunum and ileum; expression was significantly lower in the colon and was absent in the stomach. This expression pattern is consistent with a major role for FATP4 in absorption of fatty acids in the human gut.

### Example 10

### Expression of hsFATP1 and hsFATP4 Promotes Transport of Fatty Acids

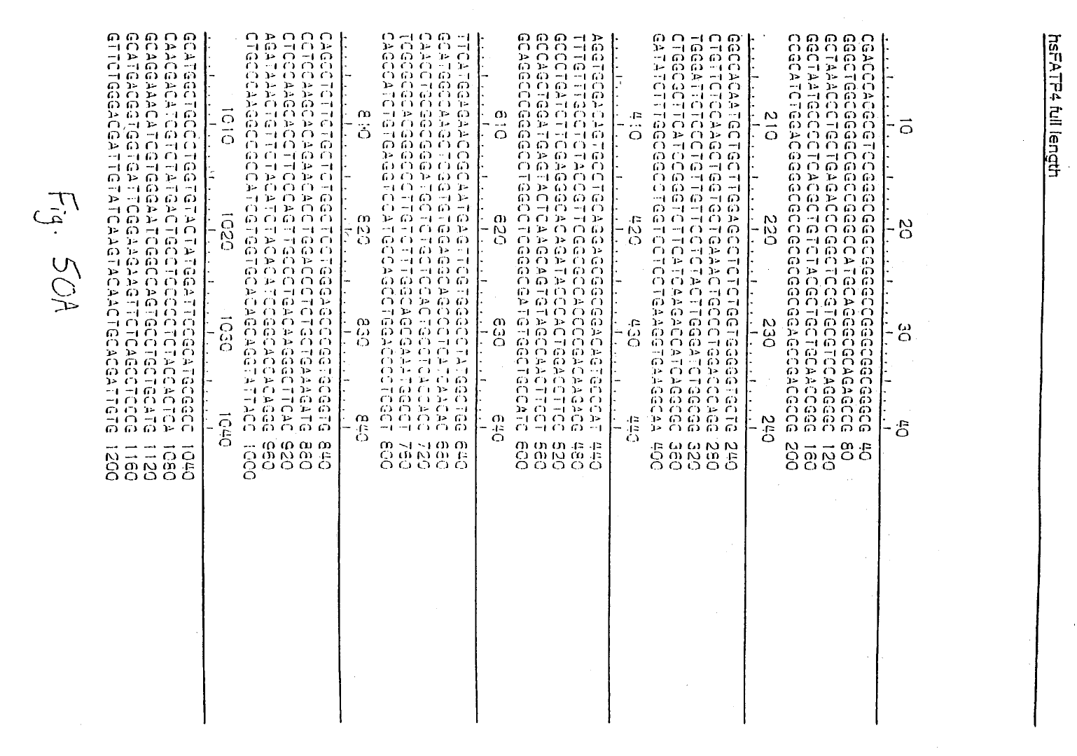

COS cells were cotransfected using lipofectamine with the mammalian expression vector pCDNA-CD2 in combination with one of the FATP-containing expression vectors (pMET7-hsFATP1 or pMET7-hsFATP4) or an insertless expression vector (pMET7, control) as described in Materials and Methods for Examples 6-10. COS cells were gated on forward scatter and side scatter. Cells exhibiting more than 400 CD2 fluorescence units representing −30% of all cells were deemed CD2-positive. The percent of CD2-positive cells exhibiting a BODIPY-fluorescence of >300 is plotted for the three different vectors tested (FIG. 37).

### Example 11

### Stable Expression of Human FATP4 in 293 Cells

Stable cell lines were generated as follows. A DNA fragment containing the entire hsFATP4 coding sequence as well as 100 nucleotides of 5′ and 50 nucleotides of 3′ untranslated region was inserted into the vector pIRES-neo (Clontech, Palo Alto, Calif.) using standard cloning techniques. The resulting construct or a vector control (pIRES-neo) was transfected into 293 cells using the lipofectamine method (Gibco BRL, Rockville, Md.) according to the manufacturer's directions. Cells that had taken up the DNA were selected with 1 mg/ml G418 (Gibco BRL, Rockville, Md.). Single colonies were picked 1 to 2 weeks after transfection and grown in medium containing 0.8 mg/ml G418. Colonies were screened for the ability to take up fatty acids by measuring uptake of a fluorescently labeled fatty acid (BODIPY-FA). About 40 colonies transfected with the pIRES-neo containing FATP4 and ˜20 colonies transfected with pIRES-neo control were analyzed. All 20 of the vector control clones showed amounts of BODIFY-FA uptake similar to each other and to untransfected 293 cells. In contrast, among the 40 FATP4 transfected clones, 3 had a 5- to 10-fold increased BODIPY-FA uptake compared to any of the vector controls, and a large number (˜20) showed an approximately two-fold increase in BODIPY-FA levels. This distribution is consistent with FATP4 conferring increased fatty acid uptake in these cells. One of the cell lines with the highest amount of BODIPY-FA uptake was selected to be used for measuring uptake of tritiated fatty acid.

The uptake of tritiated oleate over time by either FATP4 expressing or control cells was assayed over time. Expression of FATP4 increases the rate of fatty acid uptake by over 3-fold, demonstrating that FATP4 is, like the other FATPs, a functional fatty acid transporter (FIG. 38).

### Example 12

### Immuno-Staining with FATP4-Specific Antiserum

A polyclonal antiserum against the C-terminus of mmFATP4 was raised using a GST-fusion protein having mmFATP4-specific amino acid sequence 552-643 (AVASP . . . GEEKL). In western blot experiments, the purified antibody reacted strongly with a synthetic peptide matching the C-terminus of mmFATP4, but not with a corresponding region of mmFATP2, mmFATP3, or mmFATP5. The mmFATP4 specific polyclonal antiserum detects, in western blot experiments with enterocyte lysates from 3 different mice, a ˜70 kDa protein, which is in accordance with mmFATP4's predicted molecular weight of 72 kDa. The binding is specific for mmFATP4, since it can be completely abolished by preincubation of the antiserum with the GST-fusion peptide used to raise the antibody.

Immunofluorescence experiments were performed using the anti-mmFATP4 antiserum on fresh frozen sections of murine small intestine. The antibody binding demonstrates strong expression of mmFATP4 in enterocytes, confirming the results of the in situ hybridization experiments. At higher magnifications it is apparent that mmFATP4 is expressed at the apical side of the enterocyte, indicating that the transporter is present in the brush border membrane, which is known to mediate the uptake of fatty acids from the intestinal lumen.

Immuno-electron microscopy studies were performed on fresh frozen murine intestinal cells. The gold particles used, appearing as black specks on the electron micrographs, indicate the subcellular localization of mmFATP4 to be on the microvilli of the enterocyte. It can be seen from electron micrographs that mmFATP4 is localized exclusively in membranes, preferentially the apical plasma membrane, confirming that it is indeed a membrane protein.

Methods for Immunofluorescence and Immunogold Electron Microscopy

Unfixed mouse small intestine was washed with Hank's buffered salt solution containing 1 mM EDTA, infused with 2.3 M sucrose solution, and embedded in O.C.T., 4583 compound. The material was thick sectioned (15 μM -40 μM). The sections were washed in PBS containing 1% BSA and 0.075% glycine to block non-specific binding. Primary and secondary antibodies were diluted in PBS with 10% FCS and incubated for 1 h. The sections were mounted in 90% glycerol/PBS containing 1 mg/ml paraphenylinediamine, and examined with a Bio-Rad MRC 600 confocal, mounted on a Zeiss Axioscop.

For the immunogold labeling, the tissue was fixed with 2% paraformaldehyde in PBS for 10 minutes, after which it was cryoprotected by infiltration with 2.3 M sucrose in 0.1 M phosphate buffer (pH 7.4) containing 20% polyvinylpyrrolidone, and then mounted on aluminum cryo nails and frozen in liquid nitrogen (Tokuyasu, K. T., J. Microscop. 143:139-149, 1986). Ultrathin sections were collected on carbon/formvar-coated nickel grids. The primary antibody (anti-FATP4) was diluted in 10% FCS in PBS and incubated overnight at 4° C., followed by donkey anti-rabbit IgG-gold (12 nm) (Jackson Labs) for 1 h. The sections were stained in 2% neutral uranyl acetate (20 minutes) and absorption stained with 2% uranyl acetate in 0.2% methylcellulose containing 3.2% polyvinyl alcohol. The sections were examined with a Philips EM 410 electron microscope.

### Example 13

### Inhibition of Fatty Acid Uptake Specific to FATP4 Demonstrated in Isolated Mouse Enterocytes

Phosphorothioate derivatives of the following oligonucleotides were synthesized:

| | | |
|---|---|---|
| FATP4-AS2 | CCCCCACCAGAGAGGCTCC | (SEQ ID NO:103) |
| FATP4-AS2MM | CCACCCCCGGAAAGCCTGC | (SEQ ID NO:104) |
| FATP4-S2 | GGAGCCTCTCTGGTGGGGG | (SEQ ID NO:105) |

FATP4 AS2 is the antisense oligo; it is designed to be complementary to the sequence extending from nucleotide 10 to nucleotide 28 of the mouse FATP4 coding sequence. FATP4-AS2MM is a control oligo; in the oligo every third nucleotide was changed creating mismatches; the overall nucleotide composition is identical to FATP4-AS2 (same number of G, A, T, C). FATP4-S2 is the sense control.

Enterocytes were isolated from the small intestine of mice and incubated for 48 h in tissue culture (FIG. 40) either without oligonucleotides (squares) or with 100 μM FATP4 specific sense (circles) or antisense (diamonds) oligonucleotides. The uptake over time of 25 μM oleate was then measured. While the FATP4 sense oligonucleotide did not significantly influence the uptake, the antisense oligonucleotide inhibited fatty acid uptake by ˜50%.

The effect of either FATP4 sense, antisense or mismatch sequence oligonucleotides on the uptake of fatty acids was measured in enterocytes. Isolated enterocytes were incubated with increasing concentrations of FATP4 antisense oligonucleotides (solid bars in FIG. 41), or a mismatch control oligonucleotide with identical nucleotide composition (stippled bars), or with 100 μM of the FATP4 sense-oligonucleotide (lined bar). The medium for this incubation was Dulbecco's modified Eagle's medium with 4.5 g/L glucose, I mM sodium pyruvate, 0.01 mg/ml human transferrin and 10% fetal bovine serum. After 48 hours of incubation the uptake of oleate by enterocytes was measured over a 5 minute time interval. Measurements were done in quadruplicate. The uptake assay was done in Hank's buffered salt solution with 10 mM taurocholate. Only the enterocytes given FATP4 antisense oligonucleotide showed a concentration dependent decrease of fatty acid uptake, inhibiting it at a 100 μM concentration by ˜50%. This effect was FATP4 specific, since only the antisense oligonucleotide which can bind to the FATP4 mRNA and block its translation inhibited uptake, but not a control oligonucleotide differing only in the sequence but not the nucleotide content, ruling out a toxic or otherwise nonspecific inhibitory effect of this oligonucleotide due to its chemical composition.

As a further control experiment, the uptake of oleate was measured along with the uptake of methionine in the same cultured enterocytes. Antisense oligonucleotide, mismatch sequence oligonucleotide, or no oligonucleotide was added to a concentration of 100 μM to cultures of enterocytes. After incubation for 48 hours, the uptake of both ³H-labeled oleate and ³⁵S-labeled methionine was assayed. Results are shown in FIG. 42. Fatty acid uptake is at the left side of the paired bars; methionine uptake is on the right side of the paired bars. The fact that amino acid uptake was not influenced by the antisense oligonucleotide treatment further supports the conclusion that the antisense oligonucleotide causes a specific reduction in translation of FATP4-specific mRNA.

### Example 14

### mmFATP2 is Expressed in Proximal Renal Tubule Epithelium

Northern analysis showed that mmFATP1, mmFATP2, and mmFATP4 are present in the kidney. In situ hybridization (methods as for Example 6) was performed to determine which cell type(s) of the kidney these mRNAs are expressed in. mmFATP1 mRNA was present in virtually all cells throughout the kidney with no obvious preference for a particular cell type. In contrast, mmFATP2 was expressed only in the renal cortex. Within the cortex, expression of mmFATP2 was restricted to the epithelial cells of the proximal renal tubules. The primary function of proximal renal tubule cells is the reabsorption of filtered salts and nutrients (e.g., glucose), a process that requires mitochondrial oxidation and that can utilize fatty acids as energy substrates. Based on the localization of mmFATP2, it is possible that mmFATP2 is important for reabsorption in the kidney by allowing uptake of an energy source (fatty acids) from the blood into renal epithelial cells. Alternatively, if fatty acids need to be reabsorbed in the kidney, similarly to glucose, FATP2 could be involved in the reabsorption of fatty acids. Determination of the subcellular localization of FATP2 will distinguish between these two possibilities.

TABLE 5
Mouse FATP mRNA Expression

| Mouse Probes | mFATP1 | mFATP2 | mFATP3 | mFATP4 | mFATP5 |
|---|---|---|---|---|---|
| E18.5 embryo | everywhere, | liver | — | Brain, small | Mouse Probes |
| expression | brain = thymus> | (hepatocytes) | | intestine, | |
| | heart> brown | | | superior | |
| | fat, others | | | cervical | |
| | | | | ganglion | |
| | | | | (SCG), dorsal | |
| | | | | root ganglion | |
| | | | | (DRG), other | |
| | | | | regions have | |
| | | | | lower | |
| | | | | expression | |
| Duodenum | — | villi (surface | — | villi (surface | |
| | | epithelium) | | | epithelium) |
| Jejunum | — | villi (surface | — | villi (surface | |
| | | epithelium) | | | epithelium) |
| Ileum | — | villi (surface | — | villi (surface | |
| | | epithelium) | | | epithelium) |
| Colon | low expression | very low level | — | | |
| | in the crypt | in the crypt | | | |
| Kidney | cortex and | proximal | — | | |
| | medulla | tubules | | | |
| Liver | — | hepatocytes | hepatocytes | — | hepatocytes |
| Pancreas | exocrine | exocrine | — | — | — |
| | secretory units | secretory units | | | |
| | or acinar cells; | or acinar cells; | | | |
| | endocrine | endocrine | | | |
| | pancreas (islet) | pancreas (islet) | | | |
| | are negative | are negative | | | |
| Brain | Neuronal | — | — | Neuronal | — |
| | expression | | | expression | |
| | throughout the | | | throughout the | |
| | brain including | | | brain including | |
| | hypothalamus | | | hypothalamus | |
| Heart | myocytes | — | — | | |
| Testis | seminiferous | — | seminiferous | | |
| | tubules | | tubules | | |
| Lung | bronchiole | — | — | | |
| Adipose | adipocyte | adipocyte | — | | |

### Example 15

### Isolation of Full-Length Human FATP3

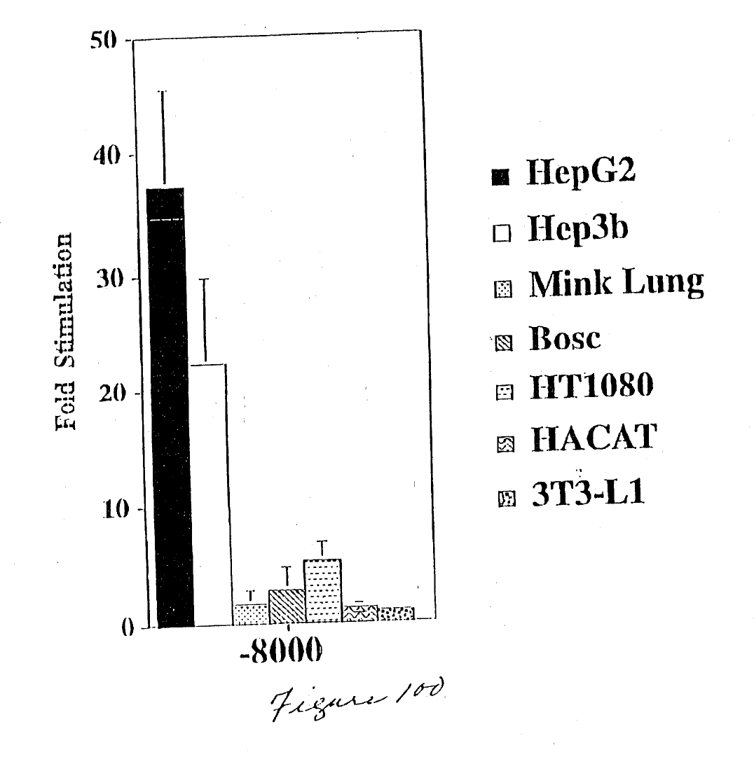

Full-length clones encoding human FATP3 were identified by searching databases for sequences similar to the murine FATP 1-5 coding regions using the BlastX algorithm (Altschul et al., J. Mol. Biol. 215: 403-410, 1990). Human clones with similarity to the 5′ end of murine FATP sequences were sequenced completely. A clone encoding full-length human FATP3 was obtained from a human bone library constructed in the mammalian expression vector pMET7 (Tartaglia, L. A. et al., Cell 83: 1263-1271, 1995). To identify human cDNA clones encoding FATP family members, databases were searched for sequences similar to murine FATP1-5 coding regions. One clone was found to encode the human ortholog of mmFATP3 and was designated hsFATP3. The DNA and predicted protein sequences of hsFATP3 are shown in FIGS. 94A and 94B. hsFATP3 is predicted to encode a 702 amino acid 75.6 kD protein with multiple membrane-spanning domains. A comparison of the DNA sequences of mouse and human FATP3 shows that the mouse and human orthologs are 81% identical to each other within the coding region. At the amino acid level, hsFATP3 is ˜86% identical to mm FATP3 within the coding region. The sequence identities between mouse and human FATP3 are considerably higher than those observed between different FATP family members within one species (˜40%) and are present in the N-terminal part of the protein, a region that is poorly conserved between different FATP family members.

### Example 16

### Substrate Specificity of Fatty Acid Transport in hsFATP-Transfected Clones

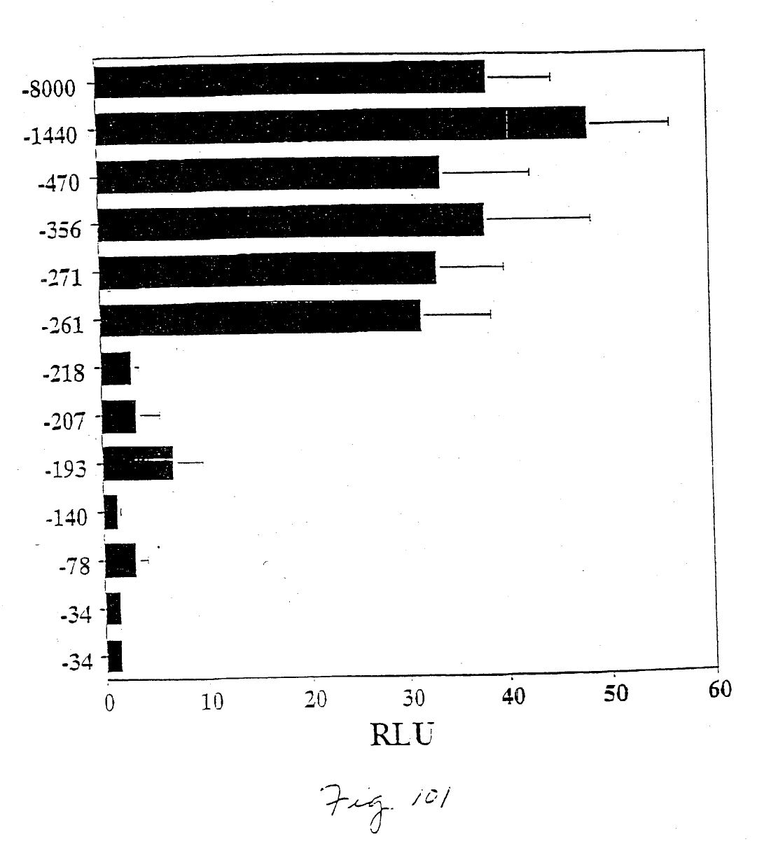

Using a mammalian expression vector, we generated 40 stable 239 cell lines expressing hsFATP4 and 20 cell lines transfected with a control plasmid. The ability of the different cell lines to take up FA, as assessed by uptake assays using the fluorescently labeled Bodipy-palmitate, correlated well with their FATP4 expression levels determined by Western blotting (FIG. 95). All 20 vector control clones showed amounts of Bodipy-FA uptake similar to each other and to untransfected 239 cells. In contrast, among the 40 FATP4 transfected clones, a large number (˜20) showed an approximately 2-fold increase in Bodipy-FA uptake compared to any of the vector controls, and three had a 5- to 10-fold increase in Bodipy-FA uptake.

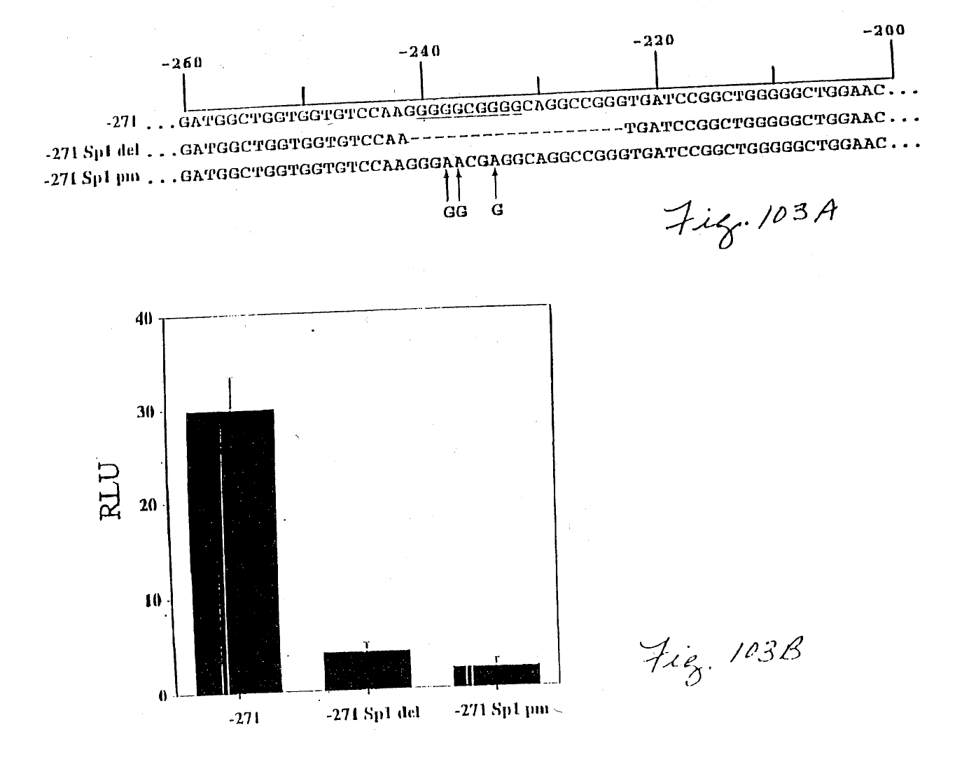

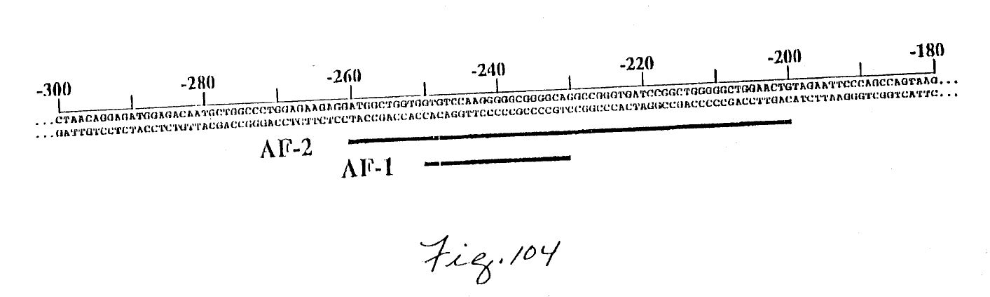

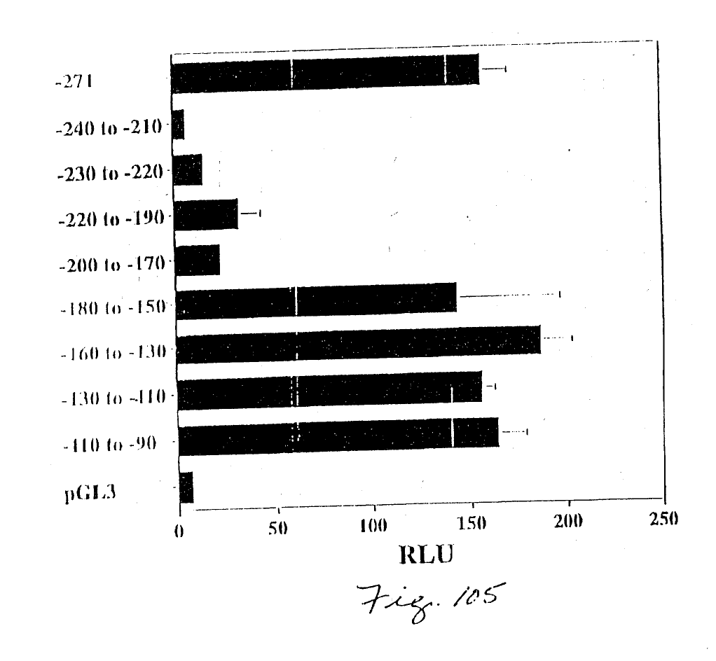

Several of the cell lines with the highest amount of Bodipy-FA uptake as well as isolated primary enterocytes were used to measure the uptake of radiolabeled FAs. Short-term uptake by 293 cells and enterocytes of all FAs tested was linear (FIG. 97). hsFATP4 expression enhanced the rate of palmitate uptake approximately 3 fold over 293 cells transfected with vector alone (FIG. 97) and also accelerated the uptake of oleate but not of linolate, arachidonate, octanoate, butyrate or cholesterol (Table 6). Isolated primary enterocytes showed a similar preference for palmitate and oleate, and absence of transport of arachidonate, octanoate, and butyrate, but displayed a more robust transport of linolate and cholesterol than the transfected 293 cells.

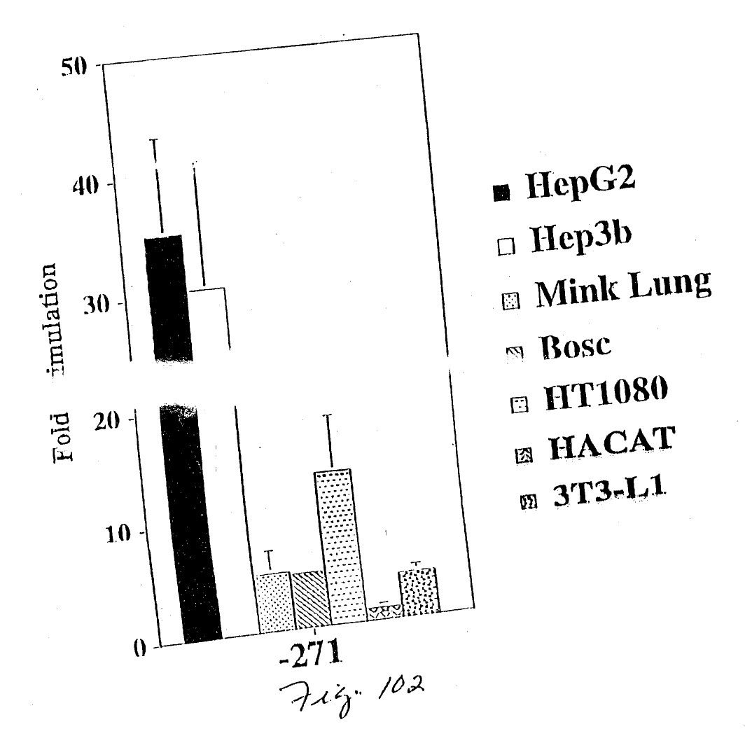

To further characterize the substrate specificity of FATP4, we measured the uptake by stably transfected 293 cells of 5 μM Bodipy-FA in the presence of a 20 fold molar excess (i.e., 100 μM) of FAs, FA-derivatives and lipid soluble vitamins and hormones. Both saturated and non-saturated fatty acids containing 10 to 26 C atoms strongly competed for uptake of Bodipy-palmitate (FIG. 96 and Table 7) and thus are presumed to be substrates of FATP4. In contrast, fatty acids with eight or fewer C atoms did not compete and thus are presumed not to be FATP4 substrates. Similarly, esters of long chain FAs and other hydrophobic molecules tested had no effect on uptake of Bodipy-palmitate.

LCFA Uptake Assays (Methods)

Bodipy-FA uptake assays using FACS were performed, adapted to a 96-well format. LCFA uptake assays with enterocytes or with stably transfected 293 cells were done as follows. Mixed micelles of radiolabeled FA (NEN) and taurocholate (Sigma) in HBS were generated by brief sonication at 37° C. Equal volumes of cells and micelle solution were mixed, resulting in a final FA concentration of 25 μM for antisense assays and 10 μM for substrate specificity assays. Final taurocholate concentration was 5 mM. Cells were incubated for the indicated amount of time at 37° C. The assay was stopped by transferring the cells onto filter paper followed by extensive washes with ice-cold HBS containing 0.1% BSA using a cell harvester (Brandell). Incorporated oleate was then determined by β-scintillation counting (Beckman).

TABLE 6
Uptake of Different Substrates by FATP4
Expressing Cell Lines and Enterocytes

| | | 293 Cells | | |
|---|---|---|---|---|
| | | Stably | | |
| | 293 Cells | Expressing | FATP4 | |
| Fatty Acid | Control* | FATP4 | specific | Enterocytes* |
| Palmitate | 564 | 1695 | 1131 | 3036 |
| Oleate | 662 | 1122 | 459 | 117 |
| Linolate | 640 | 673 | 33 | 116 |
| Arachidonate | 3 | 5 | 2 | 0 |
| Octanoate | 0 | 0 | 0 | 5 |
| Butyrate | 0 | 50 | 50 | 73 |
| Cholesterol | 319 | 345 | 26 | 531 |

Uptake of different substrates by enterocytes and by control and stable FATP4-expressing 293 cells. The rates of uptake for the indicated fatty acids was measured over 4 min taking measurements every 30 s. All fatty acids were at a concentration of 10 μM in HBS containing 5 mM taurocholate.
*Uptake measured as pmol/min 10⁶ cells

TABLE 7
Competition of Bodipy-FA Uptake by FATP4 Expressing Cells

| Fatty Acids | Formula | Competition |
|---|---|---|
| Butyric Acid | C₄H₈O₂ | − |
| Caproic Acid | C₆H₁₂O₂ | − |
| Caprylic Acid | C₈H₁₆O₂ | − |
| Capric Acid | C₁₀H₂₀O₂ | ++ |
| Lauric Acid | C₁₂H₂₄O₂ | ++ |
| Myristic Acid | C₁₄H₂₈O₂ | ++ |
| Palmitic Acid | C₁₆H₃₂O₂ | ++ |
| Stearic Acid | C₁₈H₃₆O₂ | + |
| Oleic Acid | C₁₈H₃₄O₂ | ++ |
| Linoleic Acid | C₁₈H₃₂O₂ | ++ |
| Arachidic Acid | C₂₀H₄₀O₂ | ++ |
| Lignoceric Acid | C₂₄H₄₈O₂ | ++ |
| Cerotic Acid | C₂₆H₅₂O₂ | ++ |

Fatty Acid Derivatives

| | | |
|---|---|---|
| Palmitic Acid Methyl | C₁₇H₃₄O₂ | − |
| Ester | | |
| Stearic Acid Methyl Ester | C₁₉H₃₈O₂ | − |
| Oleic Acid Ethyl Ester | C₂₀H₃₈O₂ | − |
| Oleic Acid Oley Ester | C₃₆H₆₈O₂ | − |
| Oleoyl CoA | C₃₉H₆₈N₇O₁₇P₃S | − |
| Cholesteryl Oleate | C₄₅H₇₈O₂ | − |

Lipid-Soluble Vitamins & Hormones

| | | |
|---|---|---|
| Retinoic Acid (Pro-Vitamin A) | C₂₀H₂₈O₂ | ± |
| Ergocalciferol (Vitamin D2) | C₂₈H₄₄O₂ | − |
| Tocopherol (Vitamin E) | C₂₉H₅₀O₂ | − |
| 3-Phytylamenadione (Vitamin | C₃₁H₄₆O₂ | − |
| K1) | | |
| Prostaglandin E2 | C₂₀H₃₂O₅ | − |

Competition for Bodipy-FA uptake by FATP4 expressing cells by different hydrophobic compounds. The uptake of 5 μM Bodipy-FA, C1-Bodipy-C12 was measured in the presence of a 20-fold molar excess (i.e., 100 μM) of the indicated fatty acids or fatty acid derivatives. The maximal 100% inhibition was defined as the amount of Bodipy-FA incorporated in the presence of 200 μM lauric acid which was on average 18% ± 5% that of untreated cells.
−: 0%-30% inhibition by the indicated substance
±: 30%-50% inhibition
+: 50%-70% inhibition
++: 70%-100% inhibition

### Example 17

### Identification and Characterization of the FATP5 Promoter

Methods

BAC Isolation and Luciferase Constructs

An arrayed BAC library was screened by PCR for FATP5 genomic clones. PCR primers designed by a program from the Whitehead Institute's Genome Center specifically amplified a single band of the correct size from mouse genomic DNA. Two putative BACs containing the FATP5 genomic sequence were identified and the presence of FATP5 sequence was confirmed by dot hybridization of the BAC with the mmFATP5 cDNA.

After isolation of positive BACs, large amounts of bacteria were grown and DNA prepared using a Qiagen maxi-prep kit (Qiagen, Venlo, The Netherlands). The BAC was digested with Sac I and ligated into pZero-2 (Invitrogen, Carlsbad, Calif.). Inserts containing mmFATP5 genomic sequence were identified by screening colony lifts of the ligation with an β-³²P-ATP radiolabeled, random primed (Boehringer-Mannheim, Indianapolis, Ind.) mmFATP5 cDNA as a probe. Positive colonies were picked and restriction analysis with Sac I revealed them to contain an identical, large insert of 8-10 kb. Digestion of the Sac I fragment with BstX I yielded three pieces that were subsequently subcloned into pZero and sequenced using an ABI sequencer (Research Genetics). A 1.3 kb piece containing sequence immediately upstream of the FATP5 initiator methionine was subcloned into the Xho I and Bgl II sites of the promoter-less pGL3 luciferase reporter vector (Promega Corp., Madison, Wis.). 7 kb of additional upstream sequence was subcloned into the Xho I and Sac I sites of the prior construct to yield a final construct containing approximately 8 kb of genomic sequence upstream of the initiator methionine. Deletions of the FATP5 promoter were constructed using PCR with the 1.3 promoter construct as the template. Products were amplified with primers containing Hind III (5′ primer) and Xho 1 (3′ primer) sites using Elongase (Gibco, Rockville, Md.). The resulting fragments were cut with Hind III and Xho I and subcloned into the corresponding sites of the promoter-less pGL3 luciferase reporter vector. The internal 30 base pair deletions, GC box mutations, and 10 nucleotide linker scan were all created with the Quickchange mutagenesis kit (Stratagene, La Jolla, Calif.) according to the manufacturer's instructions. At least two different bacterial colonies were picked for each construct. The inserts from both colonies were sequenced to check for unintended point mutations and both constructs were assayed for luciferase activity.

Cell Culture, Transfection, and Luciferase Measurements

HepG2, Hep3B, HT1080, 3T3-L1, BOSC, and HACAT cells were grown in DMEM supplemented with 10% fetal calf serum, 1× penicillin-streptomycin and glutamine (Gibco, Rockville, Md.). Mink lung cells were grown in MEM supplemented with 10% fetal calf serum, 1× minimal essential amino acids, 1× penicillin-streptomycin and glutamine. The evening prior to transfection, cells were plated at 50-60% confluence in 24 well dishes. The following morning, cells were placed in 2 mls of fresh media and 250 μL of a CaPO₄ solution (Invitrogen, Carlsbad, Calif.) containing 2 μg of a luciferase reporter construct and 0.5 μg of pCMV-p-gal was added to the cells. pCMV-β-gal constitutively expresses β-galactosidase and was used to normalize transfection efficiency (Hua et al., 1998). After 12 hours, the cells were washed twice with DMEM and placed in fresh media. Thirty six hours later, the media over the cells was removed and 250 μL of 1× reporter lysis buffer (Promega Corp., Madison, Wis.) was added. After vigorous shaking for 15 minutes at room temperature, the supernatants were transferred to Eppendorf tubes and briefly centrifuged to remove particulates. 20 μL from these tubes was used for determination of luciferase activity (Promega Corp., Madison, Wis.) and 20 μL was used for the measurement of β-galactosidase activity (Clontech, Palo Alto, Calif.). All luciferase values were normalized to β-galactosidase to control for transfrection efficiency and expressed as relative luciferase units (RLU). For experiments comparing different cell lines, promoter activity was computed as a fold induction by dividing the RLU activity of either the −8 or −271 promoter constructs by the RLU activity a promoter-less construct. Each data point was done in triplicate and each experiment was repeated a minimum of three times.

Northern Blots, Preparation of Nuclear Extracts, and Gel Shift Assays

Human poly-A northern blots were purchased from a commercial vendor (Clontech, Palo Alto, Calif.) and probed with a piece of the human FATP5 3′ untranslated region specific for FATP5. Nuclear lysates from HepG2 and BOSC cells were essentially prepared according to the method of Hua et al. and stored at −80° C. (Hua et al., 1998). Probes for gel shift assays were end labeled using T4 polynucleotide kinase (Boehringer-Mannheirn, Indianapolis, Ind.) and gel purified. Gel shifts were performed at room temperature in 30 μL reactions comprised of 6 μL 5 × binding buffer (100 mM Tris 8.0, 300 mM KCl, 5 mM EDTA, 8 mM MgCl₂, and 36% glycerol), 0.5 μL of 100 mM DTT, 1 μL of 10 mg/ml BSA, 2 μL of 2 mg/ml poly dI/dC, and 5 μL nuclear lysate. Ten minutes after the addition of nuclear lysate, 40,000 cpm of ³²P-labeled probe were added. After 20 minutes at room temperature, loading dye was added and the reaction run on a 4% non-denaturing gel.

Results

Human FATP5 mRNA is Only Expressed in Adult Liver

We had previously reported that mmFATP5 mRNA was only expressed in the liver (Hirsch et al., 1998). To determine if the human isoform of FATP5 was also liver specific, we performed northern analysis using a probe from the 3′ transcribed but untranslated region of the human gene. Similar to the mouse homolog, hsFATP5 is liver specific. Interestingly, hsFATP5 was not expressed in fetal liver suggesting that it may be developmentally regulated.

Identification of a FATP5 Promoter

We next set out to determine the cis-acting elements responsible for liver specific expression of FATP5. We identified BACs containing the FATP5 genomic locus and subcloned a 10 kb Sac I fragment which was subsequently sequenced. The Sac I fragment contains approximately 8 kb of genomic sequence upstream of the FATP5 initiator methionine. Blast searches using the 5′ end of the Sac I sequence revealed that it contained coding sequence for an unknown gene immediately upstream of FATP5. Since the FATP5 promoter is unlikely to overlap the coding sequence of another gene, we hypothesized that the 10 kb Sac I fragment contained the FATP5 promoter. To test this hypothesis, 8 kb of genomic DNA upstream of the translational initiator of FATP was subcloned into the promoter-less pGL3 luciferase reporter vector. This construct was transiently transfected into the HepG2 liver cell line and luciferase activity was determined. The −8 kb piece of DNA resulted in a 35 fold induction of luciferase activity when compared to a pGL3 vector without the FATP5 genomic sequence (FIG. 100). To determine if this activity reflected tissue specific transcription, the −8 kb luciferase reporter construct was transfected into a variety of additional cell types. While promoter activity was also detected in the Hep3b hepatoma cell line, non-liver cell lines did not express luciferase above the level of the promoter-less vector. Thus, the 8 kb upstream genomic element recapitulated liver specific expression in vitro.

The FATP5 Promoter Resides within the 261 Base Pairs Upstream of the Initiator Methionine and Requires a Single GC Box

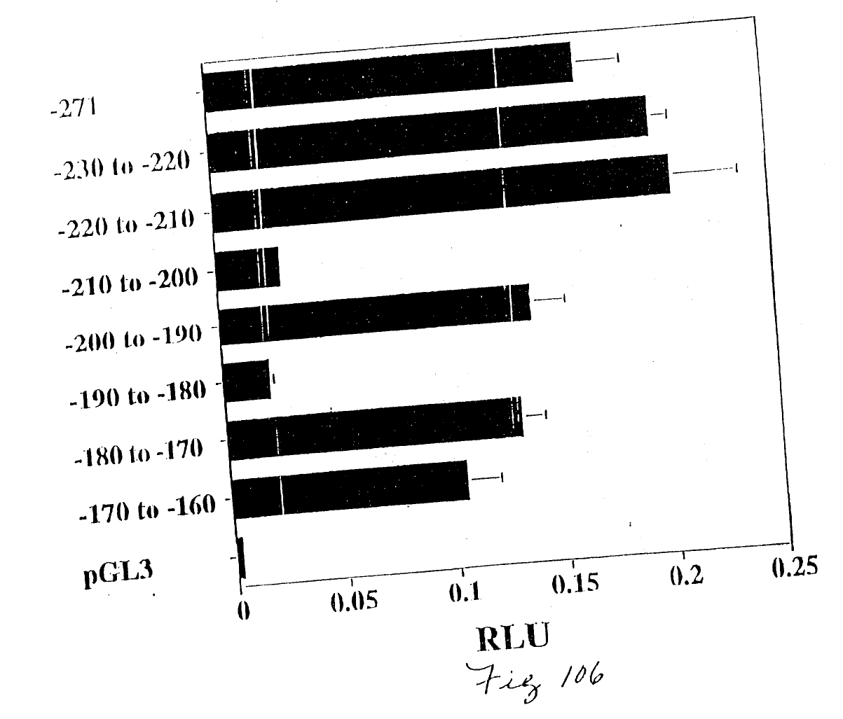

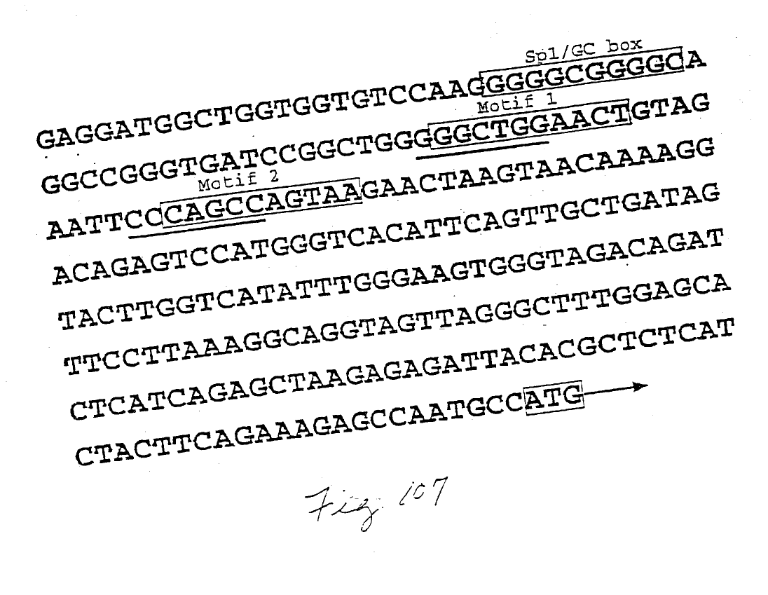

To determine the cis-acting elements in the −8 kb of genomic sequence responsible for transcriptional activity, serial 5′ deletions of the promoter were constructed and transfected into HepG2 cells. Surprisingly, greater than 90% of the −8 kb was dispensable for promoter activity. A construct containing only 261 base pairs upstream of the initiator methionine resulted in promoter activity equivalent to that of the −8 kb construct (FIG. 101). Identical results were obtained when the deletion series was transfected into Hep3b cells (data not shown). We next determined if promoter activity of a small genetic element was tissue specific. Transfection of a construct containing 271 base pairs upstream of the initiator methionine into a variety of cell lines essentially replicated the results of the −8 kb construct in that expression was observed only in liver derived cell lines (FIG. 102).

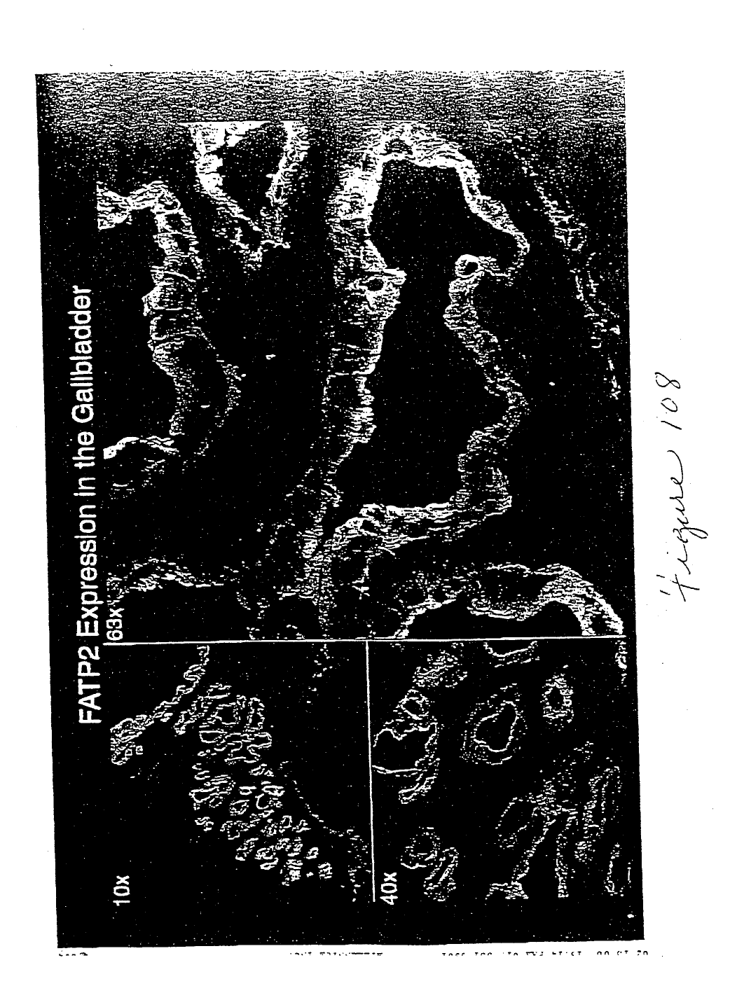

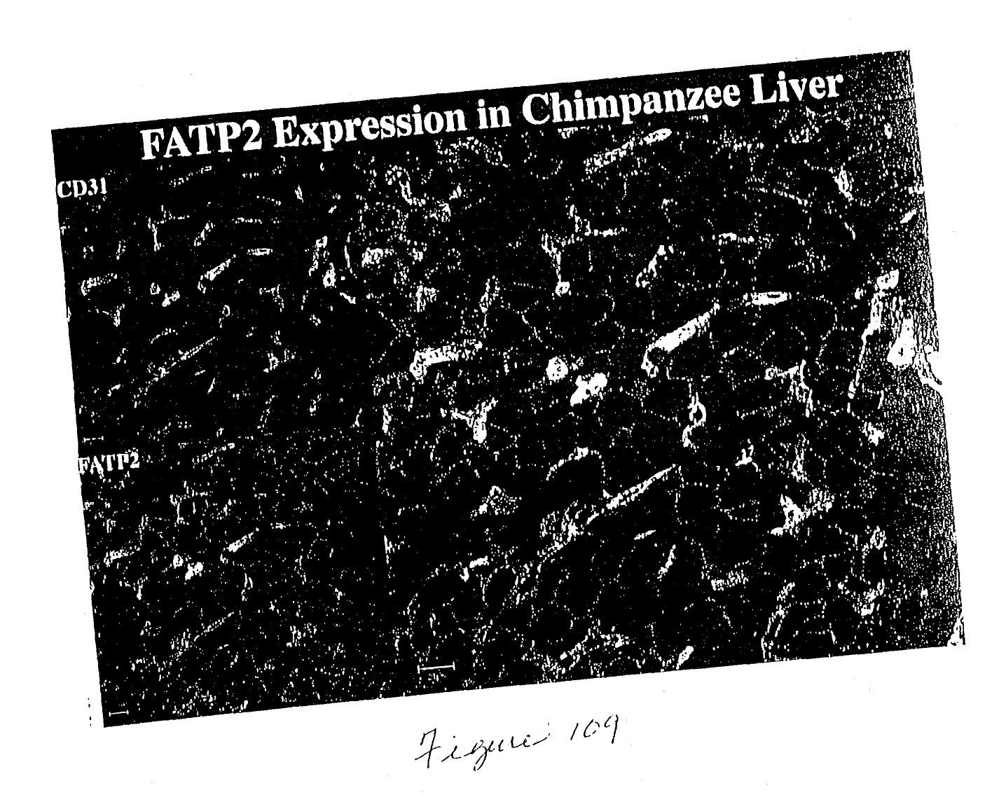

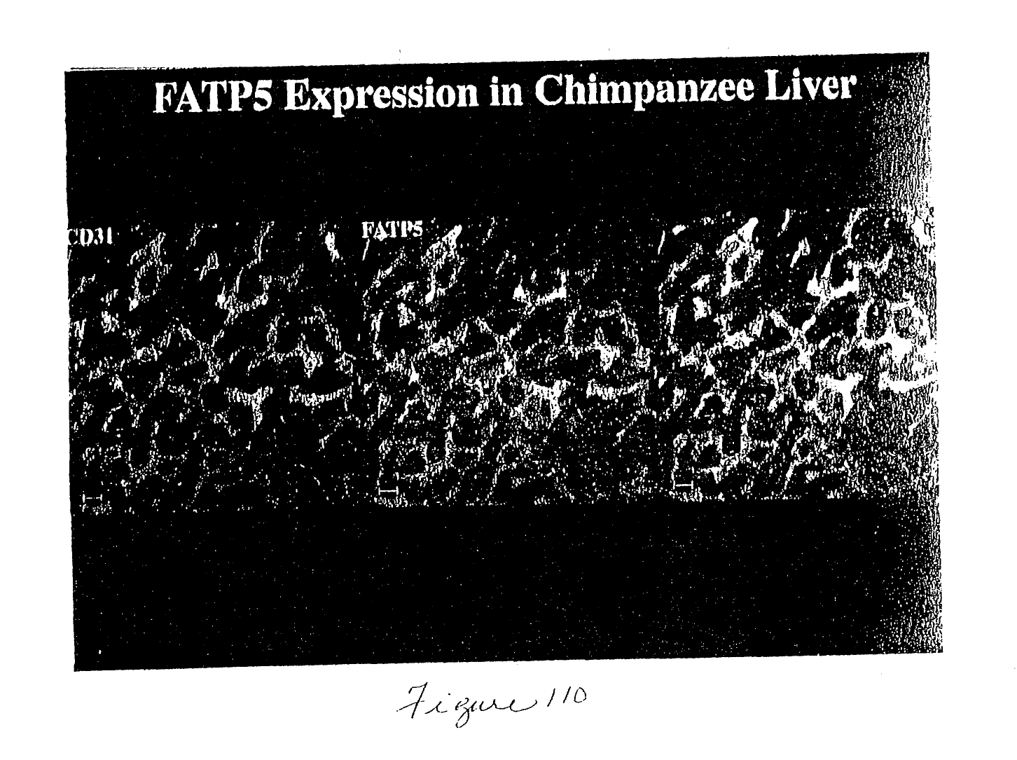

Since deletion analysis revealed that bases between −261 and −218 were required for promoter activity, we closely examined this region for binding sites of known transcription factors and found the sequence GGGGCGGGG between nucleotides −241 and −232 (FIG. 103A). This sequence binds the Sp1 family of transcription factors and is termed a GC box. To determine if the activity of the −271 construct required the GC box, we mutated the GC box. The first construct deleted nucleotides −241 to −222 which removed the GC box and additional downstream sequence which, although less optimal, might also bind the Sp1 family of transcription factors(SEQ ID NO: 107). The second construct had three G to A point mutations in the GC box between nucleotides −241 to −232(SEQ ID NO: 108). Such mutations had previously been shown to abolish transcriptional activity of GC boxes (Rodenburg et al., 1997). In contrast to the wild type −271 promoter, both of the mutated constructs were transcriptionally inactive in HepG2 cells (FIG. 103B). Identical results were also obtained in Hep3B cells (data not shown). This suggests that the GC box between −241 to −232 is essential for transcriptional activity of the FATP5 promoter. We next examined whether the sequences necessary for luciferase activity also bound proteins in nuclear extracts from HepG2 cells. Two different oligonucleotides were used for gel shift analysis. One oligonucleotide (AF-1) contained nucleotides −250 to −230(SEQ ID NO: 111) and the other (AF-2) spanned nucleotides ˜260 to ˜−200(SEQ ID NO: 109) (FIG. 104). Both oligonucleotides yielded three significant complexes from HepG2 nuclear extracts. All complexes were specific as 100 fold excess of the same unlabeled oligonucleotide could compete for binding of the radiolabeled oligonucleotide. Mutant AF-1 oligonucleotides containing three point mutations in the GC box did not bind any proteins in HepG2 nuclear extracts or compete for binding of nuclear proteins to the AF-1 or AF-2 oligonucleotides (data not shown). Oligonucleotides AF-1 and AF-2 also bound recombinant Spi (Promega Corp, Madison, Wis., data not shown). However, nuclear extract from BOSC cells, a kidney cell line, and HepG2 cells had identical patterns of complex formation (data not shown).

Identification of Novel Sequences Required for Transcriptional Activity of the FATP5 Promoter

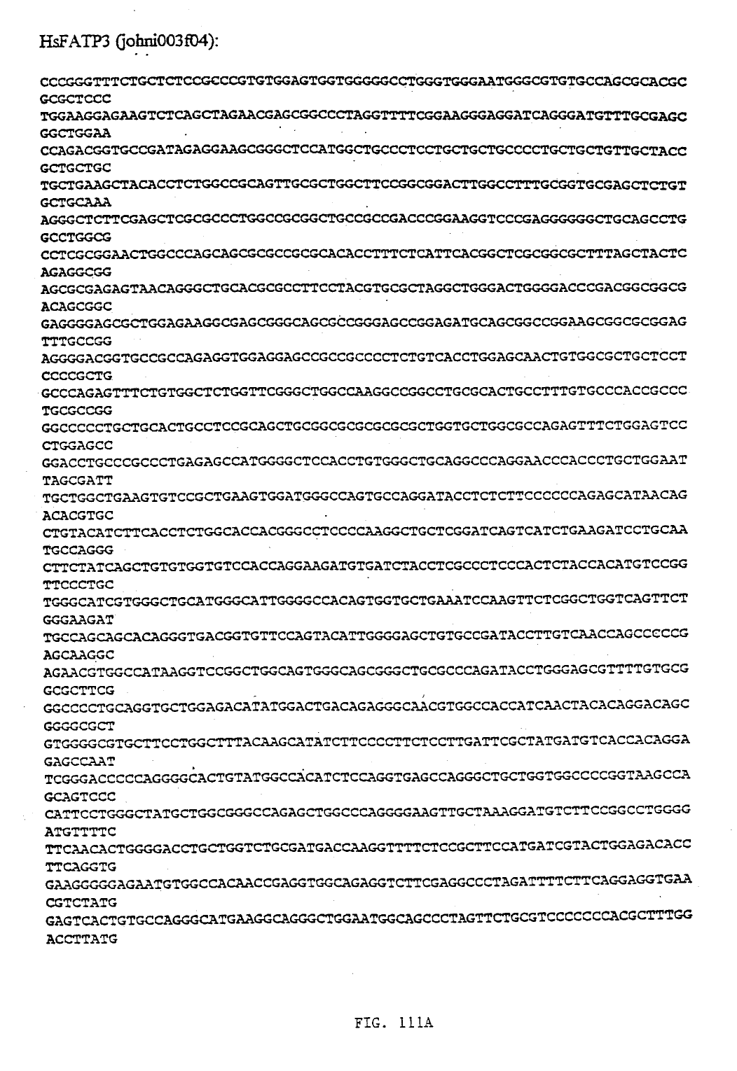

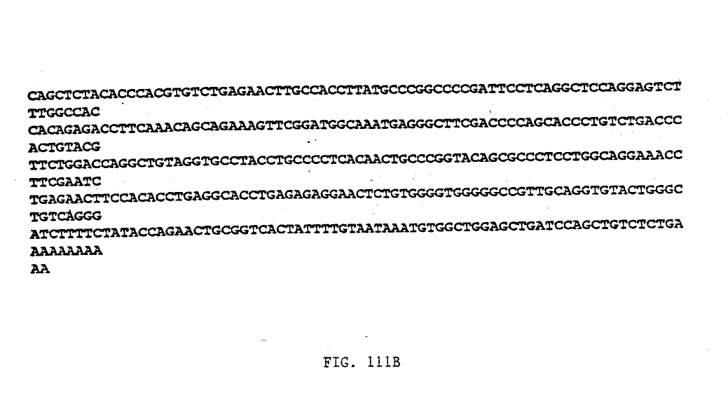

While the GC box between nucleotides 241 and 232 is essential for transcriptional activity, additional sequences downstream of the GC box might also be required for transcription. To determine if such sequences existed, we created 30 base pair internal deletions in the ˜−271 construct downstream of the GC box. Constructs that had deletions in sequences between 240 and 180 nucleotides upstream of the FATP5 translational initiator had greatly reduced transcriptional activity in HepG2 cells (FIG. 105). To identify the specific sequences within this region required for FATP5 transcription, a 10 nucleotide linker (CTAACAGGAG) (SEQ ID NO: 1-13) was exchanged for wild type sequence within the context of the −271 base pair construct (FIG. 106). Inadvertently, the 210 to 200 construct had a single nucleotide insertion and the 190 to 180 construct had a two nucleotide insertion relative to the wild type sequence. However, several other linker constructs that also had equivalent insertions (230 to 220 or 170 to 160 for example) had high levels of luciferase activity. Thus the decrease in luciferase activity in the 190 to 180 and 210 to 200 constructs is due to changes in the nucleotide sequence and not the result of the nucleotide additions. Transfection of these DNA into HepG2 cells revealed two regions important for transcription. Mutating sequences between nucleotides −210 and ˜−200 or between nucleotides −190 and −180 drastically reduced luciferase activity (FIG. 106).

In both humans and mice, FATP5 is only expressed in the liver. To determine the promoter elements mediating liver specific transcription, we isolated a BAC encoding the mouse FATP5 genomic locus and sequenced 10 kb upstream of the transcriptional start. Since this 10 kb of genomic DNA did not contain either a TATA box or GC rich regions found in TATA-less promoters, FATP5 may utilize non-canonical sequences for transcription initiation. Unfortunately, attempts to identify the transcriptional start using primer extension were unsuccessful, perhaps due to secondary structure in the 5′ UTR. Since we did not unambiguously determine the transcriptional start site, the nucleotide numbering in all of the promoter constructs refers to the distance from the translational start codon.

GC Box and Sp1 Transcription Factors

Since another gene was situated approximately 8 kb upstream of the FATP5 initiator methionine, we hypothesized that promoter elements were likely within this region of DNA. A luciferase reporter construct containing this sequence was transcriptionally active in two liver cell lines but was inactive in cell lines derived from lung, muscle, kidney, skin, or fibroblasts. Deletion analysis of the −8 kb reporter construct revealed that the FATP5 promoter was contained within the 261 nucleotides upstream of the initiator methionine. Promoter activity in this −261 base pair piece required the presence of a single GC box. Gel shift assays with oligonucleotides containing this GC box revealed the presence of three distinct complexes that required a functional GC box for binding. GC boxes bind the Sp1 family of transcription factors and the multiple complexes could reflect the binding of different members of the Sp1 protein family or different post-translational modifications of Sp1 in HepG2 cells (Rodenburg et al., 1997). Although the Sp1 family of transcription factors is widely expressed, Sp1 has been shown to be important for the transcription of several liver specific genes and is upregulated in liver after birth (Rodenburg et al., 1997). In some cases, Sp1 will facilitate the binding of a tissue specific transcription factor to DNA. For example, Sp1 binding to DNA enhances the binding of C/EBPβ to an adjacent site in the liver specific CYP2D5 promoter (Lee et al., 1994). Since the C/EBPβ binding site in the CYP2D5 promoter is suboptimal, C/EBPβ binding to this site requires the presence of Sp 1 or nuclear extract. A similar situation could occur in the FATP5 promoter. Although mutations in the 10 nucleotides downstream of the GC box had no effect on luciferase activity, we did not test mutations immediately upstream of the GC box for effects on promoter activity. It is also possible that Sp1 might bind an unknown liver specific transcription factor and recruit it to the FATP5 promoter. Although, there is no experimental evidence for this, Sp1 has recently been shown to bind to a transcriptional activator so additional interacting proteins are possible (Ryu et al., 1999). Other liver specific transcription factors

Alternatively, since the Sp1 gene family is important for the transcription of many genes which are not liver specific, liver specific promoter elements in the FATP5 promoter might be located elsewhere (Boisclair et al., 1993; Rongnoparut et al., 1991; Sorensen and Wintersberger, 1999). Analysis of the sequence downstream of the GC box using TFSearch (http://pdap1.trc.rwcp.orjp/research/db/TFSEARCH.html) did not reveal any additional transcription factor binding sites of relevance (Heinemeyer et al., 1999; Heinemeyer et al., 1998). Further, we were unable to visually identify binding sites for known liver specific transcription factors in this sequence (De Simone and Cortese, 1992; Hanson and Reshef, 1997; Lai, 1992). Thus, we looked experimentally for additional promoter elements by mutating the sequence downstream of the GC box and identified two additional sites downstream of the GC box that were essential for FATP5 transcription. The sequences of these sites do not conform to any known transcription factor binding sites suggesting the either novel proteins bind these elements or that these elements bind known proteins in a novel manner. Preliminary gel shift data using oligonucleotides spanning these site suggests that these two elements may comprise a binding site for a single complex. Further additional data suggests that the complex which binds to these two sites interacts with the GC box 30 base pairs upstream. Interestingly, we noted a palindromic sequence equally split between these two sites (FIG. 107). Since many transcription factors bind palindromic DNA elements, it is intriguing to speculate that these two sequences contribute to the binding site for a novel transcription factor. Current investigations are focused on identifying the proteins binding to these novel elements and how this element interacts with the GC box.

Several studies have shown that the FATP gene family is regulated by a variety of substances including LPS, cytokines, insulin, and diet (Frohnert et al., 1999; Hui et al., 1998; Memon et al., 1999). Especially intriguing has been a recent report that FATP1 is upregulated by PPARα ligands in liver cell lines (Martin et al., 1997; Motojima et al., 1998). Since fatty acids may be endogenous activators of PPAR's, transcriptional regulation of FATP1 by PPAR's may represent a physiologic feedback loop (Gottlicher et al., 1992; Grimaldi et al., 1999; Schoonjans et al., 1996). Given that liver also expresses FATP5, it will be interesting to see whether this genes is also regulated by PPARα and the tools developed here should help address this question.

Several factors make the FATP5 promoter amenable to further study. First, liver specific transcription of FATP5 can be recapitulated using immortalized cell lines in vitro. Second, the minimal required promoter element that confers liver specific transcription is very small. Third, transcriptional activity of this promoter is very robust. Thus, further study of the FATP5 promoter may provide additional insight into the mechanisms of liver specific transcription and regulation of the FATP gene family.

### Example 18

Materials and Methods

Polyclonal antibodies were raised against proteins containing the N-terminal domain of mouse FATP2 or the C-terminal domain of mouse FATP5 fused to glutathione-S-transferase (GS). Tissues for immunofluorescence were collected from 8 week old mice and a 2 year old chimpanzee. Tissues were fresh frozen, cut on a cryostat and mounted on slides. Immunofluorescence was performed as previously described (Stahl et al., 1999). Pictures were taken on a Zeiss confocal microscope.

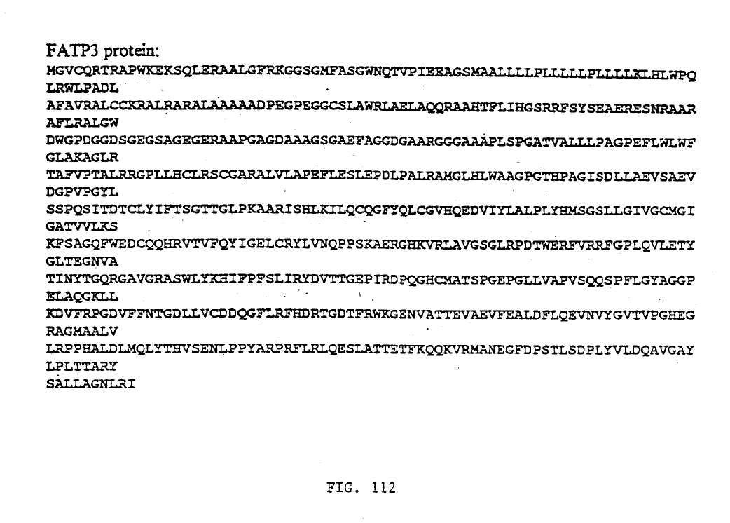

To determine FATP2 expression in the gall bladder, mouse gall bladder was incubated with anti-FATP2 antibody as the primary antibody and rhodamine-labeled anti-rabbit IG as the secondary antibody. FATP2 antibody clearly stained the gall bladder epithelium, but did not result in significant staining of other cell types. (FIG. 108)

To further study FATP2 expression, chimpanzee liver was costained with anti-FATP2 antibody(green) and anti CD31 antibody(red). CD31 is expressed on endothelial cells and is used as a marker for blood vessels. FATP2 immunoreactivity was present in large patches which overlap with CD31 positive areas, suggesting that FATP2 protein was present in the space of Diss, the area where hepatocytes exchange nutrients with the blood. This implicates FATP2 in the uptake of fatty acids into hepatocytes. In addition to areas which overlap with CD31 immunoreactivity, FATP2 protein was also present on the cell surface of hepatocytes in a small bead pattern. Immunoelectronmicroscopy of similar sections showed that FATP2 immunoreactivity was localized in the walls of bile caniculi which are formed by the liver cells. (FIG. 109) The presence of FATP2 in bile caniculi in the liver as well as its presence in the gall bladder epithelium suggests a role for FATP2 in either absorption or secretion of fatty acids into the bile. The levels of free fatty acids in the bile have been associated with the frequency of all stone formation.

To further study FATP5 expression, chimpanzee liver was costained with anti-FATP5 antibody(green) and anti CD31 antibody(red). CD31 is expressed on endothelial cells and is used as a marker for blood vessels. FATP5 immunoreactivity was present in large patches which overlap with CD31 positive areas, suggesting that FATP5 protein was present in the space of Diss, the area where hepatocytes exchange nutrients with the blood. (FIG. 110) This implicates FATP5 in the uptake of fatty acids into hepatocytes.

### Example 19

### Identification and Characterization of Human FATP3 Proteins

Isolation of Additional HumanFATP3 Clones

An additional clone encoding human FATP3 was identified by searching for sequences similar to murine or human FATP3 coding regions using the BlastX algorithm in a proprietary database, (Altschul, et al, J. Mol. Bio. 215: 403-410, 1990). One clone, which was identified by random library sequencing, is described as johni003f04 (SEQ ID NO:116) extends the open reading frame of the hsFATP3 polypeptide sequence by 30 amino acids at the N-terminus when compared to previously discovered sequences. The DNA sequence of this clone is shown in FIGS. 111A and 111B, and the predicted protein sequence (SEQ ID NO: 117) is shown in FIG. 112. The open reading frame of this clone begins at the initial nucleotide and includes nucleotide 2240. The first ATG is located at nucleotide number 51, resulting in a predicted protein which includes 730 amino acids. An FATP signature sequence (see Hirsch et al., PNAS, 95:8625-8629, 1998) is clearly present between amino acids 331 and 640 of hsFATP3. Within this signature sequence hsFATP3 is 48% identical to hsFATP1 at the amino acid level. A consensus AMP-binding motif has been identified (amino acid 333-334). Thus, hsFATP3 is clearly a member of the fatty acid family.

Functional Analysis of FATP3 Clones

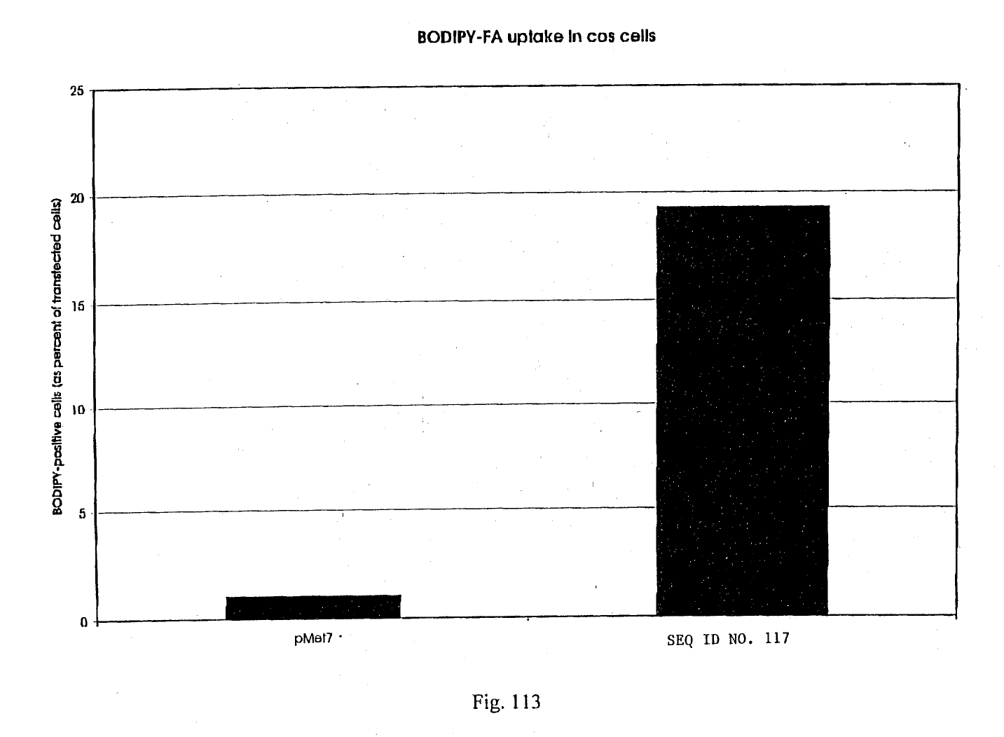

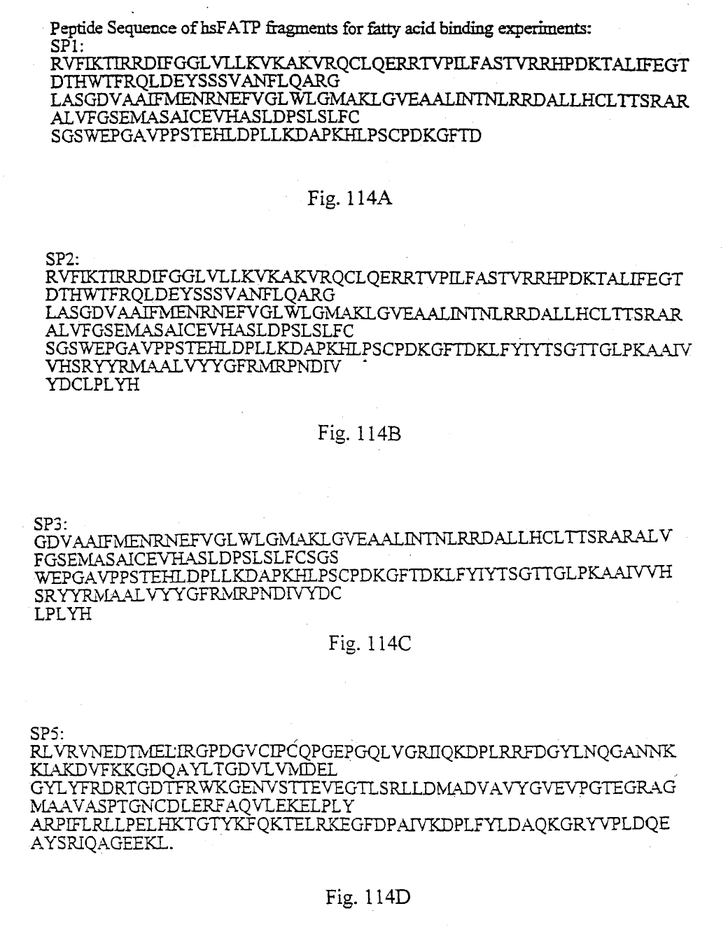

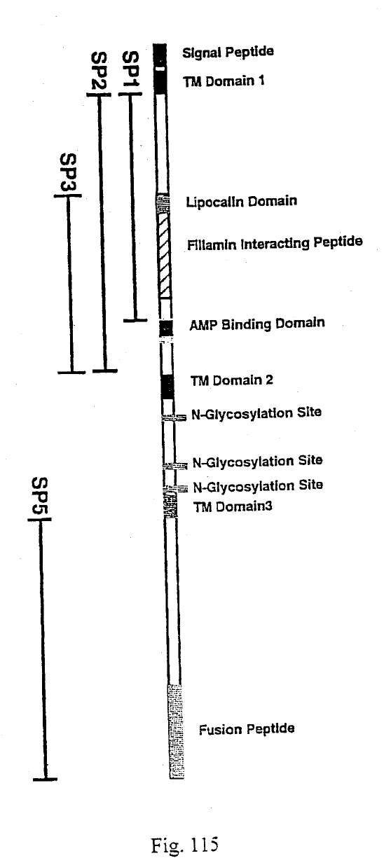

SEQ ID NO: 116 is contained in the mammalian expression vector pMET7 (Tartaglia, et a.., Cell, 83: 1263-1271, 1995). To determine if the protein encoded by this DNA sequence can mediate fatty acid uptake, SEQ ID NO: 116 was transfected into COS cells. Uptake of a BODIPY-labeled fatty acid was determined as described in previous experiments (Hirsch, et al., PNAS, 95: 8625-8629, 1998). Transfection with SEQ ID NO: 116 resulted in a dramatic increase in fatty acid uptake when compared to transfection with vector control. In this experiment, CD31 served as a marker for transfected cells. Only CD31 positive cells were considered for analysis (see Hirsch, et al., PNAS, 95: 8625-8629, 1998 for details). The results (FIG. 113) demonstrate that SEQ ID NO: 116 encodes a functional fatty acid transport protein.

Tissue Distribution of Human FATP3

Polyclonal antibodies were raised by immunizing rabbits with GST fused to the most C-terminal 89 amino acids of mmFATP3-(RPPQALNLVQLYSHVSENLPPYARPRFLRLQESLATTETFKQQKVRMANEGFDP SVLSDPLYVLDQDIGAYLPLTPARYSALLSGDLRI) (SEQ ID NO: 120). Western blotting experiments with murine tissue lysates using the anti-FATP3 antiserum closely confirmed the unique expression pattern of FATP3 as judged by northern blot experiments. This, together with the fact that the serum reacted only weakly with lysates from cell lines expressing either FATP1, -2, -4 or -5, indicates that the antibody recognizes preferentially FATP3, but not other FATP family members.

FATP3 protein was detected in mouse liver, spleen, heart, kidney, testis, white adipose tissue, and most notably in the lung. Further FATP3 expression in the lung was examined by immunofluorescence microscopy. 5 to 10 μM thick fresh frozen unfixed sections of murine and chimpanzee lungs were blocked with 10% FCS/1% donkey serum/1% BSA in HBS and incubated overnight with anti-FATP3 serum in blocking solution. After washing the sections Alexa 488 conjugated donkey anti-rabbit secondary antibodies were used to detect bound anti-FATP3 primary antibodies and nuclei were stained TOTO3. In later experiments, chimpanzee lung was incubated with a mixture of rabbit anti-FATP3 and mouse monoclonal anti-CD31 to visualize FATP3 as well as blood vessels. Sections were imaged on a Zeiss LSM510 confocal microscope. Experiments carried out once with mouse and three times with chimpanzee lung tissue showed that FATP3 is present at high levels in type-II pneumocytes, a cell type responsible for secretion of surfactant, a phospholipid-rich film critical for lung function. The exact function of FAT3 in type II pneumocytes is not yet clear. One hypothesis is that FATP3 is responsible for supplying fatty acid substrates for the symthesis of surfactant.

PCR-based experiments showed that the exocrine as well as endocrine pancreas expresses FATP3. This fact was confirmed by immunofluorescence performed as described above for the lung sections, on chimpanzee pancreas which showed FATP3 localized to the plasma membrane of acinar cells and a punctate expression pattern on the plasma membrane and in the cytosol of alpha and beta cells of the pancreatic islands. The identification of a fatty acid transporter in the insulin producing cells of the pancreas has potentially broad implications for the treatment of type II diabetes and obesity. In both diseases, fatty acid levels in the blood are elevated and, in later stages of the disease, lead to diminished insulin secretion by the pancreas due to the induction of apoptosis in insulin-producing beta cells (Shimabukuro, et al., PNAS, 95: 2498-2502, 1988). Blocking fatty acid uptake into the beta cells could possibly prevent apoptosis and maintain insulin secretion thus preventing the progression from obesity to diabetes.

### Example 20

### Identification of a Fatty Acid Binding Domain in FATP4

GST fusion proteins were constructed in pGEX for four regions of hsFATP4 (SEQ ID NO: 52; FIG. 51) which were generated by PCR and verified by sequencing. The first three fusion proteins were constructed from regions near the N-terminal portion of the protein. SP1 (SEQ ID NO: 121) contained amino acid residues 43-239 of the hsFATP4 sequence as shown in FIG. 114A. This portion of hsFATP4 contains a lipocalin domain (as shown in FIG. 117) as well as a number of residues which in hsFATP4 are upstream of the lipocalin domain. SP2 (SEQ ID NO: 122) contained residues 43-290 of the hsFATP4 sequence as shown in FIG. 114B. This portion of the hsFATP4 contains a lipocalin domain and an AMP binding domain as well as a number of residues which are upstream of the lipocalin domain. SP3 (SEQ ID NO: 123) contained amino acid residues 125-290 of the hsFATP4 sequence as shown in FIG. 114C). This portion of the hsFATP4 contains a lipocalin domain and an AMP binding domain, but does not contain the upstream residues. The fourth fusion protein was constructed from a region at the C-terminal end of the hsFATP4 polypeptide. SP5 contained amino acid residues 417-643 of hsFATP4 polypeptide as show in FIG. 114D (SEQ ID NO: 124).

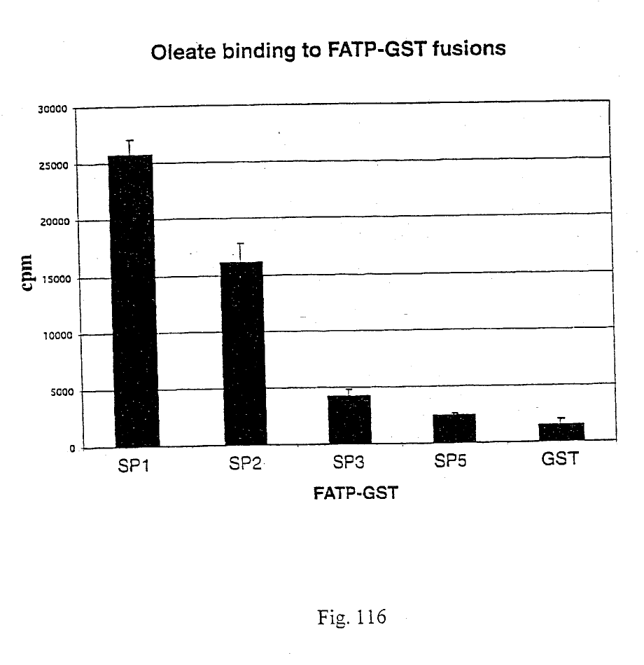

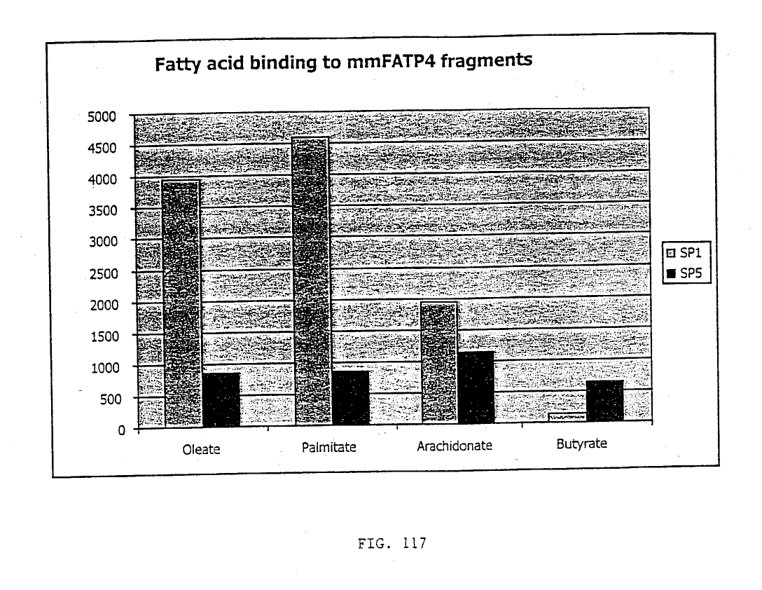

Proteins were expressed in E. coli and purified on glutathione affinity beads using standard techniques. To determine fatty acid binding, beads were mixed with 100 μM 14C-labeled fatty acids in mixed micelles with taurocholate (10 mM, Sigma) and incubated for 30 minutes at room temperature. The beads were subsequently washed with PBS containing 10 mM taurocholate and radioactivity associated with beads was assessed by scintillation counting. A fusion to the C-terminal domain of hsFATP4 (SP5) did not show any oleate (ARC) binding compared to GST protein alone, while 2 N-terminal fusions (SP1 and 2) bound significant amounts of oleate. (FIG. 116).

| FATTY ACID | SP1 | SP2 | SP3 | SP5 | GST |
|---|---|---|---|---|---|
| Oleate | 25772 ± 1326 | 16172 ± 1639 | 4206 ± 631 | 2413 ± 186 | 1511 ± 525 |

Similar results were obtained using maltose-binding protein fusions. MBP fusion constructs were generated by digesting the pGEX-SP constructs with EcoRI/XhoI and ligated into pMAL digested with EcoRI/SaII. MBP fusion proteins were expressed in E. coli and were purified under non-denaturing conditions following the manufacturer's instructions. To determine fatty acid binding, beads were mixed with 100 μM 14C-labeled fatty acids in mixed micelles with taurocholate (10 mM, Sigma) and incubated for 30 minutes at room temperature. The beads were subsequently washed with HBS containing 10 mM taurocholate. The proteins were subsequently eluted from the resin with maltose and the amount of fatty acid binding to MBP-SP1, -2, -3, and -5 was assessed by determining the radioactivity associated with the elute by β-scintillation counting.

Unlike GST fusion proteins, MBP fusion proteins are not self-dimerizing. Further, long-chain fatty acids (such as oleate and palmitate), but not short-chain fatty acids (such as butyrate), were specifically bound by SP1 (FIG. 117). This selective binding is consistent with previous reports of the substrate specificity of FATP4 (Stahl, et al., Mol. Cell, 4, 299-308, 1999). The identification of a fatty acid binding domain in FATP4 will be useful in the development of small molecules that inhibit the binding and transport of fatty acids by FATP4 and may provide useful information on the mechanism of fatty acid transport.

Results of Fatty Acid Binding

| | | binding to | |
|---|---|---|---|
| FATTY ACID | Composition | MBP-SP1 | binding to MBP-SP5 |
| Oleate | C18H3402 | 3968 | 2800 |
| Palmitate | C16H3202 | 4588 | 844 |
| Arachidonate | C20H4002 | 1942 | 1147 |
| Butyrate | C4H802 | 142 | 633 |

These experiments demonstrate that the FATPs of the present invention contain domains that bind various long chain fatty acids. Thus, polypeptides containing these domains can be prepared and utilized to assess the modulation of binding and transport function by a variety of agents. The polypeptides with the highest binding capacities were shown to be those containing a lipocalin domain (such as those shown in FIG. 118) with additional upstream residues, such as those associated with this domain in the N-terminal portion of hsFATP4. Polypeptides containing domains in addition to the lipocalin domain (for example, those containing an AMP binding domain) were also shown to bind fatty acids at significant levels.

FIG. 118 contains an alignment depicting the consensus sequences for the six human FATP, hsFATP1, hsFATP2, hsFATP3, hsFATP4, hsFATP5 and hsFATP6 polypeptides. A lipocalin domain and an AMP binding domain for each polypeptide are both identifed and compared. A search using the lipocalin signature sequence [DENG]-X-[DENQGSTARK]-X(0,2)-[DENQARK]-[LIVFY]-{CP}-G-{C}-W-[FYWLRH-X]-[LIVMTA] conducted on a public database (www.ebi.ac.uk/interpro/), indicated that the lipocalin domains of hsFATP1 and hsFATP4 are identical to the lipocalin signature sequence. In addition, a search directed to identifying sequences having at least 80% identity to the lipocalin signature sequence identified three additional human FATPs, hsFATP3, hsFATP5 and hsFATP6.

The following is the result of comparing individual hsFATP protein sequences with the lipocalin domain identified for hsFATP 1 and hsFATP4. The comparison was made using the BLAST Network Service at the National Center for Biotechnology Information. (Capitalized AA agree with the lipocalin signature sequence.)

| | | |
|---|---|---|
| FATP6: | 114 to 125 NEpDFVhVWFGL. 76% similarity | (SEQ ID NO: 138) |
| | AATGAGCCGGACTTCGTTCACGTGTGGTTCGGCCTC | |
| FATP5: | 182 to 194 sQAVpaLcMWLGL. 53% similarity | (SEQ ID NO: 139) |
| | TCCCAGGCCGTTCCAGCCCTGTGTATGTGGCTGGGGCTG | |
| FATP4: | 134 to 146 ENRNEFVGLWLGM. Identity | (SEQ ID NO: 129) |
| | GAGAACCGCAATGAGTTCGTGGGCCTATGGCTGGGCATG | |
| FATP3: | 221 to 234 IPAGPEFLwLWTGL. 69% similarity | (SEQ ID NO: 140) |
| | CTCCCCGCTGGCCCAGAGTTTCTGTGGCTCTGGTTCGGGCTG | |
| FATP2: | 112 to 124 GNEPAYVwLWLGL. 80% similarity | (SEQ ID NO: 127) |
| | GGTAACGAGCCGGCCTACGTGTGGCTGTGGCTGGGGCTG | |
| FATP1: | 136 to 148 EGRPEFVGLWLGL. Identity | (SEQ ID NO: 126) |
| | GAGGGCCGGCCGGAGTTCGTGGGGCTGTGGCTGGGCCTG | |

References

Abumrad, N., Coburn, C., and Ibrahimi, A. (1999). Membrane proteins implicated in long-chain fatty acid uptake by mammalian cells: CD36, FATP and FABPm. Biochim Biophys Acta 1441, 4-13.

Berk, P. D., Bradbury, M., Zhou, S. L., Stump, D., and Han, N. I. (1996). Characterization of membrane transport processes: lessons from the study of BSP, bilirubin, and fatty acid uptake. Semin Liver Dis 16, 107-20.

Berk, P. D., and Stump, D. D. (1999). Mechanisms of cellular uptake of long chain free fatty acids. Mol Cell Biochem 192, 17-31.

Boisclair, Y. R., Brown, A. L., Casola, S., and Rechler, M. M. (1993). Three clustered Sp1 sites are required for efficient transcription of the TATA-less promoter of the gene for insulin-like growth factor-binding protein-2 from the rat. J Biol Chem 268, 24892-901.

De Simone, V., and Cortese, R. (1992). Transcription factors and liver-specific genes. Biochim Biophys Acta 1132, 119-26.

Fitscher, B. A., Elsing, C., Riedel, H. D., Gorski, J., and Stremmel, W. (1996). Protein-mediated facilitated uptake processes for fatty acids, bilirubin, and other amphipathic compounds [see comments]. Proc Soc Exp Biol Med 212, 15-23.

Frohnert, B. I., Hui, T. Y., and Bernlohr, D. A. (1999). Identification of a functional peroxisome proliferator-responsive element in the murine fatty acid transport protein gene. J Biol Chem 274, 3970-7.

Glatz, J. F., Luiken, J. J., van Nieuwenhoven, F. A., and Van der Vusse, G. J. (1997). Molecular mechanism of cellular uptake and intracellular translocation of fatty acids. Prostaglandins Leukot Essent Fatty Acids 5 7, 3-9.

Gottlicher, M., Widmark, E., Li, Q., and Gustafsson, J. A. (1992). Fatty acids activate a chimera of the clofibric acid-activated receptor and the glucocorticoid receptor. Proc Natl Acad Sci U S A 89, 4653-7.

Grimaldi, P. A., Teboul, L., Gaillard, D., Armengod, A. V., and Amri, E. Z. (1999). Long chain fatty acids as modulators of gene transcription in preadipose cells. Mol Cell Biochem 192, 63-8.

Hamilton, J. A. (1998). Fatty acid transport: difficult or easy? J Lipid Res 39, 467-81.

Hanson, R. W., and Reshef, L. (1997). Regulation of phosphoenolpyruvate carboxykinase (GTP) gene expression. Annu Rev Biochem 66, 581-611.

Heinemeyer, T., Chen, X., Karas, H., Kel, A. E., Kel, O. V., Liebich, I., Meinhardt, T., Reuter, I., Schacherer, F., and Wingender, E. (1999). Expanding the TRANSFAC database towards an expert system of regulatory molecular mechanisms. Nucleic Acids Res 27, 318-22.

Heinemeyer, T., Wingender, E., Reuter, I., Hermjakob, H., Kel, A. E., Kel, O. V., Ignatieva, E. V., Ananko, E. A., Podkolodnaya, O. A., Kolpakov, F. A., Podkolodny, N. L., and Kolchanov, N. A. (1998). Databases on transcriptional regulation: TRANSFAC, TRRD and COMPEL. Nucleic Acids Res 26, 362-7.

Hirsch, D., Stahl, A., and Lodish, H. F. (1998). A family of fatty acid transporters conserved from mycobacterium to man. Proc Natl Acad Sci U S A 95, 8625-9.

Hua, X., Liu, X., Ansari, D. O., and Lodish, H. F. (1998). Synergistic cooperation of TFE3 and smad proteins in TGF-beta-induced transcription of the plasminogen activator inhibitor-1 gene. Genes Dev 12, 3084-95.

Hui, T. Y., Frohnert, B. I., Smith, A. J., Schaffer, J. E., and Bemlohr, D. A. (1998). Characterization of the murine fatty acid transport protein gene and its insulin response sequence. J Biol Chem 273, 27420-9.

Lai, E. (1992). Regulation of hepatic gene expression and development. Semin Liver Dis 12, 246-51.

Lee, Y. H., Yano, M., Liu, S. Y., Matsunaga, E., Johnson, P. F., and Gonzalez, F. J. (1994). A novel cis-acting element controlling the rat CYP2D5 gene and requiring cooperativity between C/EBP beta and an Sp1 factor. Mol Cell Biol 14, 1383-94.

Martin, G., Schoonjans, K., Lefebvre, A. M., Staels, B., and Auwerx, J. (1997). Coordinate regulation of the expression of the fatty acid transport protein and acyl-CoA synthetase genes by PPARalpha and PPARgamma activators. J Biol Chem 272, 28210-7.

Memon, R. A., Fuller, J., Moser, A. H., Smith, P. J., Grunfeld, C., and Feingold, K. R. (1999). Regulation of putative fatty acid transporters and Acyl-CoA synthetase in liver and adipose tissue in ob/ob mice. Diabetes 48, 121-7.

Motojima, K., Passilly, P., Peters, J. M., Gonzalez, F. J., and Latruffe, N. (1998). Expression of putative fatty acid transporter genes are regulated by peroxisome proliferator-activated receptor alpha and gamma activators in a tissue- and inducer-specific manner. J Biol Chem 273, 16710-4.

Rodenburg, R. J., Holthuizen, P. E., and Sussenbach, J. S. (1997). A functional Sp1 binding site is essential for the activity of the adult liver-specific human insulin-like growth factor II promoter. Mol Endocrinol 11, 237-50.

Rongnoparut, P., Verdon, C. P., Gehnrich, S. C., and Sul, H. S. (1991). Isolation and characterization of the transcriptionally regulated mouse liver (B-type) phosphofructokinase gene and its promoter. J Biol Chem 266, 8086-91.

Ryu, S., Zhou, S., Ladumer, A. G., and Tjian, R. (1999). The transcriptional cofactor complex CRSP is required for activity of the enhancer-binding protein Sp1. Nature 397, 446-50.

Schaffer, J., and Lodish, H. F. (1995). Molecular mechanisms of long-chain fatty acid uptake. Trends in Cardiovascular Medicine 5, 218-224.

Schaffer, J. E., and Lodish, H. F. (1994). Expression cloning and characterization of a novel adipocyte long chain fatty acid transport protein [see comments]. Cell 79, 427-36.

Schoonjans, K., Staels, B., and Auwerx, J. (1996). The peroxisome proliferator activated receptors (PPARS) and their effects on lipid metabolism and adipocyte differentiation. Biochim Biophys Acta 1302, 93-109.

Sorensen, P., and Wintersberger, E. (1999). Sp1 and NF-Y are necessary and sufficient for growth-dependent regulation of the hamster thymidine kinase promoter [In Process Citation]. J Biol Chem 274, 30943-9.

Stahl, A., Hirsch, D. J., Gimeno, R. E., Punreddy, S., Ge, P., Watson, N., Patel, S., Kotler, M., Raimondi, A., Tartaglia, L. A., and Lodish, H. F. (1999). Identification of the major intestinal fatty acid transport protein [In Process Citation]. Mol Cell 4, 299-308.

Stremmel, W. (1989). Mechanism of hepatic fatty acid uptake. Journal of Hepatology 9, 374-382.

All references cited herein are incorporated by reference in their entirety.

While this invention has been particularly shown and described with references to preferred embodiments thereof, it will be understood by those skilled in the art that various changes in form and details may be made therein without departing from the spirit and scope of the invention as defined by the appended claims.

What is claimed is:

1. An isolated nucleic acid comprising the nucleotide sequence of SEQ ID NO.:116 or its complement.

2. An isolated nucleic acid comprising the coding sequence of SEQ ID NO.: 116.

3. An isolated nucleic acid which encodes a polypeptide comprising the amino acid sequence of SEQ ID NO.:117 or its complement.

4. An isolated nucleic acid which hybridizes under stringency conditions of 6× SSC at 65° C., followed by at least one wash in 0.2× SSC/0.5% SDS at 65° C., to the nucleic acid comprising the nucleotide sequence of SEQ ID NO.: 116.

5. An isolated nucleic acid consisting of a nucleotide sequence having at least 95% identity to a nucleotide sequence of claim 1.

6. An isolated nucleic acid consisting of a nucleotide sequence having at least 90% identity to a nucleotide sequence of claim 1.

7. An isolated nucleic acid encoding a fusion polypeptide, wherein the isolated nucleic acid comprises a nucleotide sequence of SEQ ID NO.:116.

8. A vector comprising a nucleic acid of claim 1.

9. A vector comprising a nucleic acid of claim 2.

10. A vector comprising a nucleic acid of claim 3.

11. A vector comprising a nucleic acid of claim 4.

12. A vector comprising a nucleic acid of claim 5.

13. A vector comprising a nucleic acid of claim 6.

14. A vector comprising a nucleic acid of claim 7.

15. An isolated host cell transfected with the vector of claim 8.

16. An isolated host cell transfected with the vector of claim 9.

17. An isolated host cell transfected with the vector of claim 10.

18. An isolated host cell transfected with the vector of claim 11.

19. An isolated host cell transfected with the vector of claim 12.

20. An isolated host cell transfected with the vector of claim 13.

21. An isolated host cell transfected with the vector of claim 14.

22. A method of producing a polypeptide comprising the step of culturing the host cell of claim 15 under conditions in which the nucleic acid is expressed, thereby producing the polypeptide.

23. A method of producing a polypeptide comprising the step of culturing the host cell of claim 16 under conditions in which the nucleic acid is expressed, thereby producing the polypeptide.

24. A method of producing a polypeptide comprising the step of culturing the host cell of claim 17 under conditions in which the nucleic acid is expressed, thereby producing the polypeptide.

25. A method of producing a polypeptide comprising the step of culturing the host cell of claim 18 under conditions in which the nucleic acid is expressed, thereby producing the polypeptide.

26. A method of producing a polypeptide comprising the step of culturing the host cell of claim 19 under conditions in which the nucleic acid is expressed, thereby producing the polypeptide.

27. A method of producing a polypeptide comprising the step of culturing the host cell of claim 20 under conditions in which the nucleic acid is expressed, thereby producing the polypeptide.

28. A method of producing a polypeptide comprising the step of culturing the host cell of claim 21 under conditions in which the nucleic acid is expressed, thereby producing the polypeptide.

29. An isolated nucleic acid comprising at least 30 contiguous nucleotides of the nucleotide sequence of SEQ ID NO.:116.

30. An isolated nucleic acid comprising at least 200 contiguous nucleotides of the nucleotide sequence of SEQ ID NO. :116.

31. An isolated polypeptide comprising the amino acid sequence of SEQ ID NO. :117.

32. An isolated naturally occurring allelic variant of a polypeptide consisting of the amino acid sequence of claim 31.

33. An isolated polypeptide consisting of an amino acid sequence having at least 95% identity to the amino acid sequence of claim 31.

34. An isolated polypeptide consisting of an amino acid sequence having at least 90% identity to the amino acid sequence of claim 31.

35. An isolated polypeptide encoded by a nucleic acid that hybridizes to a nucleic acid consisting of the nucleotide sequence of SEQ ID NO.:117 under stringency conditions of 6× SSC at 65° C., followed by at least two washes in 0.2× SSC/0.5% SDS at 65° C.

36. A fusion protein comprising a polypeptide consisting of the amino acid sequence of SEQ ID NO.:117.

37. The fusion protein of claim 36, wherein the fusion protein transports fatty acids across a cell membrane or an artificial cell membrane system.

38. An isolated polypeptide comprising at least 15 contiguous amino acid residues of SEQID NO.:117.

39. An isolated polypeptide comprising at least 50 contiguous amino acid residues of SEQ ID NO.:117.

40. An isolated polypeptide comprising at least 360 contiguous amino acid residues of SEQ ID NO.:117.

41. An isolated polypeptide comprising an amino acid sequence having at least 15 contiguous amino acid residues of SEQ ID NO.:117, wherein the isolated polypeptide transports fatty acids across a cell membrane or an artificial cell membrane.

42. An isolated polypeptide encoded by a nucleic acid that hybridizes to a nucleic acid consisting of the nucleotide sequence of SEQ ID NO.:116 under stringency conditions of 6× SSC at 65° C., followed by at least two washes in 0.2× SSC/0.5% SDS at 65° C.

43. A method for identifying an agent which binds to a protein comprising an amino acid sequence of SEQ ID NO.:117 comprising the steps of contacting the agent with the isolated protein under conditions appropriate for binding of the agent to the isolated protein, and detecting a resulting agent-protein complex.

44. An agent identified by the method of claim 43.

45. A method for identifying an agent which is an inhibitor of fatty acid uptake by a protein encoded by a polynucleotide comprising a nucleotide sequence which encodes a protein consisting of the amino acid sequence of SEQ ID NO.:1 17, comprising the steps of:
a) maintaining test cells expressing said polynucleotide in the presence of a fatty acid and an agent to be tested as an inhibitor of fatty acid, uptake;
b) measuring uptake of the fatty acid in the test cells; and
c) comparing uptake of the fatty acid in the test cells with uptake of the fatty acid in suitable control cells;
wherein lower uptake of the fatty acid in the test cells compared to uptake of the fatty acid in the control cells is indicative that the agent is an inhibitor of fatty acid uptake by said protein.

46. An inhibitor of fatty acid uptake identified by the method of claim 45.

47. The method of claim 45 further comprising the steps of:
a) administering the agent to one or more test animals;
b) measuring exogenously supplied fatty acids in one or more samples of tissue or bodily fluid from said test animals;
c) measuring exogenously supplied fatty acids in one or more comparable samples of tissue or bodily fluid from suitable control animals;
d) comparing the fatty acids of b) with the fatty acids of c);
whereby, lower fatty acids in step b) than in step c) is indicative that the agent is an inhibitor of said protein.

48. An inhibitor of fatty acid uptake identified by the method of claim 47.

49. The method of claim 45, wherein the nucleotide sequence which encodes a protein consists of a nucleotide sequence with 95% identity to a nucleotide sequence which encodes the polypeptide with SEQ ID NO.: 117.

50. A method for identifying an agent which is an inhibitor of a protein encoded by a polynucleotide comprising a nucleotide sequence which encodes a protein comprising the amino acid sequence in SEQ ID NO.: 117 comprising the steps of:
(a) introducing into host cells one or more vectors comprising a polynucleotide expressing said protein;
(b) culturing a first aliquot of the host cells with fatty acid substrate of said protein and with an agent being tested as an inhibitor of said protein;
(c) culturing a second aliquot of the host cells with fatty acid substrate of said protein;
(d) measuring, in the first and second aliquots, uptake of the fatty acid substrate of the host cells;
wherein less uptake of the fatty acid substrate in the first aliquot compared to the second aliquot is indicative that the agent is an inhibitor of said protein.

51. An inhibitor of fatty acid uptake identified by the method of claim 52.

52. The method of claim 50 further comprising the steps of:
a) administering the agent to one or more test animals;
b) measuring exogenously supplied fatty acids in one or more samples of tissue or bodily fluid from suitable control animals;
c) measuring exogenously supplied fatty acids in one or more comparable samples of tissue or bodily fluid from suitable control animals; and
d) comparing the fatty acids of b) with the fatty acids of c),
whereby, lower fatty acids in step b) than in step c) is indicative that the agent is an inhibitor of said protein.

53. A method for identifying an agent which binds to a protein comprising an amino acid sequence of SEQ ID NO.:1 17 comprising the steps of contacting the agent with the isolated protein under conditions appropriate for binding of the agent to the isolated protein, and detecting a resulting agent-protein complex.

54. A method for identifying an agent which inhibits interaction between an isolated protein comprising an amino acid sequence of SEQ ID NO.:117, and further comprising a ligand of said protein, comprising:
(a) combining:
(1) said isolated protein;
(2) the ligand of said protein; and
(3) a candidate agent to be assessed for its ability to inhibit interaction between said protein of (1) and the ligand of (2), under conditions appropriate for interaction between the said protein of (1) and the ligand of (2);
(b) determining the extent to which said protein of (1) and the ligand of (2) interact; and
(c) comparing the extent determined in (b) with the extent to which interaction of said protein of (1) and the ligand of (2) occurs in the absence of the candidate agent to be assessed and under the same conditions appropriate for interaction of said protein of (1) with the ligand of (2);
wherein if the extent to which interaction of said protein of (1) and the ligand of (2) occurs is less in the presence of the candidate agent than in the absence of the candidate agent, the candidate agent is an agent which inhibits interaction between said protein and the ligand of said protein.

55. A method for detecting, in a sample of cells, a nucleic acid molecule consisting of a nucleotide sequence with at least 90% sequence identity to SEQ ID NO.: 116, comprising:
a) purifying nucleic acid from the cells;
b) hybridizing 1) purified nucleic acid from the cells to 2) purified nucleic acid comprising SEQ ID NO.:116, under conditions that allow hybridization between 1) and 2) if the sequences of 1) and 2) have at least 90% sequence identity; and
c) detecting resulting hybrid nucleic acids in the hybridization; wherein, if hybrid nucleic acids are detected at a significant level compared to a suitable control hybridization, then a nucleic acid molecule comprising at least 90% sequence identity to SEQ ID NO: 116, has been detected.

56. A method for identifying (1) nucleic acid molecules in fixed cells which specifically interact with a (2) nucleic acid molecule comprising the nucleotide sequence in SEQ ID NO.:116, said method comprising the steps of:
a) adding to the fixed cells the nucleic acid molecule comprising a nucleotide sequence in SEQ ID NO.:116;
b) incubating the fixed cells under conditions allowing hybridization of (1) with (2);
c) removing the nucleic acid molecule of step a) that has not hybridized; and
d) detecting hybrid molecules comprising (1) and (2).

57. A method for detecting FATP3 in a sample of cells, comprising the steps of adding an agent that specifically binds to FATP3 to the sample, and detecting agent specifically bound to the FATP3.

58. The method of claim 57 wherein the agent is an antibody which binds to FATP3.

59. A method for detecting FATP3 in a sample of cell lysate, comprising the steps of adding an agent that specifically binds to FATP3 or FATP4 to the sample, and detecting agent specifically bound to the FATP3 or FATP4.

60. The method of claim 59 wherein the agent is an antibody which binds to FATP3.

61. An isolated antibody which binds to a polypeptide having an amino acid sequence consisting of at least 95% amino acid sequence identity with the amino acid sequence of SEQ ID NO.:117.

62. An isolated antibody which binds to a fatty acid transport protein having the amino acid sequence of SEQ ID NO.:117.

63. A method for detecting, in a sample of cells, a nucleic acid molecule comprising at least 90% sequence identity to SEQ ID NO.:116 comprising:
a) purifying nucleic acid from the cells;
b) hybridizing 1) purified nucleic acid from the cells to 2) purified nucleic acid comprising SEQ ID NO.:116 or SEQ ID NO.:52, under conditions that allow hybridization between 1) and 2) if the sequences of 1) and 2) have at least 90% sequence identity; and
c) detecting resulting hybrid nucleic acids in the hybridization; wherein, if hybrid nucleic acids are detected at a significant level compared to a suitable control hybridization, then a nucleic acid molecule having at least 90% sequence identity to SEQ ID NO.:116 or SEQ ID NO.:52, has been detected.

64. A method for detecting, in a sample of purified nucleic acid, a nucleic acid molecule comprising at least 90% sequence identity to SEQ ID NO.: 116 comprising:
a) hybridizing 1) the sample of purified nucleic acid to 2) purified nucleic acid comprising SEQ ID NO.:116 or SEQ ID NO.:52, under conditions that allow hybridization between 1) and 2) if the sequences of 1) and 2) have at least 90% sequence identity; and
b) detecting resulting hybrid nucleic acids in the hybridization; wherein, if hybrid nucleic acids are detected at a significant level compared to a suitable control hybridization, then a nucleic acid molecule having at least 90% sequence identity to SEQ ID NO.:116 or SEQ ID NO.:52, has been detected.

65. A method for detecting FATP3 in a sample of cells, comprising the steps of adding an agent that specifically binds to FATP3 to the sample, and detecting agent specifically bound to the FATP3.

66. The method of claim 65 wherein the agent is an antibody which binds to FATP3.

67. A vector comprising a FATP regulatory sequence and at least one targeting sequence directed to the regulatory region of a nucleic acid with a nucleotide sequence selected from the group consisting of:
a) SEQ ID NO.:46
b) SEQ ID NO.:48
c) SEQ ID NO.:116
d) SEQ ID NO.:52
e) SEQ ID NO.:54 and
f) SEQ ID NO.:56

68. An isolated host cell transfected with a vector of claim 67.

69. A method of producing a polypeptide comprising culturing the host cell of claim 68 under conditions in which the nucleic acid is expressed, thereby producing the polypeptide.

70. An isolated nucleic acid comprising a nucleotide sequence encoding a functional portion of a FATP polypeptide comprising a lipocalin domain.

71. The isolated nucleic acid of claim 70 further comprising a nucleotide sequence encoding upstream amino acid residues.

72. An isolated nucleic acid comprising a nucleotide sequence encoding a portion of a FATP protein containing a lipocalin domain, wherein the nucleotide sequence is selected from the group consisting of portions of:
a) SEQ ID NO.:46
b) SEQ ID NO.:48
c) SEQ ID NO.:116
d) SEQ ID NO.:52
e) SEQ ID NO.:54 and
f) SEQ ID NO.:56.

73. An isolated nucleic acid of claim 72 further comprising at least about 90 nucleotides of the sequence upstream of the lipocalin domain.

74. A vector comprising a nucleic acid of claim 73.

75. An isolated host cell comprising the vector of claim 74.

76. A method of producing a polypeptide comprising the step of culturing the host cell of claim 75 under conditions in which the nucleic acid is expressed, thereby producing the polypeptide.

77. A functional portion of a FATP polypeptide comprising a lipocalin domain.

78. The FATP polypeptide of claim 77 further comprising upstream amino acid residues.

79. An isolated polypeptide comprising an amino acid sequence containing a FATP lipocalin domain, wherein the amino acid sequence is selected from the group consisting of portions of:
a) SEQ ID NO.:47;
b) SEQ ID NO.:49;
c) SEQ ID NO.:117;
d) SEQ ID NO.:53;
e) SEQ ID NO.:55; and
f) SEQ ID NO.:57.

80. A functional portion of a FATP polypeptide comprising an amino acid sequence selected from the group consisting of:
a) SEQ ID NO.:126;
b) SEQ ID NO.:127;
c) SEQ ID NO.:128;
d) SEQ ID NO.:129;
e) SEQ ID NO.:130; and
f) SEQ ID NO.:131.

81. A fusion protein comprising a polypeptide consisting of a FATP polypeptide containing a lipocalin domain.

82. The fusion protein of claim 81 further comprising upstream sequences.

83. The fusion protein of claim 82, wherein the upstream sequences comprise at least about 30 amino acid residues of an upstream sequence.

84. A fusion protein comprising a polypeptide consisting of a FATP polypeptide containing a lipocalin domain, wherein the polypeptide consists of an amino acid sequence selected from the group consisting of portions of:
a) SEQ ID NO.:47;
b) SEQ ID NO.:49;
c) SEQ ID NO.:117;
d) SEQ ID NO.:53;
e) SEQ ID NO.:55; and
f) SEQ ID NO.:57.

85. The fusion protein of claim 84 further comprising upstream sequences.

86. A method for identifying an agent which binds to a polypeptide, wherein the polypeptide comprises a FATP lipocalin domain, comprising the steps of contacting the agent with the polypeptide under conditions appropriate for binding of the agent to the polypeptide, and detecting a resulting agent-polypeptide complex.

87. The agent identified by the method of claim 86.

88. A method for identifying an agent which binds to a polypeptide, wherein the polypeptide comprises a FATP lipocalin domain and about 30 amino acid residues of an upstream sequence, comprising the steps of contacting the agent with the polypeptide under conditions appropriate for binding of the agent to the polypeptide, and detecting a resulting agent-polypeptide complex.

89. The agent identified by the method of claim 88.

90. A method for identifying an agent which binds to a polypeptide, wherein the polypeptide comprises a FATP lipocalin domain and consists of an amino acid sequence selected from the group consisting of portions of:
a) SEQ ID NO.:47;
b) SEQ ID NO.:49;
c) SEQ ID NO.:117;
d) SEQ ID NO.:53;
e) SEQ ID NO.:55; and
f) SEQ ID NO.:57,
comprising the steps of contacting the agent with the polypeptide under conditions appropriate for binding of the agent to the polypeptide, and detecting a resulting agent-polypeptide complex.

91. An agent identified by the method of claim 90.

92. A method for identifying an agent which binds to a polypeptide, wherein the polypeptide comprises an amino acid sequence selected from the group consisting of:
a) SEQ ID NO.:126;
b) SEQ ID NO.:127;
c) SEQ ID NO.:128;
d) SEQ ID NO.:129;
e) SEQ ID NO.:130; and
f) SEQ ID NO.:131,
comprising the steps of contacting the agent with the polypeptide under conditions appropriate for binding of the agent to the polypeptide, and detecting a resulting agent-polypeptide complex.

93. An agent identified by the method of claim 92.

94. A method for identifying an agent which binds to a polypeptide comprising a FATP lipocalin domain, wherein the polypeptide is encoded by a nucleotide sequence consisting of portions of:
a) SEQ ID NO.:46;
b) SEQ ID NO.:48;
c) SEQ ID NO.:116;
d) SEQ ID NO.:52;
e) SEQ ID NO.:54; and
f) SEQ ID NO.:56.
comprising the steps of contacting the agent with the polypeptide under conditions appropriate for binding of the agent to the polypeptide, and detecting a resulting agent-polypeptide complex.

95. An agent identified by the method of claim 94.

96. A method for identifying an agent which binds to a polypeptide comprising a FATP lipocalin domain and upstream sequences, wherein the polypeptide is encoded by a nucleotide sequence consisting of portions of:
1. SEQ ID NO.:46;
2. SEQ ID NO.:48;
3. SEQ ID NO.:116;
4. SEQ ID NO.:52;
5. SEQ ID NO.:54; and
6. SEQ ID NO.:56.
comprising the steps of contacting the agent with the polypeptide under conditions appropriate for binding of the agent to the polypeptide, and detecting a resulting agent-polypeptide complex.

97. An agent identified by the method of claim 96.
